SEQUENCE LISTING (19 sequences)

SEQ ID NO: 1 - DNA, 2457 bp, Mus musculus
ctattaacttgttcaaaaaagtatcaggagttgtcaaggcagagaagagagtgtttgcaaaaagggaaaagtactttgctgcctctttaagactagggctgggagaaagaagaggagagagaaagaaaggagagaagtttggagcccgaggcttaagcctttccaaaaactaatcacaacaatcgcggcggcccgaggaggagagcgcctgttttttcatcccaattgcacttcgcccgtctcgagctccgcttccccccaactattctccgccagatctccgcgcagggccgtgcacgccgaggcccccgcccgcggcccctgcatcccggcccccgagcgcggcccccacagtcccggccgggccgagggttggcggccgccggcgggccgcgcccgcccagcgcccgcatgtataacatgatggagacggagctgaagccgccgggcccgcagcaagcttcggggggcggcggcggaggaggcaacgccacggcggcggcgaccggcggcaaccagaagaacagcccggaccgcgtcaagaggcccatgaacgccttcatggtatggtcccgggggcagcggcgtaagatggcccaggagaaccccaagatgcacaactcggagatcagcaagcgcctgggcgcggagtggaaacttttgtccgagaccgagaagcggccgttcatcgacgaggccaagcggctgcgcgctctgcacatgaaggagcacccggattataaataccggccgcggcggaaaaccaagacgctcatgaagaaggataagtacacgcttcccggaggcttgctggcccccggcgggaacagcatggcgagcggggttggggtgggcgccggcctgggtgcgggcgtgaaccagcgcatggacagctacgcgcacatgaacggctggagcaacggcagctacagcatgatgcaggagcagctgggctacccgcagcacccgggcctcaacgctcacggcgcggcacagatgcaaccgatgcaccgctacgacgtcagcgccctgcagtacaactccatgaccagctcgcagacctacatgaacggctcgcccacctacagcatgtcctactcgcagcagggcacccccggtatggcgctgggctccatgggctctgtggtcaagtccgaggccagctccagcccccccgtggttacctcttcctcccactccagggcgccctgccaggccggggacctccgggacatgatcagcatgtacctccccggcgccgaggtgccggagcccgctgcgcccagtagactgcacatggcccagcactaccagagcggcccggtgcccggcacggccattaacggcacactgcccctgtcgcacatgtgagggctggactgcgaactggagaaggggagagattttcaaagagatacaagggaattgggaggggtgcaaaaagaggagagtaggaaaaatctgataatgctcaaaaggaaaaaaaatctccgcagcgaaacgacagctgcggaaaaaaaccaccaatcccatccaaattaacgcaaaaaccgtgatgccgactagaaaacttttatgagagatcttgggacttctttttgggggactatttttgtacagagaaaacctgagggcggcggggagggcgggggaatcggaccatgtatagatctggaggaaaaaaactacgcaaaacttttttttaaagttctagtggtacgttaggcgcttcgcagggagttcgcaaaagtctttaccagtaatatttagagctagactccgggcgatgaaaaaaaagttttaatatttgcaagcaacttttgtacagtatttatcgagataaacatggcaatcaaatgtccattgtttataagctgagaatttgccaatatttttcgaggaaagggttcttgctgggttttgattctgcagcttaaatttaggaccgttacaaacaaggaaggagtttattcggatttgaacattttagttttaaaattgtacaaaaggaaaacatgagagcaagtactggcaagaccgttttcgtggtcttgtttaaggcaaacgttctagattgtactaaatttttaacttactgttaaaggcaaaaaaaaaatgtccatgcaggttgatatcgttggtaatttataatagcttttgttcaatcctaccctttcattttgttcacataaaaaatatggaattactgtgtttgaaatattttcttatggtttgtaatatttctgtaaattgtgatattttaaggtttttccccccttttattttccgtagttgtattttaaaagattcggctctgttattggaatcaggctgccgagaatccatgtatatatttgaactaataccatccttataacagctacattttcaacttaagtttttactccattatgcacagtttgagataaataaatttttgaaatatggacactgaaa

SEQ ID NO: 2 - DNA, 2518 bp, Homo sapiens
ctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaaaaaaaaaa

SEQ ID NO: 3 - protein, 503 aa, Homo sapiens
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYICHNRTVIHHRVPNEEDPSLDRPFISEGTTLKDLIYDMTTSGSGSGLPLLVQRTIARTIVLQESIGKGRFGEVWRGKWRGEEVAVKIFSSREERSWFREAEIYQTVMLRHENILGFIAADNKDNGTWTQLWLVSDYHEHGSLFDYLNRYTVTVEGMIKLALSTASGLAHLHMEIVGTQGKPAIAHRDLKSKNILVKKNGTCCIADLGLAVRHDSATDTIDIAPNHRVGTKRYMAPEVLDDSINMKHFESFKRADIYAMGLVFWEIARRCSIGGIHEDYQLPYYDLVPSDPSVEEMRKVVCEQKLRPNIPNRWQSCEALRVMAKIMRECWYANGAARLTALRIKKTLSQLSQQEGIKM

SEQ ID NO: 4 - protein, 503 aa, Homo sapiens; identical to SEQ ID NO: 3

SEQ ID NO: 5 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 6 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 7 - DNA, 4145 bp, Homo sapiens
caaacaagtgcggccatttcaccagcccaggctggcttctgctgttgactggctgtggcacctcaagcagcccctttcccctctagcctcagtttatcaccgcaagagctaccattcatctagcacaacctgaccatcctcacactggtcagttccaaccttcccaggaatcttctgtggccatgttcactccggttttacagaacagagaacagaagctcagagaagtgaagcaacttgcccagctatgagagacagagccaggatttgaaaccagatgaggacgctgaggcccagagagggaaagccacttgcctagggacacacagcggggagaggtggagcagggcctctatttcgagacccctgactccacacctggtgtttgtgccaagaccccaggctgcctcccaggtcctctgggacagcccctgccttctaccaggaccatgggtagcaacaagagcaagcccaaggatgccagccagcggcgccgcagcctggagcccgccgagaacgtgcacggcgctggcgggggcgctttccccgcctcgcagacccccagcaagccagcctcggccgacggccaccgcggccccagcgcggccttcgcccccgcggccgccgagcccaagctgttcggaggcttcaactcctcggacaccgtcacctccccgcagagggcgggcccgctggccggtggagtgaccacctttgtggccctctatgactatgagtctaggacggagacagacctgtccttcaagaaaggcgagcggctccagattgtcaacaacacagagggagactggtggctggcccactcgctcagcacaggacagacaggctacatccccagcaactacgtggcgccctccgactccatccaggctgaggagtggtattttggcaagatcaccagacgggagtcagagcggttactgctcaatgcagagaacccgagagggaccttcctcgtgcgagaaagtgagaccacgaaaggtgcctactgcctctcagtgtctgacttcgacaacgccaagggcctcaacgtgaagcactacaagatccgcaagctggacagcggcggcttctacatcacctcccgcacccagttcaacagcctgcagcagctggtggcctactactccaaacacgccgatggcctgtgccaccgcctcaccaccgtgtgccccacgtccaagccgcagactcagggcctggccaaggatgcctgggagatccctcgggagtcgctgcggctggaggtcaagctgggccagggctgctttggcgaggtgtggatggggacctggaacggtaccaccagggtggccatcaaaaccctgaagcctggcacgatgtctccagaggccttcctgcaggaggcccaggtcatgaagaagctgaggcatgagaagctggtgcagttgtatgctgtggtttcagaggagcccatttacatcgtcacggagtacatgagcaaggggagtttgctggactttctcaagggggagacaggcaagtacctgcggctgcctcagctggtggacatggctgctcagatcgcctcaggcatggcgtacgtggagcggatgaactacgtccaccgggaccttcgtgcagccaacatcctggtgggagagaacctggtgtgcaaagtggccgactttgggctggctcggctcattgaagacaatgagtacacggcgcggcaaggtgccaaattccccatcaagtggacggctccagaagctgccctctatggccgcttcaccatcaagtcggacgtgtggtccttcgggatcctgctgactgagctcaccacaaagggacgggtgccctaccctgggatggtgaaccgcgaggtgctggaccaggtggagcggggctaccggatgccctgcccgccggagtgtcccgagtccctgcacgacctcatgtgccagtgctggcggaaggagcctgaggagcggcccaccttcgagtacctgcaggccttcctggaggactacttcacgtccaccgagccccagtaccagcccggggagaacctctaggcacaggcgggcccagaccggcttctcggcttggatcctgggctgggtggcccctgtctcggggcttgccccactctgcctgcctgctgttggtcctctctctgtggggctgaattgccaggggcgaggcccttcctctttggtggcatggaaggggcttctggacctagggtggcctgagagggcggtgggtatgcgagaccagcacggtgactctgtccagctcccgctgtggccgcacgcctctccctgcactccctcctggagctctgtgggtctctggaagaggaaccaggagaagggctggggccggggctgagggtgcccttttccagcctcagcctactccgctcactgaactccttccccacttctgtgccacccccggtctatgtcgagagctggccaaagagcctttccaaagaggagcgatgggcccctggccccgcctgcctgccaccctgccccttgccatccattctggaaacacctgtaggcagaggctgccgagacagaccctctgccgctgcttccaggctgggcagcacaaggccttgcctggcctgatgatggtgggtgggtgggatgagtaccccctcaaaccctgccctccttagacctgagggacccttcgagatcatcacttccttgcccccatttcacccatggggagacagttgagagcggggatgtgacatgcccaaggccacggagcagttcagagtggaggcgggcttggaacccggtgctccctctgtcatcctcaggaaccaacaattcgtcggaggcatcatggaaagactgggacagcccaggaaacaaggggtctgaggatgcattcgagatggcagattcccactgccgctgcccgctcagcccagctgttgggaacagcatggaggcagatgtggggctgagctggggaatcagggtaaaaggtgcaggtgtggagagagaggcttcaatcggcttgtgggtgatgtttgaccttcagagccagccggctatgaaagggagcgagcccctcggctctggaggcaatcaagcagacatagaagagccaagagtccaggaggccctggtcctggcctccttccccgtactttgtcccgtggcatttcaattcctggccctgttctcctccccaagtcggcaccctttaactcatgaggagggaaaagagtgcctaagcgggggtgaaagaggacgtgttacccactgccatgcaccaggactggctgtgtaaccttgggtggcccctgctgtctctctgggctgcagagtctgccccacatgtggccatggcctctgcaactgctcagctctggtccaggccctgtggcaggacacacatggtgagcctagccctgggacatcaggagactgggctctggctctgttcggcctttgggtgtgtggtggattctccctgggcctcagtgtgcccatctgtaaaggggcagctgacagtttgtggcatcttgccaagggtccctgtgtgtgtgtatgtgtgtgcatgtgtgcgtgtctccatgtgcgtccatatttaacatgtaaaaatgtcccccccgctccgtcccccaaacatgttgtacatttcaccatggccccctcatcatagcaataacattcccactgccaggggttcttgagccagccaggccctgccagtggggaaggaggccaagcagtgcctgcctatgaaatttcaacttttcctttcatacgtctttattacccaagtcttctcccgtccattccagtcaaatctgggctcactcaccccagcgagctctcaaatccctctccaactgcctaaggccctttgtgtaaggtgtcttaatactgtcctttttttttttttaacagtgttttgtagatttcagatgactatgcagaggcctgggggacccctggctctgggccgggcctggggctccgaaattccaaggcccagacttgcggggggtgggggggtatccagaattggttgtaaatactttgcatattgtctgattaaacacaaacagacctcagaaaaaaaaaaaaaaaaaaaaaaaaaaaaaaaaaaaaaaaaaaaaaaaa

SEQ ID NO: 8 - DNA, 3057 bp, Mus musculus
agttccccggccaagagagcgagcgcggctccgggcgcgcggggagcagaggcggtggcgggcggcggcggcacccggagccgccgagtgcccctccccgcccctccagccccccacccagcaacccgcccgtgacccgcgcccatggccgcgcgcacccggcacagtccccaggactccgcaccccgcgccaccgcccagctcgcagttccgcgccaccgcggccattctcacctggcggcgccgcccgcccaccgcccggaccacagcccccgcgccgccgacagccacagtggccgcgacaacggtgggggacactgctgagtccaagagcgtgcagcctggccatcggacctacttatctgccttgctgattgtctatttttataagagtttacaacttttctaagaatttttgtatacaaaggaacttttttaaagacatcgccggtttatattgaatccaaagaagaaggatctcgggcaatctgggggttttggtttgaggttttgtttctaaagtttttaatcttcgttgactttggggctcaggtacccctctctcttcttcggactccggaggaccttctgggcccccacattaatgaggcagccacctggcgagtctgacatggctgtcagcgacgctctgctcccgtccttctccacgttcgcgtccggcccggcgggaagggagaagacactgcgtccagcaggtgccccgactaaccgttggcgtgaggaactctctcacatgaagcgacttcccccacttcccggccgcccctacgacctggcggcgacggtggccacagacctggagagtggcggagctggtgcagcttgcagcagtaacaacccggccctcctagcccggagggagaccgaggagttcaacgacctcctggacctagactttatcctttccaactcgctaacccaccaggaatcggtggccgccaccgtgaccacctcggcgtcagcttcatcctcgtcttccccggcgagcagcggccctgccagcgcgccctccacctgcagcttcagctatccgatccgggccgggggtgacccgggcgtggctgccagcaacacaggtggagggctcctctacagccgagaatctgcgccacctcccacggcccccttcaacctggcggacatcaatgacgtgagcccctcgggcggcttcgtggctgagctcctgcggccggagttggacccagtatacattccgccacagcagcctcagccgccaggtggcgggctgatgggcaagtttgtgctgaaggcgtctctgaccacccctggcagcgagtacagcagcccttcggtcatcagtgttagcaaaggaagcccagacggcagccaccccgtggtagtggcgccctacagcggtggcccgccgcgcatgtgccccaagattaagcaagaggcggtcccgtcctgcacggtcagccggtccctagaggcccatttgagcgctggaccccagctcagcaacggccaccggcccaacacacacgacttccccctggggcggcagctccccaccaggactacccctacactgagtcccgaggaactgctgaacagcagggactgtcaccctggcctgcctcttcccccaggattccatccccatccggggcccaactaccctcctttcctgccagaccagatgcagtcacaagtcccctctctccattatcaagagctcatgccaccgggttcctgcctgccagaggagcccaagccaaagaggggaagaaggtcgtggccccggaaaagaacagccacccacacttgtgactatgcaggctgtggcaaaacctataccaagagttctcatctcaaggcacacctgcgaactcacacaggcgagaaaccttaccactgtgactgggacggctgtgggtggaaattcgcccgctccgatgaactgaccaggcactaccgcaaacacacagggcaccggccctttcagtgccagaagtgtgacagggccttttccaggtcggaccaccttgccttacacatgaagaggcacttttaaatcccacgtagtggatgtgacccacactgccaggagagagagttcagtatttttttttctaacctttcacactgtcttcccacgaggggaggagcccagctggcaagcgctacaatcatggtcaagttcccagcaagtcagcttgtgaatggataatcaggagaaaggaagagttcaagagacaaaacagaaatactaaaaacaaacaaacaaaaaaacaaacaaaaaaaacaagaaaaaaaaatcacagaacagatggggtctgatactggatggatcttctatcattccaataccaaatccaacttgaacatgcccggacttacaaaatgccaaggggtgactggaagtttgtggatatcagggtatacactaaatcagtgagcttggggggagggaagaccaggattcccttgaattgtgtttcgatgatgcaatacacacgtaaagatcaccttgtatgctctttgccttcttaaaaaaaaaaaaagccattattgtgtcggaggaagaggaagcgattcaggtacagaacatgttctaacagcctaaatgatggtgcttggtgagtcgtggttctaaaggtaccaaacgggggagccaaagttctccaactgctgcatacttttgacaaggaaaatctagttttgtcttccgatctacattgatgacctaagccaggtaaataagcctggtttatttctgtaacatttttatgcagacagtctgttatgcactgtggtttcagatgtgcaataatttgtacaatggtttattcccaagtatgcctttaagcagaacaaatgtgtttttctatatagttccttgccttaataaatatgtaatataaatttaagcaaacttctattttgtatatttgtaaactacaaagtaaaaaaaaatgaacattttgtggagtttgtattttgcatactcaaggtgagaaataagttttaaataaacctataatattttatctgaacgacaaaaaaaaaaaaaaa

SEQ ID NO: 9 - DNA, 2949 bp, Homo sapiens
agtttcccgaccagagagaacgaacgtgtctgcgggcgcgcggggagcagaggcggtggcgggcggcggcggcaccgggagccgccgagtgaccctcccccgcccctctggccccccaccctcccacccgcccgtggcccgcgcccatggccgcgcgcgctccacacaactcaccggagtccgcgccttgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctgaaaaaaaaaaaaaaaaaa

SEQ ID NO: 10 - DNA, 1346 bp, Mus musculus
aaccgtccctaggtgagccgtctttccaccaggcccccggctcggggtgcccaccttccccatggctggacacctggcttcagacttcgccttctcacccccaccaggtgggggtgatgggtcagcagggctggagccgggctgggtggatcctcgaacctggctaagcttccaagggcctccaggtgggcctggaatcggaccaggctcagaggtattggggatctccccatgtccgcccgcatacgagttctgcggagggatggcatactgtggacctcaggttggactgggcctagtcccccaagttggcgtggagactttgcagcctgagggccaggcaggagcacgagtggaaagcaactcagagggaacctcctctgagccctgtgccgaccgccccaatgccgtgaagttggagaaggtggaaccaactcccgaggagtcccaggacatgaaagccctgcagaaggagctagaacagtttgccaagctgctgaagcagaagaggatcaccttggggtacacccaggccgacgtggggctcaccctgggcgttctctttggaaaggtgttcagccagaccaccatctgtcgcttcgaggccttgcagctcagccttaagaacatgtgtaagctgcggcccctgctggagaagtgggtggaggaagccgacaacaatgagaaccttcaggagatatgcaaatcggagaccctggtgcaggcccggaagagaaagcgaactagcattgagaaccgtgtgaggtggagtctggagaccatgtttctgaagtgcccgaagccctccctacagcagatcactcacatcgccaatcagcttgggctagagaaggatgtggttcgagtatggttctgtaaccggcgccagaagggcaaaagatcaagtattgagtattcccaacgagaagagtatgaggctacagggacacctttcccagggggggctgtatcctttcctctgcccccaggtccccactttggcaccccaggctatggaagcccccacttcaccacactctactcagtcccttttcctgagggcgaggcctttccctctgttcccgtcactgctctgggctctcccatgcattcaaactgaggcaccagccctccctggggatgctgtgagccaaggcaagggaggtagacaagagaacctggagctttggggttaaattcttttactgaggagggattaaaagcacaacaggggtggggggtgggatggggaaagaagctcagtgatgctgttgatcaggagcctggcctgtctgtcactcatcattttgttcttaaataaagactgggacacacagtagatagct

SEQ ID NO: 11 - DNA, 1411 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 12 - DNA, 58 bp, Artificial Sequence
  source: /note=“Description of Artificial Sequence Synthetic oligonucleotide”
ccggcctgagctataagcaggttaactcgagttaacctgcttatagctcaggtttttg

SEQ ID NO: 13 - DNA, 58 bp, Artificial Sequence
  source: /note=“Description of Artificial Sequence Synthetic oligonucleotide”
ccggcgagataaacatggcaatcaactcgagttgattgccatgtttatctcgtttttg

SEQ ID NO: 14 - protein, 319 aa, Mus musculus
MYNMMETELKPPGPQQASGGGGGGGNATAAATGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQEQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMAQHYQSGPVPGTAINGTLPLSHM

SEQ ID NO: 15 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 16 - protein, 483 aa, Mus musculus
MRQPPGESDMAVSDALLPSFSTFASGPAGREKTLRPAGAPTNRWREELSHMKRLPPLPGRPYDLAATVATDLESGGAGAACSSNNPALLARRETEEFNDLLDLDFILSNSLTHQESVAATVTTSASASSSSSPASSGPASAPSTCSFSYPIRAGGDPGVAASNTGGGLLYSRESAPPPTAPFNLADINDVSPSGGFVAELLRPELDPVYIPPQQPQPPGGGLMGKFVLKASLTTPGSEYSSPSVISVSKGSPDGSHPVVVAPYSGGPPRMCPKIKQEAVPSCTVSRSLEAHLSAGPQLSNGHRPNTHDFPLGRQLPTRTTPTLSPEELLNSRDCHPGLPLPPGFHPHPGPNYPPFLPDQMQSQVPSLHYQELMPPGSCLPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 17 - protein, 479 aa, Homo sapiens
MRQPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 18 - protein, 352 aa, Mus musculus
MAGHLASDFAFSPPPGGGDGSAGLEPGWVDPRTWLSFQGPPGGPGIGPGSEVLGISPCPPAYEFCGGMAYCGPQVGLGLVPQVGVETLQPEGQAGARVESNSEGTSSEPCADRPNAVKLEKVEPTPEESQDMKALQKELEQFAKLLKQKRITLGYTQADVGLTLGVLFGKVFSQTTICRFEALQLSLKNMCKLRPLLEKWVEEADNNENLQEICKSETLVQARKRKRTSIENRVRWSLETMFLKCPKPSLQQITHIANQLGLEKDVVRVWFCNRRQKGKRSSIEYSQREEYEATGTPFPGGAVSFPLPPGPHFGTPGYGSPHFTTLYSVPFPEGEAFPSVPVTALGSPMHSN

SEQ ID NO: 19 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

(19) United States
(12) Patent Application Publication
(10) Pub. No.: US 2012/0021519 A1
(43) Pub. Date: Jan. 26, 2012

(54) EFFICIENT INDUCTION OF PLURIPOTENT STEM CELLS USING SMALL MOLECULE COMPOUNDS

(75) Inventors: Justin Ichida, Boston, MA (US); Joel Blanchard, Pownal, ME (US); Lee Rubin, Wellesley, MA (US); Kevin Eggan, Boston, MA (US); Kelvin Lam, Arlington, MA (US)

(73) Assignee: PRESIDENTS AND FELLOWS OF HARVARD COLLEGE, Cambridge, MA (US)

(21) Appl. No.: 13/119,848

(22) PCT Filed: Sep. 21, 2009
(86) PCT No.: PCT/US09/57669

§ 371 Date: Oct. 11, 2011

Related U.S. Application Data
(60) Provisional application No. 61/098,683, filed on Sep. 19, 2008.

(51) Int. Cl. C12N 5/071

(52) U.S. Cl. 435/377

(57) ABSTRACT

The disclosure features a method of producing a reprogrammed cell (e.g. an induced pluripotent stem cell or an undifferentiated cell) from a differentiated (e.g. somatic) cell. In some embodiments, the methods includes contacting a differentiated (e.g. somatic cell) with a TGFBR1 inhibitor or anti-TGF-β-antibody to produce a reprogrammed cell (e.g. pluripotent stem cell or undifferentiated cell). Embodiments of the present invention relate to a reprogrammed cell and methods and compositions for producing a chemically produced reprogrammed cell or populations thereof.

### CROSS REFERENCE TO RELATED APPLICATIONS

This application claims the benefit under 35 U.S.C. 119(e) of U.S. Provisional Patent Application Ser. No. 61/098,683 filed Sep. 19, 2008, the contents of which is incorporated herein in its entirety by reference.

### GOVERNMENT SUPPORT

This invention is made with Government Support under Grant No: HD046732-01A1 awarded by the National Institutes of Health (NIH). The Government has certain rights in the invention.

### FIELD OF INVENTION

The invention relates to methods and compositions for reprogramming a differentiated cell into an undifferentiated cell, e.g., an induced pluripotent cell or a partially induced pluripotent cell. Embodiments of the present invention relate to a reprogrammed cell and methods and compositions for producing a chemically produced reprogrammed cell.

### BACKGROUND OF THE INVENTION

One goal of regenerative medicine is to be able to convert an adult differentiated cell into other cell types for tissue repair and regeneration. Retroviral transduction with three genes: Sox2, Oct4, and Klf4, has been shown to directly reprogram mouse or human differentiated cells (e.g. somatic cells) to a pluripotent stem cell state (1-5). Unfortunately, the resulting induced pluripotent stem (iPS) cells are suboptimal for uses in transplantation medicine and disease modeling because the viral transgenes they contain may spontaneously re-activate, a process that has lead to tumor formation in mice generated from iPS cells (6). Furthermore, in two gene therapy trials, the retroviral vectors used for delivery of reprogramming genes were themselves shown to be intrinsically oncogenic (7, 8).

While generation of iPS cells using non-integrating DNA-based methods (9-13) have been reported and are an improvement over retroviral delivery of reprogramming factors, use of such methods in therapeutic transplantation medicine and disease models is limited because these vectors are still considered to cause permanent alterations in chromosomal DNA (13, 14) that may be difficult to detect. Therefore, the clinical implementation of reprogramming technology would optimally avoid viral transduction and the introduction of any transgenic DNA in general. While transduction with recombinant protein factors has been reported to be capable of reprogramming mouse embryonic fibroblasts (15), use of protein factors is limited due to this process is highly inefficient and too laborious and expensive to implement at a large-scale. Furthermore, this methodology requires the use of valproic acid (VPA), a histone deacetylase (HDAC) inhibitor that can cause long-lasting, heritable changes in the expression of imprinted and cancer-related genes in mammalian cells (16, 17).

The identification of small molecules that can efficiently reprogram patient cells without the use of DNA expression vectors or large-scale protein preparations might most reproducibly allow the efficient generation of pluripotent stem cells that would be genetically unmodified, and as a result, most suitable for use in cell therapies. Small molecules that globally alter chromatin structure, including the DNA methyltransferase inhibitor 5-aza-cytidine (AZA) and the HDAC inhibitor VPA, can increase reprogramming efficiency and even reduce the number of factors required for reprogramming (18-21). Treatment with these inhibitors relaxes the structure of chromatin and in turn lowers the barrier to activation of endogenous pluripotency associated genes. However, Oct4 and Sox2 not only collaborate in reprogramming by activating genes required for pluripotency, they also function to repress genes promoting differentiation. It is therefore unlikely that this class of small molecules will alone be sufficient to replace all of the transgenic factors required for reprogramming. As a result, there is a significant need to identify additional small molecules that can function in reprogramming either independently or in concert with chemicals modulating chromatin structure. These reprogramming molecules might function through two broad mechanisms: to either activate expression of the endogenous reprogramming genes or to complement the omission of a transgenic factor, for instance by activating alternative genes that can substitute for them. Finally, identification of small molecules capable of reprogramming cells may provide additional insight regarding cytoplasmic signaling pathways that regulate pluripotency (22).

Reprogramming differentiated cells to a pluripotent state could generate a rich supply of patient-specific stem cells for regenerative medicine. Recent work has demonstrated that exogenous nucleic acid expression of four transcription factors—Sox-2, Oct-4, Klf-4, and c-Myc, or Sox-2, Oct-4, Nanog, and Lin-28, can directly reprogram differentiated cells to a pluripotent stem cell state.

Neural stem cells (NSCs) that already express endogenous Sox-2 can be reprogrammed without retrovirally delivered Sox-2, but in the presence of the other exogenous expressed transcription factors. This approach may be capable of partially eliminating the viral transgenes, but it is unlikely that these or any other Sox-2-expressing cells will be readily accessible from patients.

It has been shown that small molecule inhibitors of DNA methyltransferases such as 5-aza-Cytidine (5azaC) or histone deacetylases (HDACs) such as valproic acid (VPA), can increase reprogramming efficiency with all four factors or just three of the factors. However, in reprogramming experiments, these small molecules do not appear to replace the reprogramming factors, but instead increase their overall efficiency. Therefore, it may not be possible to replace all four reprogramming genes with these types of chemicals. Instead, it will likely require small molecules that perturb specific cell signaling pathways that result in the endogenous expression of the reprogramming genes or genes that substitute for them.

### SUMMARY OF THE INVENTION

The present invention relates to methods and compositions and compounds for reprogramming a differentiated cell. In particular, the present invention relates to methods and compositions to reprogram a differentiated cell by contacting the differentiated cell with a molecule, such as a small molecule, without the need to use exogenous transcription factors. In general, reprogramming of cells or production of iPS cells is achieved by delivery of transcription factors into adult somatic cells. Described herein are methods for reprogramming differentiated cells by treating differentiated cells with a variety of small molecules in place of the exogenously supplied transcription factors. Also described herein are reprogrammed cell populations, compositions according to the methods described herein, reprogrammed cell compositions comprising a differentiated cell in an admixture with at least one small molecule for reprogramming the differentiated cell, and kits for producing chemically induced reprogrammed cells.

Accordingly, one aspect of the present invention relates to the production of reprogrammed cells from differentiated cells using small molecules. Such reprogrammed cells are referred to herein simply as reprogrammed cells or chemically induced reprogrammed cells. In such embodiments, one or more small molecules or other agents are used in the place of (e.g. to replace or substitute) exogenously supplied transcription factors, either supplied as a nucleic acid encoding the transcription factor or a protein or polypeptide of the exogenously supplied transcription factor, which are typically used in reprogramming cells and the production of iPS cells. Thus, the inventors have discovered methods and compositions to replace the use of Sox2, Oct4, Klf4, Lin-28, Nanog, c-myc and other transcription factors typically used in the reprogramming of differentiated cells. As discussed herein, “exogenous” or “exogenously supplied” refers to addition of a nucleic acid encoding a reprogramming transcription factor (e.g. nucleic acids encoding Sox2, c-myc, Klf4 and Oct4) or a polypeptide of a reprogramming transcription factor (e.g. proteins of Sox2, c-myc, Klf4 and Oct4 or biologically active fragments thereof) which is normally used in production of iPS cells.

One aspect of the present invention relates to the production of a reprogrammed cell by contacting a cell with one or more agents, such as small molecules, where the agent (e.g. small molecules) replace the need to reprogram the differentiated cell with exogenous Sox2, Klf4 and Oct4 transcription factor.

In one embodiment, replacement of exogenous transcription factor Sox2 is by an agent which is an inhibitor of the TGFβ cell signalling pathway, such as a TGFBR1 inhibitor. In some embodiments, replacement of exogenous transcription factor Sox2 is by any compound with the formula selected from Formulas I, III-VII. In some embodiments, where a differentiated cell is contacted with an inhibitor of the TGF pathway, or an inhibitor of TGFBR1, or a compound with the Formula selected from Formulas I, III-VII, the cell is not contacted with an exogenous Sox, such as Sox2 transgene or Sox2 protein. In some embodiments, replacement of exogenous transcription factor Sox2 is by any compound with Formula I such as Repsox (E-616452) or E-616451. In another embodiment, replacement of exogenous transcription factor Sox2 is by any compound with Formula III such as SB431542 (Formula III). In some embodiments, one can reprogram a differentiated cell using at least one compound or any compound with Formula I such as Repsox (E-616452) or E-616451, or any compound with Formula III such as SB431542 (Formula III) in the absence of Sox2 or c-myc. Accordingly, the compounds of Formula I and III can function to replace two reprogramming transcription factors, Sox2 and c-myc.

In one embodiment, replacement of exogenous transcription factor Sox2 is by an agent which is an inhibitor of the SRC signalling pathway, such as a SRC inhibitor. In some embodiments, replacement of exogenous transcription factor Sox2 is by any compound with the Formula II. In some embodiments, where a differentiated cell is contacted with an inhibitor of the SRC pathway, or a compound with the Formula II, the cell is not contacted with an exogenous Sox, such as Sox2 transgene or Sox2 protein. In some embodiments, replacement of exogenous transcription factor Sox2 is by any compound with Formula II such as EI-275.

In some embodiments, contact of a differentiated cell with an agent which replaces Sox2 transcription factor, (e.g. inhibitor of TGF signalling, such as a TGFB1 inhibitor, or a SRC inhibitor, or any compound with Formulas I-VII, including but not limited to Repsox (E-616452), E-616451, SB431542 and EI-275, enables reprogramming of differentiated cells by only 3 transcription factors, such as Oct-4, Klf-4 and c-Myc without the need for Sox-2 (e.g. in the absence of exogenous Sox2 transcription factor).

In some embodiments, contact of a differentiated cell with an agent which replaces Sox2 requires only 2 transcription factors, Oct-4 and Klf-4 without the need for c-Myc or Sox-2 transcription factors. Stated another way, in one embodiment, contacting a differentiated cell with an agent which replaces Sox2 transcription factor, (e.g. contacting the cell with an inhibitor of TGF signalling, such as a TGFB1 inhibitor, or a SRC inhibitor, or any compound with Formulas I-VII, including but not limited to Repsox (E-616452), E-616451, SB431542 and EI-275) replaces two transcription factors Sox2 and c-Myc, and thus, enables reprogramming of differentiated cells by contacting with only 2 transcription factors, Oct-4 and Klf-4 (in the absence of Sox2 and c-Myc).

As shown herein, the inventors demonstrated reprogrammed (e.g. iPS) colonies from mouse fibroblasts (MEFs) infected by Oct-4 and Klf-4 retroviruses together with RepSox treatment. The number and percentage of iPS colonies was comparable to those in the addition of the Sox-2 transgene (e.g. nucleic acid encoding Sox2 transcription factor). Thus, the 3-factor reprogramming efficiency by RepSox treatment is comparable to the induction rate for mouse fibroblasts infected by 4 factors (Oct-4, Klf-4, c-Myc and Sox-2), demonstrating that RepSox treatment effectively replaced the need for exogenous Sox-2 transcription factor. Thus, described herein are methods for producing reprogrammed cells from differentiated cells (e.g. from fibroblasts e.g., MEFs) without using the oncogenes, for example c-Myc or Sox-2.

In some embodiments, a differentiated cell which is contacted with an agent which replaces Sox2, (e.g. inhibitor of TGF signalling, such as a TGFB1 inhibitor, or a SRC inhibitor, or any compound with Formulas I-VII, including Repsox (E-616452), E-616451, SB431542 and EI-275, can be reprogrammed with small molecules or other agents which replace exogenous supplied Oct-4 and Klf-4, as disclosed herein.

Thus, described herein are methods for producing reprogrammed cells from differentiated cells without using the oncogenes, for example c-Myc or oncogenes associated with introduction of nucleic acid sequences encoding the transcription factors Sox-2, Oct-4 or Klf-4 into the differentiated cell to be reprogrammed (e.g. viral oncogenes). For example, the chemical mediated reprogramming of differentiated cells makes it possible to create reprogrammed cells (e.g. iPS cells or partially reprogrammed cells) from small numbers of differentiated cells (e.g., such as those obtained from hair follicle cells from patients, blood samples, adipose biopsy, fibroblasts, skin cells, etc). In one embodiment, the addition of small molecules compounds (e.g., chemicals) allows successful and safe generation of reprogrammed cells (e.g. iPS cells or partially reprogrammed cells) from human differentiated cells, such as skin biopsies (fibroblasts or other nucleated cells) as well as from differentiated cells from all and any other cell type.

In one embodiment, the inventors have discovered that an inhibitor (e.g., a small molecule inhibitor) of TGF-beta signaling pathway (e.g., a TGFBR1 inhibitor) or an inhibitor of SRC signaling pathway (e.g., an SRC kinase inhibitor) can replace Sox-2 in the direct reprogramming of mouse fibroblasts, for example to induce pluripotent stem cells (e.g., iPS cells or partially reprogrammed cells). The addition of the compound (e.g., a TGFBR1 inhibitor or an inhibitor of SRC signaling pathway e.g., an SRC kinase inhibitor) to fibroblasts expressing Oct-4 and Klf-4 can generate comparable number and percentage of iPS colonies to those in the addition of the Sox-2 transgene. In some embodiments, this effect is independent of other compounds, for example other small molecules such as HDAC inhibitor (e.g., VPA) or inhibitors of DNA methyltransferase (e.g., 5azaC). In some embodiments, the effect is not dependent on the presence of the expression of c-Myc.

In some embodiments, the method comprises contacting a differentiated cell with an inhibitor of Transforming Growth Factor beta (TGFβ) signaling pathway e.g., Transforming Growth Factor Receptor type I (TGFBR1) kinase inhibitor (e.g., a compound described herein such as RepSox or SB-431542) or an anti-TGF-β-antibody, or a nucleic acid agent such as an siRNA to thereby produce a primitive precursor or a less differentiated cell, e.g., pluripotent stem cell (or a population thereof) or to reprogram the differentiated cell. In some preferred embodiments, the method includes contacting a differentiated cell with RepSox.

In one embodiment, the method comprises contacting a plurality of differentiated cells with an inhibitor of TGF-beta signaling pathway e.g., a TGFBR1 inhibitor (e.g., RepSox) to thereby produce a plurality of iPS cells from the differentiated cells.

In some embodiments, the inhibitors of TGFβ signaling pathway include small molecules, antibodies against one or more component(s) in the TGFβ signaling pathway or nucleic acid reagents (e.g., a double stranded RNA agent such as siRNA) targeting one or more component(s) in the TGFβ signaling pathway, or any combination thereof.

Another aspect of the present invention relates replacement of exogenous transcription factor Klf-4 by an agent which is an agonist of Mek/Erk cell signalling, such as any compound with Formula VIII, such as prostaglandin J2 or an inhibitor of Ca²⁺/calmodulin signalling or EGF receptor tyrosine kinase inhibitor, such as any compound with Formula XI, such as HDBA.

Another aspect of the present invention relates replacement of exogenous transcription factor Oct-4 by an agent which is an inhibitor of Na⁺ channels, such as any compound with Formula X, such as Simomenine, or an agonist or ATP-dependent potassium channel, such as any compound with Formula X, such as Simomenine or an agonist of MAPK signalling pathway, such as any compound with Formula XI, such as Ropivocaine or Bupivacaine.

Accordingly, the invention includes methods of using a compound such as a small molecule modulator of a cell signaling pathway, to replace one or more of the iPS transgenes used to reprogram a differentiated cell to an iPS cell. In some embodiments, one or more compounds described herein (e.g., a small molecule modulator of a cell signaling pathway) is combined to provide for the reprogramming of a differentiated cell into in iPS cell without the use of a transgene e.g., a viral transgene.

Disclosed herein are methods of perturbation of a broadly known cell signaling pathway e.g., by a small molecule modulator of a cell signaling pathway, which can functionally replace the forced over expression of an iPS transgene in the direct reprogramming process (e.g., the reprogramming of a differentiated cell into an iPS cell). This process does not require procurement of a highly specialized or scarce cell populations or use of generally acting chemicals that may produce undesirable effects on the recipient cells. Furthermore, in some embodiments, treatment with the small molecule modulator of a cell signaling pathway can be as effective as transduction with a Sox-2 retrovirus, which demonstrates that efficiency is not compromised by small molecule replacement of exogenous transcription factors.

The methods described herein can be used, for example, to optimize the production of a more primitive precursor or a less differentiated cell, such as an undifferentiated cell or an iPS cell, from a more differentiated cell, e.g., a somatic cell, by replacing one or more exogenously supplied transcription factor(s) used to produce the more primitive precursor (e.g. a reprogrammed cell or undifferentiated cell) with a compound such as small molecule or antibody. The methods can be used, for example in the creation of reprogrammed cells (e.g. iPS cells) from human biopsies, such as blood, skin, fat, hair follicle, mucus, etc. In some embodiments, the reprogrammed cells can be used to generate clonal cell lines (e.g. iPS lines or partially reprogrammed cell lines), which can be used for multiple purposes, for example to study differentiation and disease mechanisms/pathology, and or to produce differentiated cells (from the reprogrammed cells), for example of a specified morphology (e.g., neuron cells, pancreatic cells, etc.).

Accordingly, aspects the invention provides methods of producing undifferentiated cells, reprogrammed cells, primitive precursors, or a less differentiated cells, e.g., a multipotent or pluripotent stem cell (or a population thereof) from a differentiated cell or a stable intermediate cell using small molecules, and in some embodiments, in the absence of one or more exogenous transcription factors; Sox2, Oct4, Klf4, and cMyc.

### BRIEF DESCRIPTION OF THE DRAWINGS

FIG. 1A-1F shows identification of Small Molecules That Replace of Sox2. FIG. 1A shows an overview of chemical screen for replacement of Sox2. FIG. 1B shows a PO colony from Oct4, Klf4, and cMyc-infected MEFs plus RepSox that displays a mES-like morphology and is Oct4::GFP+. Scale bars=200 μm. FIG. 1C is a table which shows the number of Oct4::GFP+ colonies detected for each hit in the primary screen after transduction of Oct4, Klf4, and cMyc and VPA treatment. FIG. 1D shows the chemical structures of E-616452 (Repsox), E-616451 (a TGFβR1 inhibitor), and EI-275 (a Src inhibitor), with the optimal concentrations for reprogramming differentiated cells also shown. FIG. 1E is a histogram showing quantification of small molecule replacement of Sox2 in Oct4, Klf4, and cMyc infected MEFs with and without VPA treatment. Repsox is shown to reprogram differentiated cells in the absence of VPA. Colonies were counted at 30 days post-infection. FIG. 1F is a graph showing Sox2 replacement by RepSox is not dependent on cMyc. Quantization of Oct4::GFP+colonies induced by RepSox in Oct and Klf-4-infected MEFs in the absence of c-myc and absence of VPA treatment. Colonies were counted at 30 days post-infection.

FIGS. 2A-2C show RepSox-reprogrammed Cells Are in a Pluripotent state. FIG. 2A shows microarray scatter plots showing that the global gene expression profile of Oct4, Klf4, c-myc (OKM)+RepSox line 1 cells is very different from that of differentiated MEFs (left panel) and highly similar to that of mES line V6.5 (middle panel) and an iPS line generated with transduction with Oct4, Klf4, cMyc, and Sox2 (OKMS-iPS) (right panel). FIG. 2B shows teratomas containing cells of the three germ layers (mesoderm, endoderm, ectoderm) formed by injection of OK+RepSox cells into nude mice. FIG. 2C shows an 8 week old chimeric mouse formed by injection of OK+RepSox line 1 cells (C57BL6 genetic background) into an ICR blastocyst.

FIGS. 3A-3G shows RepSox Specifically Replaces Sox2 by Inhibiting Tgf-βSignaling. FIG. 3A shows the chemical structure of SB431542, an inhibitor of Tgfbr1 activity, with the optimal concentration for reprogramming a differentiated cell is also shown. FIG. 2B shows inhibition of Tgf-β signaling by treatment of Oct4, Klf4, cMyc-infected MEFs with either Sox2 transfection, two different anti TGF-β neutralizing antibodies (anti-TGF-B antibodies), SB431542 or Repsox. TGF-βneutralizing antibodies replaces Sox2. Colonies were counted at 30 days post-infection. FIG. 3C shows RepSox does not increase the efficiency of Oct4::GFP+colony induction in Oct4, Klf4, cMyc, and Sox2-infected MEFs. Shown are the numbers of colonies per 7500 infected cells plated. Colonies were counted at 30 days post-infection. FIG. 3D shows inhibition of Tgf-βsignaling by TGF-βneutralizing antibodies does not increase the efficiency of Oct4::GFP+colony induction in Oct4, Klf4, cMyc, and Sox2-infected MEFs. Shown are the numbers of colonies per 7500 infected cells plated. Colonies were counted at 30 days post-infection. FIG. 3E shows Repsox is specific for Sox Replacement and RepSox does not replace transgenic Oct4 or transgenic Klf4 in reprogramming. No Oct4::GFP+colonies were observed in RepSox-treated Klf-4-infected MEFs or Oct-4-infected MEFs out of 30,000 cells plated both with and without VPA treatment. Typically, 30-40 Oct4::GFP+ colonies were observed when the same number of Oct4, Klf4, cMyc-infected MEFs were plated and treated with RepSox. Colonies were counted at 30 days post-infection. FIG. 2F shows RepSox can replace cMyc in reprogramming. Cells were transduced with Oct4, Klf4, and Sox2 and treated with RepSox continuously starting at day 5 post-infection. Colonies were counted at 30 days post-infection. Reprogrammed cells were only detected when the cells were further transduced with c-myc or treated with Repsox. FIG. 2G shows inhibition of Tgf-β signaling can replace cMyc in reprogramming. Cells were transduced with Oct4, Klf4, and cMyc and treated with inhibitors of Tgf-βsignaling (Repsox at 1 μm, 25 μM, SB431542 (2 μM) and a pan-specific antibody) continuously starting at day 5 post-infection. Colonies were counted at 30 days post-infection.

FIGS. 4A-5B show that a short pulse of RepSox is sufficient for Sox2 replacement and most effective at later time points post-infection. FIG. 4B shows a schematic of a time course of RepSox treatment showing the number of Oct4::GFP+colonies induced by various timings of RepSox treatment of Oct4, cMyc, and Klf-4-infected MEFs in serum-containing mES medium. Colonies were counted at 24 days postinfection. Cells were treated with Repsox for between 1 day (24 hrs) and 9 days. Optimal reprogramming of cells with Repsox occurs when differentiated cells are treated at least 3 days post transfection Oct4, cMyc, and Klf-4-infected MEFs and for at least 3 days in duration. FIG. 4B shows a graph of the time course of RepSox treatment showing the number of Oct4::GFP+colonies induced by a 24-hr pulse of RepSox treatment on Oct4, cMyc, and Klf-4-infected MEFs in serum-free mES medium with knockout serum replacement (KSR mES). Optimal reprogramming was observed when differentiated cells were treated with Repsox treatment 11 days post transduction with Oct4, cMyc, and Klf4. Colonies were counted at 24 days post-infection. Shown are average colony numbers +/− the standard deviation.

FIGS. 5A-5F shows a stable intermediate can be reprogrammed by RepSox. FIG. 5A shows 2 of 10 stable, non-pluripotent intermediate cell lines derived from MEFs transduced with Oct4, Klf4, and cMyc can be reprogrammed with RepSox treatment but none can be reprogrammed with AZA treatment. In some instances, the non-pluripotent intermediate cell lines can be multipotent cells. FIG. 5B shows a western blot for phospho-Smad3 showing that RepSox inhibits Tgf-βsignaling in Oct4, cMyc and Klf-4-infected MEFs (OKM 10) cells. OKM 10 cells are a non-pluripotent intermediate cell line. Lysates were generated from cells treated with 25 μM RepSox for 48 hours in mES media. FIG. 5C shows RepSox does not increase the proliferation of OKM 10 cells. Cells were treated with 1 or 25 μM RepSox in mES media without feeders and were harvested and counted on the indicated days. FIG. 5D shows Repsox does no increase proliferation of intermediate cells. Shown in FIG. 5D is a stable, non-pluripotent intermediate cell line (OKM 10) derived from MEFs transduced with Oct4, Klf4, and cMyc can be reprogrammed with RepSox treatment but not with AZA or 21, indicating it is distinct from cell lines that can be reprogrammed by AZA or 21. “KSR mES” means media with knockout-serum replacement instead of fetal bovine serum. FIG. 5E shows partially reprogrammed cell lines from OKMS-transduced MEFS respond differently to RepSox and AZA treatment. Shown in FIG. 5E is Oct4::GFP-negative cell lines derived from Oct4::GFP-negative colonies in Oct4, Klf4, cMyc and Sox2-infected MEF cultures can be reprogrammed by RepSox or by AZA, but lines responsive to RepSox are not responsive to AZA alone and lines responsive to AZA are not responsive to RepSox alone, indicating the presence of two different types of stable intermediates in the reprogramming cultures. Oct4::GFP-negative colonies were picked at day 14 post-infection, propagated, treated with 25 μM RepSox, 500 AZA or both for 48 hours at passage 4, and scored for Oct4::GFP+colonies 12 days after RepSox treatment.

FIGS. 6A-6F shows RepSox replaces Sox2 by inducing Nanog expression. FIG. 6A shows 10 hr, 24 hr and 48 hr RepSox treatment of line OKMS 6 (Oct4, Klf4, cMyc and Sox2-infected MEF) strongly increases Nanog mRNA levels. Data was generated by microarray analysis and are relative to untreated controls. Nanog is induced faster and more significantly than when the cells are treated with Sox2, demonstrating Nanog is upregulated before fully reprogrammed cells form. FIG. 6B shows inhibition of Tgf-βsignaling increases Nanog expression in stable intermediate line OKMS 7 (Oct4, Klf4, cMyc and Sox2-infected MEF, line 7). FIG. 6C shows a 48 hr pulse of RepSox induces a persistent increase in Nanog expression in intermediate line OKM 10. OKM 10 cells were treated with 25 μM RepSox for 48 hours and RNA samples were taken at 0, 48, and 96 hours (48 hours after removal of RepSox) and analyzed by RTPCR analysis for Nanog expression. FIG. 6D shows shRNA-mediated knockdown of Nanog in OKM 10 cells blocks replacement of Sox2 by RepSox. OKM 10 cells or Oct4, Klf4, cMyc-transduced MEFs were transduced with a lentivirus encoding either an shRNA for Nanog or an empty vector, treated with 4 ug/ml puromycin for 3 days to enrich for cells that had been transduced, and treated with 25 μM RepSox for 9 days before GFP+colonies were scored on day 9 of RepSox treatment. FIG. 6E shows Nanog reprograms 3-factor intermediate line OKM 10. Nanog transduction can reprogram a stable, non-pluripotent intermediate cell line derived from Oct4, Klf4, and cMyc-transduced MEFs (OKM 10) at a similar efficiency as Sox2 transduction. Oct4::GFP+colonies were counted at 9 days post transduction. FIG. 6G shows Nanog can substitute for Sox2 in reprogramming of differentiated somatic fibroblasts. Oct4, Klf4, and cMyc-transduced MEFs were transfected with Sox2 or Nanog. Oct4::GFP+colonies were counted at 9 days post-transduction.

FIG. 7A-7C shows Oct4::GFP+colony formation in Oct4, Klf4, cMyc-infected MEFs as a function of compound concentration. FIG. 7A shows Oct4, Klf4, cMyc-infected MEFs formed at different concentrations of E-616452 (RepSox). Optimal concentrations for Repsox is greater than 10. Optimal concentrations for Repsox is greater than 10 μM, such as 25 μM or above. FIG. 7B shows Oct4, Klf4, cMyc-infected MEFs formed at different concentrations of E-616451. Optimal concentrations for E-616451 is greater than 1 μM, such as 3 μM, or between 1 μM-10 μM. FIG. 7C shows Oct4, Klf4, cMyc-infected MEFs formed at different concentrations of EI-275. Optimal concentrations for EI-275 is greater than 0.5 μM, such as 3 μM, or between 0.5 μM-10 μM. 2 mM of VPA was used in all wells.

FIG. 8 shows a RepSox-reprogrammed cell line contains transgenic Oct4, Klf4, and cMyc, but not Sox2. PCR with primers specific for the transgenic versions of Oct4, Klf4, cMyc and Sox2 (52) was performed on genomic DNA isolated from a control iPS cell line generated with Oct4, Klf4, cMyc, and Sox2 and a RepSox-reprogrammed cell line generated with Oct4, Klf4, and cMyc+RepSox.

FIG. 9 shows a RepSox-reprogrammed cell line is karyotypically normal. Shown is the karyotype of a passage 8 cell from Oct4, Klf4, and cMyc+RepSox line 1. 20 cells were counted and 5 cells were karyotype by GTL banding. All cells were karyotypically normal 40, XY.

FIGS. 10A-10B show OKM+RepSox line 1 and OK+RepSox line 1 cells form embryoid bodies in vitro. FIG. 12 A shows OKM+RepSox line 1 and FIG. 12B shows OK+RepSox line 1 cells form embryoid bodies after 3 days in suspension culture. Scale bars=500 μm

FIG. 11 shows OKM+Rep Sox line 1 and OK+Rep Sox line 1 cells efficiently differentiate into HB9+ motor neurons in vitro, and with higher efficiency than OKMS-iPS cell line 1 or mouse ES cells.

FIG. 12 shows Oct4::GFP-positive OKM+Rep Sox line 1 cells injected into 8-cell stage embryos migrate appropriately to the inner cell mass in the developing blastocyst.

FIG. 13 shows RepSox increases L-Myc mRNA expression in MEFs. MEFs were treated with 25 μM RepSox for 7 days and mRNA expression was determined by microarray analysis. Fold-induction is relative to untreated control samples.

FIG. 14 shows early and Late addition of Repsox induces reprogramming with similar timing. The number of Oct4::GFP+reprogrammed cells appear with similar timing whether RepSox treatment is initiated at day 7 or day 10 post-transduction.

FIG. 15A-15B shows RepSox does not increase proliferation of intermediate OKM 10 cells or mES cells. OKM 10 intermediate cells or R1 mES cells were treated with 25 μM RepSox in mES media without feeders and harvested and counted on the indicated days. RepSox treatment was initiated on day 0.

FIG. 16 shows RepSox treatment of intermediate line OKMS 6 increases the expression of Id1, Id2, and Id3, genes that are repressed by Tgf-βsignaling. Cells were treated with 0 or 25 μM RepSox for 10 hrs, 24 hours or 48 hours before RNA was harvested and analyzed by microarray. Shown are the fold-inductions of the genes with RepSox over without RepSox.

FIG. 17A-17B shows effect of Repsox treatment on Sox-family transcription factor mRNA expression in the stable 4-factor line OKMS 6. RepSox treatment for 10 hrs, 1 day, or 2 days is relative to untreated, time-matched controls. FIG. 17A shows expression levels of Sox1, 2, 5, 7 and 9 on RepSox treatment for 10 hrs, 1 day, or 2 days is relative to untreated, time-matched controls. FIG. 17B shows expression levels of Sox12, 13, 15, 21 and 30 on RepSox treatment for 10 hrs, 1 day, or 2 days is relative to untreated, time-matched controls Sox-3, 4, 6, 8, 17, and 18 were included in the microarray but are not represented in FIG. 17A or 17B because they did not change significantly enough to generate a low enough P value.

FIGS. 18A-18B show shRNA-mediated knockdown of Sox2 or Sox1 do not inhibit reprogramming by RepSox. FIG. 18A shows cells transduced once with a lentivirus encoding an shRNA specific for Sox2. FIG. 18B shows 5 different Sox1-specific shRNA vectors or an empty vector control. OKM 10 cells were used for the Sox2 shRNA while Oct4, Klf4, cMyc-transduced MEFs were used for the Sox1 shRNA. KSOM MEFs=Klf4, Sox2, Oct4, and cMyc-transduced MEFs.

FIG. 19 shows RepSox does not increase Nanog expression in intermediate lines OKMS 9 and OKM 9. Cells were treated with RepSox for 2 days before RNA was harvested.

FIGS. 20A-29B show Bmp signaling increases in response to RepSox treatment. FIG. 20A shows a western blot for phospho-Smad1/5/8 shows an increase in the amount of the phosphorylated protein after a 48-hr RepSox treatment. FIG. 20B shows mRNA expression analysis shows that Bmp-3 levels increase upon RepSox treatment. Data are relative to untreated controls.

FIG. 21 shows LIF receptor expression relative to MEFs. mRNA Expression analysis shows that non-pluripotent stable intermediate cell lines derived from Oct4, Klf4, and cMyc-transduced (OKM 10) and Oct4, Klf4, cMyc, and Sox2-transduced MEFs express the LIF receptor at the same level as mES cells. MEFs freshly infected with Oct4, Klf4, and cMyc (OKM MEFs day 7) have much lower levels of the LIF receptor.

FIG. 22 shows Nanog mRNA levels in MEFs freshly transduced with Oct4, Klf4, and cMyc (within 7 days) do not increase upon RepSox treatment.

FIG. 23A-23B shows endogenous pluripotency genes are activated in cell lines generated with Oct4, Klf4, cMyc and Nanog. FIG. 23A shows qPCR analysis showing relative expression of endogenous expression of pluripotency factors as compared to MEFs. FIG. 23B shows qPCR analysis showing relative expression of transgenic expression of pluripotency factors as compared to MEFs, mES or MONK cells.

FIG. 24A-24B shows Oct4::GFP-positive Oct4, Klf4, cMyc-infected MEF cells with combinations of compounds, Repsox (compound A), E-616451 (compound B) and EI-275 (compound C). FIG. 24A shows GFP positive reprogrammed cells with combinations of Repsox (compound A), E-616451 (compound B) and EI-275 (compound C) in the presence of VPA. The effect of Repsox (compound A) can be enhanced with combination of E-616451 (compound B) or EI-275 (compound C). FIG. 24B shows GFP positive reprogrammed cells with combinations of Repsox (compound A), E-616451 (compound B) and EI-275 (compound C) in the absence of VPA. The effect of Repsox (compound A) is not enhanced with combination of E-616451 (compound B) or EI-275 (compound C).

FIG. 25A-25B shows Oct4::GFP-positive Oct4, Klf4, cMyc-infected MEF cells with different TGFβ inhibitors. FIG. 25A shows Oct4::GFP-positive Oct4, Klf4, cMyc-infected MEF cells with different compounds, transduction with Sox-2, TGFβ receptor neutralizing antibodies (anti-TGF-B pan and anti-TGF-B (II)), Repsox and TGFβR inhibitor SB431542 in the presence or absence of VPA. FIG. 25B shows Oct4::GFP-positive cells with Klf4, cMyc, Sox2-infected MEF cells in the presence or absence of VPA, or a comparison of Oct4::GFP-positive cells with Klf4, oct4, cMyc-infected MEF cells with Repsox treatment or Sox2 transduction. Repsox is more efficient at reprogramming Klf4, oct4, cMyc-infected MEF cell as compared to sox2 transduction.

FIG. 26A-26E shows small molecule Replacement of Klf4. FIG. 26A shows a schematic overview of chemical screen for replacement of Klf4. FIG. 26B shows the chemical structure of Prostaglandin J2, with the optimal concentration for reprogramming differentiated cells shown. FIG. 26C shows the chemical structure of HDBA, with the optimal concentration for reprogramming differentiated cells shown. FIG. 26D show quantification of small molecule replacement of Klf4 in Oct4, cMyc and Sox2-infected MEFs with prostaglandin J2 and HBDA treatment, in the presence and absence of VPA treatment. Colonies were counted at 30 days post-infection. FIG. 27 E shows an Oct4::GFP+iPS line that was derived from a culture of Prostaglandin J2 treated Oct4, cMyc, Sox2-infected MEFs (OMS+Prostaglandin line 1) displays the characteristic mES-morphology and self-renewal properties. Passage 5. Scale bars=500 μm.

FIGS. 27A-27D show small Molecule Replacement of Oct4. FIG. 27A shows a schematic overview of chemical screen for replacement of Oct4. FIG. 27B shows the chemical structures of Sinomenine, Ropivacaine, and Bupivacaine, with the optimal concentration for reprogramming differentiated cells shown. FIG. 27C shows quantification of small molecule replacement of Oct4 in Klf4, cMyc, and Sox2-infected MEFs with and without VPA and 5-aza-cytidine treatment. Colonies were counted at 30 days post-infection. FIG. 27D shows an Oct4::GFP+iPS line that was derived from a culture of Bupivacaine treated Klf4, cMyc, and Sox2-infected MEFs (KMS+Bupivacaine line 1) displays the characteristic mES-like morphology and self-renewal properties, Passage 5 (top panels) and an Oct4::GFP+iPS line that was derived from a culture of Ropivocaine treated Klf4, cMyc, and Sox2-infected MEFs (KMS+Ropivocaine line 1), in the presence of VPA and AZA, which also displays the characteristic mES-like morphology and self-renewal properties (bottom panels). Scale bars=200 μm.

FIG. 28A-28C shows the reprogramming compounds do not act by increasing reprogramming efficiency. FIG. 28A shows the number of GFP positive cells in the presence of VPA is not enhanced with the presence of Sox2 replacement compounds; Repsox, E-616451. FIG. 28B shows the number of GFP positive cells in the presence of VPA is not enhanced with the presence of Oct4 replacement compound Simomenine or Bupivacaine. FIG. 28C shows the reprogramming efficiency with Klf4 replacement chemicals prostaglandin J2 and HBDA in the presence of all iPS factors, Oct4, Klf4, c-Myc and Sox2. Each compound was incubated with MEFs infected with Oct4, Klf4, cMyc, and Sox2 in conditions in which it was found to be most potent in reprogramming Concentrations: RepSox-25 μM, E-616451-3 μM (+VPA), EI-275-3 μM (+VPA), Bupivacaine-25 μM, Sinomenine-1 μM (+VPA), Prostaglandin J2-3 μM (KOSR media), HDBA-6 μM (KOSR media).

FIG. 29A-29C is similar to FIGS. 28A-28C, but shows the efficiency of reprogramming compounds in the presence of all iPS factors, Oct4, Klf4, c-Myc and Sox2. FIG. 29A shows the number of GFP positive cells in the presence of Sox2 replacement compounds; Repsox, E-616451. FIG. 29B shows the number of GFP positive cells in the presence of Oct4 replacement compounds Simomenine or Bupivacaine, or in the presence of VPA. FIG. 29C shows the reprogramming efficiency with Klf4 replacement chemicals prostaglandin J2 and HBDA, or in the presence of VPA. Each compound was incubated with MEFs infected with Oct4, Klf4, cMyc, and Sox2 in conditions in which it was found to be most potent in reprogramming. Concentrations: RepSox-25 μM, E-616451-3 μM (+VPA), EI-275-3 μM (+VPA), Bupivacaine-25 μM, Sinomenine-1 μM (+VPA), Prostaglandin J2-3 μM (KOSR media), HDBA-6 μM (KOSR media).

FIGS. 30A-30B shows chemically reprogrammed cell lines uniformly express the embryonic stem cell marker alkaline phosphatase. FIG. 30A shows alkaline phosphatese expression in Oct4, Klf4, cMyc-infected MEFs treated with RepSox line, (in the absence of exogenous Sox2 transcription factor). FIG. 30B shows alkaline phosphatese expression in Klf4, cMyc, Sox2-infected MEFs treated with Bupivacaine (in the absence of exogenous Oct4 transcription factor). FIG. 30C shows alkaline phosphatese expression in Oct4, cMyc, Sox2-infected MEFs treated with Prostaglandin J2 (in the absence of exogenous Klf4 transcription factor). All cell lines are passage 5 or higher. Scale bars=(A) 500 μm (B) 200 μm (C) 200 μm.

FIGS. 31A-32B shows microarray scatter plots showing that the global gene expression profile indicates that chemically reprogrammed cell lines are very similar to mES and iPS cell lines and dissimilar to MEFs with respect to gene expression. FIG. 31A shows the Bupivacaine cell line (+transgenic Klf4, cMyc, Sox2, (KMS)) is very different from that of differentiated MEFs (left panel) and highly similar to that of mES line V6.5 (middle panel) and an iPS line generated with transduction with Oct4, Klf4, cMyc, and Sox2 (OKMS-iPS) (right panel). FIG. 31B shows the Prostaglandin cell line (+transgenic Oct4, cMyc, Sox2 (OMS)) is very different from that of differentiated MEFs (left panel) and highly similar to that of mES line V6.5 (middle panel) and an iPS line generated with transduction with Oct4, Klf4, cMyc, and Sox2 (OKMS-iPS) (right panel).

### DETAILED DESCRIPTION OF THE INVENTION

The present invention relates to methods and compositions and compounds for reprogramming a differentiated cell. In particular, the present invention relates to methods and compositions for reprogramming a differentiated cell by contacting the differentiated cell a molecule, such as a small molecule, without the need to use exogenous transcription factors.

Accordingly, one aspect of the present invention relates to the production of reprogrammed cells from differentiated cells using small molecules. Such reprogrammed cells are referred to herein simply as reprogrammed cells or chemically induced reprogrammed cells. In such embodiments, one or more small molecules or other agents are used in the place of (e.g. to replace or substitute) exogenously supplied transcription factors, either supplied as a nucleic acid encoding the transcription factor or a protein or polypeptide of the exogenously supplied transcription factor, which are typically used in reprogramming cells and the production of iPS cells. Thus, the inventors have discovered a method and compositions to replace the use of Sox2, Oct4, Klf4, Lin-28, Nanog, c-myc and other transcription factors typically used in the reprogramming of differentiated cells. As discussed herein, “exogenous” or “exogenous supplied” refers to addition of a nucleic acid encoding a reprogramming transcription factor (e.g. nucleic acids encoding Sox2, Klf4 and Oct4) or a polypeptide of a reprogramming factor (e.g. proteins of Sox2, Klf4 and Oct4 or biologically active fragments thereof) which is often used in production of iPS cells.

As described herein, a component such as a TGFBR1 inhibitor(s), for example, a compound described herein (e.g., a small molecule such as RepSox or SB-431542) or an anti-TGF-β-antibody can be employed to efficiently generate induced pluripotent stem (iPS) cells from fibroblast or other cell types.

Reprogramming differentiated cells to a pluripotent state could generate a rich supply of patient-specific stem cells for regenerative medicine. Recent work has demonstrated that exogenous expression of four transcription factors—Sox-2, Oct-4, Klf-4, and c-Myc, or Sox-2, Oct-4, Nanog, and Lin-28, can directly reprogram differentiated cells to a pluripotent stem cell state [1-7].

It has been shown that small molecule inhibitors of DNA methyltransferases such as 5-aza-Cytidine (5azaC, or AZA) or histone deacetylases (HDACs) such as valproic acid (VPA), can increase reprogramming efficiency with all four factors or just three of the factors. However, in direct reprogramming experiments they do not appear to replace the reprogramming factors, but instead increase their overall efficiency. Therefore, it may not be possible to replace all four reprogramming genes with these types of chemicals. Instead, it will likely require small molecules that perturb specific cell signaling pathways that result in the endogenous expression of the reprogramming genes or genes that substitute for them.

As described herein, small molecules that modulate cell signaling pathways could specifically replace the transgenic iPS factors in reprogramming. In a screen of 680 small molecules that perturb various biological targets, the inventors identified 3 compounds that induce Oct-4-GFP+colonies from mouse fibroblasts transduced with only Klf-4, Oct-4, and c-Myc. Two of the compounds inhibit the Transforming Growth Factor Receptor type I (TGFBR1) kinase, and one induces Oct-4-GFP+colonies in mouse fibroblasts at the same rate as Sox-2 transduction. Using this TGFBR1 inhibitor, the inventors have generated iPS cells from mouse fibroblasts with Klf-4, Oct-4, and c-Myc, or just Klf-4 and Oct-4. These cells are indistinguishable from embryonic stem cells and Klf-4, Oct-4, Sox-2, c-Myc iPS cells with respect to self-renewal, gene expression, and differentiation. The inhibition of TGF-beta signaling does not significantly increase the efficiency of reprogramming in the presence of all four iPS transgenes and does not induce reprogramming when Oct-4 is absent, indicating that this small molecule specifically replaces Sox-2 instead of generally increasing the efficiency of reprogramming. These results demonstrate that treatment with a small molecule modulator of a broadly known cell signaling pathway can specifically replace one of the iPS transgenes in reprogramming and suggest that a cocktail of small molecules may be able to replace all four iPS transgenes, enabling the production of therapeutic-grade patient-specific reprogrammed cells (e.g. undifferentiated cells or induced pluripotent stem cells).

### DEFINITIONS

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

The term “reprogramming” as used herein refers to a process that alters or reverses the differentiation state of a differentiated cell (e.g. a somatic cell). Stated another way, reprogramming refers to a process of driving the differentation of a cell backwards to a more undifferentated or more primitive type of cell. The cell to be reprogrammed can be either partially or terminally differentiated prior to reprogramming. In some embodiments, reprogramming encompasses complete reversion of the differentiation state of a differentated cell (e.g. a somatic cell) to a pluripotent state. In some embodiments, reprogramming also encompasses partial reversion of the differentiation state of a differentated cell (e.g. a somatic cell) to a multipotent state. In some embodiments, reprogramming encompasses complete or partial reversion of the differentiation state of a differentated cell (e.g. a somatic cell) to an undifferentated cell. Reprogramming also encompasses partial reversion of the differentiation state of a somatic cell to a state that renders the cell more susceptible to complete reprogramming to a pluripotent state when subjected to additional manipulations such as those described herein. Such contacting may result in expression of particular genes by the cells, which expression contributes to reprogramming. In certain embodiments of the invention, reprogramming of a differentated cell (e.g. a somatic cell) causes the differentated cell to assume an undifferentated state (e.g. is an undifferentated cell). In some embodiments, reprogramming of a differentated cell (e.g. a somatic cell) causes the differentated cell to assume a pluripotent-like state. The resulting cells are referred to herein as “reprogrammed cells”, or “chemically induced reprogrammed cells” or “undifferentated cells”.

Reprogramming involves alteration, e.g., reversal, of at least some of the heritable patterns of nucleic acid modification (e.g., methylation), chromatin condensation, epigenetic changes, genomic imprinting, etc., that occur during cellular differentiation as a zygote develops into an adult. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods of the invention may also be of use for such purposes. Certain of the compositions and methods of the present invention contribute to establishing the pluripotent state. The methods may be practiced on cells that fully differentiated and/or restricted to giving rise only to cells of that particular type, rather than on cells that are already multipotent or pluripotent.

The term “reprogrammed cell” as used herein refers to a cell which has been reprogrammed from a differentated cell according to the methods as disclosed herein. In some embodiments, a reprogrammed cell is a cell which has undergone epigenetic reprogramming. The term “reprogrammed cell” encompasses an undifferentiated cell. The term “reprogrammed cell” also includes a partially reprogrammed cell except where it specifically indicates it does not include a partially reprogrammed cell.

The term “partially reprogrammed cell” as referred to herein refers to a cell which has been reprogrammed from a differentiated cell, by the methods as disclosed herein, wherein the partially reprogrammed cell has not been completely reprogrammed to pluripotent state but rather to a non-pluripotent stable intermediate state. A partially reprogrammed cell can differentiate into one or two of three germ layers, but cannot differentiate into all three of the germ layers. In some embodiments, a partially reprogrammed cell expresses at least one or at least two or at least three but not all of the following markers; alkaline phosphatase (AP), NANOG, OCT-4, SOX-2, SSEA4, TRA-1-60 or TRA-1-81. In some embodiments, a partially reprogrammed cell expresses markers from one or two germ cell layers, but not markers from all three embryonic germ layers (i.e. a partially reprogrammed cell does not express markers from all three layers of endoderm, mesoderm or ectoderm layers). Markers of endoderm cells include, Gata4, FoxA2, PDX1, Nodal, Sox7 and Sox17. Markers of mesoderm cells include, Brachycury, GSC, LEF1, Mox1 and Tiel. Markers of ectoderm cells include criptol, EN1, GFAP, Islet 1, LIM1 and Nestin. In some embodiments, a partially reprogrammed cell is an undifferentiated cell. In some embodiments, the methods as disclosed herein can be used to generate a partially reprogrammed cell (or population thereof) by contacting a differentiated cell with any compound selected from compounds of Formulas I-XI which replace one or two of the following reprogramming genes selected from the group of; Sox2, Oct3/4 or Klf4.

The term a “reprogramming gene”, as used herein, refers to a gene whose expression, contributes to the reprogramming of a differentiated cell, e.g. a somatic cell to an undifferentiated cell, e.g. a cell of a pluripotent state or partially pluripotent state. A reprogramming gene can be, for example, genes encoding transcription factors Sox2, Oct3/4, Klf4, Nanog, Lin-38, c-myc and the like.

The term “epigenetic reprogramming” as used herein refers to the alteration of the pattern of gene expression in a cell via chemical modifications that do not change the genomic sequence or a gene's sequence of base pairs in the cell.

The term “epigenetic” as used herein refers to “upon the genome”. Chemical modifications of DNA that do not alter the gene's sequence, but impact gene expression and may also be inherited. Epigenetic modification to DNA are important in imprinting and cellular reprogramming.

The term “contacting” or “contact” as used herein as in connection with contacting a differentiated cell with a compound as disclosed herein (e.g. of Formula I-XI), includes subjecting the cell to a culture media which comprises that agent (e.g. a compound of Formula I-XI). Where the differentiated cell is in vivo, contacting the differentiated cell with a compound includes administering the compound in a composition to a subject via an appropriate administration route such that the compound contacts the differentiated cell in vivo.

The term “pluripotent” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. In some embodiments, a pluripotent cell is an undifferentiated cell.

The term “pluripotency” or a “pluripotent state” as used herein refers to a cell with the ability to differentiate into all three embryonic germ layers: endoderm (gut tissue), mesoderm (including blood, muscle, and vessels), and ectoderm (such as skin and nerve), and typically has the potential to divide in vitro for a long period of time, e.g., greater than one year or more than 30 passages.

The term “multipotent” when used in reference to a “multipotent cell” refers to a cell that is able to differentiate into some but not all of the cells derived from all three germ layers. Thus, a multipotent cell is a partially differentiated cell. Multipotent cells are well known in the art, and examples of multipotent cells include adult stem cells, such as for example, hematopoietic stem cells and neural stem cells. Multipotent means a stem cell may form many types of cells in a given lineage, but not cells of other lineages. For example, a multipotent blood stem cell can form the many different types of blood cells (red, white, platelets, etc. . . . ), but it cannot form neurons.

The term “multipotency” refers to a cell with the degree of developmental versatility that is less than totipotent and pluripotent.

The term “totipotency” refers to a cell with the degree of differentiation describing a capacity to make all of the cells in the adult body as well as the extra-embryonic tissues including the placenta. The fertilized egg (zygote) is totipotent as are the early cleaved cells (blastomeres)

The term “differentiated cell” is meant any primary cell that is not, in its native form, pluripotent as that term is defined herein. The term a “differentiated cell” also encompasses cells that are partially differentiated, such as multipotent cells, or cells that are stable non-pluripotent partially reprogrammed cells. In some embodiments, a differentiated cell is a cell that is a stable intermediate cell, such as a non-pluripotent partially reprogrammed cell, such as OKMS6 cell line or OKM10 cell line as disclosed herein in the Examples 5-7. It should be noted that placing many primary cells in culture can lead to some loss of fully differentiated characteristics. Thus, simply culturing such cells are included in the term differentiated cells and does not render these cells non-differentated cells (e.g. undifferentiated cells) or pluripotent cells. The transition of a differentiated cell (including stable non-pluripotent partially reprogrammed cell intermediates) to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture. In some embodiments, the term “differentiated cell” also refers to a cell of a more specialized cell type derived from a cell of a less specialized cell type (e.g., from an undifferentiated cell or a reprogrammed cell) where the cell has undergone a cellular differentiation process.

As used herein, the term “somatic cell” refers to any cell other than a germ cell, a cell present in or obtained from a pre-implantation embryo, or a cell resulting from proliferation of such a cell in vitro. Stated another way, a somatic cell refers to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for reprogramming a differentiated cell can be performed both in vivo and in vitro (where in vivo is practiced when an differentiated cell is present within a subject, and where in vitro is practiced using isolated differentiated cell maintained in culture). In some embodiments, where a differentiated cell or population of differentiated cells are cultured in vitro, the differentiated cell can be cultured in an organotypic slice culture, such as described in, e.g., meneghel-Rozzo et al., (2004), Cell Tissue Res, 316(3); 295-303, which is incorporated herein in its entirety by reference.

As used herein, the term “adult cell” refers to a cell found throughout the body after embryonic development.

As used herein, the terms “iPS cell” and “induced pluripotent stem cell” are used interchangeably and refers to a pluripotent cell artificially derived (e.g., induced by complete or partial reversal) from an undifferentiated cell (e.g. a non-pluripotent cell), typically an adult differentiated cell, for example, by contacting the cell with at least one compound of any compounds selected from Formulas I-VII. In some embodiments, a differentiated cell is contacted with a composition comprising one or more of compound of Formula I, such as exemplarily compound RepSOX (E-616452), and in some embodiments, the composition comprises at least one additional compound, such as any compound selected from Formulas VIII-XI.

The term “progenitor cell” is used herein to refer to cells that have a cellular phenotype that is more primitive (e.g., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art.

In the context of cell ontogeny, the term “differentiate”, or “differentiating” is a relative term meaning a “differentiated cell” is a cell that has progressed further down the developmental pathway than its precursor cell. Thus in some embodiments, a reprogrammed cell as this term is defined herein, can differentiate to lineage-restricted precursor cells (such as a mesodermal stem cell), which in turn can differentiate into other types of precursor cells further down the pathway (such as an tissue specific precursor, for example, a cardiomyocyte precursor), and then to an end-stage differentiated cell, which plays a characteristic role in a certain tissue type, and may or may not retain the capacity to proliferate further.

The term “embryonic stem cell” is used to refer to the pluripotent stem cells of the inner cell mass of the embryonic blastocyst (see U.S. Pat. Nos. 5,843,780, 6,200,806, which are incorporated herein by reference). Such cells can similarly be obtained from the inner cell mass of blastocysts derived from somatic cell nuclear transfer (see, for example, U.S. Pat. Nos. 5,945,577, 5,994,619, 6,235,970, which are incorporated herein by reference). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “adult stem cell” or “ASC” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue.

The term “phenotype” refers to one or a number of total biological characteristics that define the cell or organism under a particular set of environmental conditions and factors, regardless of the actual genotype.

As used herein, the term “transcription factor” refers to a protein that binds to specific parts of DNA using DNA binding domains and is part of the system that controls the transfer (or transcription) of genetic information from DNA to RNA.

The term “expression” refers to the cellular processes involved in producing RNA and proteins and as appropriate, secreting proteins, including where applicable, but not limited to, for example, transcription, translation, folding, modification and processing. “Expression products” include RNA transcribed from a gene and polypeptides obtained by translation of mRNA transcribed from a gene.

The term “genetically modified” or “engineered” cell as used herein refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (e.g., sequences naturally found in the cells) but in a non-naturally occurring arrangement (e.g., a coding region linked to a promoter from a different gene), or altered versions of native sequences, etc. The process of transferring the nucleic into the cell is referred to as “transducing a cell” and can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell.

The term “agent” as used herein means any compound or substance such as, but not limited to, a small molecule, nucleic acid, polypeptide, peptide, drug, ion, etc. An “agent” can be any chemical, entity or moiety, including without limitation synthetic and naturally-occurring proteinaceous and non-proteinaceous entities. In some embodiments, an agent is nucleic acid, nucleic acid analogues, proteins, antibodies, peptides, aptamers, oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof etc. In certain embodiments, agents are small molecule having a chemical moiety. For example, chemical moieties included unsubstituted or substituted alkyl, aromatic, or heterocyclyl moieties including macrolides, leptomycins and related natural products or analogues thereof. Compounds can be known to have a desired activity and/or property, or can be selected from a library of diverse compounds.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The term “exogenous” refers to a substance present in a cell other than its native source. The terms “exogenous” when used herein refers to a nucleic acid (e.g. a nucleic acid encoding a sox2 transcription factor) or a protein (e.g., a sox2 polypeptide) that has been introduced by a process involving the hand of man into a biological system such as a cell or organism in which it is not normally found or in which it is found in lower amounts. A substance (e.g. a nucleic acid encoding a sox2 transcription factor, or a protein, e.g., a sox2 polypeptide) will be considered exogenous if it is introduced into a cell or an ancestor of the cell that inherits the substance. In contrast, the term “endogenous” refers to a substance that is native to the biological system or cell (e.g. differentated cell).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from. In some embodiments, the isolated population is an isolated population of reprogrammed cells which is a substantially pure population of reprogrammed cells as compared to a heterogeneous population of cells comprising reprogrammed cells and cells from which the reprogrammed cells were derived.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of reprogrammed cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not reprogrammed cells or their progeny as defined by the terms herein. In some embodiments, the present invention encompasses methods to expand a population of reprogrammed cells, wherein the expanded population of reprogrammed cells is a substantially pure population of reprogrammed cells.

As used herein, “proliferating” and “proliferation” refer to an increase in the number of cells in a population (growth) by means of cell division. Cell proliferation is generally understood to result from the coordinated activation of multiple signal transduction pathways in response to the environment, including growth factors and other mitogens. Cell proliferation may also be promoted by release from the actions of intra- or extracellular signals and mechanisms that block or negatively affect cell proliferation.

The terms “enriching” or “enriched” are used interchangeably herein and mean that the yield (fraction) of cells of one type is increased by at least 10% over the fraction of cells of that type in the starting culture or preparation.

The terms “renewal” or “self-renewal” or “proliferation” are used interchangeably herein, and refers to a process of a cell making more copies of itself (e.g. duplication) of the cell. In some embodiments, reprogrammed cells are capable of renewal of themselves by dividing into the same undifferentiated cells (e.g. pluripotent or non-specialized cell type) over long periods, and/or many months to years. In some instances, proliferation refers to the expansion of reprogrammed cells by the repeated division of single cells into two identical daughter cells.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

The term “lineages” as used herein describes a cell with a common ancestry or cells with a common developmental fate. By way of an example only, a cell that is of endoderm origin or is “endodermal linage” this means the cell was derived from an endodermal cell and can differentiate along the endodermal lineage restricted pathways, such as one or more developmental lineage pathways which give rise to definitive endoderm cells, which in turn can differentiate into liver cells, thymus, pancreas, lung and intestine.

The term “modulate” is used consistently with its use in the art, e.g., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “DNA” is defined as deoxyribonucleic acid.

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (e.g. the succession of letters used as abbreviations for bases) that biochemically characterizes a specific nucleic acid. A polynucleotide sequence presented herein is presented in a 5′ to 3′ direction unless otherwise indicated.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. ScL USA 87:22264-2268, 1990) modified as in Karlin and Altschul, Proc. Natl. Acad. ScL USA 90:5873-5877, 1993 is incorporated into the NBLAST and XBLAST programs of Altschul et al. (Altschul, et al., J. MoI. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI.

As used herein, the term “xenogeneic” refers to cells that are derived from different species.

As used herein, the term “TGF-β signaling pathway” is used to describe the downstream signaling events attributed to TGF-β and TGF-β like ligands. For example, in one signaling pathway a TGF-β ligand binds to and activates a Type II TGF-β receptor. The Type II TGF-β receptor recruits and forms a heterodimer with a Type I TGF-β receptor. The resulting heterodimer permits phosphorylation of the Type I receptor, which in turn phosphorylates and activates a member of the SMAD family of proteins. A signaling cascade is triggered, which is well known to those of skill in the art, and ultimately leads to control of the expression of mediators involved in cell growth, cell differentiation, tumorigenesis, apoptosis, and cellular homeostasis, among others. Other TGF-β signaling pathways are also contemplated for manipulation according to the methods described herein. TGF-beta regulates growth and proliferation of cells, blocking growth of many cell types. The TGF-beta receptor includes type 1 and type 2 subunits that are serine-threonine kinases and that signal through the SMAD family of transcriptional regulators (see Miyazono K, ten Dijke P, Heldin C H., Adv Immunol 2000; 75:115-57, TGF-beta signaling by Smad proteins, which is incorporated herein in its entirety by reference). Prior to activation, receptor regulated SMADs are anchored to the cell membrane by factors like SARA (SMAD Anchor for Receptor Activation) that brings the SMADs into proximity of the TGF receptor kinases. Binding of TGF induces phosphorylation and activation of the TGF-beta R1 receptor by the TGF-beta R2 receptor. The activated TGF-beta R1 phosphorylates SMAD2 and SMAD3, which bind to the SMAD4 mediator to move into the nucleus and form complexes that regulate transcription. SMADs regulate transcription in several ways, including binding to DNA, interacting with other transcription factors, and interacting with transcription corepressors and coactivators like p300 and CBP. SMAD-7 represses signaling by other SMADs to down-regulate the system. Other signaling pathways like the MAP kinase-ERK cascade are activated by TGF-beta signaling, modulate SMAD activation. SnoN also regulates TGF-beta signaling, by binding to SMADs to block transcriptional activation. TGF-beta signaling causes degradation of SnoN, releasing SMADs to regulate transcription, and also activates expression of SnoN, to down-regulate SMAD signaling at later times.

The term “inhibitor of the TGF-β signaling pathway” as used herein, refers to inhibition of at least one of the proteins involved in the signal transduction pathway for TGF-β. It is contemplated herein that an inhibitor of the TGF-β signaling pathway can be, for example, a TGF-β receptor inhibitor (e.g., a small molecule, an antibody, an siRNA), a TGF-β sequestrant (e.g., an antibody, a binding protein), an inhibitor of receptor phosphorylation, an inhibitor of a SMAD protein, or a combination of such agents.

In one embodiment, the TGF-β signaling pathway inhibitor comprises or consists essentially of a TGF-β receptor inhibitor. One of skill in the art can easily test a compound to determine if it inhibits TGF-β receptor signaling by assessing, for example, phosphorylation status of the receptor or expression of downstream proteins controlled by TGF-β in cultured cells and comparing the results to cells not treated with a TGF-β receptor inhibitor. An agent is determined to be a TGF-β signaling pathway inhibitor if the level of phosphorylation of the Type I TGF-β receptor in a culture of cells is reduced by at least 20% compared to the level of phosphorylation of the Type I TGF-β receptor in cells that are cultured in the absence of a TGF-β signaling pathway inhibitor; preferably the level of phosphorylation is reduced by at least 30%, at least 40%, at least 50%, at least 60%, at least 70%, at least 80%, at least 90%, at least 95%, at least 99%, or even 100% (no phosphorylation) in the presence of a TGF-β signaling pathway inhibitor.

As used herein, the term “Alk5” is used to denote a TGF-beta receptor type I having serine/threonine protein kinase activity (also referred to herein as TGFβR-1). The term “TGF-beta receptor” or “TGFβR” is used herein to encompass all three sub-types of the TGFβR family (e.g., TGFβR-1, TGFβR-2, TGFβR-3). The TGFβ receptors are characterized by serine/threonine kinase activity and exist in several different isoforms that can be homo- or heterodimeric.

The term “Src signaling pathway” as used herein is used to describe the downstream signaling events attributed to Src, or Src like ligands.

The term “src inhibitor” or “inhibitor Src signaling pathway” are used interchangeably herein and refers to any agent which reduces the expression or activity of Src ligand, reduces the phosphorylation of the src phosphorylated site, particularly on EGFR, or reduces the signal of the src kinase cascade. An agent can be a small molecule, such as a chemical entity, a peptide, an antibody, antibody fragment or other such agent, etc. An agent which is a src inhibitor may include a kinase inhibitor, phosphatase, etc.

The term “Mek/Erk Signaling pathway” is also known in the art as the “Raf-MEK-ERK” signal transduction cascade, and refers to a conserved pathway which regulates cell growth, proliferation, differentiation, and apoptosis in response to growth factors, cytokines, and hormones. This pathway operates downstream of Ras. Activated Ras activates the protein kinase activity of RAF kinase. RAF kinase phosphorylates and activates MEK. MEK phosphorylates and activates a mitogen-activated protein kinase (MAPK). RAF, MEK and MAPK are all serine/threonine-selective protein kinases. MAPK was originally called “extracellular signal-regulated kinases” (ERKs) and microtubule-associated protein kinase (MAPK).

The term “agonist Mek/Erk signaling pathway” as used herein refers to any agent which increases or enhances the expression or activity of Raf, Mek or MAPK or increases their downstream signaling pathway. An agent can be a small molecule, such as a chemical entity, a peptide, an antibody, antibody fragment or other such agent, etc. An agent which is an agonist of Raf, Mek or MAPK may include a kinase inhibitor, phosphatase, etc.

The term “Ca²⁺/Calmodulin signaling pathway” is used to describe the downstream signaling events attributed to Ca²⁺/calmodulin-dependent protein kinases or CaM kinases, which are serine/threonine-specific protein kinases that are primarily regulated by the Ca2+/calmodulin complex. Calmodulin is the primary receptor for calcium present in all cells. The binding of its calcium ligand results in a conformational change in calmodulin, which allows the calcium-calmodulin complex to interact with many different targets. The Ca²⁺/Calmodulin signaling pathway is disclosed in Means et al., Molecular Endocrinology 22 (12): 2759-2765, which is incorporated herein in its entirety by reference.

The term “inhibitor Ca2+/Calmodulin signaling pathway” as used herein refers to any agent which reduces or decreases the expression or activity of calmodulin or its downstream signaling pathway. An agent can be a small molecule, such as a chemical entity, a peptide, an antibody, antibody fragment or other such agent, etc. An agent which is a calmodulin inhibitor may include a kinase inhibitor, phosphatase, etc.

The term “EGF signaling pathway” is used to describe the downstream signaling events attributed to the epidermal growth factor (EGF) peptide. EGF induces cellular proliferation through the EGF receptor, which has a tyrosine kinase cytoplasmic domain, a single transmembrane domain and an extracellular domain involved in EGF binding and receptor dimerization. Binding of EGF results in EGF receptor dimerization, autophosphorylation of the receptor, and tyrosine phosphorylation of other proteins. The EGF receptor activates ras and the MAP kinase pathway, ultimately causing phosphorylation of transcription factors such as c-Fos to create AP-1 and ELK-1 that contribute to proliferation. Activation of STAT-1 and STAT-3 transcription factors by JAK kinases in response to EGF contributes to proliferative signaling. Phosphatidylinositol signaling and calcium release induced by EGF activate protein kinase C, another component of EGF signaling. Crosstalk of EGF signaling with other pathways make the EGF receptor a junction point between signaling systems.

The term “inhibitor EGF signaling pathway” as used herein refers to any agent which reduces or decreases the expression or activity of EGF or its downstream signaling pathway. An agent can be a small molecule, such as a chemical entity, a peptide, an antibody, antibody fragment or other such agent, etc. An agent which is an EGF inhibitor may include a kinase inhibitor, phosphatase, etc.

The term “MAPK signalling pathway” is used to describe the downstream signaling events attributed to Mitogen-activated protein (MAP) kinases. The mitogen-activated protein kinase (MAP kinase) pathways consist of four major groupings and numerous related proteins which constitute interrelated signal transduction cascades activated by stimuli such as growth factors, stress, cytokines and inflammation. The four major groupings are the Erk, JNK or SAPK, p38 (green) and the MAPK or ERK5 cascades. Signals from cell surface receptors such as GPCRs and growth factor receptors are transduced, directly or via small G proteins such as ras and rac, to multiple tiers of protein kinases that amplify these signals and/or regulate each other. Mitogen-activated protein (MAP) kinases are important players in signal transduction pathways activated by a range of stimuli and mediate a number of physiological and pathological changes in cell function. There are three major subgroups in the MAPK family: ERK, p38, and JNK/SAPK. ERK is activated mainly by mitogenic stimuli, whereas p38 and JNK/SAPK are activated mainly by stress stimuli or inflammatory cytokines. MAP kinases are part of a three-tiered phosphorylation cascade and MAP kinase phosphorylation on a threonine and tyrosine residue located within the activation loop of kinase subdomain VIII results in activation. However, this process is reversible even in the continued presence of activating stimuli, indicating that protein phosphatases provide an important mechanism for MAP kinase control. Dual specificity phosphatases (DSP's) from tyrosine phosphatase (PTP) gene superfamily are selective for dephosphorylating the critical phosphothreonine and phosphotyrosine residues within MAP kinases. Ten members of dual specificity phosphatases specifically acting on MAPKs, termed MAPK phosphatases (MKPs), have been reported. They share sequence homology and are highly specific for MAPK's but differ in the substrate specificity, tissue distribution, subcellular localization, and inducibility by extracellular stimuli. MKPs have been shown to play important roles in regulating the function of the MAPK family. DSP gene expression is induced strongly by various growth factors and/or cellular stresses. Expression of some gene family members, including CL100/MKP-1, hVH-2/MKP-2, and PAC1, is dependent at least in part on MAP kinase activation providing negative feedback for the inducing MAP kinase or for regulatory cross talk between parallel MAP kinase pathways. DSPs are localized to different subcellular compartments and certain family members appear highly selective for inactivating distinct MAP kinase isoforms. This enzymatic specificity is due to catalytic activation of the DSP phosphatase after tight binding of its amino-terminal to the target MAP kinase. Thus, DSP phosphatases provide a sophisticated mechanism for targeted inactivation of selected MAP kinase activities. p38 MAPKs are members of the MAPK family that are activated by a variety of environmental stresses and inflammatory cytokines. Stress signals are delivered to this cascade by members of small GTPases of the Rho family (Rac, Rho, Cdc42). As with other MAPK cascades, the membrane-proximal component is a MAPKKK, typically a MEKK or a mixed lineage kinase (MLK). The MAPKKK phosphorylates and activated MKK3/5, the p38 MAPK kinase. MKK3/6 can also be activated directly by ASK1, which is stimulated by apoptotic stimuli. P38 MAK is involved in regulation of Hsp27 and MAPKAP-2 and several transcription factors including ATF2, STAT1, THE Max/Myc complex, MEF-2, ELK-1 and indirectly CREB via activation of MSK1. (see Lewis, T. S. et al (1998) Signal transduction through MAP kinase cascades. Adv. Cancer Res. 74, 49-139, which is incorporated in its entirety herein by reference)

The term “MAPK agonist” as used herein refers to any agent which increases or enhances the expression or MAPK or its downstream signaling pathway. An agent can be a small molecule, such as a chemical entity, a peptide, an antibody, antibody fragment or other such agent, etc. An agent which is an agonist of MAPK may include a kinase inhibitor, phosphatase, etc.

The term an “agonist” refers to an agent that binds to a polypeptide or polynucleotide and stimulates, increases, activates, facilitates, enhances activation, sensitizes or up regulates the activity or expression of the polypeptide or polynucleotide. An agonist may inhibit or activate signaling pathways according to its action. An agonist can also be termed an “activator” which is an agent that, e.g., induces or activates the expression of a polypeptide or polynucleotide or binds to, stimulates, increases, opens, activates, facilitates, enhances activation, DNA binding or enzymatic activity, sensitizes or upregulates the activity of a polypeptide or polynucleotide, e.g., agonists. Activation is achieved when the activity value of a polypeptide or polynucleotide relative to the control is 110%, optionally 150%, optionally 200-500%, or 1000-3000% higher.

The term an “antagonist” refers to an agent that inhibits expression of a polypeptide or polynucleotide or binds to, partially or totally blocks stimulation, decreases, prevents, delays activation, inactivates, desensitizes, or down regulates the activity of the polypeptide or the polynucleotide. Inhibitors are agents that, e.g., inhibit expression, e.g., translation, post-translational processing, stability, degradation, or nuclear or cytoplasmic localization of a polypeptide, or transcription, post transcriptional processing, stability or degradation of a polynucleotide of the invention or bind to, partially or totally block stimulation, DNA binding, transcription factor activity or enzymatic activity, decrease, prevent, delay activation, inactivate, desensitize, or down regulate the activity of a polypeptide or polynucleotide of the invention, e.g., antagonists. Inhibitors or antagonists may act directly or indirectly. Inhibition is achieved when the activity value of a polypeptide or polynucleotide relative to the control is about 80%, optionally 50% or 25-1%.

Agonists, activators, inhibitors or antagonists can be naturally occurring and synthetic ligands, antagonists, agonists, small chemical molecules, antibodies, inhibitory RNA molecules (i.e., siRNA or antisense RNA) and the like. Assays to identify inhibitors and activators include, e.g., applying putative modulator compounds to cells, in the presence or absence of a polypeptide or polynucleotide and then determining the functional effects on a polypeptide or polynucleotide.

An “RNA interference molecule” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

The term “differentiation” as used herein refers to the cellular development of a cell from a primitive stage towards a more mature (i.e. less primitive) cell.

The term “directed differentiation” as used herein refers to forcing differentiation of a cell from an undifferentiated (e.g. more primitive cell) to a more mature cell type (i.e. less primitive cell) via genetic and/or environmental manipulation. In some embodiments, a reprogrammed cell as disclosed herein is subject to directed differentiation into specific cell types, such as neuronal cell types, muscle cell types and the like.

The term “functional assay” as used herein is a test which assesses the properties of a cell, such as a cell's gene expression or developmental state by evaluating its growth or ability to live under certain circumstances. In some embodiments, a reprogrammed cell can be identified by a functional assay to determine the reprogrammed cell is a pluripotent state as disclosed herein.

The term “disease modeling” as used herein refers to the use of laboratory cell culture or animal research to obtain new information about human disease or illness. In some embodiments, a reprogrammed cell produced by the methods as disclosed herein can be used in disease modeling experiments.

The term “drug screening” as used herein refers to the use of cells and tissues in the laboratory to identify drugs with a specific function. In some embodiments, the present invention provides drug screening methods of differentated cells to identify compounds or drugs which reprogram a differentated cell to a reprogrammed cell (e.g. a reprogrammed cell which is in a pluripotent state or a reprogrammed cell which is a stable intermediate, partially reprogrammed cell, as disclosed herein). In some embodiments, the present invention provides drug screening methods of stable intermediate partially reprogrammed cells to identify compounds or drugs which reprogramming differentated cells into fully reprogrammed cells (e.g. reprogrammed cells which are in a pluripotent state). In alternative embodiments, the present invention provides drug screening on reprogrammed cells (e.g. human reprogrammed cells) to identify compounds or drugs useful as therapies for diseases or illnesses (e.g. human diseases or illnesses).

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

The term “selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

In some embodiments the selectable marker confers a proliferation and/or survival advantage on cells that express it relative to cells that do not express it or that express it at significantly lower levels. Such proliferation and/or survival advantage typically occurs when the cells are maintained under certain conditions, e.g., “selective conditions”. To ensure an effective selection, a population of cells can be maintained for a under conditions and for a sufficient period of time such that cells that do not express the marker do not proliferate and/or do not survive and are eliminated from the population or their number is reduced to only a very small fraction of the population. The process of selecting cells that express a marker that confers a proliferation and/or survival advantage by maintaining a population of cells under selective conditions so as to largely or completely eliminate cells that do not express the marker is referred to herein as “positive selection”, and the marker is said to be “useful for positive selection”. Negative selection and markers useful for negative selection are also of interest in certain of the methods described herein. Expression of such markers confers a proliferation and/or survival disadvantage on cells that express the marker relative to cells that do not express the marker or express it at significantly lower levels (or, considered another way, cells that do not express the marker have a proliferation and/or survival advantage relative to cells that express the marker). Cells that express the marker can therefore be largely or completely eliminated from a population of cells when maintained in selective conditions for a sufficient period of time.

The term “retrovirus” as used herein refers to a specific type of virus with a RNA-genome that can be engineered to integrate new genetic material into host target cells.

The term “infection” as used herein refers to expose target cells to a mixture of viral particles that contain new genetic material one wishes to functionally evaluate

The term “lentivirus” as used herein refers to a specific type of virus with an RNA genome (such as HIV) that can be engineered to deliver and integrate new genetic material into target cells. Lentivirus has certain advantages over other retroviruses including that it can deliver its genetic payload to the nucleus of non-dividing target cells. The term “transcriptional profile” as used herein refers to the state of gene expression in a given cell or tissue type

The term “transduction” as used herein refers to the use of viral particles to introduce new genetic material into a cell

The term “transfection” as used herein refers the use of chemical methods, most often lipid containing vesicles, to introduce new genetic material into a cell

The term “transformation” as used herein refers to when a cell becomes functionally abnormal in the process of malignancy, often obtaining a new capacity to multiply indefinitely or under new circumstances

The term “oncogene” refers to a gene initially identified via its role in certain cancers. Oncogenes may cause or contribute to cancer. Oncogenes encompassed herein also include abnormal genes or non-functional or truncated genes as a result of the insertional inactivation (e.g. for example, insertion of viral genetic material into the gene sequence) which cause or contribute to cancer. Examples of oncogenes are c-myc and sox and the like.

The terms “subject” and “individual” are used interchangeably herein, and refer to an animal, for example, a human from whom cells can be obtained (e.g. differentiated cells can be obtained which are reprogrammed) and/or to whom treatment, including prophylactic treatment, with the reprogrammed cells (or their differentiated progeny) as described herein, is provided. For treatment of conditions or disease states which are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like are also encompassed in the term subject.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment may improve the disease condition, but may not be a complete cure for the disease. As used herein, the term “treatment” includes prophylaxis. Alternatively, treatment is “effective” if the progression of a disease is reduced or halted. In some embodiments, the term “treatment” can also mean prolonging survival as compared to expected survival if not receiving treatment. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management. The subject is usually ill or injured, or at an increased risk of becoming ill relative to an average member of the population and in need of such attention, care, or management.

The terms “treat”, “treating”, “treatment”, etc., as used herein can refer to administration to the subject of a composition comprising one or more reprogramming factors (e.g. any agent selected from any of the compounds of Formulas I-VIII, such as an exemplary compound RepSOX (E-616452)), or alternatively, administration of a reprogrammed cell or a differentiated progeny thereof (or isolated populations thereof) to a subject.

As used herein, the terms “administering,” “introducing” and “transplanting” are used interchangeably in the context of the placement of reprogrammed cells as disclosed herein, or their differentiated progeny into a subject, by a method or route which results in at least partial localization of the reprogrammed cells, or their differentiated progeny at a desired site. The reprogrammed cells, or their differentiated progeny can be administered directly to a tissue of interest, or alternatively be administered by any appropriate route which results in delivery to a desired location in the subject where at least a portion of the reprogrammed cells or their progeny or components of the cells remain viable. The period of viability of the reprogrammed cells after administration to a subject can be as short as a few hours, e.g. twenty-four hours, to a few days, to as long as several years.

The term “transplantation” as used herein refers to introduction of new cells (e.g. reprogrammed cells), tissues (such as differentated cells produced from reprogrammed cells), or organs into a host (i.e. transplant recipient or transplant subject)

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of cardiovascular stem cells and/or their progeny and/or compound and/or other material other than directly into the central nervous system, such that it enters the animal's system and, thus, is subject to metabolism and other like processes, for example, subcutaneous administration.

The term “tissue” refers to a group or layer of specialized cells which together perform certain special functions. The term “tissue-specific” refers to a source of cells from a specific tissue.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “halo” refers to any radical of fluorine, chlorine, bromine or iodine.

The term “acyl” refers to an alkylcarbonyl, cycloalkylcarbonyl, arylcarbonyl, heterocyclylcarbonyl, or heteroarylcarbonyl substituent, any of which may be further substituted by substituents. Exemplary acyl groups include, but are not limited to, (C₁-C₆)alkanoyl (e.g., formyl, acetyl, propionyl, butyryl, valeryl, caproyl, t-butylacetyl, etc.), (C₃-C₆)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions.

The term “alkyl” refers to saturated non-aromatic hydrocarbon chains that may be a straight chain or branched chain, containing the indicated number of carbon atoms (these include without limitation propyl, allyl, or propargyl), which may be optionally inserted with N, O, S, SS, SO₂, C(O), C(O)O, OC(O), C(O)N or NC(O). For example, C₁-C₆ indicates that the group may have from 1 to 6 (inclusive) carbon atoms in it.

The term “alkenyl” refers to an alkyl that comprises at least one double bond. Exemplary alkenyl groups include, but are not limited to, for example, ethenyl, propenyl, butenyl, 1-methyl-2-buten-1-yl and the like.

The term “alkynyl” refers to an alkyl that comprises at least one triple bond.

The term “alkoxy” refers to an —O-alkyl radical.

The term “aminoalkyl” refers to an alkyl substituted with an amino. The term “mercapto” refers to an —SH radical.

The term “thioalkoxy” refers to an —S-alkyl radical.

The term “aryl” refers to monocyclic, bicyclic, or tricyclic aromatic ring system wherein 0, 1, 2, 3, or 4 atoms of each ring may be substituted by a substituent. Exemplary aryl groups include, but are not limited to, phenyl, naphthyl, anthracenyl, azulenyl, fluorenyl, indanyl, indenyl, naphthyl, phenyl, tetrahydronaphthyl, and the like.

The term “arylalkyl” refers to alkyl substituted with an aryl.

The term “cyclyl” or “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “heteroarylalkyl” refers to an alkyl substituted with a heteroaryl.

The term “heterocyclyl” refers to a nonaromatic 5-8 membered monocycle, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Exemplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups include, but are not limited to chloromethyl, bromoethyl, trifluoromethyl, and the like.

The term “optionally substituted” means that the specified group or moiety, such as an aryl group, heteroaryl group and the like, is unsubstituted or is substituted with one or more (typically 1-4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, heteroaryl, acyl, amino group at any atom of that group. Suitable substituents include, without limitation, halo, hydroxy, oxo, nitro, haloalkyl, alkyl, alkenyl, alkynyl, alkaryl, aryl, aralkyl, alkoxy, aryloxy, amino, acylamino, alkylcarbanoyl, arylcarbanoyl, aminoalkyl, alkoxycarbonyl, carboxy, hydroxyalkyl, alkylthio, CF₃, N-morphilino, phenylthio, alkanesulfonyl, arenesulfonyl, alkanesulfonamido, arenesulfonamido, aralkylsulfonamido, alkylcarbonyl, acyloxy, cyano or ureido. In some embodiments substituent can itself be optionally substituted. In some cases, two substituents, together with the carbons to which they are attached to can form a ring.

In many cases, protecting groups are used during preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, for example, are disclosed by Beaucage et al. (Tetrahedron 1992, 48, 2223-2311). Further hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y, 1991. Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p′-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate.

Nitrogen- or amino-protecting groups stable to acid treatment are selectively removed with base treatment, and are used to make reactive amino groups selectively available for substitution. Exemplary amino-protecting groups include, but are not limited to, carbamate protecting groups, such as 2-trimethylsilylethoxycarbonyl (Teoc), 1-methyl-1-(4-biphenyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### Methods of Reprogramming Cells

As described herein, a reprogrammed cell can be produced by contacting a cell with one or more small molecules which replace one or more of the reprogramming transcription factors which encode transcription factors selected from the Sox family (e.g. Sox2), the Klf family (e.g. Klf4) and the Oct family (e.g. Oct3/4).

In some embodiments, a reprogrammed cell is produced by contacting a cell with at least one small molecule which replaces a transcription factor from the Sox family of transcription factors. For example, contacting a differentiated cell with a compound of Formula I, such as RepSox or SB431542 enables reprogramming of differentiated cells by only 3 transcription factors, Oct-4, Klf-4 and c-Myc without the need for Sox-2, or only 2 transcription factors, Oct-4 and Klf-4 without the need for c-Myc or Sox-2. As disclosed herein, the inventors demonstrate production of reprogrammed cells (e.g. iPS colonies) from mouse embryonic fibroblasts (MEFs) which expressed exogenous transcription factors (by retroviral expression) of Oct-4 and Klf-4 together with RepSox treatment. The number and percentage of reprogrammed cells (e.g. iPS colonies) was comparable to those in the addition of the exogenous Sox-2 transgene. In addition, the 3-factor reprogramming efficiency using RepSox treatment with Oct-4 and Klf4 is comparable to the induction rate for mouse fibroblasts infected by 4 factors (Oct-4, Klf-4, c-Myc and Sox-2), demonstrating RepSox treatment effectively replaced the need for exogenous Sox-2 transcription factor.

In some embodiments, a reprogrammed cell is produced by contacting a cell with two or more small molecule which replaces a transcription factor from the Sox family of transcription factors (such as Sox2), and a transcription from the Klf family of transcription factors (such as Klf4). In some embodiments, a reprogrammed cell is produced by contacting a cell with two or more small molecule which replaces a transcription factor from the Sox family of transcription factors (such as Sox2), and a transcription from the Oct family of transcription factors (such as Oct 3/4). In another embodiment, a reprogrammed cell is produced by contacting a cell with three or more small molecule which replaces a transcription factor from the Sox family of transcription factors (such as Sox2), and a transcription from the Klf family of transcription factors (such as Klf4), and a transcription factor from the Oct family of transcription factors, (such as Oct 3/4).

In some embodiments, a reprogrammed cell is produced by contacting a cell with at least one small molecule which replaces a transcription factor from the Klf family of transcription factors. In some embodiments, a reprogrammed cell is produced by contacting a cell with two or more small molecule which replaces a transcription factor from the Klf family of transcription factors (such as Kfl4), and a transcription from the Oct family of transcription factors (such as Oct4).

In another embodiment, a reprogrammed cell is produced by contacting a cell with at least one small molecule which replaces a transcription factor from the Oct family of transcription factors. In some embodiments, a reprogrammed cell is produced by contacting a cell with two or more small molecule which replaces a transcription factor from the Oct4 family of transcription factors (such as Oct4), and a transcription from the Klf4 family of transcription factors (such as Klf4).

In some embodiments, a reprogrammed cell is produced by contacting a cell with two or more small molecule which replaces a transcription factor from the Sox family of transcription factors (such as Sox2), and a transcription from the Oct family of transcription factors (such as Oct 3/4).

In some embodiments, the present invention also provides a method for reprogramming a differentiated cell comprising contacting the differentiated cell with at least one compound selected from a group of compounds of Formulas I-XI (e.g., a TGFBR1 inhibitor(s) of Formulas I, 111-VII, including RepSox and/or SB-431542), Src inhibitors (e.g. compounds of Formula II), agonist of MEK or Erk cell signaling (e.g. compounds with Formula VIII, such as Prostaglandin 2); inhibitors of Ca²⁺/calmodulin signaling or EGF receptor tyrosine kinase inhibitor (e.g. any compound with Formula XI, such as HBDA); inhibitors of Na²⁺ channels or ATP-dependent potassium channel (e.g. compounds with Formula X, such as Sinimenine), or agonists of MAPK signaling pathway (e.g. compounds with Formula XI, such as Ropivocaine or Bupivacaine). In some embodiments, a differentiated cell can also be contacted with a gene product (e.g. nucleic acid or polypeptide) of one or more kinds of the following transcription factor genes: Oct3/4, Sox2, Klf4, Nanog, Lin-28, and c-Myc.

Another aspect relates to a composition comprising any combination of compounds selected from the group of Formulas I-XI (e.g., a TGFBR1 inhibitor(s) of Formulas I, III-VII, including RepSox and/or SB-431542), Src inhibitors (e.g. compounds of Formula II), agonist of MEK or Erk cell signaling (e.g. compounds with Formula VIII, such as Prostaglandin 2); inhibitors of Ca2+/calmodulin signaling or EGF receptor tyrosine kinase inhibitor (e.g. any compound with Formula XI, such as HBDA); inhibitors of Na²⁺ channels or ATP-dependent potassium channel (e.g. compounds with Formula X, such as Sinimenine), or agonists of MAPK signaling pathway (e.g. compounds with Formula XI, such as Ropivocaine or Bupivacaine), or small molecules and/or substances as described herein. In some embodiments, the composition further comprises one or more factors improving the efficiency of reprogramming a differentiated cell to a reprogrammed cell (e.g. an iPS cell), such as one or more small molecules such as VPA or HDAC inhibitors.

The present invention also provides a method for improving ability of differentiation and/or growth of a cell, which comprises the step of contacting the differentiated cell with a compound selected from any or a combination of compounds of Formulas I-XI (e.g., a TGFBR1 inhibitor(s) of Formulas I, III-VII, including RepSox and/or SB-431542), Src inhibitors (e.g. compounds of Formula II), agonist of MEK or Erk cell signaling (e.g. compounds with Formula VIII, such as Prostaglandin 2); inhibitors of Ca2+/calmodulin signaling or EGF receptor tyrosine kinase inhibitor (e.g. any compound with Formula XI, such as HBDA); inhibitors of Na⁺ channels or ATP-dependent potassium channel (e.g. compounds with Formula X, such as Sinimenine), or agonists of MAPK signaling pathway (e.g. compounds with Formula XI, such as Ropivocaine or Bupivacaine), and further provides a reprogrammed cell obtained by the methods as disclosed herein, and a differentiated-reprogrammed cell as that term is defined herein by inducing differentiation of a chemically-reprogrammed cell obtained by the methods as disclosed herein.

In some embodiments, the present invention further provides a method for stem cell therapy, which comprises the step of transplanting a reprogrammed cell that has been differentiated in to a certain cell type, wherein the cell is obtained by reprogramming of a differentiated cell into a reprogrammed cell (e.g. an iPS cell or partially reprogrammed cell) according to the methods as disclosed herein, wherein the differentiated cell used for reprogramming was isolated and collected from a subject, such as a human subject, and then transplanted back into the same or a different subject. Several kinds of, preferably approximately 200 kinds of chemically induced reprogrammed cells produced by the methods as disclosed herein can be prepared from differentiated cells derived from healthy humans can be stored in an iPS cell bank as a library of reprogrammed cells, and one kind or more kinds of the reprogrammed cells (e.g. iPS cells or partially reprogrammed cells) in the library can be used for preparation of somatic cells, tissues, or organs that are free of rejection by a subject to be subjected to stem cell therapy. In some embodiments, the chemically induced reprogrammed cells produced by the methods as disclosed herein can be partially reprogrammed cells (e.g. cells which are not fully reprogrammed to a pluripotent state, such as the stable intermediate non-pluripotent cells as disclosed herein in the Examples 5-7) derived from healthy humans can be stored in an iPS cell bank.

Another aspect of the present invention also relates to a method for evaluating a physiological function or toxicity of a compound, a medicament, a poison or the like by using various cells obtained by reprogramming a differentiated cell to become a chemically induced reprogrammed cell (e.g. an iPS cell or partially reprogrammed cells) according to the methods as disclosed herein.

Chemical Replacement of Sox family of transcription factors

One aspect of the present invention relates to a method to produce a reprogrammed cell by contacting a differentiated cell with at least one small molecule, selected from any compound with Formula I-VII which replaces an exogenous transcription factor from the Sox family of transcription factors. Examples of the Sox family of transcription factors include, for example, Sox1, Sox2, Sox3, Sox7, Sox15, Sox17 and Sox18, and a preferred example includes Sox2. Sox2, expressed in an early development process, is a gene encoding a transcription factor (Avilion et al., Genes Dev. 17:126-40, 2003). The NCBI accession numbers of Sox family genes are follows: Sox1: Sox1 SRY-box containing gene 1, NM_{—}009233 (mouse), NM_{—}005986 (human); Sox2: NM_{—}011443 (mouse) (SEQ ID NO:1), NM_{—}003106 (human) (SEQ ID NO:2); Sox3 SRY-box containing gene 3, NM_{—}009237 (mouse), NM_{—}005634 (human); Sox7 SRY-box containing gene 7 NM_{—}011446 (mouse), NM_{—}031439 (human); Sox15 SRY-box containing gene 15 NM_{—}009235, (mouse) NM_{—}006942 (human); Sox17 SRY-box containing gene 17 NM_{—}011441 (mouse), NM_{—}022454 (human); Sox18 SRY-box containing gene 18 NM_{—}009236 (mouse), NM_{—}018419 (human).

In one embodiment, any compound selected from any of Formula I-VII, such as any compound which is a TGFBR1 inhibitor, such as from Formula I, III-VII (e.g. Repsox, E-616541 or SB431542), or any compound which is a Src signaling pathway inhibitor (such as a compound of formula II, such as EI-275) can be used to reprogram a differentiated cell, and can be used in any combination of members from one or more transcription factors gene families. For example, a combination of one or more gene products of Oct3/4, Klf4, and c-Myc.

In one embodiment, any compound from Formula I-VII, can be with or without a Myc family gene transcription factor. In a preferred embodiment, a member of the Myc family of transcription factors is absent when a compound of Formulas I-VII is used in the reprogramming of a differentiated cell. Examples of the Myc family gene include c-Myc, N-Myc, L-Myc and the like. c-Myc is a transcription control factor involved in differentiation and proliferation of cells (Adhikary & Eilers, Nat. Rev. Mol. Cell. Biol. 6:635-45, 2005), and is also reported to be involved in the maintenance of pluripotency (Cartwright et al., Development 132:885-96, 2005). The accession numbers of members of the myc family are: c-Myc myelocytomatosis oncogene, NM_{—}010849 (mouse), NM_{—}002467 (human); N-Myc v-Myc myelocytomatosis viral related oncogene, NM_{—}008709 (mouse), NM_{—}005378 (human); neuroblastoma derived (avian) L-Myc v-Myc myelocytomatosis viral oncogene, NM_{—}008506 (mouse), NM_{—}005376 (human).

In one embodiment, replacement of exogenous transcription factor Sox2 is by an agent which is an inhibitor of the TGFβ signaling pathway, such as a TGFBR1 inhibitor. In some embodiments, replacement of exogenous transcription factor Sox2 is by any compound with the formula selected from Formulas I, III-VII. In some embodiments, where a differentiated cell is contacted with an inhibitor of the TGF pathway, or an inhibitor of TGFBR1, or a compound with the Formula selected from Formulas I, III-VII, the cell is not contacted with an exogenous Sox, such as Sox2 transgene or Sox2 protein. In some embodiments, replacement of exogenous transcription factor Sox2 is by any compound with Formula I such as Repsox (E-616452) or E-616451. In another embodiment, replacement of exogenous transcription factor Sox2 is by any compound with Formula III such as SB431542 (Formula III).

In one embodiment, replacement of exogenous transcription factor Sox2 is by an agent which is an inhibitor of the SRC signaling pathway, such as a SRC inhibitor. In some embodiments, replacement of exogenous transcription factor Sox2 is by any compound with the Formula II. In some embodiments, where a differentiated cell is contacted with an inhibitor of the SRC pathway, or a compound with the Formula II, the cell is not contacted with an exogenous Sox, such as Sox2 transgene or Sox2 protein. In some embodiments, replacement of exogenous transcription factor Sox2 is by any compound with Formula II such as EI-275.

In some embodiments, contact of a differentiated cell with an agent which replaces Sox2, (e.g. inhibitor of TGF signaling, such as a TGFB1 inhibitor, or a SRC inhibitor, or any compound with Formulas I-VII, including but not limited to Repsox (E-616452), E-616451, SB431542 and EI-275, enables reprogramming of differentiated cells by only 3 transcription factors, such as Oct-4, Klf-4 and c-Myc without the need for Sox-2. In some embodiments, contact of a differentiated cell with an agent which replaces Sox2 requires only 2 transcription factors, Oct-4 and Klf-4 without the need for c-Myc or Sox-2.

In some embodiments, a differentiated cell which is contacted with an agent which replaces Sox2, (e.g. where a differentiated cell is contacted with an inhibitor of TGF signaling, such as a TGFB1 inhibitor, or a SRC inhibitor, or any compound with Formulas I-VII, including Repsox (E-616452), E-616451, SB431542 and EI-275), can be reprogrammed with small molecules or other agents which replace transcription factors from the Oct and Klf family of transcription factors as disclosed herein, thus if is not necessary to contact the differentiated cell with exogenous Oct-4 and Klf-4 transcription factors as disclosed herein.

Thus, described herein are methods for producing reprogrammed cells from differentiated cells (e.g. from fibroblasts e.g., MEFs) without using the oncogenes, for example c-Myc or oncogenes associated with introduction of nucleic acid sequences encoding the transcription factors Sox-2, Oct-4 or Klf-4 into the differentiated cell to be reprogrammed (e.g. viral oncogenes). For example, the chemical mediated reprogramming of differentiated cells makes it possible to create reprogrammed cells (e.g. iPS cells or partially reprogrammed cells) from small numbers of differentiated cells (e.g., such as those obtained from hair follicle cells from patients, blood samples, adipose biopsy, fibroblasts, skin cells, etc). In one embodiments, the addition of small molecules compounds (e.g., chemicals) allows successful and safe generation of reprogrammed cells (e.g. iPS cells or partially reprogrammed cells) from human differentiated cells, such as skin biopsies (fibroblasts or other nucleated cells) as well as from differentiated cells from all and any other cell type.

Inhibitors of TGF β-Receptor Cell Signaling

In some embodiments, a chemically-induced reprogrammed cell be produced by contacting a differentiated cell with an inhibitor of TGFβ cell signaling. The Transforming growth factor beta (TGFβ) signaling pathway is involved in many cellular processes in both the adult organism and the developing embryo including cell growth, cell differentiation, apoptosis, cellular homeostasis and other cellular functions. In spite of the wide range of cellular processes that the TGFβ signaling pathway regulates, the process is relatively simple. TGF β superfamily ligands bind to a type II receptor, which recruits and phosphorylates a type I receptor. The type I receptor then phosphorylates receptor-regulated SMADs (R-SMADs) which can now bind the coSMAD SMAD4. R-SMAD/coSMAD complexes accumulate in the nucleus where they act as transcription factors and participate in the regulation of target gene expression.

TGFβ receptors are single pass serine/threonine kinase receptors. They exist in several different isoforms that can be homo- or heterodimeric. The number of characterized ligands in the TGFβ superfamily far exceeds the number of known receptors, suggesting the promiscuity that exists between the ligand and receptor interactions.

TGF can be found in many different tissue types, including brain, heart, kidney, liver and testes. Over-expression of TGF can induce renal fibrosis, causing kidney disease, as well as diabetes, and ultimately end-stage renal disease (ESRD). Recent developments have found that, using certain types of protein antagonists against TGFβ receptors, can halt and in some cases reverse the effects of renal fibrosis.

Three TGF-β receptor types can be distinguished by their structural and functional properties. Receptor types I and II have similar ligand binding affinities and can only be distinguished from each other by peptide mapping, both receptor types I and II have a high affinity for TGF-β1 and low affinity for TGF-β2. TGF-β receptor type III has a high affinity for both TGF-β1 and -β2 and in addition TGF-β1.2.

Transforming growth factor, beta receptor I (herein termed “TGFBR1”) (activin A receptor type II-like kinase, 53 kDa) is a TGF beta receptor. TGFBR1 is its human gene. The protein encoded by this gene forms a heteromeric complex with type II TGF-beta receptors when bound to TGF-beta, transducing the TGF-beta signal from the cell surface to the cytoplasm. The encoded protein is a serine/threonine protein kinase. Mutations in this gene have been associated with Loeys-Dietz aortic aneurysm syndrome (LDAS).

Transforming growth factor, beta receptor II (70/80 kDa) is a TGF beta receptor. TGFBR2 is its human gene. This gene encodes a member of the Ser/Thr protein kinase family and the TGFB receptor subfamily. The encoded protein is a transmembrane protein that has a protein kinase domain, forms a heterodimeric complex with another receptor protein, and binds TGF-beta. This receptor/ligand complex phosphorylates proteins, which then enter the nucleus and regulate the transcription of a subset of genes related to cell proliferation. Mutations in this gene have been associated with Marfan Syndrome, Loeys-Deitz Aortic Aneurysm Syndrome, Osler-Weber-Rendu syndrome, and the development of various types of tumors. Alternatively spliced transcript variants encoding different isoforms have been characterized.

TGFβ Receptor

The TGF-β receptors contemplated for use in the methods described herein for the replacement of Sox can be any TGF-β receptor including those from the Activin-like kinase family (ALK), the Bone Morphogenic Protein (BMP) family, the Nodal family, the Growth and Differentiation Factors family (GDF), and the TGF-β receptor family of receptors. TGF-β receptors are serine/threonine kinase receptors that effect various growth and differentiation pathways in the cell.

In one embodiment, a TGF-β receptor useful for the methods described herein for the replacement of Sox2 is an ALK4, ALK5, or ALK7 receptor. In another embodiment, the TGF-β receptor inhibited by the methods described herein for the replacement of Sox2 is an ALK5 receptor. In another embodiment, downstream effectors of any of the aforementioned TGF-beta receptor signaling pathways can be targeted directly to effect cell reprogramming with the methods described herein.

If desired, one of skill in the art can locate the protein sequence of any of the TGF-β receptors by simply searching “transforming growth factor beta receptor” in a protein sequence database such as NCBI. Some non-limiting examples of protein sequence accession numbers for TGF-β receptors are P36897.1 (SEQ ID NO: 3), Q5T7S2 (SEQ ID NO: 4), Q61R47, P37173 (SEQ ID NO: 5), Q6A176 (not shown), Q706C0 (not shown), Q706C1 (not shown), and Q03167.2 (SEQ ID NO: 6), among others.

TGF-β1 is a prototypic member of a family of cytokines including the TGF-βs, activins, inhibins, bone morphogenetic proteins and Mullerian-inhibiting substance, that signal through a family of single transmembrane serine/threonine kinase receptors. These receptors can be divided into two classes, the type I or activin like kinase (ALK) receptors and type H receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The G_{S} domain is at the amino terminal end of the intracellular kinase domain and is critical for activation by the type II receptor. Several studies have shown that TGF-β signaling requires both the ALK and type H receptors. Specifically, the type II receptor phosphorylates the GS domain of the type I receptor for TGF-β, ALK5, in the presence of TGF-β. The ALK5, in turn, phosphorylates the cytoplasmic proteins Smad2 and Smad3 at two carboxy terminal serines. The phosphorylated Smad proteins translocate into the nucleus and activate genes that contribute to e.g., the production of extracellular matrix.

Activin ligands transduce signals in a manner similar to TGF-β ligands. Activins bind to and activate ALK receptors, which in turn phosphorylate Smad proteins such as Smad2 and Smad3. The consequent formation of a hetero-Smad complex with Smad4 results in the activin-induced regulation of gene transcription.

Smad proteins are exemplary downstream signal transduction factors in the TGF-beta pathway and therefore can be activated or inhibited directly to effect reprogramming (e.g., by treating a cell with an activator or inhibitor of a Smad protein). In one embodiment, an activator of Smad 7 is used to effect cell reprogramming. In another embodiment, inhibition of Smad 2, 3, or 5 is used to effect cell reprogramming

TGF-Beta Receptor (TGFβR) Inhibitors

As used herein, the term “TGF-β signaling inhibitor” or “TGFβR inhibitor” or “TGFBR inhibitor” is any agent or small molecule (e.g. a compound) that inhibits TGF-β signal transduction by inhibiting any of the factors constituting the TGF-β signal transduction system pathway, such as TGF-β ligand, TGF-β Type I receptors, TGF-β Type II receptors, TGF-β Type III receptors (β-glycan and endoglin), soluble forms of the TGF-β receptors, Smad proteins (1-8). A TGFBR inhibitor is any agent, including small molecules, antibodies against receptors and ligands implicated in the signaling pathway, nucleic acid based molecules (e.g., antisense, siRNA, aptamers and ribozymes) targeting the pathway members, or a combination thereof.

An “inhibitor” of a TGFβR, as the term is used herein, can function in a competitive or non-competitive manner, and can function, in one embodiment, by interfering with the expression of the TGFβR polypeptide. A TGFβR inhibitor includes any chemical or biological entity that, upon treatment of a cell, results in inhibition of a biological activity caused by activation of the TGFβR in response to binding of its natural ligand. While any TGF-β signaling pathway inhibitor can potentially be used in the methods described herein, it is preferable that a TGF-β signaling pathway inhibitor is either selective for, or specific for, a member of the TGF-β signaling pathway. By “specific” is meant that at the dose necessary for the inhibiting agent to inhibit the TGF-β signaling pathway, the inhibiting agent does not have any other substantial pharmacological action in the cell or host. By “selective” is meant that the dose of the inhibitor necessary for inhibition of the TGF-β signaling pathway is at least 2-fold lower than the dose necessary for activation or inhibition of another pharmacological action as measured by the ED₅₀ or EC₅₀ of the agent for each pharmacological effect; preferably the dose of inhibitor necessary for TGF-β pathway inhibition is at least 5-fold lower, at least 10 fold lower, at least 20-fold lower, at least 30-fold lower, at least 40-fold lower, at least 50-fold lower, at least 60-fold lower, at least 70-fold lower, at least 80-fold lower, at least 90-fold lower, at least 100-fold lower, at least 500-fold lower, at least 1000 fold lower or more, than the dose necessary for another pharmacological action. Thus, to be clear, the agents useful for the methods described herein primarily inhibit the TGF-β signaling pathway with only minor, if any, effects on other pharmacological pathways, and the dose used for inhibition of the TGF-β signaling pathway is sub-clinical or sub-threshold for other pharmacological responses.

Such an inhibitor can act by binding to the intracellular domain of the receptor and blockade of its serine/threonine kinase activity (e.g., ATP binding site). Alternatively, such an inhibitor can act by occupying or sterically hindering the ligand binding site (or a portion thereof) of the TGFβR, thereby rendering the receptor inaccessible to binding by the natural ligand, which prevents activation by that ligand. In addition, the TGFβR inhibitor can also bind to a non-ligand binding site and, for example, produce a conformational shift in the TGFβR, such that a ligand of the TGFβR can no longer access the binding site. An inhibitor can be, for example, a competitive inhibitor, a non-competitive inhibitor, an inverse agonist or a partial agonist of the TGFβR.

Alternatively, such an inhibitor can act by modulating the heterodimerization of TGFβR polypeptides, the interaction of TGFβR with other proteins, or the ubiquitination or endocytic degradation of the receptor. TGFβR inhibitors, include, but are not limited to small molecules, antibodies or antigen-binding antibody fragments, antisense constructs, siRNAs and ribozymes.

The receptor activity of a TGF-β receptor can be measured, for example, as described by Laping, N.J., et al (2002) Molecular Pharmacology 62(1):58-64, which is herein incorporated by reference in its entirety. In addition, the dose-response curve for a TGF-β receptor inhibitor can be determined by measuring TGF-β receptor activity over a variety of inhibitor concentrations using the method of Laping, N.J., et al (2002).

Small Molecule Inhibitors of TGFβ (TGFBR1 Inhibitors)

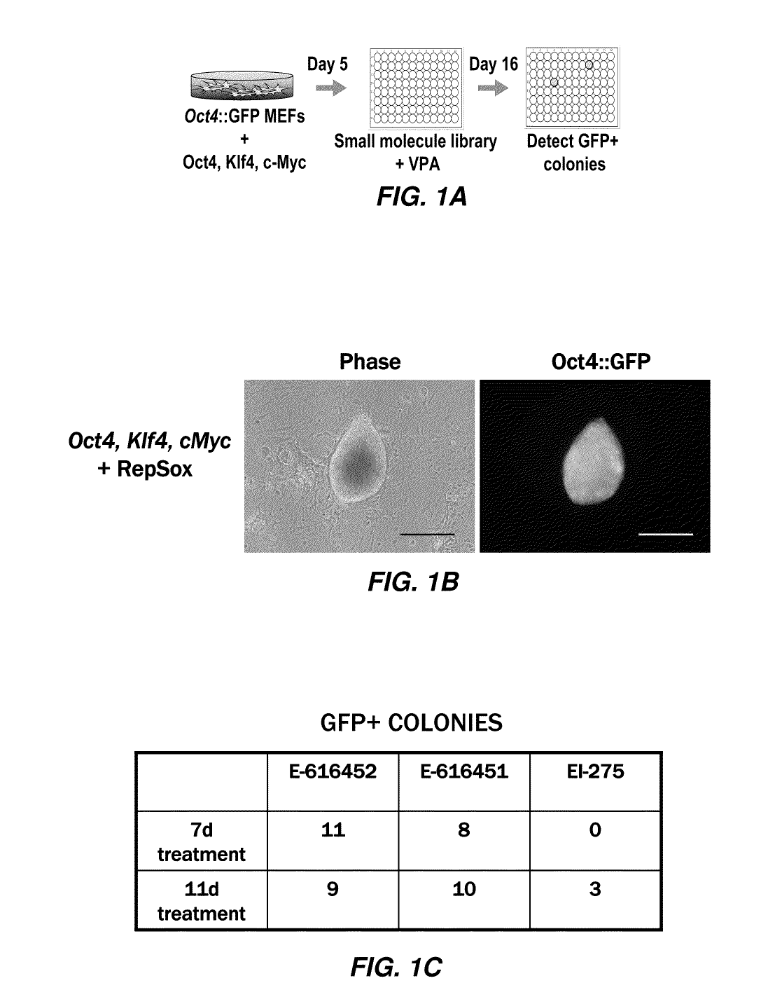

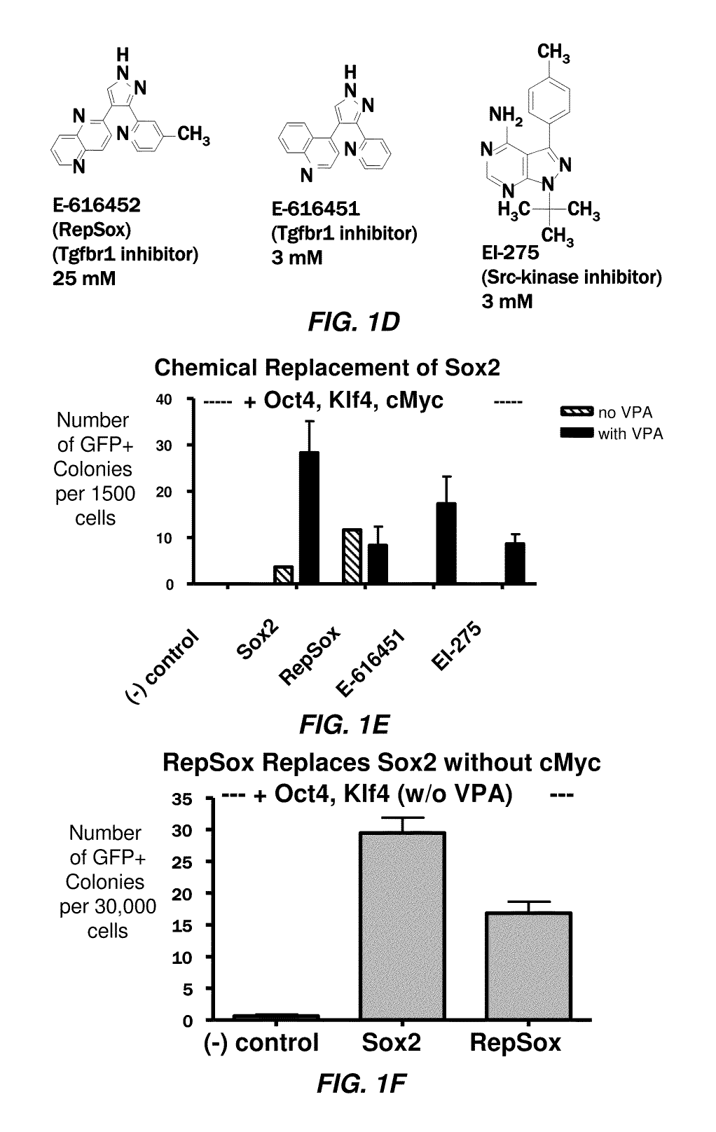

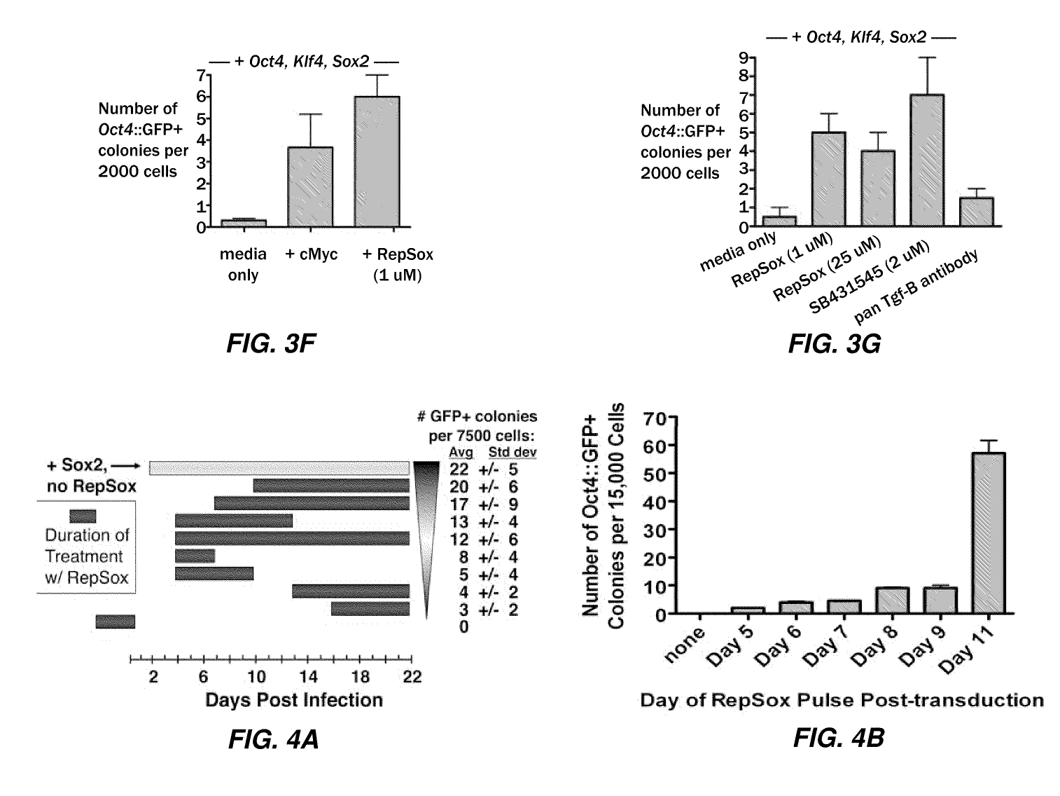

Described herein are compounds that can be used in the methods and kits described herein for the replacement of sox2, for example, in methods of producing a reprogrammed cell (e.g. iPS cell or partially reprogrammed cells) from a differentiated cell. Exemplary compounds for use in the methods and kits described herein as TGFβ inhibitors, such as TGFBR1 inhibitors include those described generically (e.g., the compounds of Formula (I), and (III), (IVa), (IVb), (V), (VI), (VI), (VII), or (VIII)) and also those described specifically, e.g., the compounds depicted in FIG. 1D (E-616452, also described herein as RepSox), FIG. 1D (compound B, E-616451) and FIG. 3A (described herein as SB431542).

Formula I

In one aspect, the disclosure features a method of producing a reprogrammed cell (e.g. iPS cell or partially reprogrammed cell) from a differentiated cell, the method comprising:

contacting an isolated differentiated cell with a compound of formula (I)

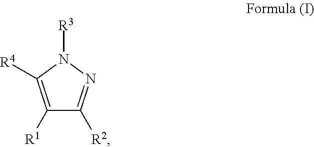

wherein

R¹ cyclyl, heterocyclcyl, aryl or heteroaryl, each of which can be optionally substituted;

R² cyclyl, heterocyclcyl, aryl or heteroaryl, each of which can be optionally substituted;

R³ is H, C₁-C₆ alkyl, arylC₁-C₆, or a nitrogen protecting group, each of which can be optionally substituted;

R⁴ is H, optionally substituted C₁-C₆ alkyl, optionally substituted C₂-C₆ alkenyl, optionally substituted C₂-C₆ alkynyl, or R³ and R⁴ together with the atoms they are attached to form a cyclyl, heterocyclyl, aryl or heteroaryl, each of which can be optionally substituted;

to thereby produce a reprogrammed cell (e.g. iPS cell or partially reprogrammed cells) from the differentiated cell.

In one embodiment, the method comprises contacting a plurality of differentiated cells with a compound of formula (I) to thereby produce a plurality of reprogrammed cells (e.g. iPS cells or partially reprogrammed cell) from the differentiated cells.

In some embodiments, R¹ is aryl, e.g., a substituted aryl. In some embodiments, R¹ is substituted with two substituents. In some embodiments, R¹ is substituted with two substituents, which together with the carbons to which they are attached form a ring. In some embodiments, R¹ is a substituted phenyl. In some embodiments, R¹ is a nitrogen containing heteroaryl (e.g., including 1, 2, or 3 nitrogens (e.g., 1 or)). In some embodiments, R¹ is a bicyclic heteroaryl. In some embodiments, R¹ is a 6-6 fused heteroaryl. In some embodiments, R¹ is pyridyl, pyrimidyl, pyridazinyl, pyrazinyl, quinolinyl, naphthyridinyl (e.g., 1,5-naphthyridinyl), quinazolinyl, 5,6,7,8-tetrahydroquinazolinyl, 1,3-benzodioxlyl, 1,2,3-benzotriazolyl, benzoxazolyl, benzothiazolyl, 2,1,3-benzooxadiazole, imidazo[1,2-a]pyridinyl, pyrazolo[1,5-a]pyridinyl, [1,2,4]triazolo[1,5-a]pyridinyl, pyrazolo[1,5-a]pyrimidinyl, [1,2,4]triazolo[1,5-a]pyrimidinyl, [1,2,3]triazolo[1,5-a]pyrimidinyl, [1,2,4]triazolo[4,3-a]pyrimidinyl, [1,2,4]triazolo[4,3-a]pyridazinyl. In some embodiments, R¹ is

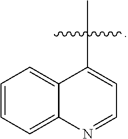

In some embodiments, R¹ is

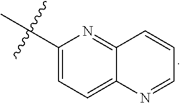

In some embodiment, R² is aryl, e.g., a substituted aryl. In some embodiments, R² is a nitrogen comprising heteroaryl (e.g., including 1, 2 or 3 nitrogens (e.g., 1 or 2)). In some embodiments, R² is an optionally substituted monocyclic heteroaryl (e.g., a six membered heteroaryl such as pyridyl, pyrimidyl, pyridazinyl or pyrazinyl). In some embodiments, R² is substituted. Exemplary substituents include halo, C₁-C₆ alkyl, haloC₁-C₆alkyl, C₁-C₆ alkoxy, OH, haloC₁-C₆alkoxy. In some embodiments, R² is monosubstituted. In some embodiments, R² is substituted with methyl. In one embodiment, R² is an optionally substituted pyridyl.

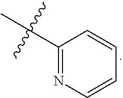

In some embodiments, R² is

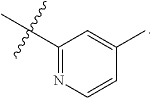

In some embodiments, R² is

In one embodiment, R⁴ is H.

In one embodiment, the compound of formula (I) has the structure shown in formula (Ia):

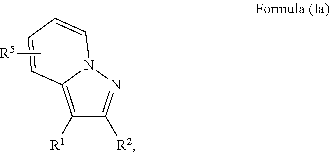

wherein R⁵ is H, benzyl, aryl, heteroaryl, C₁-C₆alkyl, alkenyl, alkynyl, halogen, amino, —C(O)-amino, —SO₂-alkyl, —O-alkyl or acyl, each of which can be optionally substituted.

In some embodiments, R⁵ is H.

In one embodiment, the compound of formula (I) has the structure shown in formula (Ib):

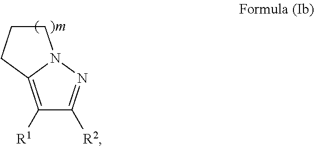

wherein m is 1, 2 or 3.

Exemplary compounds of formula (I) include:

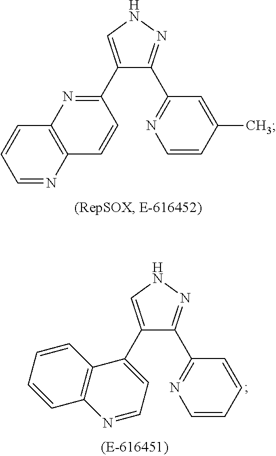

4-[2-(6-Ethyl-pyridin-2-yl)-pyrazolo[1,5-a]pyridin-3-yl]-quinoline;
[2-(6-Methyl-pyridin-2-yl)-pyrazolo[1,5-a]pyridin-3-yl]-quinoline-7-carboxylic acid methyl ester;
4-[2-(6-Methyl-pyridin-2-yl)-pyrazolo[1,5-a]pyridin-3-yl]-quinoline-6-carboxylic acid methyl ester;
4-(5-Benzyl-2-pyridin-2-yl-pyrazolo[1,5-a]pyridin-3-yl)-quinoline-7-carboxylic acid methyl ester;
3-(4-Fluoro-phenyl)-2-(6-methyl-pyridin-2-yl)-pyrazolo[1,5-a]pyridine-6-carboxylic acid (2-dimethylamino-ethyl)-amide;
4-[2-(6-Methyl-pyridin-2-yl)-pyrazolo[1,5-a]pyridin-3-yl]-quinoline-6-carboxylic acid (2-dimethylamino-ethyl)-amide;
4-[2-(6-Methyl-pyridin-2-yl)-pyrazolo[1,5-a]pyridin-3-yl]-quinoline-7-carboxylic acid (2-dimethylamino-ethyl)-amide;
5-[2-(6-Methyl-pyridin-2-yl)-pyrazolo[1,5-a]pyridin-3-yl]-benzofuran-2-carboxylic acid (2-dimethyl amino-ethyl)-amide;
4-[2-(6-Methyl-pyridin-2-yl)-pyrazolo[1,5-a]pyridin-3-yl]-quinoline-7-carboxylic acid [3-(4-methyl-piperazin-1-yl)-propyl]-amide;
4-[2-(6-Methoxy-pyridin-2-yl)-pyrazolo[1,5-a]pyridin-3-yl]-quinoline, 4-[2-(6-Ethoxy-pyridin-2-yl)-pyrazolo[1,5-a]pyridin-3-yl]-quinoline;
3-(4-Fluoro-phenyl)-2-(6-methoxy-pyridin-2-yl)-pyrazolo[1,5-a]pyridine;
2-(6-Ethoxy-pyridin-2-yl)-3-(4-fluoro-phenyl)-pyrazolo[1,5-a]pyridine;
7-Benzyl-4-[2-(6-methyl-pyridin-2-yl)-pyrazolo[1,5-a]pyridin-3-yl]-quinoline;
3-{4-[2-(6-Methyl-pyridin-2-yl)-pyrazolo[1,5-a]pyridin-3-yl]-quinolin-7-yl}-acrylic acid methyl ester;
3-{4-[2-(6-Methyl-pyridin-2-yl)-pyrazolo[1,5-a]pyridin-3-yl]-quinolin-7-yl}-acrylic acid;
4-[2-(6-Ethylsulfanyl-pyridin-2-yl)-pyrazolo[1,5-a]-pyridin-3-yl]-quinoline;
4-[2-(6-Phenylsulfanyl-pyridin-2-yl)-pyrazolo[1,5-a]pyridin-3-yl]-quinoline;
4-[2-(6-Morpholin-4-yl-pyridin-2-yl)-pyrazolo[1,5-a]pyridin-3-yl]-quinoline;
3-(4-Fluoro-phenyl)-2-(6-methylsulfanyl-pyridin-2-yl)-pyrazolo[1,5-a]pyridine;
3-(4-Methylsulfanyl-phenyl)-2-(6-methylsulfanyl-pyridin-2-yl)-pyrazolo[1,5-a]pyridine;
Dimethyl-{4-[2-(6-methyl-pyridin-2-yl)-pyrazolo[1,5-a]pyridin-3-yl]-quinolin-7-ylsulfanyl}-ethyl)-amine;
2-(Pyridin-2-yl)-3-(quinolin-4-yl)-pyrazolo[1,5-a]pyridine-5-carboxylic acid dimethylamide;
2-(Pyridin-2-yl)-3-(quinolin-4-yl)-pyrazolo[1,5-a]pyridine-6-carboxylic acid dimethylamide;
4-[2-(6-Vinyl-pyridin-2-yl)-pyrazolo[1,5-a]pyridin-3-yl]-quinoline, 6-[2-(6-Methyl-pyridin-2-yl)-pyrazolo[1,5-a]pyridin-3-yl]-imidazo[1,2-a]pyridin-2-yl-amine;
6-[2-(6-Methyl-pyridin-2-yl)-pyrazolo[1,5-a]pyridin-3-yl]-1H-benzoimidazol-2-yl-amine;
[3-(4-Fluoro-phenyl)-2-(6-methyl-pyridin-2-yl)-pyrazolo[1,5-a]pyridin-6-yl]-methanol, 6-Allyloxymethyl-3-(4-fluoro-phenyl)-2-(6-methyl-pyridin-2-yl)-pyrazolo[1,5-a]pyridine;
4-[2-(6-Methyl-pyridin-2-yl)-pyrazolo[1,5-a]pyridin-3-yl]-quinoline-7-carboxylic acid (3-pyrrolidin-1-yl-propyl)-amide;
3-{4-[2-(6-Methyl-pyridin-2-yl)-pyrazolo[1,5-a]pyridin-3-yl]-quinolin-7-yl}-propionamide;
3-{4-[2-(6-Methyl-pyridin-2-yl)-pyrazolo[1,5-a]pyridin-3-yl]-quinolin-7-yl}-N-(3-pyrrolidin-1-yl-propyl)-propionamide;
N-(Dimethylamino-ethyl)-3-{4-[2-(6-methyl-pyridin-2-yl)-pyrazolo[1,5-a]pyridin-3-yl]-quinolin-7-yl}-propionamide;
2-Pyridin-2-yl-3-quinolin-4-yl-pyrazolo[1,5-a]pyridine-5-carboxylic acid (3-dimethylamino-propyl)-amide;
4-[2-(6-Methyl-pyridin-2-yl)-pyrazolo[1,5-a]pyridin-3-yl]-quinoline-7-carboxylic acid (2-hydroxy-ethyl)-amide;
4-[2-(6-Methyl-pyridin-2-yl)-pyrazolo[1,5-a]pyridin-3-yl]-quinoline-7-carboxylic acid hydrazide;
4-[2-(6-Methyl-pyridin-2-yl)-pyrazolo[1,5-a]pyridin-3-yl]-quinoline-7-carboxylic acid (3-hydroxy-propyl)-amide;
4-[2-(6-Methyl-pyridin-2-yl)-pyrazolo[1,5-a]pyridin-3-yl]-quinoline-7-carboxylic acid methylamide;
4-[2-(6-Methyl-pyridin-2-yl)-pyrazolo[1,5-a]pyridin-3-yl]-quinoline-7-carboxylic acid (3-ethoxy-propyl)-amide;
4-[2-(6-Methyl-pyridin-2-yl)-pyrazolo[1,5-a]pyridin-3-yl]-quinoline-7-carboxylic acid (3-morpholin-4-yl-propyl)-amide;
4-[2-(6-Methyl-pyridin-2-yl)-pyrazolo[1,5-a]pyridin-3-yl]quinoline-7-carboxylic acid (3-imidazol-1-yl-propyl)-amide;
4-[2-(6-Methyl-pyridin-2-yl)-pyrazolo[1,5-a]pyridin-3-yl]-quinoline-7-carboxylic acid (3-dimethylamino-propyl)-amide;
4-[2-(6-Methyl-pyridin-2-yl)-pyrazolo[1,5-a]pyridin-3-yl]-quinoline-7-carboxylic acid [2-(2-methoxy-phenyl)-ethyl]-amide;
4-[2-(6-Methyl-pyridin-2-yl)-pyrazolo[1,5-a]pyridin-3-yl]-quinoline-7-carboxylic acid (2-morpholin-4-yl-ethyl)-amide;
4-[2-(6-Methyl-pyridin-2-yl)-pyrazolo[1,5-a]pyridin-3-yl]quinoline-7-carboxylic acid amide;
Dimethyl-{4-[2-(6-methyl-pyridin-2-yl)-pyrazolo[1,5-a]pyridin-3-yl]-quinolin-7-yloxy}-propyl)-amine;
4-[2-(6-Methyl-pyridin-2-yl)-pyrazolo[1,5-a]pyridin-3-yl]-7-(2-morpholin-4-yl-ethoxy)-quinoline;
Diisopropyl-(2-{4-[2-(6-methyl-pyridin-2-yl)-pyrazol[, 1,5-a]pyridin-3-yl]-quinolin-7-yloxy}-ethyl)-amine;
4-[2-(6-Methyl-pyridin-2-yl)-pyrazolo[1,5-a]pyridin-3-yl]-7-(2-pyrrol-1-yl-ethoxy)-quinoline;
Dimethyl-(1-methyl-2-{4-[2-(6-methyl-pyridin-2-yl)-pyrazolo[1,5-a]pyridin-3-yl]-quinolin-7-yloxy}ethyl)-amine;
Methyl-(3-{4-[2-(6-methyl-pyridin-2-yl)-pyrazolo[1,5-a]pyridin-3-yl]-quinolin-7-yl-oxy}-propyl)-amine;
4-[2-(6-Methyl-pyridin-2-yl)-pyrazolo[1,5-a]pyridin-3-yl]-7-(2-piperidin-1-yl-ethoxy)-quinoline;
Diethyl-(2-{4-[2-(6-methyl-pyridin-2-yl-pyrazolo[1,5-a]pyridin-3-yl]-quinolin-7-yloxy}-ethyl)-amine;
Dimethyl-{3-[4-(2-pyridin-2-yl-pyrazolo[1,5-a]pyridin-3-yl)-quinolin-7-yloxy]-propyl}-amine;
7-(2-Morpholin-4-yl-ethoxy)-4-(2-pyridin-2-yl-pyrazolo[1,5-a]pyridin-3-yl)-quinoline;
Diisopropyl-{2-[4-(2-pyridin-2-yl-pyrazolo[1,5-a]pyridin-3-yl)-quinolin-7-yloxy]-ethyl}-amine;
4-[2-(6-Methyl-pyridin-2-yl)-pyrazolo[1,5-a]pyridin-3-yl]-7-(3-morpholin-4-yl-propoxy)-quinoline;
i. (3-{4-[2-(6-Methyl-pyridin-2-yl)-pyrazolo[1,5-a]pyridine-3-yl]-quinolin-7-yloxy}-propyl)-1,3-dihydro-benzoimidazol-2-one 3-{4-[2-(6-Methyl-pyridin-2-yl)-pyrazolo[1,5-a]pyridin-3-yl]-quinolin-7-yl}-propionic acid methyl ester;
Diethyl-3-[4-(2-pyridin-2-yl-pyrazolo[1,5-a]pyridin-3-yl)-quinolin-7-yloxy]-propyl}-amine;
Ethyl-methyl-{3-[4-(2-pyridin-2-yl-pyrazolo[1,5-a]pyridin-3-yl)-quinolin-7-yloxy]-propyl}-amine;
4-(2-Pyridin-2-yl-pyrazolo[1,5-a]pyridin-3-yl)-7-(3-pyrrolidin-1-yl-propoxy)-quinoline;
7-(3-Piperidin-1-yl-propoxy)-4-(2-pyridin-2-yl-pyrazolo[1,5-a]pyridin-3-yl)-quinoline;
Diethyl-{2-[4-(2-pyridin-2-yl-pyrazolo[1,5-a]pyridin-3-yl)-quinolin-7-yloxy]-ethyl}-amine;
Dimethyl-{2-[4-(2-pyridin-2-yl-pyrazolo[1,5-a]pyridin-3-yl)-quinolin-7-yloxy]-ethyl}-amine;
6-Bromo-4-(2-pyridin-2-yl-5,6-dihydro-4H-pyrrolo[1,2-b]pyrazol-3-yl)-quinoline;
3-Pyridin-4-yl-2-pyridin-2-yl-5,6-dihydro-4H-pyrrolo[1,2-b]pyrazole;
2-(6-Methyl-pyridin-2-yl)-3-p-tolyl-5,6-dihydro-4H-pyrrolo[1,2-b]pyrazole;
4-[3-(6-Methyl-pyridin-2-yl)-5,6-dihydro-4H-pyrrolo[1,2-b]pyrazol-2-yl]-quinoline;
2-(6-Methyl-pyridin-2-yl)-3-naphthalen-1-yl-5,6-dihydro-4H-pyrrolo[1,2-b]pyrazole;
(6-Methyl-pyridin-2-yl)-3-pyridin-3-yl-5,6-dihydro-4H-pyrrolo[1,2-b]pyrazole;
3-(4-Fluoro-naphthalen-1-yl)-2-(6-methyl-pyridin-2-yl)-5,6-dihydro-4H-pyrrolo[1,2-b]pyrazole;
3-(3,4-Difluoro-phenyl)-2-(6-methyl-pyridin-2-yl)-5,6-dihydro-4H-pyrrolo[1,2-b]pyrazole;
i. [2-(4-Methanesulfonyl-phenyl)-1-(6-methyl-pyridin-2-yl)-ethylideneamino]-pyrrolidin-2-one;
7-Methoxy-4-(2-pyridin-2-yl-5,6-dihydro-4H-pyrrolo[1,2-b]pyrazol-3-yl)-quinoline;
7-Benzyloxy-6-methoxy-4-(2-pyridin-2-yl-5,6-dihydro-4H-pyrrolo[1,2-b]pyrazol-3-yl)-quinoline;
6-(2-Pyridin-2-yl-5,6-dihydro-4H-pyrrolo[1,2-b]pyrazol-3-yl)-quinoline;
6-[2-(6-Methyl-pyridin-2-yl)-5,6-dihydro-4H-pyrrolo[1,2-b]pyrazol-3-yl]-quinoline;
3-Naphthalen-2-yl-2-pyridin-2-yl-5,6-dihydro-4H-pyrrolo[1,2-b]pyrazole;
2-(6-Methyl-pyridin-2-yl)-3-naphthalen-2-yl-5,6-dihydro-4H-pyrrolo[1,2-b]pyrazole;
3-(4-Fluoro-phenyl)-2-(6-trifluoromethyl-pyridin-2-yl)-5,6-dihydro-4H-pyrrolo[1,2-b]pyrazole;
4-(Quinolin-4-yl)-3-(5-fluoropyridin-2-yl)-5,6-dihydro-4H-pyrrolo[1,2-b]pyrazole;
4-(7-Bromoquinolin-4-yl)-3-(pyridin-2-yl)-5,6-dihydro-4H-pyrrolo[1,2-b]pyrazole;
(Quinolin-4-yl)-3-(2,4-difluorophenyl)-5,6-dihydro-4H-pyrrolo[1,2-b]pyrazole;
4-(2-Pyrazin-2-yl-5,6-dihydro-4H-pyrrolo[1,2-b]pyrazol-3-yl)-quinoline;
4-(5-Methyl-2-pyridin-2-yl-5,6-dihydro-4H-pyrrolo[1,2-b]pyrazol-3-yl)-quinoline;
6-Bromo-4-[2-(6-methyl-pyridin-2-yl)-5,6-dihydro-4H-pyrrolo[1,2-b]pyrazol-3-yl]-quinoline;
4-[2-(6-Methyl-pyridin-2-yl)-5,6-dihydro-4H-pyrrolo[1,2-b]pyrazol-3-yl]-6-trifluoromethyl-quinoline;
3-(3-Chloro-4-fluoro-phenyl)-2-(6-methyl-pyridin-2-yl)-5,6-dihydro-4H-pyrrolo[1,2-b]pyrazole;
3-(2-Chloro-4-fluoro-phenyl)-2-(6-methyl-pyridin-2-yl)-5,6-dihydro-4H-pyrrolo[1,2-b]pyrazole;
3-(4-Fluoro-3-trifluoromethyl-phenyl)-2-(6-methyl-pyridin-2-yl)-5,6-dihydro-4H-pyrrolo[1,2-b]pyrazole;
2-(6-Methyl-pyridin-2-yl)-3-(2,4,5-trifluoro-phenyl)-5,6-dihydro-4H-pyrrolo[1,2-b]pyrazole;
8-Fluoro-4-[2-(6-methyl-pyridin-2-yl)-5,6-dihydro-4H-pyrrolo[1,2-b]pyrazol-3-yl]-quinoline;
7-Bromo-4-[2-(6-methyl-pyridin-2-yl)-5,6-dihydro-4H-pyrrolo[1,2-b]pyrazol-3-yl]-quinoline;
4-[2-(6-Methyl-pyridin-2-yl)-5,6-dihydro-4H-pyrrolo[1,2-b]pyrazol-3-yl]-6-trifluoromethoxy-quinoline;
4-[2-(6-Methyl-pyridin-2-yl)-5,6-dihydro-4H-pyrrolo[1,2-b]pyrazol-3-yl]-7-trifluoromethyl-quinoline;
7-Methoxy-4-[2-(6-methyl-pyridin-2-yl)-5,6-dihydro-4H-pyrrolo[1,2-b]pyrazol-3-yl]-quinoline;
3-(2-Chloro-pyridin-4-yl)-2-pyridin-2-yl-5,6-dihydro-4H-pyrrolo[1,2-b]pyrazole;
[2-(6-Methyl-pyridin-2-yl)-3-quinolin-4-yl-5,6-dihydro-4H-pyrrolo[1,2-b]pyrazol-6-yl]-methanol;
[3-(7-Bromo-quinolin-4-yl)-2-(6-methyl-pyridin-2-yl)-5,6-dihydro-4H-pyrrolo[1,2-b]pyrazol-6-yl]-methanol;
4-[2-(6-Chloro-pyridin-2-yl)-5-(4-fluorophenyl)-5,6-dihydro-4H-pyrrolo[1,2-b]pyrazol-3-yl]-quinoline;
4-[2-(6-Ethoxy-pyridin-2-yl)-5-(4-fluoro-phenyl)-5,6-dihydro-4H-pyrrolo[1,2-b]pyrazol-3-yl]-quinoline;
(S)-4-[6-Benzyloxymethyl-2-(6-methyl-pyridin-2-yl)-5,6-dihydro-4H-pyrrolo[1,2-b]pyrazol-3-yl]-7-chloro-quinoline;
(S)-4-[6-Benzyloxymethyl-2-(6-chloro-pyridin-2-yl)-5,6-dihydro-4H-pyrrolo[1,2-b]pyrazol-3-yl]-quinoline;
4-[2-(6-Methyl-pyridin-2-yl)-3-quinolin-4-yl-5,6-dihydro-4H-pyrrolo[1,2-b]pyrazol-5-yl]-benzoic acid ethyl ester;
3-(4-Fluoro-phenyl)-5,5-dimethyl-2-(6-methyl-pyridin-2-yl)-5,6-dihydro-4H-pyrrolo[1,2-b]pyrazole;
(R)-6-Benzyloxymethyl-3-(4-fluoro-phenyl)-2-(6-methyl-pyridin-2-yl)-5,6-dihydro-4H-pyrrolo[1,2-b]pyrazole;
5-(4-Chloro-phenyl)-3-(4-fluoro-phenyl)-2-(6-methyl-pyridin-2-yl)-5,6-dihydro-4H-pyrrolo[1,2-b]pyrazole;
4-[2-(3-Trifluoromethyl-phenyl)-4,5,6,7-tetrahydro-pyrazolo[1,5-a]pyridin-3-yl]-quinoline;
4-[2-(4-Trifluoromethyl-phenyl)-4,5,6,7-tetrahydro-pyrazolo[1,5-a]pyridin-3-yl]-quinoline;
4-[2-(4-Chloro-phenyl)-4,5,6,7-tetrahydro-pyrazolo[1,5-a]pyridin-3-yl]-quinoline;
4-[2-(3-Chloro-phenyl)-4,5,6,7-tetrahydro-pyrazolo[1,5-a]pyridin-3-yl]-quinoline;
4-[2-(3-Fluoro-5-trifluoromethyl-phenyl)-5,6-dihydro-4H-pyrrolo[1,2-b]pyrazol-3-yl]-quinoline;
4-[2-(3-Fluoro-5-trifluoromethyl-phenyl)-4,5,6,7-tetrahydro-pyrazolo[1,5-a]pyridin-3-yl]-quinoline;
4-(2-Phenyl-4,5,6,7-tetrahydro-pyrazolo[1,5-a]pyridin-3-yl)-quinoline;
4-(2-Pyridin-2-yl-4,5,6,7-tetrahydro-pyrazolo[1,5-a]pyridin-3-yl)-[1,10]phenanthroline;
4-[2-(4-Fluoro-phenyl)-4,5,6,7-tetrahydro-pyrazolo[1,5-a]pyridin-3-yl]-quinoline;
4-[2-(3-Trifluoromethoxy-phenyl)-4,5,6,7-tetrahydro-pyrazolo[1,5-a]pyridin-3-yl]-quinoline;
4-[2-(2-Fluoro-phenyl)-4,5,6,7-tetrahydro-pyrazolo[1,5-a]pyridin-3-yl]-quinoline;
4-(2-Quinolin-2-yl-4,5,6,7-tetrahydro-pyrazolo[1,5-a]pyridin-3-yl)-quinoline;
4-[2-(4-Ethyl-pyridin-2-yl)-4,5,6,7-tetrahydro-pyrazolo[1,5-a]pyridin-3-yl]-quinoline;
4-(2-Quinolin-2-yl-5,6-dihydro-4H-pyrrolo[1,2-b]pyrazol-3-yl)-quinoline;
2-(3-Quinolin-4-yl-4,5,6,7-tetrahydro-pyrazolo[1,5-a]pyridin-2-yl)-[1,8]naphthyridine;
4-[5-(4-Fluoro-phenyl)-2-pyridin-2-yl-5,6-dihydro-4H-pyrrolo[1,2-b]pyrazol-3-yl]-quinoline;
4-(6-Hydroxymethyl-2-pyridin-2-yl-5,6-dihydro-4H-pyrrolo[1,2-b]pyrazol-3-yl)-quinoline;
4-(3-Pyridin-2-yl-5,6-dihydro-4H-pyrrolo[1,2-b]pyrazol-2-yl)-quinoline;
4-(4-Methyl-2-pyridin-2-yl-5,6-dihydro-4H-pyrrolo[1,2-b]pyrazol-3-yl)-quinoline;
4-(5-Benzyl-2-pyridin-2-yl-5,6-dihydro-4H-pyrrolo[1,2-b]pyrazol-3-yl)-quinoline;
4-(5-Phenethyl-2-pyridin-2-yl-5,6-dihydro-4H-pyrrolo[1,2-b]pyrazol-3-yl)-quinoline;
4-(5-Phenyl-2-pyridin-2-yl-5,6-dihydro-4Hpyrrob[1,2-b]pyrazol-3-yl)-quinoline;
4-[2-(3-Trifluoromethylphenyl)-5,6-dihydro-4H-pyrrolo[1,2-b]pyrazol-3-yl]-quinoline;
4-[2-(4-Trifluoromethyl-phenyl)-5,6-dihydro-4H-pyrrolo[1,2-b]pyrazol-3-yl]-quinoline;
4-(2-Phenyl-5,6-dihydro-4H-pyrrolo[1,2-b]pyrazol-3-yl)-quinoline;
2-Chloro-4-(2-pyridin-2-yl-5,6-dihydro-4H-pyrrolo[1,2-b]pyrazol-3-yl)-quinoline;
6,8-Dimethoxy-4-[2-(6-methyl-pyridin-2-yl)-5,6-dihydro-4H-pyrrolo[1, 2b]pyrazol-3-yl]-quinoline;
4-[2-(6-Bromo-pyridin-2-yl)-5,6-dihydro-4H-pyrrolo[1,2-b]pyrazol-3-yl]-quinoline;
6,8-Dimethoxy-4-[2-pyridin-2-yl-5,6-dihydro-4H-pyrrolo[1, 2b]pyrazol-3-yl]-quinoline;
3-(4-Fluorophenyl)-2-pyridin-2-yl-5,6-dihydro-4H-pyrrolo[1,2-b]pyrazole;
3-(4-Methoxy-phenyl)-2-pyridin-2-yl-5,6-dihydro-4H-pyrrolo[1,2-b]pyrazole;
3-(4-Fluorophenyl)-2-(6-methylpyridin-2-yl)-5,6-dihydro-4H-pyrrolo[1,2-b]pyrazole;
3-(4-Methoxyphenyl)-2-(6-methylpyridin-2-yl)-5,6-dihydro-4H-pyrrolo[1,2-b]pyrazole;
4-(2-Thiophen-2-yl-5,6-dihydro-4H-pyrrolo[1,2-b]pyrazol-3-yl)quinoline;
4-[2-(6-Propylpyridin-2-yl)-5,6-dihydro-4H-pyrrolo[1,2-b]pyrazol-3-yl]-quinoline;
4-[2-(6-Isopropylpyridin-2-yl)-5,6-dihydro-4H-pyrrolo[1,2-b]pyrazol-3-yl]quinoline;
4-[2-(6-Ethyl-pyridin-2-yl)-5,6-dihydro-4H-pyrrolo[1,2-b]pyrazol-3-yl]quinoline;
4-[2-(6-Methyl-pyridin-2-yl)-5,6-dihydro-4H-pyrrolo[1,2-b]pyrazol-3-yl]-quinoline;
4-[2-(3-Fluorophenyl)-5,6-dihydro-4H-pyrrolo[1,2-b]pyrazol-3-yl]-quinoline;
4-[2-(2-Fluoro-phenyl)-5,6-dihydro-4H-pyrrolo[1,2-b]pyrazol-3-yl]-quinoline;
4-[2-(4-Fluoro-phenyl)-5,6-dihydro-4H-pyrrolo[1,2-b]pyrazol-3-yl]-quinoline;
4-[2-(3-Trifluoromethoxy-phenyl)-5,6-dihydro-4H-pyrrolo[1,2-b]pyrazol-3-yl]-quinoline;
4-[2-(4-Chloro-pyridin-2-yl)-5,6-dihydro-4H-pyrrolo[1,2-b]pyrazol-3-yl]-quinoline;
4-[2-(4-Fluoro-3-trifluoromethyl-phenyl)-5,6-dihydro-4H-pyrrolo[1,2-b]pyrazol-3-yl]quinoline;
4-[2-(2-Fluoro-3-trifluoromethyl-phenyl)-5,6-dihydro-4H-pyrrolo[1,2-b]-pyrazol-3-yl]-quinoline;
4-[5-(3-Methoxy-phenyl)-2-pyridin-2-yl-5,6-dihydro-4H-pyrrolo[1,2-b]pyrazol-3-yl]-quinoline;
4-[2-(4-Fluoro-3-trifluoromethyl-phenyl)-5-(3-methoxy-phenyl)-5,6-dihydro-4H-pyrrolo[1,2-b]pyrazol-3-yl]-quinoline;
4-(7-Chloro-quinolin-4-yl)-3-(6-methylpyridin-2-yl)-5,6-dihydro-4H-pyrrolo[1,2-b]pyrazole;
4-(7-Ethoxyquinolin-4-yl)-3-(6-methylpyridin-2-yl)-5,6-dihydro-4H-pyrrolo[1,2-b]pyrazole;
6-(3-Quinolin-4-yl-5,6-dihydro-4H-pyrrolo[1,2-b]pyrazol-2-yl)-pyridine-2-carboxylic acid hydrochloride;
6,7-Difluoro-4-[2-(6-methyl-pyridin-2-yl)-5,6-dihydro-4H-pyrrolo[1,2-b]pyrazol-3-yl]-quinoline;
6,7-Dimethoxy-4-[2-(6-methyl-pyridin-2-yl)-5,6-dihydro-4H-pyrrolo[1,2-b]pyrazol-3-yl]-quinoline;
3-Benzo[1, 3]dioxol-5-yl-2-(6-methyl-pyridin-2-yl)-5,6-dihydro-4H-pyrrolo[1,2-b]pyrazole;
6-(4-Fluoro-phenyl)-4-[2-(6-methyl-pyridin-2-yl)-5,6-dihydro-4H-pyrrolo[1,2-b]pyrazol-3-yl]-quinoline;
6-Benzo[1, 3]dioxol-5-yl-4-[2-(6-methyl-pyridin-2-yl)-5,6-dihydro-4H-pyrrolo[1,2-b]pyrazol-3-yl]-quinoline;
4-[2-(6-Methyl-pyridin-2-yl)-5,6-dihydro-4H-pyrrolo[1,2-b]pyrazol-3-yl]-6-thiophen-2-yl-quinoline;
4-[2-(6-Methyl-pyridin-2-yl)-5,6-dihydro-4H-pyrrolo[1,2-b]pyrazol-3-yl]-6-phenyl-quinoline;
8-[2-(6-Methyl-pyridin-2-yl)-5,6-dihydro-4H-pyrrolo[1,2-b]pyrazol-3-yl]-quinoline;
3-Benzo[b]thiophen-2-yl-2-(6-methyl-pyridin-2-yl)-5,6-dihydro-4H-pyrrolo[1,2-b]pyrazole;
4-(2-Pyridin-2-yl-5,6-dihydro-4H-pyrrolo[1,2-b]pyrazol-3-yl)-quinoline-6-carboxylic acid methyl ester;
4-[2-(6-Methyl-pyridin-2-yl)-5,6-dihydro-4H-pyrrolo[1,2-b]pyrazol-3-yl]-quinoline-6-carboxylic acid methyl ester;
4-[2-(6-Methyl-pyridin-2-yl)-5,6-dihydro-4H-pyrrolo[1,2-b]pyrazol-3-yl]-quinoline-7-carboxylic acid methyl ester;
4-[2-Pyridin-2-yl-5,6-dihydro-4H-pyrrolo[1,2-b]pyrazol-3-yl]-quinoline-7-carboxylic acid methyl ester;
2-Pyridin-2-yl-3-quinolin-4-yl-pyrazolo[5,1-c]morpholine;
2-Pyridin-2-yl-3-quinolin-4-yl-pyrazolo[5,1-c]morpholin-4-one;
Dimethyl-{3-[4-(2-pyridin-2-yl-5,6-dihydro-4H-pyrrolo[1,2-b]pyrazol-3-yl)-quinolin-7-yloxy]-propyl}-amine;
{3-[6-Methoxy-4-(2-pyridin-2-yl-5,6-dihydro-4H-pyrrolo[1,2-b]pyrazol-3-yl)-quinolin-7-yloxy]-propyl}-dimethyl-amine;
Cyclopropylmethyl-propyl-{3-[4-(2-pyridin-2-yl-5,6-dihydro-4H-pyrrolo[1,2-b]pyrazol-3-yl)-quinolin-7-yloxy]-propyl}-amine;
Diethyl-{3-[4-(2-pyridin-2-yl-5,6-dihydro-4H-pyrrolo[1,2-b]pyrazol-3-yl)-quinolin-7-yloxy]-propyl}-amine;
Ethyl-methyl-{3-[4-(2-pyridin-2-yl-5,6-dihydro-4H-pyrrolo[1,2-b]pyrazol-3-yl)-quinolin-7-yloxy]-propyl}-amine)jjjjj) 3-[4-(2-Pyridin-2-yl-5,6-dihydro-4H-pyrrolo[1,2-b]pyrazol-3-yl)-quinolin-7-yloxy]-propylamine;
7-[3-(4-Methyl-piperazin-1-yl)-propoxy]-4-(2-pyridin-2-yl-5,6-dihydro-4H-pyrrolo[1,2-b]pyrazol-3-yl)-quinoline;
Benzyl-methyl-{3-[4-(2-pyridin-2-yl-5,6-dihydro-4H-pyrrolo[1,2-b]pyrazol-3-yl)-quinolin-7-yloxy]-propyl}-amine;
7-(3-Piperidin-1-yl-propoxy)-4-(2-pyridin-2-yl-5,6 dihydro-4H-pyrrolo[1,2-b]pyrazol-3-yl)-quinoline;
4-(2-Pyridin-2-yl-5,6-dihydro-4H-pyrrolo[1,2-b]pyrazol-3-yl)-7-(3-pyrrolidin-1-yl-propoxy)-quinoline;
7-(3-Azepan-1-yl-propoxy)-4-(2-pyridin-2-yl-5,6-dihydro-4H-pyrrolo[1,2-b]pyrazol-3-yl)-quinoline;
7-(3-Imidazol-1-yl-propoxy)-4-(2-pyridin-2-yl-5,6-dihydro-4H-pyrrolo[1,2-b]pyrazol-3-yl)-quinoline;
7-(3-Pyrazol-1-yl-propoxy)-4-(2-pyridin-2-yl-5,6-dihydro-4H-pyrrolo[1,2-b]pyrazol-3-yl)-quinoline;
1′-{3-[4-(2-Pyridin-2-yl-5,6-dihydro-4H-pyrrolo[1,2-b]pyrazol-3-yl)-quinolin-7-yloxy]-propyl}-[1, 4′]bipiperidinyl;
Cyclopropyl-(1-methyl-piperidin-4-yl)-f 3-[4-(2-pyridin-2-yl-5,6-dihydro-4H-pyrrolo[1,2-b]pyrazol-3-yl)-quinolin-7-yloxy]-propyl}-amine;
4-(2-Pyridin-2-yl-5,6-dihydro-4H-pyrrolo[1,2-b]pyrazol-3-yl)-7-(3-[1,2,3]triazol-1-yl-propoxy)-quinoline;
Dimethyl-(3-{4-[2-(6-methyl-pyridin-2-yl)-5,6-dihydro-4H-pyrrolo[1,2-b]pyrazol-3-yl]-quinolin-7-yloxy}-propyl)-amine;
Diethyl-(3-{4-[2-(6-methyl-pyridin-2-yl)-5,6-dihydro-4H-pyrrolo[1,2-b]pyrazol-3-yl]-quinolin-7-yloxy}-propyl)-amine;
Cyclopropylmethyl-(3-{4-[2-(6-methyl-pyridin-2-yl)-5,6-dihydro-4H-pyrrolo[1,2-b]pyrazol-3-yl]-quinolin-7-yloxy}-propyl)-propyl-amin;
Ethyl-methyl-(3-{4-[2-(6-methyl-pyridin-2-yl)-5,6-dihydro-4H-pyrrolo[1,2-b]pyrazol-3-yl]-quinolin-7-yloxy}-propyl)-amine;
Dimethyl-{2-[4-(2-pyridin-2-yl-5,6-dihydro-4H-pyrrolo[1,2-b]pyrazol-3-yl)-quinolin-7-yloxy]-ethyl}-amine;
Diethyl-{2-[4-(2-pyridin-2-yl-5,6-dihydro-4H-pyrrolo[1,2-b]pyrazol-3-yl)-quinolin-7-yloxy]-ethyl}-amine;
7-(2-Piperidin-1-yl-ethoxy)-4-(2-pyridin-2-yl-5,6-dihydro-4H-pyrrolo[1,2-b]pyrazol-3-yl)-quinoline;
Ethyl-methyl-{2-[4-(2-pyridin-2-yl-5,6-dihydro-4H-pyrrolo[1,2-b]pyrazol-3-yl)-quinolin-7-yloxy]ethyl}-amine;
4-(2-Pyridin-2-yl-5,6-dihydro-4H-pyrrolo[1,2-b]pyrazol-3-yl)-7-(2-pyrrolidin-1-yl-ethoxy)-quinoline;
7-[2-(4-Methyl-piperazin-1-yl)-ethoxy]-4-(2-pyridin-2-yl-5,6-dihydro-4H-pyrrolo[1,2-b]pyrazol-3-yl)-quinoline;
Dimethyl-{3-[1-oxy-4-(2-pyridin-2-yl-5,6-dihydro-4H-pyrrolo[1,2-b]pyrazol-3-yl)-quinolin-7-yloxy]-propyl}-amine;
7-Methylsulfanyl-4-(2-pyridin-2-yl-5,6-dihydro-4H-pyrrolo[1,2-b]pyrazol-3-yl)-quinoline;
7-Ethylsulfanyl-4-(2-pyridin-2-yl-5,6-dihydro-4H-pyrrolo[1,2-b]pyrazol-3-yl)-quinoline;
6-Methylsulfanyl-4-(2-pyridin-2-yl-5,6-dihydro-4H-pyrrolo[1,2-b]pyrazol-3-yl)-quinoline;
7-Benzylsulfanyl-4-(2-pyridin-2-yl-5,6-dihydro-4H-pyrrolo[1,2-b]pyrazol-3-yl)-quinoline;
3-[4-(2-Pyridin-2-yl-5,6-dihydro-4H-pyrrolo[1,2-b]pyrazol-3-yl)-quinolin-7-yl sulfanyl]-propan-1-ol;
Dimethyl-{2-[4-(2-pyridin-2-yl-5,6-dihydro-4H-pyrrolo[1,2-b]pyrazol-3-yl)-quinolin-7-ylsulfanyl]-ethyl}-amine;
Dimethyl[6-(3-quinolin-4-yl-5,6-dihydro-4H-pyrrolo[1,2-b]pyrazol-2-yl)-pyridin-2-yl-methyl]amine;
7-(2-Propoxy-ethoxy)-4-(2-pyridin-2-yl-5,6-dihydro-4H-pyrrolo[1,2-b]pyrazol-3-yl)-quinoline;
N,N-Dimethyl-N′-[4-(2-pyridin-2-yl-5,6-dihydro-4H-pyrrolo[1,2-b]pyrazol-3-yl)-pyridin-2-yl]-ethane-1,2-diamine;
N,N-Dimethyl-N′-[4-(2-pyridin-2-yl-5,6-dihydro-4H-pyrrolo[1,2-b]pyrazol-3-yl)-pyridin-2-yl]-propane-1,3-diamine;
3-{3-[4-(2-Pyridin-2-yl-5,6-dihydro-4H-pyrrolo[1,2-b]pyrazol-3-yl)-quinolin-7-yloxy]-propyl}-oxazolidin-2-one;
1-{3-[4-(2-Pyridin-2-yl-5,6-dihydro-4H-pyrrolo-[1,2-b]pyrazol-3-yl)-quinolin-7-yloxy]-propyl}-imidazolidin-2-one;
3-{3-[4-(2-Pyridin-2-yl-5,6-dihydro-4H-pyrrolo[1,2-b]pyrazol-3-yl)-quinolin-7-yloxy]-propyl}-3H-benzooxazol-2-one;
Dimethyl-(2-{4-[2-(6-methyl-pyridin-2-yl)-5,6-dihydro-4H-pyrrolo[1,2-b]pyrazol-3-yl]-pyridin-2-ylsulfanyl}-ethyl-amine;
4-(2-Pyridin-2-yl-5,6-dihydro-4H pyrrolo-[1,2-b]pyrazol-3-yl)-2pyrrolidin-1-yl-quinoline;
2-Phenylsulfanyl-4-(2-pyridin-2-yl-5,6-dihydro-4H-pyrrolo[1,2-b]pyrazol-3-yl)-quinoline;
2-Morpholin-4-yl-4-(2-pyridin-2-yl-5,6-dihydro-4H-pyrrolo[1,2-b]pyrazol-3-yl)-quinoline;
2-Ethylsulfanyl-4-(2-pyridin-2-yl-5,6-dihydro-4H-pyrrolo[1,2-b]pyrazol-3-yl)-quinoline;
Phenyl-[4-(2-pyridin-2-yl-5,6-dihydro-4H-pyrrolo-[1,2-b]pyrazol-3-yl)-quinolin-2-yl]-amine;
2-Methoxy-4-(2-pyridin-2-yl-5,6-dihydro-4H-pyrrolo[1,2-b]pyrazol-3-yl)-quinoline;
2-Ethoxy-4-(2-pyridin-2-yl-5,6-dihydro-4H-pyrrolo[1,2-b]pyrazol-3-yl)-quinoline;
4-[2-(6-Phenylsulfanyl-pyridin-2-yl)-5,6-dihydro-4H-pyrrolo[1,2-b]pyrazol-3-yl]-quinoline;
Phenyl-[6-(3-quinolin-4-yl-5,6-dihydro-4H-pyrrolo[1,2-b]pyrazol-2-yl)-pyridin-2-yl]-amine;
4-{2-[6-(4-Methoxy-phenyl)-pyridin-2-yl]-5,6-dihydro-4H-pyrrolo[1,2-b]pyrazol-3-yl}-quinoline;
4-[2-(6-Phenyl-pyridin-2-yl)-5,6-dihydro-4H-pyrrolo[1,2-b]pyrazol-3-yl]-quinoline;
4-[2-(6-Morpholin-4-yl-pyridin-2-yl)-5,6-dihydro-4H-pyrrolo[1,2-b]pyrazol-3-yl]-quinoline;
4-[2-(6-Pyrrolidin-1-yl-pyridin-2-yl)-5,6-dihydro-4H-pyrrolo[1,2-b]pyrazol-3-yl]-quinoline;
4-[2-(6-Methoxy-pyridin-2-yl)-5,6-dihydro-4H-pyrrolo[1,2-b]pyrazol-3-yl]-quinoline;
2-{3-[4-(2-Pyridin-2-yl-5,6-dihydro-4H-pyrrolo-[1,2-b]pyrazol-3-yl)-quinolin-7-yloxy]-propyl}-isoindole-1,3-dione;
7-(3-Fluoro-propoxy)-4-(2-pyridin-2-yl-5,6-dihydro-4H-pyrrolo[1,2-b]pyrazol-3-yl)-quinoline;
7-(3-Fluoro-propoxy)-4-(2-pyridin-2-yl-5,6-dihydro-4H-pyrrolo-[1,2-b]pyrazol-3-yl)-quinoline;
7-(3-Chloro-propoxy)-4-(2-pyridin-2-yl-5,6-dihydro-4H-pyrrolo[1,2-b]pyrazol-3-yl)-quinoline;
7-(3-Chloro-propoxy)-6-methoxy-4-(2-pyridin-2-yl-5,6-dihydro-4H-pyrrolo[1,2-b]pyrazol-3-yl)-quinoline;
7-(3-Chloro-propoxy)-4-[2-(6-methyl-pyridin-2-yl)-5,6-dihydro-4H-pyrrolo[1,2-b]pyrazol-3-yl]-quinoline;
(1-{3-[7-(2-Chloro-ethoxy)-quinolin-4-yl]-5,6-dihydro-4H-pyrrolo[1,2-b]pyrazol-2-yl}-propenyl)-methylene-amine;
N,N-Diethyl-2-[4-(2-pyridin-2-yl-5,6-dihydro-4H-pyrrolo[1,2-b]pyrazol-3-yl)-quinolin-7-yloxy]-acetamide;
7-[2-((2R)-1-Methyl-pyrrolidin-2-yl)-ethoxy]-4-(2-pyridin-2-yl-5,6-dihydro-4H-pyrrolo[1,2-b]pyrazol-3-yl)-quinoline;
Dimethyl-{4-[4-(2-pyridin-2-yl-5,6-dihydro-4H-pyrrolo[1,2-b]pyrazol-3-yl)-pyridin-2-yloxy]-butyl}-amine;
1-{3-[4-(2-Pyridin-2-yl-5,6-dihydro-4H-pyrrolo[1,2-b]pyrazol-3-yl)-pyridin-2-yloxy]-propyl}-pyrrolidin-2-one;
7-(1-Methyl-piperidin-3-ylmethoxy)-4-(2-pyridin-2-yl-5,6-dihydro-4H-pyrrolo[1,2-b]pyrazol-3-yl)-quinoline;
7-(3-N,N-Dimethylamino-2-methyl-propyloxy)-4-(2-pyridin-2-yl-5,6-dihydro-4H-pyrrolo[1,2-b]pyrazol-3-yl)-quinoline;
4-[2-(6-Methyl-pyridin-2-yl)-5,6-dihydro-4H-pyrrolo[1,2-b]pyrazol-3-yl]-7-propoxy-quinoline;
4-[6-Benzyloxymethyl-2-(6-methyl-pyridin-2-yl)-5,6-dihydro-4H-pyrrolo[1,2-b]pyrazol-3-yl]-quinoline;
{4-[2-(6-Methyl-pyridin-2-yl)-5,6-dihydro-4H-pyrrolo[1,2-b]pyrazol-3-yl]-quinolin-7-yloxy}-acetic acid methyl ester;
7-Isopropoxy-4-[2-(6-methyl-pyridin-2-yl)-5,6-dihydro-4H-pyrrolo[1,2-b]pyrazol-3-yl]-quinoline;
4-[2-(6-Methyl-pyridin-2-yl)-5,6-dihydro-4H-pyrrolo[1,2-b]pyrazol-3-yl]-7-(3-morpholin-4-yl-propoxy)-quinoline;
4-(6-Benzyloxymethyl-2-pyridin-2-yl-5,6-dihydro-4H-pyrrolo[1,2-b]pyrazol-6-yl)-quinoline;
7-Benzyloxy-2-Pyridin-2-yl-3-quinolin-4-yl-pyrazolo[1,5-a]piperidine;
2-[4-(2-pyridin-2-yl-5,6-dihydro-4H-pyrrolo[1,2-b]pyrazol-3-yl)-quinolin-7-yloxy]-acetamide;
7-(5-Phenyl-[1, 2,4]oxadiazol-3-ylmethoxy)-4-(2-pyridin-2-yl-5,6-dihydro-4H-pyrrolo[1,2-b]pyrazol-3-yl)-quinoline;
7-(2,2-Difluoro-benzo[1, 3]dioxol-5-ylmethoxy)-4-(2-pyridin-2-yl-5,6-dihydro-4H-pyrrolo[1,2-b]pyrazol-3-yl)-quinoline;
7-[2-(259-1-Methyl-pyrrolidin-2-yl)-ethoxy]-4-(2-pyridin-2-yl-5,6-dihydro-4H-pyrrolo[1,2-b]pyrazol-3-yl)-quinoline;
5-[4-(2-Pyridin-2-yl-5,6-dihydro-4H-pyrrolo[1,2-b]pyrazol-3-yl)-quinolin-7-yloxymethyl]-pyrrolidin-2-one;
4-(6-Phenoxymethyl-2-pyridin-2-yl-5,6-dihydro-4H-pyrrolo[1,2-b]pyrazol-3-yl)-quinoline;
4-(6-Methylene-2-pyridin-2-yl-5,6-dihydro-4H-pyrrolo[1,2-b]pyrazol-3-yl)-quinoline;
3-(4-Fluoro-phenyl)-6-methylene-2-(6-methyl-pyridin-2-yl)-5,6-dihydro-4H-pyrrolo[1,2-b]pyrazole;
7-(1-Methyl-piperidin-2-ylmethoxy)-4-(2-pyridin-2-yl-5,6-dihydro-4H-pyrrolo[1,2-b]pyrazol-3-yl)-quinoline hydrochloride;
7-[2-(1-Methyl-pyrrolidin-2-yl)-ethoxy]-4-(2-pyridin-2-yl-5,6-dihydro-4H-pyrrolo[1,2-b]pyrazol-3-yl)-quinoline hydrochloride;
4-[2-(6-Methyl-1-oxy-pyridin-2-yl)-5,6-dihydro-4H-pyrrolo[1,2-b]pyrazol-3-yl]-quinoline 1-oxide;
4-[2-(6-Methyl-pyridin-2-yl)-5,6-dihydro-4H-pyrrolo[1,2-b]pyrazol-3-yl]-quinoline 1-oxide;
4-[2-(6-Methyl-1-oxy-pyridin-2-yl)-5,6-dihydro-4H-pyrrolo[1,2-b]pyrazol-3-yl]-quinoline;
7-(3-Chloro-propoxy)-4-(2-pyridin-2-yl-5,6-dihydro-4H-pyrrolo[1,2-b]pyrazol-3-yl)-quinoline 1-oxide;
7-Methanesulfonyl-4-(2 pyridin-2-yl-5,6-dihydro-4H-pyrrolo[1,2-b]pyrazol-3-yl)-quinoline;
3-(4-Fluoro-phenyl)-2-(6-methyl-1-oxy-pyridin-2-yl)-5,6-dihydro-4H-pyrrolo[1,2-b]pyrazole;
4-(Quinolin-N-1-oxide-4-yl)-3-(6-methylpyridin-2-yl)-5,6-dihydro-4H-pyrrolo[1,2-b]pyrazole;
6-Methanesulfonyl-4-(2-pyridin-2-yl-5,6-dihydro-4H-pyrrolo[1,2-b]pyrazol-3-yl)-quinoline;
7-Ethanesulfonyl-4-(2-pyridin-2-yl-5,6-dihydro-4H-pyrrolo[1,2-b]pyrazol-3-yl)-quinoline;
4-(2-Pyridin-2-yl-5,6-dihydro-4H-pyrrolo[1,2-b]pyrazol-3-yl)-7-[3-(pyrimidine-2-sulfonyl)-propoxy]-quinoline;
7-[3-(1-Methyl-1H-imidazole-2-sulfonyl)-propoxy]-4-(2-pyridin-2-yl-5,6-dihydro-4H-pyrrolo[1,2-b]pyrazol-3-yl)-quinoline;
7-[3-(4-Chloro-benzenesulfonyl)-propoxy]-4-(2-pyridin-2-yl-5,6-dihydro-4H-pyrrolo[1,2-b]pyrazol-3-yl)-quinoline;
4-(2-Pyridin-2-yl-5,6-dihydro-4H-pyrrolo[1,2-b]pyrazol-3-yl)-7-[3-(pyridin-2-ylmethanesulfonyl)-propoxy]-quinoline;
4-(2-Pyridin-2-yl-5,6-dihydro-4H-pyrrolo[1,2-b]pyrazol-3-yl)-7-[3-(pyridin-2-ylmethanesulfinyl)-propoxy]-quinoline;
4-(Quinolin-1-N-oxide-4-yl)-3-(6-methylpyridin-2-yl-1-N-oxide)-5,6-dihydro-4H-pyrrolo[1,2-b]pyrazole;
3-{4-[2-(6-Methyl-pyridin-2-yl)-5,6-dihydro-4H-pyrrolo[1,2-b]pyrazol-3-yl]-quinolin-7-yl}-acrylic acid methyl ester;
3-{4-[2-(6-Methylpyridin-2-yl-5,6-dihydro-4H-pyrrolo[1,2-b]pyrazol-3-yl]quinolin-7-yl}-1-piperidin-1-yl-propenone;
3-{4-[2-(6-Methyl-pyridin-2-yl)-5,6-dihydro-4H-pyrrolo[1,2-b]pyrazol-3-yl]-quinolin-6-yl}-acrylic acid methyl ester;
4-[2-(6-Methyl-pyridin-2-yl)-5,6-dihydro-4H-pyrrolo[1,2-b]pyrazol-3-yl]-7-vinyl-quinoline;
4-[2-(6-Benzyl-pyridin-2-yl)-5,6-dihydro-4H-pyrrolo[1,2-b]pyrazol-3-yl]-quinoline;
7-Benzyl-4-[2-(6-methyl-pyridin-2-yl)-5,6-dihydro-4H-pyrrolo[1,2-b]pyrazol-3-yl]-quinoline;
4-[2-(6-Methyl-pyridin-2-yl)-5,6-dihydro-4H-pyrrolo[1,2-b]pyrazol-3-yl]-quinoline-7-carboxylic acid;
4-[2-(6-Methyl-pyridin-2-yl)-5,6-dihydro-4H-pyrrolo[1,2-b]pyrazol-3-yl]-quinoline-6-carboxylic acid;
3-{4-[2-(6-Methyl-pyridin-2-yl)-5,6-dihydro-4H-pyrrolo[1,2-b]pyrazol-3-yl]-quinolin-7-yl}-acrylic acid;
3-{4-[2-(6-Methyl-pyridin-2-yl)-5,6-dihydro-4H-pyrrolo[1,2-b]pyrazol-3-yl]-quinolin-7-yl}-propionic acid;
4-[2-(6-Methyl-pyridin-2-yl)-3-quinolin-4-yl-5,6-dihydro-4H-pyrrolo[1,2-b]pyrazol-5-yl]-benzoic acid;
4-(2-Pyridin-2-yl-5,6-dihydro-4H-pyrrolo[1,2-b]pyrazol-3-yl)-quinoline-7-carboxylic acid cyclopentylamide;
4-(2-Pyridin-2-yl-5,6-dihydro-4H-pyrrolo[1,2-b]pyrazol-3-yl)-quinoline-7-carboxylic acid (2-morpholin-4-yl-ethyl)-amide;
4-(2-Pyridin-2-yl-5,6-dihydro-4H-pyrrolo[1,2-b]pyrazol-3-yl)-quinoline-7-carboxylic acid [2-(1H-imidazol-4-yl)-ethyl]-amide;
4-(2-Pyridin-2-yl-5,6-dihydro-4H-pyrrolo[1,2-b]pyrazol-3-yl)-quinoline-7-carboxylic acid (2-methylamino-ethyl)-amide;
4-(2-Pyridin-2-yl-5,6-dihydro-4H-pyrrolo[1,2-b]pyrazol-3-yl)-quinoline-7-carboxylic acid (3-methylamino-propyl)-amide;
4-(2-Pyridin-2-yl-5,6-dihydro-4H-pyrrolo[1,2-b]pyrazol-3-yl)-quinoline-7-carboxylic acid (2-dimethylamino-ethyl)-amide;
(4-Methyl-piperazin-1-yl)-[4-(2-pyridin-2-yl-5,6-dihydro-4H-pyrrolo[1,2-b]pyrazol-3-yl)-quinolin-7-yl]-methanone;
4-(2-Pyridin-2-yl-5,6-dihydro-4H-pyrrolo[1,2-b]pyrazol-3-yl)-quinoline-7-carboxylic acid cyclobutylamide;
4-(2-Pyridin-2-yl-5,6-dihydro-4H-pyrrolo[1,2-b]pyrazol-3-yl)-quinoline-7-carboxylic acid cyclopropylamide,
4-(2-Pyridin-2-yl-5,6-dihydro-4H-pyrrolo[1,2-b]pyrazol-3-yl)-quinoline-7-carboxylic acid (1-ethyl-propyl)-amide;
4-(2-Pyridin-2-yl-5,6-dihydro-4H-pyrrolo[1,2-b]pyrazol-3-yl)-quinoline-7-carboxylic acid ethylamide;
4-(2-Pyridin-2-yl-5,6-dihydro-4H-pyrrolo[1,2-b]pyrazol-3-yl)-quinoline-7-carboxylic acid isobutyl-amide;
4-(2-Pyridin-2-yl-5,6-dihydro-4H-pyrrolo[1,2-b]pyrazol-3-yl)-quinoline-7-carboxylic acid tert-butylamide;
4-(2-Pyridin-2-yl-5,6-dihydro-4H-pyrrolo[1,2-b]pyrazol-3-yl)-quinoline-7-carboxylic acid isopropylamide;
4-(2-Pyridin-2-yl-5,6-dihydro-4H-pyrrolo[1,2-b]pyrazol-3-yl)-quinoline-7-carboxylic acid propylamide;
4-(2-Pyridin-2-yl-5,6-dihydro-4H-pyrrolo[1,2-b]pyrazol-3-yl)-quinoline-7-carboxylic acid (2-methyl-butyl)-amide;
4-(2-Pyridin-2-yl-5,6-dihydro-4H-pyrrolo[1,2-b]pyrazol-3-yl)-quinoline-7-carboxylic acid ((2S)-2-methyl-butyl)-amide;
4-(2-Pyridin-2-yl-5,6-dihydro-4H-pyrrolo[1,2-b]pyrazol-3-yl)-quinoline-7-carboxylic acid (2S)-sec-butylamide;
4-(2-Pyridin-2-yl-5,6-dihydro-4H-pyrrolo[1,2-b]pyrazol-3-yl)-quinoline-7-carboxylic acid (2R)-sec-butylamide;
4-(2-Pyridin-2-yl-5,6-dihydro-4H-pyrrolo[1,2-b]pyrazol-3-yl)-quinoline-7-carboxylic acid ((IR)-1,2-dimethyl-propyl)-amide;
4-(2-Pyridin-2-yl-5,6-dihydro-4H-pyrrolo[1,2-b]pyrazol-3-yl)-quinoline-7-carboxylic acid (pyridin-4-ylmethyl)-amide;
4-(2-Pyridin-2-yl-5,6-dihydro-4H-pyrrolo[1,2-b]pyrazol-3-yl)-quinoline-7-carboxylic acid (pyridin-3-ylmethyl)-amide;
4-(2-Pyridin-2-yl-5,6-dihydro-4H-pyrrolo[1,2-b]pyrazol-3-yl)-quinoline-7-carboxylic acid (pyridin-2-ylmethyl)-amide;
6-(3-Quinolin-4-yl-5,6-dihydro-4H-pyrrolo[1,2-b]pyrazol-2-yl)-pyridine-2-carboxylic acid amide;
1-(4-Methyl-piperazin-1-yl)-2-[4-(2-pyridin-2-yl-5,6-dihydro-4H-pyrrolo[1,2-b]pyrazol-3-yl)-quinolin-7-yloxy]-ethanone;
N-(2-dimethylamino-ethyl)-2-[4-(2-pyridin-2-yl-5,6-dihydro-4H-pyrrolo[1,2-b]pyrazol-3-yl)-quinolin-7-yloxy]-acetamide;
N-(2-dimethylamino-ethyl)-N-methyl-2-[4-(2-pyridin-2-yl-5,6-dihydro-4H-pyrrolo[1,2-b]pyrazol-3-yl)-quinolin-7-yloxy]-acetamide;
N,N-Dimethyl-3-[4-(2-pyridin-2-yl-5,6-dihydro-4H-pyrrolo[1,2-b]pyrazol-3-yl)-quinolin-7-yloxy]-benzamide;
4-(2-Pyridin-2-yl-5,6-dihydro-4H-pyrrolo[1,2-b]pyrazol-3-yl)-quinoline-7-carboxylic acid amide;
4-(2-Pyridin-2-yl-5,6-dihydro-4H-pyrrolo[1,2-H]pyrazol-3-yl)-quinoline-7-carboxylic acid (2-dimethylamino-ethyl)-methyl-amide;
4-(2-Pyridin-2-yl-5,6-dihydro-4H-pyrrolo[1,2-H]pyrazol-3-yl)-quinoline-7-carboxylic acid (3-dimethylamino-propyl)-methyl-amide;
4-(2-Pyridin-2-yl-5,6-dihydro-4H-pyrrolo[1,2-H]pyrazol-3-yl)-quinoline-7-carboxylic acid dimethylamide;
4-(2-Pyridin-2-yl-5,6-dihydro-4H-pyrrolo[1,2-H]pyrazol-3-yl)-quinoline-7-carboxylic acid methylamide;
4-(2-Pyridin-2-yl-5,6-dihydro-4H-pyrrolo[1,2-b]pyrazol-3-yl)-quinoline-7-carboxylic acid pyridin-2-ylamide;
N-(2,2-Dimethylamino-ethyl)-N-methyl-3-{4-[2-(6-methyl-pyridin-2-yl)-5,6-dihydro-4H-pyrrolo[1,2-b]pyrazol-3-yl]-quinolin-7-yl}-propionamide;
2-(6-Methyl-pyridin-2-yl)-5,6-dihydro-4H-pyrrolo[1,2-b]pyrazol-3-yl]-quinoline-6-carboxylic acid (2-dimethylamino-ethyl)-amide;
4-[2-(6-Methyl-pyridin-2-yl)-5,6-dihydro-4H-pyrrolo[1,2-b]pyrazol-3-yl]-quinoline-6-carboxylic acid (3-dimethylamino-propyl)-amide;
4-[2-(6-Methyl-pyridin-2-yl)-5,6-dihydro-4H-pyrrolo[1,2-b]pyrazol-3-yl]-quinoline-6-carboxylic acid (2-morpholin-4-yl-ethyl)-amide;
1-[2-(Quinolin-4-yl)-1-(6-methyl-pyridin-2-yl)-5,6-dihydro-4H-pyrrolo[1,2-b]pyrazol-3-yl]quinoline-7-carboxylic acid N,N-dimethylaminoethylamide;
4-[2-(6-Methylpyridin-2-yl)-5,6-dihydro-4H-pyrrolo[1,2-b]pyrazol-3-yl]quinoline-7-carbox-ylic acid (2-piperidin-1-yl-ethyl)amide;
N-(2-Dimethylamino-ethyl)-3-{4-[2-(6-methyl-pyridin-2-yl)-5,6-dihydro-4H-pyrrolo[1,2-b]pyrazol-3-yl]-quinolin-7-yl}-propionamide;
4-[2-(6-Methyl-pyridin-2-yl)-5,6-dihydro-4H-pyrrolo[1,2-b]pyrazol-3-yl]-quinoline-7-carboxylic acid (3-dimethylamino-propyl)-amide;
4-(2-(6-Methyl-pyridin-2-yl)-5,6-dihydro-4H-pyrrolo[1,2-b]pyrazol-3-yl]-quinoline-7-carboxylic acid (3-pyrrolidin-1-yl-propyl)-amide;
4-(2-(6-Methyl-pyridin-2-yl)-5,6-dihydro-4H-pyrrolo[1,2-b]pyrazol-3-yl]-quinoline-7-carboxylic acid (3-morpholin-4-yl-propyl)-amide;
3-{4-[2-(6-Methyl-pyridin-2-yl)-5,6-dihydro-4H-pyrrolo[1,2-b]pyrazol-3-yl]-quinolin-7-yl}-propionamide;
4-(2-Pyridin-2-yl-5,6-dihydro-4H-pyrrolo[1,2-b]pyrazol-3-yl)-quinoline-6-carboxylic acid (2-dimethylamino-ethyl)-amide;
4-(2-Pyridin-2-yl-5,6-dihydro-4H-pyrrolo[1,2-b]pyrazol-3-yl)-quinoline-6-carboxylic acid (2-morpholin-4-yl-ethyl)-amide;
4-(2-Pyridin-2-yl-5,6-dihydro-4H-pyrrolo[1,2-b]pyrazol-3-yl)-quinoline-6-carboxylic acid;
4-(2-Pyridin-2-yl-5,6-dihydro-4H-pyrrolo[1,2-b]pyrazol-3-yl)-quinoline-6-carboxylic acid hydrazide;
4-(2-Pyridin-2-yl-5,6-dihydro-4H-pyrrolo[1,2-b]pyrazol-3-yl)-quinoline-6-carboxylic acid amide;
4-(2-Pyridin-2-yl-5,6-dihydro-4H-pyrrolo[1,2-b]pyrazol-3-yl)-quinoline-6-carboxylic acid (3-methylamino-propyl)-amide;
4-(2-Pyridin-2-yl-5,6-dihydro-4H-pyrrolo[1,2-b]pyrazol-3-yl)-quinoline-6-carboxylic acid amide;
4-(2-Pyridin-2-yl-5,6-dihydro-4H-pyrrolo[1,2-b]pyrazol-3-yl)-quinoline-6-carboxylic acid (2-hydroxy-ethyl)-amide;
4-(2-Pyridin-2-yl-5,6-dihydro-4H-pyrrolo[1,2-b]pyrazol-3-yl)-quinoline-7-carboxylic acid hydrazide;
4-(2-Pyridin-2-yl-5,6-dihydro-4H-pyrrolo[1,2-b]pyrazol-3-yl)-quinoline-7-carboxylic acid hydroxyamide;
4-(2-Pyridin-2-yl-5,6-dihydro-4H-pyrrolo[1,2-b]pyrazol-3-yl)-quinoline-7-carboxylic acid (2-amino-ethyl)-amide;
4-(2-Pyridin-2-yl-5,6-dihydro-4H-pyrrolo[1,2-b]pyrazol-3-yl)-quinoline-7-carboxylic acid (2-hydroxy-ethyl)-amide;
4-(2-Pyridin-2-yl-5,6-dihydro-4H-pyrrolo[1,2-b]pyrazol-3-yl)-quinoline-7-sulfonic acid amide;
4-(2-Pyridin-2-yl-5,6-dihydro-4H-pyrrolo[1,2-b]pyrazol-3-yl)-quinoline-7-sulfonic acid methylamide;
4-(2-Pyridin-2-yl-5,6-dihydro-4H-pyrrolo[1,2-b]pyrazol-3-yl)-quinoline-7-sulfonic acid dimethylamide;
4-(2-Pyridin-2-yl-5,6-dihydro-4H-pyrrolo[1,2-b]pyrazol-3-yl)-quinoline-7-sulfonic acid (3-dimethylamino-propyl)-amide;
4-(2-Pyridin-2-yl-5,6-dihydro-4H-pyrrolo[1,2-b]pyrazol-3-yl)-quinoline-7-sulfonic acid diethylamide;
4-(2-Pyridin-2-yl-5,6-dihydro-4H-pyrrolo[1,2-b]pyrazol-3-yl)-quinoline-7-sulfonic acid (2-piperidin-1-yl-ethyl)-amide;
4-(2-Pyridin-2-yl-5,6-dihydro-4H-pyrrolo[1,2-b]pyrazol-3-yl)-quinoline-7-sulfonic acid (2-hydroxy-ethyl)-amide;
4-(2-Pyridin-2-yl-5,6-dihydro-4H-pyrrolo[1,2-b]pyrazol-3-yl)-quinolin-7-ylamine;
2-Dimethylamino-N-[4-(2-pyridin-2-yl-5,6-dihydro-4H-pyrrolo[1,2-b]pyrazol-3-yl)-quinolin-7-yl]acetamide;
3-Dimethylamino-N-[4-(2-pyridin-2-yl-5,6-dihydro-4H-pyrrolo[1,2-b]pyrazol-3-yl)-quinolin-7-yl]propionamide;
N-[4-(2-Pyridin-2-yl-5,6-dihydro-4H-pyrrolo[1,2-b]pyrazol-3-yl)-quinolin-7-yl]-methanesulfonamide;
N-4-(2-Pyridin-2-yl-5,6-dihydro-4H-pyrrolo[1,2-b]pyrazol-3-yl)-quinolin-7-yl]-acetamide;
4-(2-Pyridin-2-yl-5,6-dihydro-4H-pyrrolo[1,2-b]pyrazol-3-yl)-quinoline-7-carboxylic acid (2-acetylamino-ethyl)-amide;
N-{3-[4-(2-Pyridin-2-yl-5,6-dihydro-4H-pyrrolo[1,2-b]pyrazol-3-yl)-quinolin-7-yloxy]-propyl}-methanesulfonamide;
1-methyl-1H-imidazole-4-sulfonic acid{3-[4-(2-pyridin-2-yl-5,6-dihydro-4H-pyrrolo[1,2-b]pyrazol-3-yl)-quinolin-7-yloxy]-propyl}-amide;
1-(2-Dimethylamino-ethyl)-3-[4-(2-pyridin-2-yl-5,6-dihydro-4H-pyrrolo[1,2-b]pyrazol-3-yl)-quinolin-7-yl]-urea;
1-(3-Dimethylamino-propyl)-3-[4-(2-pyridin-2-yl-5,6-dihydro-4H-pyrrolo[1,2-b]pyrazol-3-yl)-quinolin-7-yl]-urea;
1-(2-Hydroxy-ethyl)-3-[4-(2-pyridin-2-yl-5,6-dihydro-4H-pyrrolo[1,2-b]pyrazol-3-yl)-quinolin-7-yl]-urea;
[4-(2-Pyridin-2-yl-5,6-dihydro-4H-pyrrolo[1,2-b]pyrazol-3-yl)-quinolin-7-yl]-carbamic acid methyl ester;
[4-(2-Pyridin-2-yl-5,6-dihydro-4H-pyrrolo[1,2-b]pyrazol-3-yl)-quinolin-7-yl]-carbamic acid 2-hydroxy-ethyl ester;
[4-(2-Pyridin-2-yl-5,6-dihydro-4H-pyrrolo[1,2-b]pyrazol-3-yl)-quinolin-7-yl]-carbamic acid 2-methoxy-ethyl ester;
1,3-Bis-[4-(2-pyridin-2-yl-5,6-dihydro-4H-pyrrolo[1,2-b]pyrazol-3-yl)-quinolin-7-yl]-urea; Dimethyl-carbamic acid 4-(2-pyridin-2-yl-5,6-dihydro-4H-pyrrolo[1,2-b]pyrazol-3-yl)-quinolin-7-yl ester;
7-Bromo-2-isopropyl-4-(2-pyridin-2-yl-5,6-dihydro-4H-pyrrolo[1,2-b]pyrazol-3-yl)-quinoline;
2-[4-(2-(6-Methyl-pyridin-2-yl-5,6-dihydro-4H-pyrrolo[1,2-b]pyrazol-3-yl]-quinolin-6-yl)-propan-2-ol;
7-(3-Chloro-propylsulfanyl)-4-(2-pyridin-2-yl-5,6-dihydro-4H-pyrrolo[1,2-b]pyrazol-3-yl)-quinoline;
7-Bromo-4-(4-chloro-2-pyridin-2-yl-5,6-dihydro-4H-pyrrolo[1,2-b]pyrazol-3-yl)-quinoline;
8-Chloro-4-(2-Pyridin-2-yl-5,6-dihydro-4H-pyrrolo[1,2-b]pyrazol-3-yl)-quinolin-7-ol;
8-Bromo-4-(2-pyridin-2-yl-5,6-dihydro-4H-pyrrolo[1,2-b]pyrazol-3-yl)-quinolin-7-ol;
3-(7-Bromo-quinolin-4-yl)-2-pyridin-2-yl-5,6-dihydro-4H-pyrrolo[1,2-b]pyrazol-4-ol;
7-Bromo-4-(4-methoxy-2-pyridin-2-yl-5,6-dihydro-4H-pyrrolo[1,2-b]pyrazol-3-yl)-quinoline;
[3-(7-Bromo-quinolin-4-yl)-2-pyridin-2-yl-5,6-dihydro-4H-pyrrolo[1,2-b]pyrazol-4-yl]-methyl-amine;
3-(7-Bromo-quinolin-4-yl)-2-pyridin-2-yl-5,6-dihydro-pyrrolo[1,2-b]pyrazol-4-one;
3-[4-(2-Pyridin-2-yl-5,6-dihydro-4H-pyrrolo[1,2-b]pyrazol-3-yl)-quinolin-7-yloxy]-benzamide;
N,N-Dimethyl-3-[4-(2-pyridin-2-yl-5,6-dihydro-4H-pyrrolo[1,2-b]pyrazol-3-yl)-quinolin-7-yloxy]-thiobenzamide;
Dimethyl-{3-[4-(2-Pyridin-2-yl-5,6-dihydro-4H-pyrrolo[1,2-b]pyrazol-3-yl)-quinolin-7-yloxy]-benzyl}-amine;
4-(2-(6-Methyl-pyridin-2-yl-5,6-dihydro-4H-pyrrolo[1,2-b]pyrazol-3-yl]-1H-quinolin-2-one;
4-(2-Pyridin-2-yl-5,6-dihydro-4H-pyrrolo[1,2-b]pyrazol-3-yl)-quinolin-7-ol;
4-[2-(6-Methyl-pyridin-2-yl)-5,6-dihydro-4H-pyrrolo[1,2-b]pyrazol-3-yl]-quinolin-7-ol;
6-Methoxy-4-(2-pyridin-2-yl-5,6-dihydro-4H-pyrrolo[1,2-b]pyrazol-3-yl)-quinolin-7-ol;
3-{4-[2-(6-Methyl-pyridin-2-yl)-5,6-dihydro-4H-pyrrolo[1,2-b]pyrazol-3-yl]-quinolin-7-yl}-propionic acid methyl ester;
4-(6-(Methyl-2-pyridin-2-yl-5,6-dihydro-4H-pyrrolo[1,2-b]pyrazol-3-yl)-quinoline;
3-{4-[2-(6-Methyl-pyridin-2-yl)-5,6-dihydro-4H-pyrrolo[1,2-b]pyrazol-3-yl]-quinolin-6-yl}-propionic acid methyl ester;
7-Amino-4-[2-(6-Methyl-pyridin-2-yl)-5,6-dihydro-4H-pyrrolo[1,2-b]pyrazol-3-yl]-quinoline;
N,N-Dimethyl-3-{4-(2-methyl-pyridin-2-yl-5,6-dihydro-4H-pyrrolo[1,2-b]pyrazol-3-yl]-quinolin-7-yl}-propionamide;
N-{3-[4-(2-Pyridin-2-yl-5,6-dihydro-4H-pyrrolo[1,2-b]pyrazol-3-yl)-quinolin-7-yloxy]-propyl}-acetamide;
N-Acetyl-N-{4-[2-(6-methyl-yridin-2-yl)-5,6-dihydro-4H-pyrrolo[1,2-b]pyrazol-3-yl]-quinolin-7-yl}-acetamide,
2-Pyridin-2-yl-3-quinolin-4-yl-pyrazolo[1,5-a]piperidin-7-ol;
7-Acetoxy-2-pyridin-2-yl-3-quinolin-4-yl-pyrazol[1,5-a]piperidine;
Methyl-{3-[4-(2-pyridin-2-yl-5,6-dihydro-4H-pyrrolo[1,2-b]pyrazol-3-yl)-quinolin-7-yloxy]-propyl}-amine;
7-(Piperidin-4-yloxy)-4-(2-pyridin-2-yl-5,6-dihydro-4H-pyrrolo[1,2-b]pyrazol-3-yl)-quinoline;
4-(6-(Methyl-2-pyridin-2-yl)-5,6-dihydro-4H-pyrrolo[1,2-b]pyrazol-3-yl]-quinoline-7-carboxylic acid (2-amino-1,1-dimethyl-ethyl)-amide;
16-[3-(4-Fluoro-phenyl)-5,6-dihydro-4H-pyrrolo[1,2-b]pyrazol-2-yl]pyridin-2-yl}-methanol, rrrrrm-rrrrr)[6-(3-Quinolin-4-yl-5,6-dihydro-4H-pyrrolo[1,2-b]pyrazol-2-yl)-pyridin-2-yl]methanol;
4-(6-(Methyl-2-pyridin-2-yl-5,6-dihydro-4H-pyrrolo[1,2-b]pyrazol-3-yl]-phenol;
7-(1-Methyl-pyrrolidin-3-ylmethoxy)-4-(2-pyridin-2-yl-5,6-dihydro-4H-pyrrolo[1,2-b]pyrazol-3-yl)-quinoline;
7-(1-Methyl-piperidin-4-ylmethoxy)-4-(2-pyridin-2-yl-5,6-dihydro-4H-pyrrolo[1,2-b]pyrazol-3-yl)-quinoline;
4-[2-(6-Methyl-pyridin-2-yl)-5,6-dihydro-4H-pyrrolo[1,2-b]pyrazol-3-yl]-quinoline-7-carboxylic acid (2-dimethylamino-1,1-dimethyl-ethyl)-amide;
(S)-[3-(4-Fluoro-phenyl)-2-(6-methyl-pyridin-2-yl)-5,6-dihydro-4H-pyrrolo[1,2-b]pyrazol-6-yl]-methanol;
(R)-[3-(4-Fluoro-phenyl)-2-(6-methyl-pyridin-2-yl)-5,6-dihydro-4H-pyrrolo[1,2-b]pyrazol-6-yl]-methanol;
(S)-[3-(4-Fluoro-phenyl)-2-(6-methyl-pyridin-2-yl)-5,6-dihydro-4H-pyrrolo[1,2-b]pyrazol-6-yl]-acetonitrile;
(R)-[3-(4-Fluoro-phenyl)-2-(6-methyl-pyridin-2-yl)-5,6-dihydro-4H-pyrrolo[1,2-b]pyrazol-6-yl]-acetonitrile;
4-(3-Pyridin-2-yl-5,6-dihydro-4H-pyrrolo[1,2-b]pyrazol-2-yl)-quinoline;
4-(6-Pyridin-2-yl-2,3-dihydro-pyrazolo[5,1-b]oxazol-7-yl)-quinoline;
3-[4-(2-Pyridin-2-yl-5,6-dihydro-4H-pyrrolo[1,2-b]pyrazol-3-yl)-quinolin-7-yl]-oxazolidin-2-one;
1-[4-(2-Pyridin-2-yl-5,6-dihydro-4H-pyrrolo[1, 2b]pyrazol-3-yl)-quinolin-7-yl]-imidazolidin-2-one;
4-(2-Pyridin-2-yl-5,6-dihydro-4H-pyrrolo[1,2-b]pyrazol-3-yl)-7-(pyridin-4-ylmethoxy)-quinoline;
4-(2-Pyridin-2-yl-5,6-dihydro-4H-pyrrolo[1,2-b]pyrazol-3-yl)-7-(3-pyridin-3-yl-propoxy)-quinoline;
7-(4,5-Dihydro-1H-imidazol-2-yl)-4-(2-pyridin-2-yl-5,6-dihydro-4H-pyrrolo[1,2-b]pyrazol-3-yl)-quinoline;
4-[5-(4-Fluoro-phenyl)-2-(6-methyl-pyridin-2-yl)-5,6-dihydro-4H-pyrrolo[1,2-b]pyrazol-3-yl]-quinoline (Enantiomer A);
4-[5-(4-Fluoro-phenyl)-2-(6-methyl-pyridin-2-yl)-5,6-dihydro-4H-pyrrolo[1,2-b]pyrazol-3-yl]-quinoline (Enantiomer B);
2-Pyridin-2-yl-3-quinolin-4-yl-pyrazolo[5,1-c]morpholine;
4-[2-(6-Vinyl-pyridin-2-yl)-5,6-dihydro-4H-pyrrolo[1,2-b]pyrazol-3-yl]-quinoline;
3-{4-[2-(6-Methyl-pyridin-2-yl)-5,6-dihydro-4H-pyrrolo[1,2-b]pyrazol-3-yl]-quinolin-6-yl}-acrylic acid;
7-(6-Methyl-pyridin-3-yloxy)-4-(2-pyridin-2-yl-5,6-dihydro-4H-pyrrolo[1,2-b]pyrazol-3-yl)-quinoline;
4-(2-Pyridin-2-yl-5,6-dihydro-4H-pyrrolo[1,2-b]pyrazol-3-yl)-7-[4-(4-pyrimidin-2-yl-piperazin-1-yl)-butoxy]-quinoline;
7-[3-[4-(2-Methoxy-phenyl)-piperazin-1-yl]-propoxy]-4-(2-pyridin-2-yl-5,6-dihydro-4H-pyrrolo[1,2-b]pyrazol-3-yl)-quinoline;
Pyridin-2-yl-{3-[4-(2-pyridin-2-yl-5,6-dihydro-4H-pyrrolo[1,2-b]pyrazol-3-yl)-quinolin-7-yloxy]-propyl}-amine;
4-(6-(Methyl-pyridin-2-yl)-5,6-dihydro-4H-pyrrolo[1,2-b]pyrazol-3-yl]-quinoline-7-carboxylic acid (2-dimethylamino-1-methyl-ethyl)-amide, rrrrnTn-rr)4-[2-(6-Methyl-pyridin-2-yl)-5,6-dihydro-4H-pyrrolo[1,2-b]pyrazol-3-yl]-quinoline-7-carboxylic acid amide;
4-(2-Pyridin-2-yl-5,6-dihydro-4H-pyrrolo[1,2-b]pyrazol-3-yl)-quinoline-7-carboxylic acid (3-dimethylamino-propyl)-amide;
4-[2-(6-Methyl-pyridin-2-yl)-5,6-dihydro-4H-pyrrolo[1,2-b]pyrazol-3-yl]-quinoline-7-carboxylic acid (2-dimethylamino-ethyl)-methyl-amide;
N,N-Dimethyl-3-{4-[2-(6-methyl-pyridin-2-yl)-5,6-dihydro-4H-pyrrolo[1,2-b]pyrazol-3-yl]-quinolin-7-yl}-acrylamide;
4-(2-Pyridin-2-yl-5,6-dihydro-4H-pyrrolo[1,2-b]pyrazol-3-yl)-quinoline 1-oxide;
7-Benzyloxy-4-[2-(6-methyl-pyridin-2-yl)-5,6-dihydro-4H-pyrrolo[1,2-b]pyrazol-3-yl]-quinoline;
4-(2-(6-Chloro-6-dihydro-4H-pyrrolo loro-pyridin-2-yl)-5[1,2-b]pyrazol-3-yl)-quinoline;
6-(3-Quinolin-4-yl-5,6-dihydro-4H-pyrrolo[1,2-b]pyrazol-2-yl)pyridine-2-carboxylic acid methyl ester;
4-(7-Chloroquinolin-4-yl)-3-(pyridin-2-yl)-5,6-dihydro-4H-pyrrolo[1,2#b]pyrazole;
4-(2-Furan-2-yl-5,6-dihydro-4H-pyrrolo[1,2-b]pyrazol-3-yl)-quinoline;
3-{4-(6-Methyl-pyridin-2-yl)-5,6-dihydro-4H-pyrrolo[1,2-b]pyrazol-3-yl]-quinolin-6-yl}-acrylic acid methyl ester;
4-[2-(2-Methyl-thiazol-4-yl)-5,6-dihydro-4H-pyrrolo[1,2-b]pyrazol-3-yl]-quinoline;
3-(4-Fluoro-phenyl)-2-(2-methyl-thiazol-4-yl)-5,6-dihydro-4H-pyrrolo[1,2-b]pyrazole;
4-[2-(2-Methyl-2H-pyrazol-3-yl)-5,6-dihydro-4H-pyrrolo[1,2-b]pyrazol-3-yl]-quinoline;
4-(2-Thiazol-2-yl-5,6-dihydro-4H-pyrrolo[1,2-b]pyrazol-3-yl)-quinoline;
4-[2-(1-Methyl-1H-imidazol-2-yl-5,6-dihydro-4H-pyrrolo[1,2-b]pyrazol-3-yl]-quinoline;
6,7-Dichloro-4-[2-(6-methyl-pyridin-2-yl)-5,6-dihydro-4H-pyrrolo[1,2-b]pyrazol-3-yl]-quinoline;
(S)-6-Benzyloxymethyl-3-(4-fluoro-phenyl)-2-(6-methyl-pyridin-2-yl)-5,6-dihydro-4H-pyrrolo[1,2-b]pyrazole;
N,N-Dimethyl-3-{4-[2-(6-methyl-pyridin-2-yl)-5,6-dihydro-4H-pyrrolo[1,2-b]pyrazol-3-yl]-quinolin-7-yl}-acrylamide;
3-methyl-6-[2-[6-methyl-(pyridin-2-yl)]-5,6-di-hydro-4H-pyrrolo[1,2-b]pyrazol-3-yl]-3H-quinazolin-4-one;
1-methyl-7-[2-[6-methyl-(pyridin-2-yl)]-5,6-di-hydro-4H-pyrrolo[1,2-b]pyrazol-3-yl]-1H-quinoxalin-2-one;
3-methyl-6-[2-(pyridin-2-yl)-5,6-dihydro-4H-pyrrolo[1,2-b]pyrazol-3-yl]-3H-quinazolin-4-one;
3-methyl-6-[2-[6-pentyl-(pyridin-2-yl)]-5,6-di-hydro-4H-pyrrolo[1,2-b]pyrazol-3-yl]-3H-quinazolin-4-one;
6-[2-[6-Methyl-(pyridin-2-yl)]-5,6-dihydro-4H-pyrrolo[1,2-b]pyrazol-3-yl]-4H-benzo[1,4]oxazin-3-one;
3-(2-Chloro-ethyl)-6-[2-[6-methyl-(pyridin-2-yl)]-5,6-dihydro-4H-pyrrolo[1,2-b]pyrazol-3-yl]-3H-quinazolin-4-one;
6-[2-[6-methyl-(pyridin-2-yl)]-5,6-dihydro-4H-pyrrolo[1,2-b]pyrazol-3-yl]-3-(2-morpholin-4-yl-ethyl)-3H-quinazolin-4-one;
3-(2-Dimethylamino-ethyl)-6-[2-[6-methyl-(py-ridin-2-yl)]-5,6-dihydro-4H-pyrrolo[1,2-b]pyrazol-3-yl]-3H-quinazolin-4-one;
6-[2-[6-Methyl-(pyridin-2-yl)]-5,6-dihydro-4H-pyrrolo[1,2-b]pyrazol-3-yl]-3-(2-piperidin-1-yl-ethyl)-3H-quinazolin-4-one;
6-[2-[6-Methyl-(pyridin-2-yl)]-5,6-dihydro-4H-pyrrolo[1,2-b]pyrazol-3-yl]-3-(2-pyrrolidin-1-yl-ethyl)-3H-quinazolin-4-one;
3-(2-Azepan-1-yl-ethyl)-6-[2-[6-methyl-(pyri-din-2-yl)]-5,6-dihydro-4H-pyrrolo[1,2-b]pyrazol-3-yl]-3H-quinazolin-4-one;
7-[2-[6-Methyl-(pyridin-2-yl)]-5,6-dihydro-4H-pyrrolo[1,2-b]pyrazol-3-yl]-1-(2-pyrrolidin-1-yl-ethyl)-3,4-dihydro-1H-quinoxalin-2-one; and
1-(2-Dimethylamino-ethyl)-7-[2-[6-methyl-(pyridin-2-yl)]-5,6-dihydro-4H-pyrrolo[1,2-b]pyrazol-3-yl]-3,4-dihydro-1H-quinoxalin-2-one.

Formula III

In one aspect, the disclosure features a method of producing a reprogrammed cell (e.g. iPS cell or partially reprogrammed cell) from a differentiated cell, the method comprising:

contacting an isolated differentiated cell with a compound of formula (III)

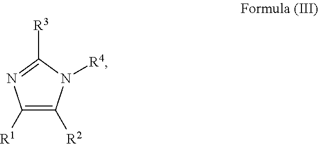

wherein

R¹ is cyclyl, heterocyclcyl, aryl or heteroaryl, each of which can be optionally substituted;

R² is cyclyl, heterocyclcyl, aryl or heteroaryl, each of which can be optionally substituted;

R³ is cyclyl, heterocyclcyl, aryl, heteroaryl or —S(O)alkyl, each of which can be optionally substituted;

R⁴ is H, optionally substituted C₁-C₆ alkyl, optionally substituted C₂-C₆ alkenyl, optionally substituted C₂-C₆ alkynyl, or R³ and R⁴ together with the atoms they are attached to form a cyclyl, heterocyclyl, aryl or heteroaryl, each of which can be optionally substituted;

to thereby produce a reprogrammed cell (e.g. iPS cell or partially reprogrammed cell) from the differentiated cell.

In one embodiment, the method comprises contacting a plurality of differentiated cells with a compound of formula (III) to thereby produce a plurality of reprogrammed cells (e.g. iPS cells or partially reprogrammed cells) from the differentiated cells.

In some embodiments, R¹ is aryl, e.g., a substituted aryl. In some embodiments, R¹ is substituted with two substituents. In some embodiments, R¹ is substituted with two substituents, which together with the carbons to which they are attached form a ring. In some embodiments, R¹ is a substituted phenyl. In some embodiments, R¹ is a nitrogen containing heteroaryl (e.g., including 1, 2, or 3 nitrogens (e.g., 1 or)). In some embodiments, R¹ is a bicyclic heteroaryl. In some embodiments, R¹ is a 6-6 fused heteroaryl. In some embodiments, R¹ is pyridyl, pyrimidyl, pyridazinyl, pyrazinyl, quinolinyl, naphthyridinyl (e.g., 1,5-naphthyridinyl), quinazolinyl, 5,6,7,8-tetrahydroquinazolinyl, 1,3-benzodioxlyl, 1,2,3-benzotriazolyl, benzoxazolyl, benzothiazolyl, 2,1,3-benzooxadiazole, imidazo[1,2-a]pyridinyl, pyrazolo[1,5-a]pyridinyl, [1,2,4]triazolo[1,5-a]pyridinyl, pyrazolo[1,5-a]pyrimidinyl, [1,2,4]triazolo[1,5-a]pyrimidinyl, [1,2,3]triazolo[1,5-a]pyrimidinyl, [1,2,4]triazolo[4,3-a]pyrimidinyl, [1,2,4]triazolo[4,3-a]pyridazinyl. In some embodiments, R¹ is

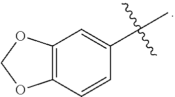

In some embodiment, R² is aryl, e.g., a substituted aryl. In some embodiments, R² is a nitrogen comprising heteroaryl (e.g., including 1, 2 or 3 nitrogens (e.g., 1 or 2)). In some embodiments, R² is an optionally substituted monocyclic heteroaryl (e.g., a six membered heteroaryl such as pyridyl, pyrimidyl, pyridazinyl or pyrazinyl). In some embodiments, R² is substituted. Exemplary substituents include halo, C₁-C₆ alkyl, haloC₁-C₆alkyl, C₁-C₆ alkoxy, OH, haloC₁-C₆alkoxy. In some embodiments, R² is monosubstituted. In some embodiments, R² is substituted with methyl. In one embodiment, R² is an optionally substituted pyridyl. In some embodiments, R² is

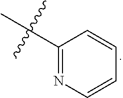

In some embodiments, R³ is aryl, e.g., a substituted aryl. In some embodiments, R³ is substituted with two substituents, which together with the carbons to which they are attached form a ring. In some embodiments, R³ is a substituted phenyl. In some embodiments, R³ is a nitrogen containing heteroaryl (e.g., including 1, 2, or 3 nitrogens (e.g., 1 or)). In some embodiments, R³ is a bicyclic heteroaryl. In some embodiments, R³ is a 6-6 fused heteroaryl. In some embodiments, R³ is pyridyl, pyrimidyl, pyridazinyl, pyrazinyl, quinolinyl, naphthyridinyl (e.g., 1,5-naphthyridinyl), quinazolinyl, 5,6,7,8-tetrahydroquinazolinyl, 1,3-benzodioxlyl, 1,2,3-benzotriazolyl, benzoxazolyl, benzothiazolyl, 2,1,3-benzooxadiazole, imidazo[1,2-a]pyridinyl, pyrazolo[1,5-a]pyridinyl, [1,2,4]triazolo[1,5-a]pyridinyl, pyrazolo[1,5-a]pyrimidinyl, [1,2,4]triazolo[1,5-a]pyrimidinyl, [1,2,3]triazolo[1,5-a]pyrimidinyl, [1,2,4]triazolo[4,3-a]pyrimidinyl, [1,2,4]triazolo[4,3-a]pyridazinyl. In some embodiments, R³ is monosubstituted. In some embodiments, R³ is an optionally substituted phenyl. In some embodiments, R³ is

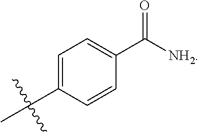

In some embodiment, R⁴ is H.

In some embodiments, the compound of formula (III) has the structure shown in formula (Ma):

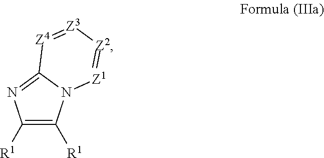

wherein z¹-z⁴ are independently CR⁵ or N; R⁵ is H, benzyl, aryl, heteroaryl, C₁-C₆alkyl, alkenyl, alkynyl, halogen, amino, —C(O)-amino, —SO₂-alkyl, —O-alkyl or acyl, each of which can be optionally substituted, provided that no two N are not next to each other.

In some embodiments, one of z² or Z³ is N.

In some embodiments, the compound of formula (III) has the structure shown in formula (IIIb):

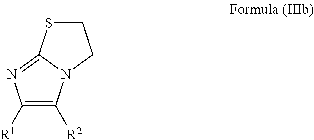

Exemplary compounds of formula (III) include

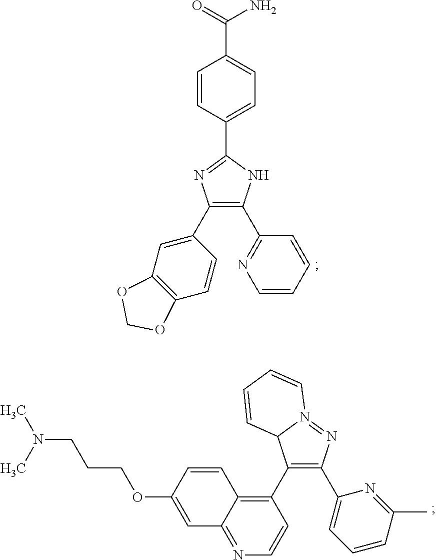

6-(2-(6-methylpyridin-2-yl)H-imidazo[1,2-a]pyridin-3-yl)-N-(3-(piperidin-1-yl)propyl)pyridin-2-amine;
3-isopropyl-6-(5-(6-methylpyridin-2-yl)-2H-1,2,3-triazol-4-yl)H-imidazo[1,2-a]pyridine;
1-(3-((pyridin-3-yl)methoxy)-4-carbamoylisothiazol-5-yl)-3-(3,5-dimethoxybenzyl)urea;
(2-Methoxy-ethyl)-{4-[2-(6-methyl-pyridin-2-yl)-imidazo-[1,2-a]pyridin-3-yl]-pyrimidin-2-yl}-amine;
(3-{4-[2-(6-Methyl-pyridin-2-yl)-imidazo[1,2-a]pyridin-3-yl]-pyrimidin-2-ylamino}-propyl)-carbamic acid tert-butyl ester;
(3-Imidazol-1-yl-propyl)-{4-[2-(6-methyl-pyridin-2-yl)-imidazo[1,2-a]pyridin-3-yl]-pyrimidin-2-yl}-amine;
(4-Methoxy-benzyl)-{4-[2-(6-methyl-pyridin-2-yl)-imidazo[1,2-a]pyridin-3-yl]-pyrimidin-2-yl}-amine;
[2-(6-Methyl-pyridin-2-yl)-3-(2-methylsulfanyl-pyrimidin-4-yl)-imidazo[1,2-a]pyridin-6-yl]-methanol;
3-(2-Methanesulfonyl-pyrimidin-4-yl)-2-(6-methyl-pyridin-2-yl)-imidazo[1,2-a]pyridine;
(4-4-[2-(6-Methyl-pyridin-2-yl)-imidazo[1,2-a]pyridin-3-yl]-pyrimidin-2-ylamino}-butyl)-carbamic acid tert-butyl ester;
(4-Amino-benzyl)-{4-[2-(6-methyl-pyridin-2-yl)-imidazo[1,2-a]pyridin-3-yl]-pyrimidin-2-yl}-amine;
(5-{4-[2-(6-Methyl-pyridin-2-yl)-imidazo[1,2-a]pyridin-3-yl]-pyrimidin-2-ylamino}-pentyl)-carbamic acid tert-butyl ester;
[3-(2-Amino-pyrimidin-4-yl)-2-(6-methyl-pyridin-2-yl)-imidazo[1,2-a]pyridin-6-yl]-methanol;
[3-(2-amino-pyrimidin-4-yl)-2-(6-methyl-pyridin-2-yl)-imidazo[1,2-a]pyridin-7-yl]-methanol;
[3-(2-Amino-pyrimidin-4-yl)-6-methyl-2-(6-methyl-pyridin-2-yl)-imidazo[1,2-a]pyridin-8-yl]-(2-morpholin-4-yl ethyl)-amine;
[3-(2-Amino-pyrimidin-4-yl)-6-methyl-2-(6-methyl-pyridin-2-yl)-imidazo[1,2-a]pyridin-8-yl]-(2-pyridin-2-yl-ethyl)-amine;
[3-(2-Amino-pyrimidin-4-yl)-6-methyl-2-(6-methyl-pyridin-2-yl)-imidazo[1,2-a]pyridin-8-yl]-(2-pyridin-3-yl-ethyl)-amine;
[3-(2-methanesulfonyl-pyrimidin-4-yl)-2-(6-methyl-pyridin-2-yl)-imidazo[1,2-a]pyridin-6-yl]-methanol;
[3-(2-Amino-pyrimidin-4-yl)-6-methyl-2-(6-methyl-pyridin-2-yl)-imidazo[1,2-a]pyridin-8-yl]-(2-pyridin-4-yl-ethyl)-amine;
[3-(2-Amino-pyrimidin-4-yl)-6-methyl-2-(6-methyl-pyridin-2-yl)-imidazo[1,2-a]pyridin-8-yl]-(3-morpholin-4-yl-propyl)-amine;
[3-(4-Methyl-piperazin-1-yl)-propyl]-{4-[2-(6-methyl-pyridin-2-yl)-imidazo[1,2-a]pyridin-3-yl]-pyrimidin-2-yl}-amine;
[3-(4-Methyl-piperidin-1-yl)-propyl]-{4-[2-(6-methyl-pyridin-2-yl)-imidazo[1,2-a]pyridin-3-yl]-pyrimidin-2-yl}-amine;
[4-(2-Pyridin-2-yl-imidazo[1,2-a]pyridin-3-yl)-pyrimidin-2-yl]-pyridin-3-ylmethyl-amine;
{4-[2-(6-Methyl-pyridin-2-yl)-imidazo[1,2-a]pyridin-3-yl]-pyrimidin-2-yl}-((R)-1-phenyl-ethyl)-amine;
{4-[2-(6-Methyl-pyridin-2-yl)-imidazo[1,2-a]pyridin-3-yl]-pyrimidin-2-yl}-((S)-1-phenyl-ethyl)-amine;
{4-[2-(6-Methyl-pyridin-2-yl)-imidazo[1,2-a]pyridin-3-yl]-pyrimidin-2-yl}-(1H-tetrazol-5-yl)-amine;
{4-[2-(6-Methyl-pyridin-2-yl)-imidazo[1,2-a]pyridin-3-yl]-pyrimidin-2-yl}-(2H-pyrazol-3-yl)-amine;
{4-[2-(6-Methyl-pyridin-2-yl)-imidazo[1,2-a]pyridin-3-yl]-pyrimidin-2-yl}-(2-morpholin-4-yl-ethyl)-amine;
{4-[2-(6-Methyl-pyridin-2-yl)-imidazo[1,2-a]pyridin-3-yl]-pyrimidin-2-yl}-(2-pyridin-2-yl-ethyl)-amine;
{4-[2-(6-Methyl-pyridin-2-yl)-imidazo[1,2-a]pyridin-3-yl]-pyrimidin-2-yl}-(2-pyridin-3-yl-ethyl)-amine;
{4-[2-(6-Methyl-pyridin-2-yl)-imidazo[1,2-a]pyridin-3-yl]-pyrimidin-2-yl}-(2-pyridin-4-yl-ethyl)-amine;
{4-[2-(6-Methyl-pyridin-2-yl)-imidazo[1,2-a]pyridin-3-yl]-pyrimidin-2-yl}-(3-morpholin-4-yl-propyl)-amine;
{4-[2-(6-Methyl-pyridin-2-yl)-imidazo[1,2-a]pyridin-3-yl]-pyrimidin-2-yl}-(3-piperidin-1-yl-propyl)-amine;
{4-[2-(6-Methyl-pyridin-2-yl)-imidazo[1,2-a]pyridin-3-yl]-pyrimidin-2-yl}-[1, 3,4]thiadiazol-2-yl-amine;
2-(6-Methyl-pyridin-2-yl)-3-(2-methylsulfanyl-pyrimidin-4-yl)-imidazo[1,2-a]pyridine;
2-(6-Methyl-pyridin-2-yl)-3-(2-methylsulfanyl-pyrimidin-4-yl)-imidazo[1,2-a]pyridine-6-carboxylic acid methyl ester;
2-(6-Methyl-pyridin-2-yl)-3-(2-methylsulfanyl-pyrimidin-4-yl)-imidazo[1,2-a]pyridine-7-carboxylic acid ethyl ester;
2-(6-Methyl-pyridin-2-yl)-3-(2-methylsulfanyl-pyrimidin-4-yl)-imidazo[1,2-a]pyrimidin-7-ylamine;
{7,7-Dimethyl-8-[5-(4-4-[2-(6-methyl-pyridin-2-yl)-imidazo[1,2-a]pyridin-3-yl]-pyrimidin-2-ylamino}-butylcarbamoyl)-pentyl]-2-oxo-4-trifluoromethyl-7,8-dihydro-2H-1-oxa-8-aza-anthracen-5-yl}-methanesulfonic acid;
2-(2,7-Difluoro-6-hydroxy-3-oxo-9,9a-dihydro-3H-xanthen-9-yl)-3,5,6-trifluoro-4-[(4-{4-[2-(6-methyl-pyridin-2-yl)-imidazo[1,2-a]pyridin-3-yl]-pyrimidin-2-ylamino}-butylcarbamoyl)-methylsulfanyl]-benzoic acid;
2-(6-Methyl-pyridin-2-yl)-3-(2-morpholin-4-yl-pyrimidin-4-yl)-imidazo[1,2-a]pyridine;
2-(6-Methyl-pyridin-2-yl)-3-(2-piperidin-1-yl-pyrimidin-4-yl)-imidazo[1,2-a]pyridine;
2-(6-Methyl-pyridin-2-yl)-3-(2-pyrrolidin-1-yl-pyrimidin-4-yl)-imidazo[1,2-a]pyridine;
2-(6-Methyl-pyridin-2-yl)-3-[2-(1H-tetrazol-5-yl]-pyrimidin-4-yl)-imidazo[1,2-a]pyridine;
2-(6-Methyl-pyridin-2-yl)-3-pyrimidin-4-yl-imidazo[1,2-a]pyridine;
2-(6-Methyl-pyridin-2-yl)-3-pyrimidin-4-yl-imidazo[1,2-a]pyrimidin-7-ylamine;
3-(2-Amino-pyrimidin-4-yl)-2-(6-methyl-pyridin-2-yl)-imidazo[1,2-a]pyridin-8-ylamine;
3-(2-Amino-pyrimidin-4-yl)-2-(6-methyl-pyridin-2-yl)-imidazo[1,2-a]pyridine-6-carbonitrile;
3-(2-Amino-pyrimidin-4-yl)-2-(6-methyl-pyridin-2-yl)-imidazo[1,2-a]pyridine-6-carboxylic acid;
3-(2-Amino-pyrimidin-4-yl)-2-(6-methyl-pyridin-2-yl)-imidazo[1,2-a]pyridine-6-carboxylic acid ([1, 4]dioxan-2-ylmethyl)-amide;
3-(2-Amino-pyrimidin-4-yl)-2-(6-methyl-pyridin-2-yl)-imidazo[1,2-a]pyridine-6-carboxylic acid ([1, 4]dioxan-2-ylmethyl)-amide;
3-(2-Amino-pyrimidin-4-yl)-2-(6-methyl-pyridin-2-yl)-imidazo[1,2-a]pyridine-6-carboxylic acid (2-dimethylamino-ethyl)-amide;
3-(2-Amino-pyrimidin-4-yl)-2-(6-methyl-pyridin-2-yl)-imidazo[1,2-a]pyridine-6-carboxylic acid (2-methoxy-ethyl)-amide;
3-(2-Amino-pyrimidin-4-yl)-2-(6-methyl-pyridin-2-yl)-imidazo[1,2-a]pyridine-6-carboxylic acid (2-thiophen-2-yl-ethyl)-amide;
3-(2-Amino-pyrimidin-4-yl)-2-(6-methyl-pyridin-2-yl)-imidazo[1,2-a]pyridine-6-carboxylic acid [3-(4-methyl-piperazin-1-yl)-propyl]-amide;
3-(2-Amino-pyrimidin-4-yl)-2-(6-methyl-pyridin-2-yl)-imidazo[1,2-a]pyridine-6-carboxylic acid amide;
3-(2-Amino-pyrimidin-4-yl)-2-(6-methyl-pyridin-2-yl)-imidazo[1,2-a]pyridine-6-carboxylic acid cyclopropylamide;
3-(2-Amino-pyrimidin-4-yl)-2-(6-methyl-pyridin-2-yl)-imidazo[1,2-a]pyridine-6-carboxylic acid ethylamide;
3-(2-Amino-pyrimidin-4-yl)-2-(6-methyl-pyridin-2-yl)-imidazo[1,2-a]pyridine-6-carboxylic acid hydroxyamide;
3-(2-Amino-pyrimidin-4-yl)-2-(6-methyl-pyridin-2-yl)-imidazo[1,2-a]pyridine-6-carboxylic acid methoxy-amide;
3-(2-Amino-pyrimidin-4-yl)-2-(6-methyl-pyridin-2-yl)-imidazo[1,2-a]pyridine-6-carboxylic acid methyl ester;
3-(2-Amino-pyrimidin-4-yl)-2-(6-methyl-pyridin-2-yl)-imidazo[1,2-a]pyridine-7-carboxylic acid;
3-(2-Amino-pyrimidin-4-yl)-2-(6-methyl-pyridin-2-yl)-imidazo[1,2-a]pyridine-7-carboxylic acid [1, 4]dioxan-2-ylmethyl)-amide;
3-(2-Amino-pyrimidin-4-yl)-2-(6-methyl-pyridin-2-yl)-imidazo[1,2-a]pyridine-7-carboxylic acid (2-amino-ethyl)-amide;
3-(2-Amino-pyrimidin-4-yl)-2-(6-methyl-pyridin-2-yl)-imidazo[1,2-a]pyridine-7-carboxylic acid (2-dimethylamino-ethyl)-amide;
3-(2-Amino-pyrimidin-4-yl)-2-(6-methyl-pyridin-2-yl)-imidazo[1,2-a]pyridine-7-carboxylic acid (2-hydroxy-ethyl)-amide;
3-(2-Amino-pyrimidin-4-yl)-2-(6-methyl-pyridin-2-yl)-imidazo[1,2-a]pyridine-7-carboxylic acid (2-oxo-2-pyridin-3-yl-ethyl)-amide;
3-(2-Amino-pyrimidin-4-yl)-2-(6-methyl-pyridin-2-yl)-imidazo[1,2-a]pyridine-7-carboxylic acid (2-thiophen-2-yl-ethyl)-amide;
3-(2-Amino-pyrimidin-4-yl)-2-(6-methyl-pyridin-2-yl)-imidazo[1,2-a]pyridine-7-carboxylic acid (piperidin-3-ylmethyl)-amide;
3-(2-Amino-pyrimidin-4-yl)-2-(6-methyl-pyridin-2-yl)-imidazo[1,2-a]pyridine-7-carboxylic acid 2,2-dimethylhydrazide;
3-(2-Amino-pyrimidin-4-yl)-2-(6-methyl-pyridin-2-yl)-imidazo[1,2-a]pyridine-7-carboxylic acid amide;
3-(2-Amino-pyrimidin-4-yl)-2-(6-methyl-pyridin-2-yl)-imidazo[1,2-a]pyridine-7-carboxylic acid cyclopropylamide;
3-(2-Amino-pyrimidin-4-yl)-2-(6-methyl-pyridin-2-yl)-imidazo[1,2-a]pyridine-7-carboxylic acid ethyl ester;
3-(2-Amino-pyrimidin-4-yl)-2-(6-methyl-pyridin-2-yl)-imidazo[1,2-a]pyridine-7-carboxylic acid ethylamide;
3-(2-Amino-pyrimidin-4-yl)-2-(6-methyl-pyridin-2-yl)-imidazo[1,2-a]pyridine-7-carboxylic acid hydroxyamide;
3-(2-Amino-pyrimidin-4-yl)-2-(6-methyl-pyridin-2-yl)-imidazo[1,2-a]pyridine-7-carboxylic acid methoxy-amide;
3-(2-Amino-pyrimidin-4-yl)-2-(6-methyl-pyridin-2-yl)-imidazo[1,2-a]pyrimidin-7-ylamine;
3-(2-Azetidin-1-yl-pyrimidin-4-yl)-2-(6-methyl-pyridin-2-yl)-imidazo[1,2-a]pyridine;
3-(2-Methanesulfonyl-pyrimidin-4-yl)-2-(6-methyl-pyridin-2-yl)-imidazo[1,2-a]pyridine-7-carboxylic acid ethyl ester;
3-(2-Methanesulfonyl-pyrimidin-4-yl)-2-(6-methyl-pyridin-2-yl)-imidazo[1,2-a]pyridine-6-carboxylic acid methyl ester;
3-(2-Methanesulfonyl-pyrimidin-4-yl)-7-methyl-2-(6-methyl-pyridin-2-yl)-imidazo[1,2-a]pyridine;
3-(2-Methanesulfonyl-pyrimidin-4-yl)-8-methyl-2-(6-methyl-pyridin-2-yl)-imidazo[1,2-a]pyridine;
3,3-Dimethyl-N-[2-(6-methyl-pyridin-2-yl)-3-(2-methylsulfanyl-pyrimidin-4-yl)-imidazo[1,2-a]pyrimidin-7-yl]-butyramide;
3-(2-Methanesulfonyl-pyrimidin-4-yl)-2-(6-methyl-pyridin-2-yl)-imidazo[1,2-a]pyridine-6-carbonitrile;
3-(2-Methylsulfanyl-pyrimidin-4-yl)-2-pyridin-2-yl-imidazo[1,2-a]pyridine;
3,6-Dichloro-N-(4-{4-[2-(6-methyl-pyridin-2-yl)-imidazo[1,2-a]pyridin-3-yl]-pyrimidin-2-ylamino}-butyl)-2-(2,4,5,7-Tetrachloro-6-hydroxy-3-oxo-9,9a-dihydro-3H-xanthen-9-yl)-terephthalamic acid;
3-[2-(2-Methyl-aziridin-1-yl)-pyrimidin-4-yl]-2-(6-methyl-pyridin-2-yl)-imidazo[1,2-a]pyridine;
3-[2-(4-Methyl-piperazin-1-yl)-pyrimidin-4-yl]-2-(6-methyl-pyridin-2-yl)-imidazo[1,2-a]pyridine;
3-{[3-(2-Amino-pyrimidin-4-yl)-2-(6-methyl-pyridin-2-yl)-imidazo[1,2-a]pyridine-6-carbonyl]-amino}-propionic acid methyl ester;
3-{[3-(2-Amino-pyrimidin-4-yl)-2-(6-methyl-pyridin-2-yl)-imidazo[1,2-a]pyridine-7-carbonyl]-amino}-propionic acid methyl ester;
3-{4-[2-(6-Methyl-pyridin-2-yl)-imidazo[1,2-a]pyridin-3-yl]pyrimidin-2-ylamino}-phenol;
4-(2-{4-[2-(6-Methyl-pyridin-2-yl)-imidazo[1,2-a]pyridin-3-yl]-pyrimidin-2-ylamino}-ethyl)-benzenesulfonamide
4-(2-Pyridin-2-yl-imidazo[1,2-a]pyridin-3-yl)-pyrimidin-2-ylamine;
4-[2-(6-Chloro-pyridin-2-yl)-imidazo[1,2-a]pyridin-3-yl]-pyrimidin-2-ylamine;
4-[2-(6-Methyl-pyridin-2-yl)-7-trifluoromethyl-imidazo[1,2-a]pyridin-3-yl]-pyrimidin-2-ylamine;
4-[2-(6-Methyl-pyridin-2-yl)-imidazo[1,2-a]pyridin-3-yl]-pyrimidin-2-ylamine;
4-[2-(6-Methyl-pyridin-2-yl)-imidazo[1,2-a]pyridin-3-yl]-pyrimidine-2-carbonitrile;
4-[2-(6-Methyl-pyridin-2-yl)-imidazo[1,2-a]pyridin-3-yl]-pyrimidine-2-carboxylic acid amide;
4-[6-Bromo-2-(6-methyl-pyridin-2-yl)-imidazo[1,2-a]pyridin-3-yl]-pyrimidin-2-ylamine;
4-[6-Chloro-2-(6-methyl-pyridin-2-yl)-imidazo[1,2-a]pyridin-3-yl]-pyrimidin-2-ylamine;
4-[6-Fluoro-2-(6-methyl-pyridin-2-yl)-imidazo[1,2-a]pyridin-3-yl]-pyrimidin-2-ylamine;
4-[6-Methyl-2-(6-methyl-pyridin-2-yl)-8-(2-morpholin-4-yl-ethylamino)-imidazo[1,2-a]pyridin-3-yl]-pyrimidin-2-ol;
4-[6-Methyl-2-(6-methyl-pyridin-2-yl)-8-(2-pyridin-2-yl-ethylamino)-imidazo[1,2-a]pyridin-3-yl]-pyrimidin-2-ol
4-[6-Methyl-2-(6-methyl-pyridin-2-yl)-8-(2-pyridin-3-yl-ethylamino)-imidazo[1,2-a]pyridin-3-yl]-pyrimidin-2-ol
4-[6-Methyl-2-(6-methyl-pyridin-2-yl)-8-(2-pyridin-4-yl-ethylamino)-imidazo[1,2-a]pyridin-3-yl]-pyrimidin-2-ol;
4-[6-Methyl-2-(6-methyl-pyridin-2-yl)-8-morpholin-4-yl-imidazo[1,2-a]pyridin-3-yl]-pyrimidin-2-ol;
4-[6-Methyl-2-(6-methyl-pyridin-2-yl)-8-morpholin-4-yl-imidazo[1,2-a]pyridin-3-yl]-pyrimidin-2-ylamine;
4-[6-Methyl-2-(6-methyl-pyridin-2-yl)-imidazo[1,2-a]pyridin-3-yl]-pyrimidin-2-ylamine;
4-[7-Aminomethyl-2-(6-methyl-pyridin-2-yl)-imidazo[1,2-a]pyridin-3-yl]-pyrimidin-2-ylamine;
4-[7-Methyl-2-(6-methyl-pyridin-2-yl)-imidazo[1,2-a]pyridin-3-yl]-pyrimidin-2-ylamine;
4-[8-Benzyloxy-2-(6-methyl-pyridin-2-yl)-imidazo[1,2-a]pyridin-3-yl]-pyrimidin-2-ol;
4-[8-Benzyloxy-2-(6-methyl-pyridin-2-yl)-imidazo[1,2-a]pyridin-3-yl]-pyrimidin-2-ylamine;
4-[8-Bromo-6-methyl-2-(6-methyl-pyridin-2-yl)-imidazo[1,2-a]pyridin-3-yl]-pyrimidin-2-ol;
4-[8-Methyl-2-(6-methyl-pyridin-2-yl)-imidazo[1,2-a]pyridin-3-yl]-pyrimidin-2-ylamine;
6-Chloro-3-(2-methanesulfonyl-pyrimidin-4-yl)-2-(6-methyl-pyridin-2-yl)-imidazo[1,2-a]pyridine;
5-Dimethylamino-naphthalene-1-sulfonic acid (4-{4-[2-(6-methyl-pyridin-2-yl)-imidazo[1,2-a]pyridin-3-yl]-pyrimidin-2-ylamino}-butyl)-amide;
6-(2,7-Difluoro-6-hydroxy-3-oxo-3H-xanthen-9-yl)-N-(4-{4-[2-(6-methyl-pyridin-2-yl)-imidazo[1,2-a]pyridin-3-yl]-pyrimidin-2-ylamino}-butyl)-isophthalamic acid;
6-Amino-9-[2-carboxy-5-(4-{4-[2-(6-methyl-pyridin-2-yl)-imidazo[1,2-a]pyridin-3-yl]-pyrimidin-2-ylamino}-butylcarbamoyl)-phenyl]-xanthen-3-ylidene-ammonium;
6-Bromo-2-(6-methyl-pyridin-2-yl)-3-(2-methylsulfanyl-pyrimidin-4-yl)-imidazo[1,2-a]pyridine;
6-Fluoro-2-(6-methyl-pyridin-2-yl)-3-(2-methylsulfanyl-pyrimidin-4-yl)-imidazo[1,2-a]pyridine;
7-Amino-4-methyl-3-[(4-{4-[2-(6-methyl-pyridin-2-yl)-imidazo[1,2-a]pyridin-3-yl]-pyrimidin-2-ylamino}-butylcarbamoyl)-methyl]-2-oxo-2H-chromene-6-sulfonic acid;
Cyclobutyl-{4-[2-(6-methyl-pyridin-2-yl)-imidazo[1,2-a]pyridin-3-yl]-pyrimidin-2-yl}-amine;
Cyclopentyl-{4-[2-(6-methyl-pyridin-2-yl)-imidazo[1,2-a]pyridin-3-yl]-pyrimidin-2-yl}-amine;
Cyclopropyl-{4-[2-(6-methyl-pyridin-2-yl)-imidazo[1,2-a]pyridin-3-yl]-pyrimidin-2-yl}-amine; Cyclopropyl-methyl-{4-[2-(6-methyl-pyridin-2-yl)-imidazo[1,2-a]pyridin-3-yl]-pyrimidin-2-yl}-amine;
Dimethyl-{4-[2-(6-methyl-pyridin-2-yl)-imidazo[1,2-a]pyridin-3-yl]-pyrimidin-2-yl}-amine;
Isopropyl-4-[2-(6-methyl-pyridin-2-yl)-imidazo[1,2-a]pyridin-3-yl]-pyrimidin-2-yl}-amine;
Methyl-{-4-[2-(6-methyl-pyridin-2-yl)-imidazo[1,2-a]pyridin-3-yl]-pyrimidin-2-yl}-amine;
N-(2-{4-[2-(6-Methyl-pyridin-2-yl)-imidazo[1,2-a]pyridin-3-yl]-pyrimidin-2-ylamino}-ethyl)-acetamide;
N-(4-{4-[2-(6-Methyl-pyridin-2-yl)-imidazo[1,2-a]pyridin-3-yl]-pyrimidin-2-ylamino}-butyl)-acetamide;
N,N-Dimethyl-N′-{4-[2-(6-methyl-pyridin-2-yl)-imidazo[1,2-a]pyridin-3-yl]-pyrimidin-2-yl}-ethane-1,2-diamine;
N-[2-(6-Methyl-pyridin-2-yl)-3-(2-methylsulfanyl-pyrimidin-4-yl)-imidazo[1,2-a]pyrimidin-7-yl]-3-pyridin-3-yl-propionamide;
N-[2-(6-Methyl-pyridin-2-yl)-3-(2-methylsulfanyl-pyrimidin-4-yl)-imidazo[1,2-a]pyrimidin-7-yl]-nicotinamide;
N-[2-(6-Methyl-pyridin-2-yl)-3-(2-methylsulfanyl-pyrimidin-4-yl)-imidazo[1,2-a]pyrimidin-7-yl]-propionamide;
N-[3-(2-Amino-pyrimidin-4-yl)-2-(6-methyl-pyridin-2-yl)-imidazo[1,2-a]pyridine-6-carbonyl]-methanesulfonamide;
N-[3-(2-Amino-pyrimidin-4-yl)-2-(6-methyl-pyridin-2-yl)-imidazo[1,2-a]pyridine-7-carbonyl]-methanesulfonamide;
N-[3-(2-Amino-pyrimidin-4-yl)-2-(6-methyl-pyridin-2-yl)-imidazo[1,2-a]pyrimidin-7-yl]-2-(3-methoxy-phenyl)-acetamide;
N-[3-(2-Amino-pyrimidin-4-yl)-2-(6-methyl-pyridin-2-yl)-imidazo[1,2-a]pyrimidin-7-yl]-3,3-dimethyl-butyramide;
N-[3-(2-Amino-pyrimidin-4-yl)-2-(6-methyl-pyridin-2-yl)-imidazo[1,2-a]pyrimidin-7-yl]-3-pyridin-3-yl-propionamide;
N-[3-(2-Amino-pyrimidin-4-yl)-2-(6-methyl-pyridin-2-yl)-imidazo[1,2-a]pyrimidin-7-yl]-acetamide;
N-[3-(2-Amino-pyrimidin-4-yl)-2-(6-methyl-pyridin-2-yl)-imidazo[1,2-a]pyrimidin-7-yl]-nicotinamide;
N-[3-(2-Methanesulfonyl-pyrimidin-4-yl)-2-(6-methyl-pyridin-2-yl)-imidazo[1,2-a]pyrimidin-7-yl]-2-(3-methoxy-phenyl)-acetamide;
N-[3-(2-Methanesulfonyl-pyrimidin-4-yl)-2-(6-methyl-pyridin-2-yl)-imidazo[1,2-a]pyrimidin-7-yl]-3,3-dimethyl-butyramide;
N-[3-(2-Methanesulfonyl-pyrimidin-4-yl)-2-(6-methyl-pyridin-2-yl)-imidazo[1,2-a]pyrimidin-7-yl]-3-pyridin-3-yl-propionamide;
N-[3-(2-Methanesulfonyl-pyrimidin-4-yl)-2-(6-methyl-pyridin-2-yl)-imidazo[1,2-a]pyrimidin-7-yl]-nicotinamide;
N-[3-(2-Methanesulfonyl-pyrimidin-4-yl)-2-(6-methyl-pyridin-2-yl)-imidazo[1,2-a]pyrimidin-7-yl]-propionamide;
N-[3-(2-Amino-pyrimidin-4-yl)-2-(6-methyl-pyridin-2-yl)-imidazo[1,2-a]pyrimidin-7-yl]-propionamide;
N-{4-[2-(6-Methyl-pyridin-2-yl)-imidazo[1,2-a]pyridin-3-yl]-pyrimidin-2-yl}-acetamide;
N1-{4-[2-(6-Methyl-pyridin-2-yl)-imidazo[1,2-a]pyridin-3-yl]-pyrimidin-2-yl}-butane-1,4-diamine;
N1-{4-[2-(6-Methyl-pyridin-2-yl)-imidazo[1,2-a]pyridin-3-yl]-pyrimidin-2-yl}-propane-1,3-diamine;
N-(4-{4-[2-(6-Methyl-pyridin-2-yl)-imidazo[1,2-a]pyridin-3-yl]-pyrimidin-2-ylamino}-butyl)-(BODIPY FL) amide; and
N-(4-{4-[2-(6-Methyl-pyridin-2-yl)-imidazo[1,2-a]pyridin-3-yl]-pyrimidin-2-ylamino}-butyl)-(Texas Red-X) amide.

Formula IV

In one aspect, the disclosure features a method of producing a reprogrammed cell (e.g. iPS cell or partially reprogrammed cell) from a differentiated cell, the method comprising:

contacting an isolated differentiated cell with a compound of formula (IVa)

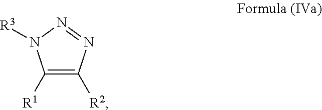

or formula (IVb)

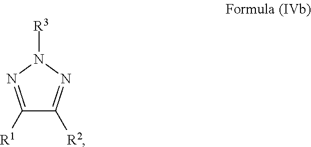

wherein:

R¹ is cyclyl, heterocyclcyl, aryl or heteroaryl, each of which can be optionally substituted;

R² is cyclyl, heterocyclcyl, aryl or heteroaryl, each of which can be optionally substituted;

R³ is R³ is H, C₁-C₆ alkyl, C₂-C₆ alkenyl, C₂-C₆ alkynyl, aryl, heteroaryl, cyclyl, heterocyclyl, acyl or a nitrogen protecting group, each of which can be optionally substituted,

to thereby produce a reprogrammed cell (e.g. iPS cell or partially reprogrammed cell) from the differentiated cell.

In one embodiment, the method comprises contacting a plurality of differentiated cells with a compound of formula (IVa) or formula (IVb) to thereby produce a plurality of reprogrammed cells (e.g. iPS cells or partially reprogrammed cells) from the differentiated cells.

In some embodiments, R¹ is a nitrogen containing heteroaryl (e.g., including 1, 2, or 3 nitrogens. In some embodiments, R¹ is a bicyclic heteroaryl. In some embodiments, R¹ is a 6-6 fused heteroaryl. In some embodiments, R¹ is pyridyl, pyrimidyl, quinolinyl, naphthyridinyl (e.g., 1,5-naphthyridinyl), quinazolinyl, 5,6,7,8-tetrahydroquinazolinyl, 1,3-benzodioxlyl, 1,2,3-benzotriazolyl, benzoxazolyl, benzothiazolyl, 2,1,3-benzooxadiazole, imidazo[1,2-a]pyridinyl, pyrazolo[1,5-a]pyridinyl, [1,2,4]triazolo[1,5-a]pyridinyl, pyrazolo[1,5-a]pyrimidinyl, [1,2,4]triazolo[1,5-a]pyrimidinyl, [1,2,3]triazolo[1,5-a]pyrimidinyl, [1,2,4]triazolo[4,3-a]pyrimidinyl, [1,2,4]triazolo[4,3-a]pyridazinyl. In some embodiments, R¹ is

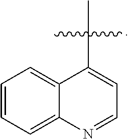

In some embodiments, R¹ is

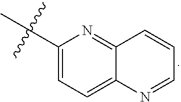

In some embodiment, R² is aryl, e.g., a substituted aryl. In some embodiments, R² is a nitrogen comprising heteroaryl (e.g., including 1, 2 or 3 nitrogens (e.g., 1 or 2)). In some embodiments, R² is an optionally substituted monocyclic heteroaryl (e.g., a six membered heteroaryl such as pyridyl, pyrimidyl, pyridazinyl or pyrazinyl). In some embodiments, R² is substituted. Exemplary substituents include halo, C₁-C₆ alkyl, haloC₁-C₆alkyl, C₁-C₆ alkoxy, OH, haloC₁-C₆alkoxy. In some embodiments, R² is monosubstituted. In some embodiments, R² is substituted with methyl. In one embodiment, R² is an optionally substituted pyridyl. In some embodiments, R² is

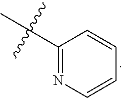

In some embodiments, R² is

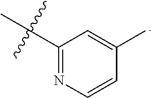

Exemplary compounds of formula (IVa) and (IVb) include:
6-[5-(6-methylpyridin-2-yl)-2H-[1,2,3]-triazol-4-yl]-quinazoline;
3-Isopropyl-6-[5-(6-methylpyridin-2-yl)-1H-[1,2,3]-triazol-4-yl]-[1,2,4]triazolo[4,3-a]pyridine;
3-Methyl-6-[5-(6-methyl-pyridin-2-yl)-2H-[1,2,3]-triazol-4-yl]-[1,2,4]triazolo[4,3-a]pyridine;
2-(4-methanesulfonylphenyl)-4-(5-(6-methyl)-py-ridin-2-yl-3H-[1,2,3]triazol-4-yl)-pyridine;
2-(4-methoxyphenyl)-4-(5-(6-methyl)-pyridin-2-yl-3H-[1,2,3]triazol-4-yl)-pyridine;
dimethyl-[2-(4-{4-[5-(6-methyl)-pyridin-2-yl-3H-[1,2,3]triazol-4-yl]-pyridin-2-yl}phenoxy)-ethyl]-amine;
4-(4-{4-[5-(6-methyl-pyridin-2-yl)-3H-[1,2,3]tria-zol-4-yl]-pyridin-2-yl}-benzyl)-morpholine;
2-(4-ethylphenyl)-4-(5-(6-methyl-pyridin-2-yl)-3H-[1,2,3]triazol-4-yl)-pyridine;
4-{4-[5-(6-methyl-pyridin-2-yl)-3H-[1,2,3]triazol-4-yl]-pyridin-2-yl}-N-(tetrahydro-pyran-4-yl)-benzamide;
2-(4-chlorophenyl)-4-(5-(6-methyl)-pyridin-2-yl-3H-[1,2,3]triazol-4-yl)-pyridine;
2-(4-trifluoromethoxyphenyl)-4-(5-(6-methyl)-py-ridin-2-yl-3H-[1,2,3]triazol-4-yl)-pyridine;
2-{4-(2-pyrrolidin-1-yl-ethoxy)-phenyl}-(5-(6-methyl)-pyridin-2-yl-3H-[1,2,3]triazol-4-yl)-pyridine;
2-(4-fluorophenyl)-4-(5-(6-methyl)-pyridin-2-yl-3H-[1,2,3]triazol-4-yl)-pyridine;
5-[5-6-Methyl-pyridin-2-yl)-1H-[1,2,3]triazol-4-yl]-benzo[1,2,5]thiadiazole;
5-[2-Ethyl-5-(6-methyl-pyridin-2-yl)-2H-[1,2,3]triazol-4-yl]-benzo[1,2,5]thiadiazole;
6-[5-(6-Methylpyridin-2-yl)-1H-[1,2,3]triazol-4-yl]-[1,2,4]triazolo[1,5a]pyridine;
2-[5-(2,3-Dihydrobenzofuran-5-yl)-3H-[1,2,3]triazol-4-yl]-6-methylpyridine;
2-[5-2,3-Dihydrobenzo[1,4]dioxin-6-yl)-2H-[1, 2,3]triazol-4-yl]-6-methylpyridine;
1-Methyl-6-[5-(6-methyl-pyridin-2-yl)-2H-[1,2, 3]triazol-4-yl]-1H-benzimidazole;
6-(2-Ethyl-5-(6-methylpyridin-2-yl)-2H-[1,2,3]triazol-4-yl)-[1,2,4]triazolo[1,5-a]pyridine;
6-(2-Methyl-5-(6-methylpyridin-2-yl)-2H-[1,2, 3]triazol-4-yl)-[1,2,4]triazolo[1,5-a]pyridine;
2-[5-(4-Methoxyphenyl)-2H-[1,2,3]triazol-4-yl]-6-methylpyridine;
2-[5-(3-Fluoro-4-methoxyphenyl)-2H-[1,2,3]triazol-4-yl]-6-methylpyridine; and
2-[5-(3-Chloro-4-methoxyphenyl)-2H-[1,2,3]triazol-4-yl]-6-methylpyridine.

Formula V

In one aspect, the disclosure features a method of producing a reprogrammed cell (e.g. iPS cell or partially reprogrammed cell) from a differentiated cell, the method comprising:

contacting an isolated differentiated cell with a compound of formula (V)

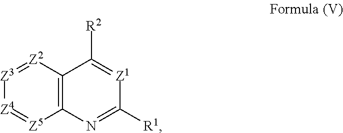

wherein:

R¹ is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;

R² is H, C₁-C₆ alkyl, C₁-C₆ alkenyl, C₁-C₆ alkynyl, aryl, heteroaryl, cyclyl, optionally substituted heterocyclyl, acyl or amino (e.g., N(R³)₂), each of which can be optionally substituted;

R³ is independently for each occurrence H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;

Z¹, Z², Z³, Z⁴ and Z⁵ are each independently N or CR³, provided that at least two of Z², Z³, Z⁴ and Z⁵ are CR³, and further provided that two adjacent Z positions are not N;

to thereby produce a reprogrammed cell (e.g. iPS cell or partially reprogrammed cell) from the differentiated cell.

In one embodiment, the method comprises contacting a plurality of differentiated cells with a compound of formula (V) to thereby produce a plurality of reprogrammed cells (e.g. iPS cells or or partially reprogrammed cell) from the differentiated cells.

In one embodiment, R¹ is aryl, e.g., a substituted aryl. In one embodiment, R¹ is phenyl. In one embodiment, R¹ is phenyl substituted with at least one halogen.

In one embodiment, R² is NHR³.

Exemplary compounds of formula (V) include:
2-phenyl-4-(4-pyridylamino)-quinazoline;
2-(2-bromophenyl)-4-(4-pyridylamino)-quinazoline;
2-(2-chlorophenyl)-4-(4-pyridylamino)-quinazoline;
2-(2-fluorophenyl)-4-(4-pyridylamino)-quinazoline;
2-(2-methylphenyl)-4-(4-pyridylamino)-quinazoline;
2-(4-fluorophenyl)-4-(4-pyridylamino)-quinazoline;
2-(3-methoxyanilyl)-4-(4-pyridylamino)-quinazoline;
2-(2,6-dichlorophenyl)-4-(4-pyridylamino)-quinazoline;
2-(2,6-dibromophenyl)-4-(4-pyridylamino)-quinazoline;
2-(2,6-difluorophenyl)-4-(4-pyridylamino)-quinazoline;
2-(2-fluorophenyl)-4-(6-pyridylamino)-6,7-dimethoxyquinazoline;
2-(4-fluorophenyl)-4-(4-pyridylamino)-6,7-dimethoxyquinazoline;
2-(2-fluorophenyl)-4-(4-pyridylamino)-6-nitroquinazoline;
2-(2-fluorophenyl)-4-(4-pyridylamino-6-aminoquinazoline;
2-(2-fluorophenyl)-4-(4-pyridylamino)-7-aminoquinazoline;
2-(2-fluorophenyl)-4-(4-pyridylamino)-6-(3-methoxybenzylamino)-quinazoline;
2-(2-fluorophen)-4-(4-pyridylamino)-6-(4-methoxybenzylamino)-quinazoline;
2-(2-fluorophenyl)-4-(4-pyridylal7nino)-6-(2-isobutylamino)-quinazoline; and
2-(2-fluorophenyl)-4-(4-pyridylamino)-6-(4-methylmercaptobenzylamino)-quinazoline.

Formula VI

In one aspect, the disclosure features a method of producing a reprogrammed cell (e.g. iPS cell or partially reprogrammed cells) from a differentiated cell, the method comprising:

contacting an isolated differentiated cell with a compound of formula (VI)

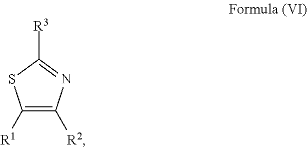

wherein:

R¹ is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;

R² is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;

R³ is H, C₁-C₆ alkyl, C₁-C₆ alkenyl, C₁-C₆ alkynyl, aryl, heteroaryl, cyclyl, optionally substituted heterocyclyl, acyl or amino, each of which can be optionally substituted,

to thereby produce a reprogrammed cell (e.g. iPS cell or partially reprogrammed cell) from the differentiated cell.

In one embodiment, the method comprises contacting a plurality of differentiated cells with a compound of formula (VI) to thereby produce a plurality of iPS cells from the differentiated cells.

In some embodiments, R¹ is a nitrogen containing heteroaryl (e.g., including 1, 2, or 3 nitrogens (e.g., 1 or)). In some embodiments, R¹ is a bicyclic heteroaryl. In some embodiments, R¹ is a 6-6 fused heteroaryl. In some embodiments, R¹ is pyridyl, pyrimidyl, pyridazinyl, pyrazinyl, quinolinyl, naphthyridinyl (e.g., 1,5-naphthyridinyl), quinazolinyl, 5,6,7,8-tetrahydroquinazolinyl, 1,3-benzodioxlyl, 1,2,3-benzotriazolyl, benzoxazolyl, benzothiazolyl, 2,1,3-benzooxadiazole, imidazo[1,2-a]pyridinyl, pyrazolo[1,5-a]pyridinyl, [1,2,4]triazolo[1,5-a]pyridinyl, pyrazolo[1,5-a]pyrimidinyl, [1,2,4]-triazolo[1,5-a]pyrimidinyl, [1,2,3]-triazolo[1,5-a]pyrimidinyl, [1,2,4]triazolo[4,3-a]pyrimidinyl, [1,2,4]triazolo[4,3-a]pyridazinyl. In some embodiments, R¹ is

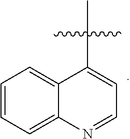

In some embodiments, R¹ is

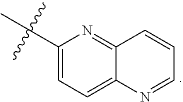

In some embodiment, R² is aryl, e.g., a substituted aryl. In some embodiments, R² is a nitrogen comprising heteroaryl (e.g., including 1, 2 or 3 nitrogens (e.g., 1 or 2)). In some embodiments, R² is an optionally substituted monocyclic heteroaryl (e.g., a six membered heteroaryl such as pyridyl, pyrimidyl, pyridazinyl or pyrazinyl). In some embodiments, R² is substituted. Exemplary substituents include halo, C₁-C₆ alkyl, haloC₁-C₆alkyl, C₁-C₆ alkoxy, OH, haloC₁-C₆alkoxy. In some embodiments, R² is monosubstituted. In some embodiments, R² is substituted with methyl. In one embodiment, R² is an optionally substituted pyridyl. In some embodiments, R^{2 is}

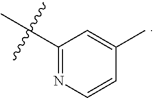

In some embodiments, R² is

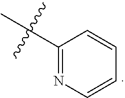

Exemplary compounds of formula (VI) include:
4-(Pyridin-2-yl)-5-quinolin-4-yl-1,3-thiazol-2-amine;
4-(6-methylpyridin-2-yl)-5-(1,5-naphthyridin-2-yl)-1,3-thiazol-2-amine;
5-([1,5]Naphthyridin-2-yl)-4-pyridin-2-yl-1,3-thiazol-2-amine;
5-[2-(4-Chlorophenyl)pyridin-4-yl]-4-pyridin-2-yl-1,3-thiazol-2-amine;
5-[2-(4-Methoxyphenyl)pyridin-4-yl]-4-pyri-din-2-yl-1,3-thiazol-2-amine;
5-[2-(4-Fluorophenyl)pyridin-4-yl]-4-pyridin-2-yl-1,3-thiazol-2-amine;
5-[2-(4-Ethylphenyl)pyridin-4-yl]-4-pyridin-2-yl-1,3-thiazol-2-amine;
5-[2-(4-Ethoxyphenyl)pyridin-4-yl]-4-pyridin-2-yl-1,3-thiazol-2-amine; and
5-[2-(Thiophen-3-yl)pyridin-4-yl]-4-pyridin-2-yl-1,3-thiazol-2-amine

Formula VII

In one aspect, the disclosure features a method of producing a reprogrammed cell (e.g. iPS cell or partially reprogrammed cells) from a differentiated cell, the method comprising:

contacting an isolated differentiated cell with a compound of formula (VII)

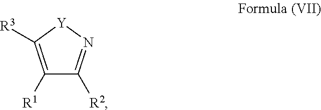

wherein:

X is O, S or CH₂;

R¹ is H, C₁-C₆ alkyl, C₁-C₆ alkenyl, C₁-C₆ alkynyl, aryl, heteroaryl, cyclyl, heterocyclyl, acyl, amino, or amide (e.g., —CO₂NH₂), each of which can be optionally substituted;

R² is C₁-C₆ alkyl, —O-alkyl, amino, acyl, aryl, heteroaryl, cyclyl or heterocyclyl, each of which can be optionally substituted;

R³ is H, C₁-C₆ alkyl, —O-alkyl, amino, amide, —NHC(O)NH-alkyl, acyl, aryl, heteroaryl, cyclyl, heterocyclyl, each of which can be optionally substituted,

to thereby produce a reprogrammed cell (e.g. iPS cell or partially reprogrammed cell) from the differentiated cell.

In one embodiment, the method comprises contacting a plurality of differentiated cells with a compound of formula (VII) to thereby produce a plurality of reprogrammed cells (e.g. iPS cells or partially reprogrammed cell) from the differentiated cells.

In some embodiments, X is S.

In some embodiments, R¹ is optionally substituted amide, e.g. —CO₂NH₂. In some embodiments, the amide is substituted with C₁-C₆ alkyl, which can also be optionally substituted.

In some embodiments, R¹ is aryl, e.g., a substituted aryl. In some embodiments, R¹ is substituted with two substituents. In some embodiments, R¹ is substituted with two substituents, which together with the carbons to which they are attached form a ring. In some embodiments, R¹ is a substituted phenyl. In some embodiments, R¹ is a nitrogen containing heteroaryl (e.g., including 1, 2, or 3 nitrogens (e.g., 1 or)). In some embodiments, R¹ is a bicyclic heteroaryl. In some embodiments, R¹ is a 6-6 fused heteroaryl. In some embodiments, R¹ is pyridyl, pyrimidyl, pyridazinyl, pyrazinyl, quinolinyl, naphthyridinyl (e.g., 1,5-naphthyridinyl), quinazolinyl, 5,6,7,8-tetrahydroquinazolinyl, 1,3-benzodioxlyl, 1,2,3-benzotriazolyl, benzoxazolyl, benzothiazolyl, 2,1,3-benzooxadiazole, imidazo[1,2-a]pyridinyl, pyrazolo[1,5-a]pyridinyl, [1,2,4]triazolo[1,5-a]pyridinyl, pyrazolo[1,5-a]pyrimidinyl, [1,2,4]triazolo[1,5-a]pyrimidinyl, [1,2,3]triazolo[1,5-a]pyrimidinyl, [1,2,4]triazolo[4,3-a]pyrimidinyl, [1,2,4]triazolo[4,3-a]pyridazinyl. In some embodiments, R¹ is

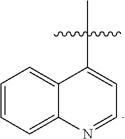

In some embodiments, R¹ is

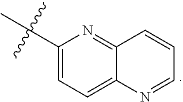

In some embodiments, R² is optionally substituted —O-alkyl. Exemplary substituents include aryl, heteroaryl, cyclyl and heterocyclyl, each of which can be optionally substituted. In some embodiments, R² is

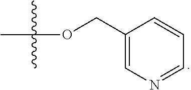

In some embodiment, R² is aryl, e.g., a substituted aryl. In some embodiments, R² is a nitrogen comprising heteroaryl (e.g., including 1, 2 or 3 nitrogens (e.g., 1 or 2)). In some embodiments, R² is an optionally substituted monocyclic heteroaryl (e.g., a six membered heteroaryl such as pyridyl, pyrimidyl, pyridazinyl or pyrazinyl). In some embodiments, R² is substituted. Exemplary substituents include halo, C₁-C₆ alkyl, haloC₁-C₆alkyl, C₁-C₆ alkoxy, OH, haloC₁-C₆alkoxy. In some embodiments, R² is monosubstituted. In some embodiments, R² is substituted with methyl. In one embodiment, R² is an optionally substituted pyridyl. In some embodiments, R² is

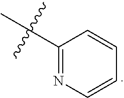

In some embodiments, R³ is optionally substituted —NHC(O)NH-alkyl. In some embodiments, substituents is aryl or heteroaryl, each of which can be optionally substituted. In one embodiment,

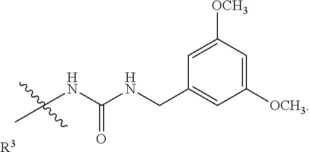

Exemplary compounds of formula (VII) include:
1-(3,5-dimethoxybenzyl)-3-(4-carbamoyl-3-(2-(pyridin-3-yl)ethyl)isothiazol-5-yl)urea;
6-[3-(6-Methyl-pyridin-2-yl)-isoxazol-4-yl]-quinoxaline;
5-[3-(2-Cyclohex-1-enyl-ethyl)-ureido]-3-(pyridin-3-ylmethoxy)-isothiazole-4-carboxylic acid amide;
5-[3-(2,5-Dimethyl-benzyl)-ureido]-3-(pyridin-3-ylmethoxy)-isothiazole-4-carboxylic acid amide;
5-[3-(3,5-Dimethoxy-benzyl)-ureido]-3-(pyridin-3-ylmethoxy)-isothiazole-4-carboxylic acid amide;
5-[3-(2-Ethoxy-benzyl)-ureido]-3-(pyridin-3-ylmethoxy)-isothiazole-4-carboxylic acid amide;
5-{3-[2-(2-Ethoxy-phenyl)-ethyl]-ureido}-3-(pyridin-3-ylmethoxy)-isothiazole-4-carboxylic acid amide;
5-{3-[2-(3,4-Dimethoxy-phenyl)-ethyl]-ureido}-3-(pyridin-3-ylmethoxy)-isothiazole-4-carboxylic acid amide;
5-(3-Phenethyl-ureido)-3-(pyridin-3-yl-methoxy)-isothiazole-4-carboxylic acid amide;
5-{3-[2-(3-Ethoxy-4-methoxy-phenyl)-ethyl]-ureido}-3-(pyridin-3-ylmethoxy)-isothiazole-4-carboxylic acid amide;
5-{3-[2-(4-Ethoxy-phenyl)-ethyl]-ureido}-3-(pyridin-3-ylmethoxy)-isothiazole-4-carboxylic acid amide;
5-{3-[2-(4-Chloro-phenyl)-ethyl]-ureido}-3-(pyridin-3-ylmethoxy)-isothiazole-4-carboxylic acid amide;
5-{3-[2-(3-Chloro-phenyl)-ethyl]-ureido}-3-(pyridin-3-ylmethoxy)-isothiazole-4-carboxylic acid amide;
5-{3-[2-(3-Methoxy-phenyl)-ethyl]-ureido}-3-(pyridin-3-ylmethoxy)-isothiazole-4-carboxylic acid amide;
5-{3-[2-(4-Methoxy-phenyl)-ethyl]-ureido}-3-(pyridin-3-ylmethoxy)-isothiazole-4-carboxylic acid amide;
5-{3-[2-(3-Bromo-4-methoxy-phenyl)-ethyl]-ureido}-3-(pyridin-3-ylmethoxy)-isothiazole-4-carboxylic acid amide;
5-{3-[2-(4-Bromo-phenyl)-ethyl]-ureido}-3-(pyridin-3-ylmethoxy)-isothiazole-4-carboxylic acid amide;
5-{3-[2-(2-Chloro-phenyl)-ethyl]-ureido}-3-(pyridin-3-ylmethoxy)-isothiazole-4-carboxylic acid amide;
5-{3-[2-(3-Chloro-phenyl)-ethyl]-ureido}-3-(pyridin-3-ylmethoxy)-isothiazole-4-carboxylic acid amide;
5-{3-[2-(2-Fluoro-phenyl)-ethyl]-ureido}-3-(pyridin-3-ylmethoxy)-isothiazole-4-carboxylic acid amide;
5-{3-[2-(3-Fluoro-phenyl)-ethyl]-ureido}-3-(pyridin-3-ylmethoxy)-isothiazole-4-carboxylic acid amide;
5-{3-[2-(4-Fluoro-phenyl)-ethyl]-ureido}-3-(pyridin-3-ylmethoxy)-isothiazole-4-carboxylic acid amide;
5-{3-[2-(4-Ethoxy-3-methoxy-phenyl)-ethyl]-ureido}-3-(pyridin-3-ylmethoxy)-isothiazole-4-carboxylic acid amide;
5-{3-[2-(3-Ethoxy-4-methoxy-phenyl]-ethyl)-ureido}-3-(pyridin-3-ylmethoxy)-isothiazole-4-carboxylic acid amide;
5-{3-[2-(2,5-Dimethoxy-phenyl)-ethyl]-ureido}-3-(pyridin-3-ylmethoxy)-isothiazole-4-carboxylic acid amide;
5-{3-[2-(3-Methoxy-phenyl)-ethyl]-ureido}-3-(pyridin-3-ylmethoxy)-isothiazole-4-carboxylic acid amide;
5-[3-(2-Dinuoromethoxy-benzyl)-ureido]-3-(pyridin-3-ylmethoxy)-isothiazole-4-carboxylic acid amide;
5-[3-(2,6-Dimethoxy-benzyl)-ureido]-3-(pyridin-3-ylmethoxy)-isothiazole-4-carboxylic acid amide;
5-[3-(2,5-Dichloro-benzyl)-ureido]-3-(pyridin-3-ylmethoxy)-isothiazole-4-carboxylic acid amide;
5-[3-(3-Morpholin-4-yl-propyl)-ureido]-3-(pyridin-3-ylmethoxy)-isothiazole-4-carboxylic acid amide;
5-[3-(2-Morpholin-4-yl-ethyl)-ureido]-3-(pyridin-3-ylmethoxy)-isothiazole-4-carboxylic acid amide;
5-[3-(2-Diethylamino-ethyl)-ureido]-3-(pyridin-3-ylmethoxy)-isothiazole-4-carboxylic acid amide;
5-[3-(3-Dimethylamino-propyl)-ureido]-3-(pyridin-3-ylmethoxy)-isothiazole-4-carboxylic acid amide;
5-{3-[2-(1-Methyl-pyrrolidin-2-yl)-ethyl]-ureido}-3-(pyridin-3-ylmethoxy)-isothiazole-4-carboxylic acid amide;
5-{3-[3-(2-Methyl-piperidin-1-yl)-propyl]-ureido}-3-(pyridin-3-ylmethoxy)-isothiazole-4-carboxylic acid amide;
(R),(R)-5-[3-(2-Hydroxy-cycloheptylm-ethyl)-ureido]-3-(pyridin-3-ylmethoxy)-isothiazole-4-carboxylic acid amide;
(R),(R)-5-[3-(2-Hydroxy-cyclooctylmethyl)-ureido]-3-(pyridin-3-ylmethoxy)-isothiazole-4-carboxylic acid amide;
5-[3-(2-Hydroxy-ethyl)-ureido]-3-(pyridin-3-ylmethoxy)-isothiazole-4-carboxylic acid amide;
5-[3-(2-Hydroxy-butyl)-ureido]-3-(pyridin-3-ylmethoxy)-isothiazole-4-carboxylic acid amide;
5-{3-[3-(2-oxo-pyrrolidin-1-yl)-propyl]-ureido}-3-(pyridin-3-ylmethoxy)-isothiazole-4-carboxylic acid amide;
5-[3-(3-Imidazol-1-yl-propyl)-ureido]-3-(pyridin-3-ylmethoxy)-isothiazole-4-carboxylic acid amide;
5-(3-Benzyl-ureido)-3-(pyridin-3-yl-methoxy)-isothiazole-4-carboxylic acid amide;
5-[3-(2,5-Difluoro-benzyl)-ureido]-3-(pyridin-3-ylmethoxy)-isothiazole-4-carboxylic acid amide;
3-(1-Pyridin-3-yl-ethoxy)-5-(3-pyridin-2-yl-methyl-ureido)-isothiazole-4-carboxylic acid amide;
5-[3-(2,6-Dimethoxy-benzyl)-ureido]-3-(1-pyridin-3-yl-ethoxy)-isothiazole-4-carboxylic acid amide;
5-(3-Cyclopropylmethyl-ureido)-3-(pyridin-3-ylmethoxy)-isothiazole-4-carboxylic acid amide;
5-(3-Methyl-ureido)-3-(pyridin-3-yl-methoxy)-isothiazole-4-carboxylic acid amide;
5-(3-Methyl-ureido)-3-(1-pyridin-3-yl-ethoxy)-isothiazole-4-carboxylic acid amide; and
5-[3-(3,5-Dichloro-benzyl)-ureido]-3-(pyri-din-3-ylmethoxy)-isothiazole-4-carboxylic acid amide.

Formula VII

In one aspect, the disclosure features a method of producing a reprogrammed cell (e.g. iPS cell or a partially reprogrammed cell) from a differentiated cell, the method comprising:

contacting an isolated differentiated cell with a compound of formula (VII)

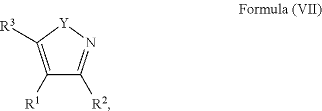

wherein:

X is O, S or CH₂;

R¹ is H, C₁-C₆ alkyl, C₁-C₆ alkenyl, C₁-C₆ alkynyl, aryl, heteroaryl, cyclyl, heterocyclyl, acyl, amino, or amide (e.g., —CO₂NH₂), each of which can be optionally substituted;

R² is C₁-C₆ alkyl, —O-alkyl, amino, acyl, aryl, heteroaryl, cyclyl or heterocyclyl, each of which can be optionally substituted;

R³ is H, C₁-C₆ alkyl, —O-alkyl, amino, amide, —NHC(O)NH-alkyl, acyl, aryl, heteroaryl, cyclyl, heterocyclyl, each of which can be optionally substituted,

to thereby produce a reprogrammed cell (e.g. iPS cell or partially reprogrammed cell) from the differentiated cell.

In one embodiment, the method comprises contacting a plurality of differentiated cells with a compound of formula (VII) to thereby produce a plurality of reprogrammed cells (e.g. iPS cells or partially reprogrammed cells) from the differentiated cells.

In some embodiments, X is S.

In some embodiments, R¹ is optionally substituted amide, e.g. —CO₂NH₂. In some embodiments, the amide is substituted with C₁-C₆ alkyl, which can also be optionally substituted.

In some embodiments, R¹ is aryl, e.g., a substituted aryl. In some embodiments, R¹ is substituted with two substituents. In some embodiments, R¹ is substituted with two substituents, which together with the carbons to which they are attached form a ring. In some embodiments, R¹ is a substituted phenyl. In some embodiments, R¹ is a nitrogen containing heteroaryl (e.g., including 1, 2, or 3 nitrogens (e.g., 1 or)). In some embodiments, R¹ is a bicyclic heteroaryl. In some embodiments, R¹ is a 6-6 fused heteroaryl. In some embodiments, R¹ is pyridyl, pyrimidyl, pyridazinyl, pyrazinyl, quinolinyl, naphthyridinyl (e.g., 1,5-naphthyridinyl), quinazolinyl, 5,6,7,8-tetrahydroquinazolinyl, 1,3-benzodioxlyl, 1,2,3-benzotriazolyl, benzoxazolyl, benzothiazolyl, 2,1,3-benzooxadiazole, imidazo[1,2-a]pyridinyl, pyrazolo[1,5-a]pyridinyl, [1,2,4]triazolo[1,5-a]pyridinyl, pyrazolo[1,5-a]pyrimidinyl, [1,2,4]triazolo[1,5-a]pyrimidinyl, [1,2,3]triazolo[1,5-a]pyrimidinyl, [1,2,4]triazolo[4,3-a]pyrimidinyl, [1,2,4]triazolo[4,3-a]pyridazinyl. In some embodiments, R¹ is

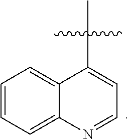

In some embodiments, R¹ is

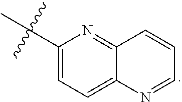

In some embodiments, R² is optionally substituted —O-alkyl. Exemplary substituents include aryl, heteroaryl, cyclyl and heterocyclyl, each of which can be optionally substituted. In some embodiments, R² is

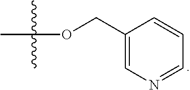

In some embodiment, R² is aryl, e.g., a substituted aryl. In some embodiments, R² is a nitrogen comprising heteroaryl (e.g., including 1, 2 or 3 nitrogens (e.g., 1 or 2)). In some embodiments, R² is an optionally substituted monocyclic heteroaryl (e.g., a six membered heteroaryl such as pyridyl, pyrimidyl, pyridazinyl or pyrazinyl). In some embodiments, R² is substituted. Exemplary substituents include halo, C₁-C₆ alkyl, haloC₁-C₆alkyl, C₁-C₆ alkoxy, OH, haloC₁-C₆alkoxy. In some embodiments, R² is monosubstituted. In some embodiments, R² is substituted with methyl. In one embodiment, R² is an optionally substituted pyridyl. In some embodiments, R^{2 is}

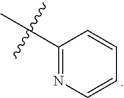

In some embodiments, R³ is optionally substituted —NHC(O)NH-alkyl. In some embodiments, substituents is aryl or heteroaryl, each of which can be optionally substituted. In one embodiment,

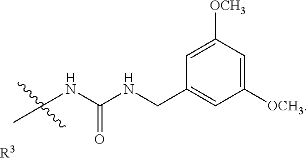

Exemplary compounds of formula (VII) include:
1-(3,5-dimethoxybenzyl)-3-(4-carbamoyl-3-(2-(pyridin-3-yl)ethyl)isothiazol-5-yl)urea;
6-[3-(6-Methyl-pyridin-2-yl)-isoxazol-4-yl]-quinoxaline;
5-[3-(2-Cyclohex-1-enyl-ethyl)-ureido]-3-(pyridin-3-ylmethoxy)-isothiazole-4-carboxylic acid amide;
5-[3-(2,5-Dimethyl-benzyl)-ureido]-3-(pyridin-3-ylmethoxy)-isothiazole-4-carboxylic acid amide;
5-[3-(3,5-Dimethoxy-benzyl)-ureido]-3-(pyridin-3-ylmethoxy)-isothiazole-4-carboxylic acid amide;
5-[3-(2-Ethoxy-benzyl)-ureido]-3-(pyridin-3-ylmethoxy)-isothiazole-4-carboxylic acid amide;
5-{3-[2-(2-Ethoxy-phenyl)-ethyl]-ureido}-3-(pyridin-3-ylmethoxy)-isothiazole-4-carboxylic acid amide;
5-{3-[2-(3,4-Dimethoxy-phenyl)-ethyl]-ureido}-3-(pyridin-3-ylmethoxy)-isothiazole-4-carboxylic acid amide;
5-(3-Phenethyl-ureido)-3-(pyridin-3-yl-methoxy)-isothiazole-4-carboxylic acid amide;
5-{3-[2-(3-Ethoxy-4-methoxy-phenyl)-ethyl]-ureido}-3-(pyridin-3-ylmethoxy)-isothiazole-4-carboxylic acid amide;
5-{3-[2-(4-Ethoxy-phenyl)-ethyl]-ureido}-3-(pyridin-3-ylmethoxy)-isothiazole-4-carboxylic acid amide;
5-{3-[2-(4-Chloro-phenyl)-ethyl]-ureido}-3-(pyridin-3-ylmethoxy)-isothiazole-4-carboxylic acid amide;
5-{3-[2-(3-Chloro-phenyl)-ethyl]-ureido}-3-(pyridin-3-ylmethoxy)-isothiazole-4-carboxylic acid amide;
5-{3-[2-(3-Methoxy-phenyl)-ethyl]-ureido}-3-(pyridin-3-ylmethoxy)-isothiazole-4-carboxylic acid amide;
5-{3-[2-(4-Methoxy-phenyl)-ethyl]-ureido}-3-(pyridin-3-ylmethoxy)-isothiazole-4-carboxylic acid amide;
5-{3-[2-(3-Bromo-4-methoxy-phenyl)-ethyl]-ureido}-3-(pyridin-3-ylmethoxy)-isothiazole-4-carboxylic acid amide;
5-{3-[2-(4-Bromo-phenyl)-ethyl]-ureido}-3-(pyridin-3-ylmethoxy)-isothiazole-4-carboxylic acid amide;
5-{3-[2-(2-Chloro-phenyl)-ethyl]-ureido}-3-(pyridin-3-ylmethoxy)-isothiazole-4-carboxylic acid amide;
5-{3-[2-(3-Chloro-phenyl)-ethyl]-ureido}-3-(pyridin-3-ylmethoxy)-isothiazole-4-carboxylic acid amide;
5-{3-[2-(2-Fluoro-phenyl)-ethyl]-ureido}-3-(pyridin-3-ylmethoxy)-isothiazole-4-carboxylic acid amide;
5-{3-[2-(3-Fluoro-phenyl)-ethyl]-ureido}-3-(pyridin-3-ylmethoxy)-isothiazole-4-carboxylic acid amide;
5-{3-[2-(4-Fluoro-phenyl)-ethyl]-ureido}-3-(pyridin-3-ylmethoxy)-isothiazole-4-carboxylic acid amide;
5-{3-[2-(4-Ethoxy-3-methoxy-phenyl)-ethyl]-ureido}-3-(pyridin-3-ylmethoxy)-isothiazole-4-carboxylic acid amide;
5-{3-[2-(3-Ethoxy-4-methoxy-phenyl)-ethyl]-ureido}-3-(pyridin-3-ylmethoxy)-isothiazole-4-carboxylic acid amide;
5-{3-[2-(2,5-Dimethoxy-phenyl)-ethyl]-ureido}-3-(pyridin-3-ylmethoxy)-isothiazole-4-carboxylic acid amide;
5-{3-[2-(3-Methoxy-phenyl)-ethyl]-ureido}-3-(pyridin-3-ylmethoxy)-isothiazole-4-carboxylic acid amide;
5-[3-(2-Dinuoromethoxy-benzyl)-ureido]-3-(pyridin-3-ylmethoxy)-isothiazole-4-carboxylic acid amide;
5-[3-(2,6-Dimethoxy-benzyl)-ureido]-3-(pyridin-3-ylmethoxy)-isothiazole-4-carboxylic acid amide;
5-[3-(2,5-Dichloro-benzyl)-ureido]-3-(pyridin-3-ylmethoxy)-isothiazole-4-carboxylic acid amide;
5-[3-(3-Morpholin-4-yl-propyl)-ureido]-3-(pyridin-3-ylmethoxy)-isothiazole-4-carboxylic acid amide;
5-[3-(2-Morpholin-4-yl-ethyl)-ureido]-3-(pyridin-3-ylmethoxy)-isothiazole-4-carboxylic acid amide;
5-[3-(2-Diethylamino-ethyl)-ureido]-3-(pyridin-3-ylmethoxy)-isothiazole-4-carboxylic acid amide;
5-[3-(3-Dimethylamino-propyl)-ureido]-3-(pyridin-3-ylmethoxy)-isothiazole-4-carboxylic acid amide;
5-{3-[2-(1-Methyl-pyrrolidin-2-yl)-ethyl]-ureido}-3-(pyridin-3-ylmethoxy)-isothiazole-4-carboxylic acid amide;
5-{3-[3-(2-Methyl-piperidin-1-yl)-propyl]-ureido}-3-(pyridin-3-ylmethoxy)-isothiazole-4-carboxylic acid amide;
(R),(R)-5-[3-(2-Hydroxy-cycloheptylm-ethyl)-ureido]-3-(pyridin-3-ylmethoxy)-isothiazole-4-carboxylic acid amide;
(R),(R)-5-[3-(2-Hydroxy-cyclooctylmethyl)-ureido]-3-(pyridin-3-ylmethoxy)-isothiazole-4-carboxylic acid amide;
5-[3-(2-Hydroxy-ethyl)-ureido]-3-(pyridin-3-ylmethoxy)-isothiazole-4-carboxylic acid amide;
5-[3-(2-Hydroxy-butyl)-ureido]-3-(pyridin-3-ylmethoxy)-isothiazole-4-carboxylic acid amide;
5-{3-[3-(2-Oxo-pyrrolidin-1-yl)-propyl]-ureido}-3-(pyridin-3-ylmethoxy)-isothiazole-4-carboxylic acid amide;
5-[3-(3-Imidazol-1-yl-propyl)-ureido]-3-(pyridin-3-ylmethoxy)-isothiazole-4-carboxylic acid amide;
5-(3-Benzyl-ureido)-3-(pyridin-3-yl-methoxy)-isothiazole-4-carboxylic acid amide;
5-[3-(2,5-Difluoro-benzyl)-ureido]-3-(pyridin-3-ylmethoxy)-isothiazole-4-carboxylic acid amide;
3-(1-Pyridin-3-yl-ethoxy)-5-(3-pyridin-2-yl-methyl-ureido)-isothiazole-4-carboxylic acid amide;
5-[3-(2,6-Dimethoxy-benzyl)-ureido]-3-(1-pyridin-3-yl-ethoxy)-isothiazole-4-carboxylic acid amide;
5-(3-Cyclopropylmethyl-ureido)-3-(pyridin-3-ylmethoxy)-isothiazole-4-carboxylic acid amide;
5-(3-Methyl-ureido)-3-(pyridin-3-yl-methoxy)-isothiazole-4-carboxylic acid amide;
5-(3-Methyl-ureido)-3-(1-pyridin-3-yl-ethoxy)-isothiazole-4-carboxylic acid amide; and
5-[3-(3,5-Dichloro-benzyl)-ureido]-3-(pyri-din-3-ylmethoxy)-isothiazole-4-carboxylic acid amide.

Anti-TGFβ antibodies

In some embodiments, the inhibitor of TGFβ cell signaling used to replace Sox2 is an anti-TGFβ antibody. Antibodies to anti-TGFβ are well known in the art, and include pan specific anti-TGFB from R&D (cat No: Ab-100 NA) and specific anti-TGFβRII from R& S systems (Cat No. AB-13 NA) as disclosed herein in the Examples.

Other Inhibitors of TGF β cell signaling

Small molecule inhibitors of TGFβ signaling pathway are known in the art e.g., Callahan J F et al., J. Med. Chem. (2002) 45: 999-1001; Sawyer J S et al., J. Med. Chem. (2003) 46, 3953-3956; Gellibert F et al., J. Med. Chem. (2004) 47: 4494-4506; Tojo Metal., Cancer Sci. (2005) 96: 791-800; Valdimarsdottir G et al., APMIS (2005) 113: 773-789 and Petersen et al., Kidney International (2008) 73: 705-715. Each of these references is incorporated by reference in its entirety. Non-limiting examples of small molecule inhibitors of TGFβ signaling pathway include Dihydropyrroloimidazole Analogues (e.g. SKF-104365), Triarylimidazole Analogues (e.g., SB-202620 and SB-203580), RL-0061425, 1,5-naphthyridine aminothiazole and pyrazole derivatives (e.g., 4-(6-Methyl-pyridin-2-yl)-5-(1,5-naphthyridin-2-yl)-1,3-thiazol-2-amine and 2-[3-(6-Methyl-pyridin-2-yl)-1H-pyrazol-4-yl]-1,5-naphthyridine), 4-(5-benzo[1,3]dioxol-5-yl-4-pyridin-2-yl-1H-imidazol-2-yl)-benzamide (SB431542), 4-(4-[3-(Pyridin-2-yl)-1H-pyrazol-4-yl]pyridin-2-yl)-N-(tetrahydro-2Hpyran-4-yl)-benzamide (GW788388), A-83-01, Decorin, Lefty1, Lefty2, Follistatin, Noggin, Chordin, Cerberus, Cerberus, Gremlin, Inhibin, and BIO (6-bromo-indirubin-3′ oxime).

In some embodiments, other non-limiting examples of small molecule inhibitors of TGFβRs which can be used to replace exogenous Sox2 include for example, 2-(3-(6-Methylpyridin-2-yl)-1H-pyrazol-4-yl)-1,5napththyridine, [3-(Pyridin-2-yl)-4-(4-quinoyl)]-1H-pyrazole, and 3-(6-Methylpyridin-2-yl)-4-(4-quinolyl)-1-phenylthiocarbamoyl-1H-pyrazole, which can be purchased from Calbiochem (San Diego, Calif.). Other small molecule inhibitors include, but are not limited to, SB-431542 (see e.g., Halder et al., 2005; Neoplasia 7(5):509-521), SM16 (see e.g., Fu, K et al., 2008; Arteriosclerosis, Thrombosis and Vascular Biology 28(4):665), and SB-505124 (see e.g., Dacosta Byfield, S., et al., 2004; Molecular Pharmacology 65:744-52), among others.

In one embodiment, the ALK5 inhibitor 2-(3-(6-Methylpyridin-2-yl)-1H-pyrazol-4-yl)-1,5napththyridine is used with the methods described herein. This inhibitor is also referred to herein as ALK5 inhibitor II and is available commercially from Calbiochem (Cat. No. 616452; San Diego, Calif.). In one embodiment, the inhibitor is SB 431542, an ALK-4, -5, -7 inhibitor, commercially available from Sigma (product no. S4317; Saint Louis, Mo.). SB 431542 is also referred to by the following chemical names: 4-[4-(1,3-Benzodioxol-5-yl)-5-(2-pyridinyl)-1H-imidazol-2-yl]-benzamide, 4-[4-(3,4-methylenedioxyphenyl)-5-(2-pyridyl)-1H-imidazol-2-yl]-benzamide, or 4-(5-benzol[1,3]dioxol-5-yl-4-pyridin-2-yl-1H-imidazol-2-yl)-benzamide hydrate.

Small molecules inhibitors of TGF-β signaling can be classified based on the basic scaffold of the molecule. For example, TGF-β signaling inhibitors can be based on the dihydropyrrlipyrazole-based scaffold, imidazole-based scaffold, pyrazolopyridine-based scaffold, pyrazole-based scaffold, imidazopyridine-based scaffold, triazole-based scaffold, pyridopyrimidine-based scaffold, pyrrolopyrazole-based scaffold, isothiazole-based scaffold and oxazole-based scaffold.

Inhibitors of TGF-β signaling are described in Callahan, J. F. et al., J. Med. Chem. 45, 999-1001 (2002); Sawyer, J. S. et al., J. Med. Chem. 46, 3953-3956 (2003); Gellibert, F. et al., J. Med. Chem. 47, 4494-4506 (2004); Tojo, M. et al., Cancer Sci. 96: 791-800 (2005); Valdimarsdottir, G. et al., APMIS 113, 773-389 (2005); Petersen et al. Kidney International 73, 705-715 (2008); Yingling, J. M. et al., Nature Rev. Drug Disc. 3, 1011-1022 (2004); Byfield, S. D. et al., Mol. Pharmacol., 65, 744-752 (2004); Dumont, N, et al., Cancer Cell 3, 531-536 (2003); WO Publication No. 2002/094833; WO Publication No. 2004/026865; WO Publication No. 2004/067530; WO Publication No. 209/032667; WO Publication No. 2004/013135; WO Publication No. 2003/097639; WO Publication No. 2007/048857; WO Publication No. 2007/018818; WO Publication No. 2006/018967; WO Publication No. 2005/039570; WO Publication No. 2000/031135; WO Publication No. 1999/058128; U.S. Pat. No. 6,509,318; U.S. Pat. No. 6,090,383; U.S. Pat. No. 6,419,928; U.S. Pat. No. 9,927,738; U.S. Pat. No. 7,223,766; U.S. Pat. No. 6,476,031; U.S. Pat. No. 6,419,928; U.S. Pat. No. 7,030,125; U.S. Pat. No. 6,943,191; U.S. Publication No. 2005/0245520; U.S. Publication No. 2004/0147574; U.S. Publication No. 2007/0066632; U.S. Publication No. 2003/0028905; U.S. Publication No. 2005/0032835; U.S. Publication No. 2008/0108656; U.S. Publication No. 2004/015781; U.S. Publication No. 2004/0204431; U.S. Publication No. 2006/0003929; U.S. Publication No. 2007/0155722; U.S. Publication No. 2004/0138188 and U.S. Publication No. 2009/0036382, the contents of each which are herein incorporated by reference in their entirety.

Oligonucleotide based modulators of TGF-β signaling, such as siRNAs and antisense oligonucleotides, are described in U.S. Pat. No. 5,731,424; U.S. Pat. No. 6,124,449; U.S. Publication Nos. 2008/0015161; 2006/0229266; 2004/0006030; 2005/0227936 and 2005/0287128, each of which are herein incorporated by reference in their entirety. Other antisense nucleic acids and siRNAs can be obtained by methods known to one of ordinary skill in the art.

Exemplary inhibitors of TGF-β signaling include, but are not limited to, AP-12009 (TGF-β Receptor type II antisense oligonucleotide), Lerdelimumab (CAT 152, antibody against TGF-β Receptor type II) GC-1008 (antibody to all isoforms of human TGF-β), ID11 (antibody to all isoforms of murine TGF-β), soluble TGF-β, soluble TGF-β Receptor type II, dihydropyrroloimidazole analogs (e.g., SKF-104365), triarylimidazole analogs (e.g., SB-202620 (4-(4-(4-fluorophenyl)-5-(pyridin-4-yl)-1H-imidazol-2-yl)benzoic acid) and SB-203580 (4-(4-Fluorophenyl)-2-(4-methylsulfinyl phenyl)-5-(4-pyridyl)-1H-imidazole)), RL-0061425, 1,5-naphthyridine aminothiazole and pyrazole derivatives (e.g., 4-(6-methyl-pyridin-2-yl)-5-(1,5-naphthyridin-2-yl)-1,3-thiazole-2-amine and 2-[3-(6-methyl-pyridin-2-yl)-1H-pyrazole-4-yl]-1,5-naphthyridine), SB-431542 (4-(5-Benzol[1,3]dioxol-5-yl-4-pyridin-2-yl-1H-imidazol-2-yl)-benzamide), GW788388 (4-(4-(3-(pyridin-2-yl)-1H-pyrazol-4-yl)pyridin-2-yl)-N-(tetrahydro-2H-pyran-4-yl)benzamide), A-83-01 (3-(6-Methyl-2-pyridinyl)-N-phenyl-4-(4-quinolinyl)-1H-pyrazole-1-carbothioamide), Decorin, Lefty 1, Lefty 2, Follistatin, Noggin, Chordin, Cerberus, Gremlin, Inhibin, BIO (6-bromo-indirubin-3′-oxime), Smad proteins (e.g., Smad6, Smad7), and Cystatin C.

Inhibitors of TGF-β signaling also include molecules which inhibit TGF-β Receptor type I. Inhibitors of TGF-β Receptor type I are described in Byfield, S. D., and Roberts, A. B., Trends Cell Biol. 14, 107-111 (2004); Sawyer J. S. et al., Bioorg. Med. Chem. Lett. 14, 3581-3584 (2004); Sawyer, J. S. et al., J. Med. Chem. 46, 3953-3956 (2003); Byfield, S. D. et al., Mol. Pharmacol. 65, 744-752 (2004); Gellibert, F. et al., J. Med. Chem. 47, 4494-4506 (2004); Yingling, J. M. et al., Nature Rev. Drug Disc. 3, 1011-1022 (2004); Dumont, N, et al., Cancer Cell 3, 531-536 (2003); Tojo, M. et al., Cancer Sci. 96: 791-800 (2005); WO Publication No. 2004/026871; WO Publication No. 2004/021989; WO Publication No. 2004/026307; WO Publication No. 2000/012497; U.S. Pat. No. 5,731,424; U.S. Pat. No. 5,731,144; U.S. Pat. No. 7,151,169; U.S. Publication No. 2004/00038856 and U.S. Publication No. 2005/0245508, contents of all of which are herein incorporated in their entireties.

Exemplary inhibitors of TGF-β Receptor type I include, but are not limited to, soluble TGF-β Receptor type I; AP-11014 (TGF-β Receptor type I antisense oligonucleotide); Metelimumab (CAT 152, TGF-β Receptor type I antibody); LY550410; LY580276 (3-(4-fluorophenyl)-5,6-dihydro-2-(6-methylpyridin-2-yl)-4H-pyrrolo[1,2-b]pyrazole); LY364947 (4-[3-(2-Pyridinyl)-1H-pyrazol-4-yl]-quinoline); LY2109761; LY573636 (N-((5-bromo-2-thienyl)sulfonyl)-2,4-dichlorobenzamide); SB-505124 (2-(5-Benzo[1,3]dioxol-5-yl-2 tert-butyl-3H-imidazol-4-yl)-6-methylpyridine); SD-208 (2-(5-Chloro-2-fluorophenyl)-4-[(4-pyridyl)amino]pteridine); SD-093; KI2689; SM16; FKBP12 protein; 3-(4-(2-(6-methylpyridin-2-yl)H-imidazo[1,2-a]pyridin-3-yl)quinolin-7-yloxy)-N,N-dimethylpropan-1-amine;

RNAi Inhibitors of TGF β Receptor

TGFBR1 mRNA has been successfully targeted using siRNAs; see for example, which can be obtained from Santacruz Biotechnology (cat No: sc-40222), which is incorporated herein by reference. Others siRNA molecules may be readily prepared by those of skill in the art based on the known sequence of the target mRNA. To avoid doubt, the sequence of a human TGFBR cDNA is provided at, for example, GenBank Accession Nos. P36897.1 (SEQ ID NO: 3), Q5T7S2 (SEQ ID NO: 4), Q61R47, P37173 (SEQ ID NO: 5), Q6A176 (not shown), Q706C0 (not shown), Q706C1 (not shown), and Q03167.2 (SEQ ID NO: 6), among others.

### Inhibitors of Src Signaling Pathway

Src family kinases are 52-62-kDa membrane-associated nonreceptor tyrosine kinases and they participate in several tyrosine phosphorylation-related signaling pathways in response to various extracellular ligands. Src, for example, contains at least three important protein interaction domains. The SH3 domain binds to polyproline motifs and the SH2 domain interacts with the phosphorylated tyrosine residues. The kinase domain reacts with the nucleotide and phosphorylates the substrate. Binding of protein ligands to the SH3 or SH2 domain can activate Src. Proteins that bind with kinase domain of Src were also reported to be capable of regulating Src activity.

Na+/K+-ATPase, the molecular machinery of the cellular sodium pump, belongs to a family of evolutionarily ancient enzymes that couple the hydrolysis of ATP to membrane ion translocation. It is now believed that the Na+/K+-ATPase has dual functions. It not only pumps Na+ and K+ across cell membranes, but also relays the extracellular CTS signal to intracellular compartments via activation of different protein kinases.

Src and Src family kinases are non-receptor tyrosine kinases that play an important role in regulation of various signaling pathways involved in control of cell growth, mobility, and muscle contraction. Moreover, our recent studies have shown that activation of Na/K-ATPase-associated Src by cardiotonic steroids protects the heart from ischemia/reperfusion injury. It also inhibits cancer cell growth and stimulates collagen synthesis in fibroblasts. Because Src family kinases are highly active in many types of cancer, pharmaceutical companies are interested in developing specific Src and Src-family kinase inhibitors. Most of the developed inhibitors are ATP analogs that directly compete with ATP.

The non-receptor protein tyrosine, Src, is a 60-kDa protein that is a member of a nine-gene family, including Src, Yes, Fyn, Lyn, Lck, Hck, Fgr, BIk, and Yrk, that plays a critical role in the regulation of many cellular processes, such as proliferation, differentiation, migration, adhesion, invasion, angiogenesis, and immune function (Yeatman T J. (2004) Nat Rev Cancer 4(6):470-80; Frame M C. (2004) J Cell Sci 117:989-98). The Src family kinase contains a poorly conserved domain and three conserved Src homology domains: SH2, SH3, and SH1 or protein tyrosine kinase domain. Critical to the regulation of Src is a COOH-terminal tyrosine (Y530) that, when phosphorylated by C-ferminal Src kinase (Csk), leads to a more inactive Src conformation Src interacts with many proteins, depending on the input signal. It further assumes its active conformation through dephosphorylation of Y530 and autophosphorylation of Y418. Src also associates with structural and signaling proteins, and the resulting complexes are critical to Src's role in diverse cellular processes. Src has been reported to be overexpressed or aberrantly activated in a number of cancers, such as colon, breast, melanomas, ovarian cancer, gastric cancer, head and neck cancers, pancreatic cancer, lung cancer, brain cancers, and blood cancers (Dehm S M and Bonham K (2004) Biochem Cell Biol 2004; 82:263-74).

Small Molecule Inhibitors of Src Signaling (Src Inhibitors)

Described herein are compounds that can be used in the methods and kits described herein for the replacement of sox2, for example, in methods of producing a reprogrammed cell (e.g. iPS cell or partially reprogrammed cell) from a differentiated cell. Exemplary compounds for use in the methods and kits described herein as inhibitors of SRC cell signaling include those described generically (e.g., the compounds of Formula (II)) and also those described specifically, e.g., the compounds depicted in FIG. 1D (EI-275).

Formula II

In one aspect, the disclosure features a method of producing a reprogrammed cell (e.g. iPS cell or a partially reprogrammed cell) from a differentiated cell, the method comprising: contacting an isolated differentiated cell with a compound of formula (II)

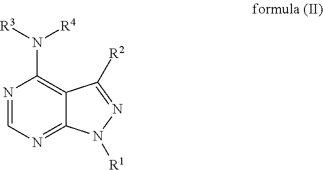

wherein,

R¹ is H, C₁-C₆ alkyl, or C₁-C₆ haloalkyl;

R² is optionally substituted aryl or heteroaryl; and

each R³ and R⁴ is independently H, C₁-C₆ alkyl, arylC₁-C₆alkyl, or a nitrogen protecting group to thereby produce a reprogrammed cell (e.g. iPS cell or partially reprogrammed cell) from the differentiated cell.

In one embodiment, the method comprises contacting a plurality of differentiated cells with a compound of formula (II) to thereby produce a plurality of reprogrammed cells (e.g. iPS cells or partially reprogrammed cells) from the differentiated cells.

In some embodiments, R¹ is a branched alkyl. In some embodiments, R¹ is t-butyl.

In some embodiments, R² is aryl (e.g., a monocyclic aryl such as phenyl). In some embodiments, R² is substituted. In some embodiments R² is monosubstituted. Exemplary substituents include halo, C₁-C₆ alkyl, C₁-C₆haloalkyl, hydroxyl, C₁-C₆alkoxy, C₁-C₆haloalkoxy (e.g., C₁-C₆ alkyl such as methyl). In some embodiments, R²

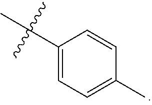

In some embodiments, each R³ and R⁴ is H.

Exemplary compounds of formula (II) include EI-275 as shown in FIG. 1D, and has the following structure:

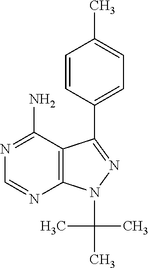

Anti-SRC Antibodies

In some embodiments, inhibitors of SRC signaling include those Src inhibitors are antibodies, including anti-Src antibodies which are commercially available. Some examples of commercially available anti-Src antibodies which can be used as Src inhibitors according to the methods as disclosed herein include for example, but not limited to: Abcam antibodies (e.g. Cat Nos: Ab79308, Ab24789, Ab4816, Ab47411); Santa Cruz antibodies such as sc-6096, sc-6098; sc-73056, and other commercial sources such as Cell Signalling, Invitrogen, Sigma, AdD Serotec and the like.

Other SRC Signaling Inhibitors

In some embodiments, inhibitors of SRC signaling include those Src inhibitors listed in International Patent Application WO/2008/054792 and WO/2008/115404, and U.S. Patent Application 20090093495 which is incorporated herein in its entirety by reference. In other embodiments, other small molecule inhibitors of src can be used to direct reprogramming of a differentiated cell into a reprogrammed cells, such as for example dasatinib (BMS354825), AZD-0530, SKI-606, PP1 (4-Amino-5-(4-methylphenyl)-7-(t-butyl)pyrazolo[3,4-d]-pyrimidine), PP2 (4-chlorophenyl)-7-(/-butyl)pyrazolo[3,4-</]-pyrimidine), PD 166326 and KX2-391.

In some embodiments, Src kinase inhibitors useful for replacing a member of Sox transcription factor (e.g. Sox2) and useful in the methods and compositions for reprogramming a differentiated cell include Src specific tyrosine kinase inhibitors, such as but not limited to, CsK, tyrphostin-derived inhibitors, derivatives of benzylidenemalonitrile, pyrazolopyrimidine (e.g., PP1), and microbial agents, such as angelmicin B; and competitive inhibitors, such as small phosphotyrosine containing ligands. Src family kinase inhibitors are described in U.S. Pat. Nos.: RE34,267; 6,316,444; 6,329,380; 6,498,165; 6,503,914; 7,285,556; 7,429,596; 6,613,776; 6,6106,677; 6,489,328; 6,506,769; 5,916,908; ,728,726; 5,795,910; 6,689,772; 6,777,417; 6,696,452; 6,653,300; 6,653,301; 6,638,926; 6,846,928; 6,369,086; 7,053,070; 6,689,778; 6,313,138; 6,306,874; 6,313,138; 6,306,874; 6,596,746; 6,635,626; 5,958,935; 6,395,734; 6,479,512; 6,420,382; 6,051,593; 6,541,503; 6,387,919; 6,130,238; 5,990,109; 6,573,293; 6,245,759; 6,337,335; 6,239,133; 6,114,371; 6,579,897; 6,624,174; 6,180,636; 6,048,866; 7,049,438; 6,235,740; 7,008,948; 6,383,790, U.S. Patent Publication Nos.: US200410014676; US2006/0122199, US2006/0004002 and US2007/0185139, and International Publication Nos. WO01/94341 and WO01/00214, all of which are incorporated herein in their entirety by reference.

Exemplary Src family kinase inhibitors useful in the methods and compositions for reprogramming a differentiated cell as disclosed herein include, but are not limited to, AZDO424, 4-(6-chloro-2,3-methylenedioxyanilino)-7-[2-(4-methylpiperazin-hypethoxy]-5-tetrahydropyran-4-yloxyquinazoline (AZDO530), AZM559756, M475271, SU6656, SU6657, TG100435, S2075, EI-274 (Damnacanthol), EI-227 (herbimycin), P-306 (peptide A), EI-271 (piceatannol), EI-275 (PP1), EI-297 (PP2), 4-Amino-7-phenylpyrazol[3,4-d]pyrimidine (PP3), EI-285 (radicicol), Gleevec®, N-(2-chloro-6-methylphenyl)-2-(6-[4-(2-hydroxyethyl)piperazin-1-yl]-2-methylpyrimidin-4-ylamino)thiazole-5-carboxamide (dasatinib), BMS-354825, Sprycel®), staurosporine, RACK1, SKI-606 (nosutinib), KX2-391, 4-(4′-phenoxyanilino)-6,7-dimethoxyquinazoline (Src Kinase Inhibitor I), Src Kinase Inhibitor II, and K252a.

RNAi Inhibitors of SRC Signaling

Inhibition of the Src signaling pathway can be by RNA interference (RNAi) according to methods commonly known by a skilled artisan. For example, a pool of four complementary siRNA oligonucleotide duplexes targeted specifically to human Src (GenBank NM_{—}005417) have been previously used to knockdown Src expression, as disclosed in Mishra et al., Mol Pharmacol 67:2049-2056, 2005, which is incorporated herein in its entirety by reference) and were obtained from Dharmacon Inc. (Lafayette, Colo.).

Src mRNA has been successfully targeted using siRNAs; see, for example, Mishra et al., Mol Pharmacol 67:2049-2056, 2005, which is incorporated herein by reference. Others siRNA molecules may be readily prepared by those of skill in the art based on the known sequence of the target mRNA. To avoid doubt, the sequence of a human Src cDNA is provided at, for example, GenBank Accession Nos. NM_{—}005417 (SEQ ID NO: 7). Src RNAi agents are also commercially available, such as, for example, from Santa-Cruz (Cat No: sc-36555; sc-36556) and also from other companies, such as Invitrogen.

### Chemical Replacement of Klf Family of Transcription Factor

Another aspect of the present invention relates to a method to produce a reprogrammed cell by contacting a differentiated cell with at least one small molecule, selected from any compound with Formula VIII or IX which replaces a transcription factor from the Klf family of transcription factors. Examples of the Klf family of transcription factors include Klf1, Klf2, Klf4, Klf5 and the like. Klf4 (Kruppel like factor-4) is reported as a tumor repressing factor (Ghaleb et al., Cell Res. 15:92-96, 2005). The accession numbers of members of the Klf family are: Klf1 Kruppel-like factor 1 (erythroid) NM_{—}010635 (mouse); NM_{—}006563 (human); Klf2 Kruppel-like factor 2 (lung) NM_{—}008452 (mouse), NM_{—}016270 (human); Klf4 Kruppel-like factor 4, NM_{—}010637 (mouse) (SEQ ID NO: 8), NM_{—}004235 (human) (SEQ ID NO: 9); Klf5 Kruppel-like factor 5, NM_{—}009769 (mouse), NM_{—}001730 (human)

In some embodiments, a reprogrammed cell is produced by contacting a cell with one or more small molecule which replaces a transcription factor from the Klf family of transcription factors (such as Kfl4), e.g. any compound with Formula VIII or IX, and with one or more compounds which replaces the transcription from the Oct family of transcription factors (such as Oct4). In one embodiment, any compound selected from any of formula VIII-1× to reprogram a differentiated cell can be used in any combination of members from one or more transcription factors gene families. For example, a combination of one or more gene products of Oct3/4, Sox2, and c-Myc.

In one embodiment, replacement of a member of the Klf family of transcription factors, such as replacement of exogenous transcription factor Klf 4 is by an agent which activates the Mek/Erk signalling pathway, such as a Mek/Erk agonist. In some embodiments, replacement of exogenous transcription factor Klf4 is by any compound with the formula selected from Formulas VIII. In some embodiments, where a differentiated cell is contacted with an agonist of Mek/Erk cell signalling, or a compound with the Formula VIII, the cell is not contacted with a member of the Klf transcription factor family, such as exogenous Klf4 transcription factor, including a nucleic acid encoding a Klf4 protein or a Kfl4 protein. In some embodiments, replacement of exogenous transcription factor Klf4 is by any compound with Formula VIII such as Prostoglandin 2.

In one embodiment, replacement of a member of the Klf family of transcription factors, such as replacement of exogenous transcription factor Klf 4 is by an agent which is an inhibitor of the EGF signalling pathway, such as a EGF inhibitor, or a EGFR kinase inhibitor. In some embodiments, replacement of exogenous transcription factor Klf4 is by any compound with the Formula IX. In some embodiments, where a differentiated cell is contacted with an inhibitor of the EGF signalling pathway, or a compound with the Formula IX, the cell is not contacted with a member of the Klf transcription factor family, such as exogenous Klf4 transcription factor, including a nucleic acid encoding a Klf4 protein or a Kfl4 protein. In some embodiments, replacement of exogenous transcription factor Klf4 is by any compound with Formula IX such as HBDA.

In one embodiment, replacement of a member of the Klf family of transcription factors, such as replacement of exogenous transcription factor Klf 4 is by an agent which is an inhibitor of the Ca²⁺/Calmodulin signalling pathway, such as a Ca²⁺/Calmodulin inhibitor. In some embodiments, replacement of exogenous transcription factor Klf4 is by any compound with the Formula IX. In some embodiments, where a differentiated cell is contacted with an inhibitor of the Ca²⁺/Calmodulin signalling pathway, or a compound with the Formula IX, the cell is not contacted with a member of the Klf transcription factor family, such as exogenous Klf4 transcription factor, including a nucleic acid encoding a Klf4 protein or a Klf4 protein. In some embodiments, replacement of exogenous transcription factor Klf4 is by any compound with Formula IX such as HBDA.

In some embodiments, contact of a differentiated cell with an agent which replaces a member of the Klf family of transcription factors, such as Klf4, (i.e. an agonist of the Mek/Erk signalling pathway, or a inhibitor of EGF signalling pathway, or a EGFR inhibitor, or a inhibitor of Ca²⁺/Calmodulin signalling pathway or any compound with Formulas VIII-IX, including but not limited to Prostaglandin 2 or HDBA), enables reprogramming of differentiated cells by only 3 transcription factors, such as Sox2, Oct-4, and c-Myc without the need for a member of the Klf4 transcription factor family such as Kfl4. In some embodiments, contact of a differentiated cell with an agent which replaces Kfl4 requires only 2 transcription factors, Sox2 and Oct-4 without the need for c-Myc or Klf4. In some embodiments, contact of a differentiated cell with an agent which replaces Klf4 can also be contacted with at least one agent, preferably two agents which replaces the 2 transcription factors, Sox2 and Oct-4, as disclosed herein, without the need for exogenous Sox-2, Oct4, c-Myc or Klf4 (i.e. where the cell is not contacted with any members of Klf, Sox, Oct or Myc transcription factor families, such as exogenous Klf4, Oct4, Sox2 or c-myc transcription factors, including nucleic acid sequences encoding any one of Klf4, Oct4, Sox2 or c-myc proteins or any combination of Klf4, Oct4, Sox2 or c-myc proteins).

For example, reprogrammed cells (e.g. a iPS cell or partially reprogrammed cells) were identified in mouse fibroblasts (MEFs) infected by Sox-2, Oct-4, and c-Myc retroviruses together with Prostaglandin 2 or HBDA treatment. The number and percentage of reprogrammed cells (i.e. iPS cells or partially reprogrammed cells) colonies was comparable to those in the addition of nucleic acid encoding the Klf4 transgene. Thus, the 3-factor reprogramming efficiency by Prostaglandin 2 or HBDA treatment is comparable to the induction rate for mouse fibroblasts infected by 4 factors (Oct-4, Klf-4, c-Myc and Sox-2), demonstrating that Prostaglandin 2 or HBDA treatment effectively replaced the need for exogenous Klf4 transcription factor, including a nucleic acid encoding a Klf4 protein or a Klf4 protein. Thus, described herein are methods for producing reprogrammed cells from differentiated cells (i.e. from fibroblasts e.g., MEFs) without using the oncogenes, for example Klf4, c-Myc or Sox-2.

In some embodiments, a differentiated cell which is contacted with an agent which replaces exogenous Klf4 transcription factor, including a nucleic acid encoding a Klf4 protein or a Kfl4 protein i.e. an agonist of the Mek/Erk signalling pathway, or a inhibitor of EGF signalling pathway, or a EGFR inhibitor, or a inhibitor of Ca²⁺/Calmodulin signalling pathway or any compound with Formulas VIII-IX, including but not limited to Prostaglandin 2 or HDBA), can be reprogrammed with small molecules or other agents which replace exogenous supplied Oct-4 and Sox2, as disclosed herein. Thus, described herein are methods for producing reprogrammed cells from differentiated cells (i.e. from fibroblasts e.g., MEFs) without using the oncogenes, for example c-Myc or oncogenes associated with introduction of nucleic acid sequences encoding the transcription factors Sox-2, Oct-4 or Klf-4 into the differentiated cell to be reprogrammed (i.e. viral oncogenes). For example, the chemical mediated reprogramming of differentiated cells makes it possible to create reprogrammed cells (i.e. iPS cells or partially reprogrammed cells) from small numbers of differentiated cells (e.g., such as those obtained from hair follicle cells from patients, blood samples, adipose biopsy, fibroblasts, skin cells, etc). In one embodiments, the addition of small molecules compounds (e.g., chemicals) allows successful and safe generation of reprogrammed cells (i.e. iPS cells) from human differentiated cells, such as skin biopsies (fibroblasts or other nucleated cells) as well as from differentiated cells from all and any other cell type.

### Agonist of Mek/Erk Cell Signaling

In some embodiments, a chemically-induced reprogrammed cell be produced by contacting a differentiated cell with an agonist of Mek/Erk cell signalling pathway. The Mek/Erk signaling pathway is involved in many cellular processes in both the adult organism and the developing embryo including cell growth, cell differentiation, apoptosis, cellular homeostasis and other cellular functions.

Thus, one aspect of the present invention relates to reprogramming a differentiated cell, where a member of the Klf family of transcription factors, such as exogenous transcription factor Klf 4 is replaced by an agent which activates the Mek/Erk signalling pathway, such as a Mek/Erk agonist. In some embodiments, replacement of exogenous transcription factor Klf4 is by any compound with the formula selected from Formulas VIII. In some embodiments, where a differentiated cell is contacted with an agonist of Mek/Erk cell signalling, or a compound with the Formula VIII, the cell is not contacted with a member of the Klf transcription factor family, such as exogenous Klf4 transcription factor, including a nucleic acid encoding a Klf4 protein or a Kfl4 protein. In some embodiments, replacement of exogenous transcription factor Klf4 is by any compound with Formula VIII such as Prostoglandin 2.

In some embodiments, where a replacement of exogenous transcription factor Klf4 is by any compound with Formula VIII such as Prostoglandin 2, the differentiated cell is not contacted with a reprogramming efficiency agent, as that term is described herein, such as for example a HDAC inhibitor such as VPA and the like.

The mitogen activated protein kinase (MAPK) signaling pathways are involved in cellular events such as growth, differentiation and stress responses (J. Biol. Chem. (1993) 268, 14553-14556). Four parallel MAPK pathways have been identified to date: ERK1/ERK2, JNK, p38 and ERK5. These pathways are linear kinase cascades in that MAPKKK phosphorylates and activates MAPKK, and MAPKK phosphorylates and activates MAPK. To date, seven MAPKK homologs (MEK1, MEK2, MKK3, MKK4/SEK, MEK5, MKK6, and MKK7) and four MAPK families (ERK1/2, JNK, p38, and ERK5) have been identified. Activation of these pathways regulates the activity of a number of substrates through phosphorylation. These substrates include: transcription factors such as TCF, c-myc, ATF2 and the AP-1 components, fos and Jun; cell surface components EGF-R; cytosolic components including PHAS-T, p90.sup.rsk, cPLA.sub.2 and c-Raf-1; and cytoskeleton components such as tau and MAP2. MAPK signaling cascades are involved in controlling cellular processes including proliferation, differentiation, apoptosis, and stress responses.

Of the known MAPK signaling pathways, the Mek/Erk pathway (also known as the RAF-MEK-ERK pathway) mediates proliferative and anti-apoptotic signaling from growth factors and oncogenic factors such as Ras and Raf mutant phenotypes that promote tumor growth, progression, and metastasis. By virtue of its central role in mediating the transmission of growth-promoting signals from multiple growth factor receptors, the Mek/Erk pathway provides molecular targets with potentially broad therapeutic applications in, for example, cancerous and non-cancerous hyperproliferative disorders, immunomodulation and inflammation.

MEK occupies a strategic downstream position in the Mek/Erk pathway catalyzing the phosphorylation of its MAPK substrates, ERK1 and ERK2. Anderson et al. “Requirement for integration of signals from two distinct phosphorylation pathways for activation of MAP kinase.” Nature 1990, v. 343, pp. 651-653. In the ERK pathway, MAPKK corresponds with MEK (MAP kinase ERK Kinase) and the MAPK corresponds with ERK (Extracellular Regulated Kinase). No substrates for MEK have been identified other than ERK1 and ERK2. Seger et al. “Purification and characterization of mitogen-activated protein kinase activator(s) from epidermal growth factor-stimulated A431 cells.” J. Biol. Chem., 1992, v. 267, pp. 14373-14381. This tight selectivity, in addition to the unique ability to act as a dual-specificity kinase, is consistent with MEK's central role in integration of signals into the MAPK pathway. MEK also appears to associate strongly with MAP kinase prior to phosphorylating it, suggesting that phosphorylation of MAP kinase by MEK may require a prior strong interaction between the two proteins. Both this requirement and the unusual specificity of MEK are suggestive that it may have enough difference in its mechanism of action to other protein kinases that selective inhibitors of MEK, possibly operating through allosteric mechanisms rather than through the usual blockade of the ATP binding site, may be found.

Any agonist of the Mek/Erk pathway which can be used to replace members of the Klf transcription factor family, such as Klf4 are listed below, and include any agent with Formula VIII, as disclosed herein, such as Prostaglandin 2.

Formula VIII

In one aspect, the disclosure features a method of producing a reprogrammed cell (i.e, an reprogrammed cells (i.e. iPS cells or partially reprogrammed cells) from a differentiated somatic cell, the method comprising:

contacting an isolated differentiated (i.e. somatic) cell with a compound of formula (VIII)

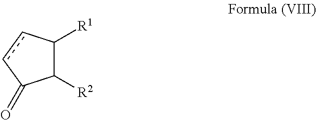

wherein,

R¹ is optionally substituted C₄-C₁₀ alkyl, C₄-C₁₀ alkenyl or C₄-C₁₀ alkynyl;

R² is optionally substituted C₄-C₁₀ alkyl, C₄-C₁₀ alkenyl or C₄-C₁₀ alkynyl; and

the dashed line ( - - - ) indicates the presence or absence of a bond;

to thereby produce an reprogrammed cells (i.e. iPS cells or partially reprogrammed cells) cell from the differentiated (i.e. somatic) cell.

In one embodiment, the method comprises contacting a plurality of differentiated cells with a compound of formula (VIII) to thereby produce a plurality of reprogrammed cells (i.e. iPS cells or partially reprogrammed cell) cells from the differentiated (i.e. somatic) cells.

In some embodiments, R¹ is C₄-C₁₀ alkenyl. In some embodiments, R¹ is C₄-C₁₀ alkenyl with one double bond. In some embodiments, R¹ is substituted. Exemplary substituents include hydroxy, oxo, COOH and COOalkyl. In some embodiments, R¹ is monosubstituted. In some embodiments, R¹ is substituted with —COOH. In some embodiments, R¹ is

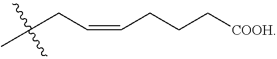

In some embodiments, R² is C₄-C₁₀ alkenyl. In some embodiments, R² is C₄-C₁₀ alkenyl with two double bonds. In some embodiments, R² is substituted. Exemplary substituents include hydroxy, oxo, COOH and COOalkyl. In some embodiments R² is unsubstituted. In some embodiments, R² is

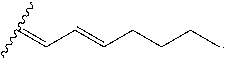

In some embodiments, the dashed line indicates the presence of a bond.

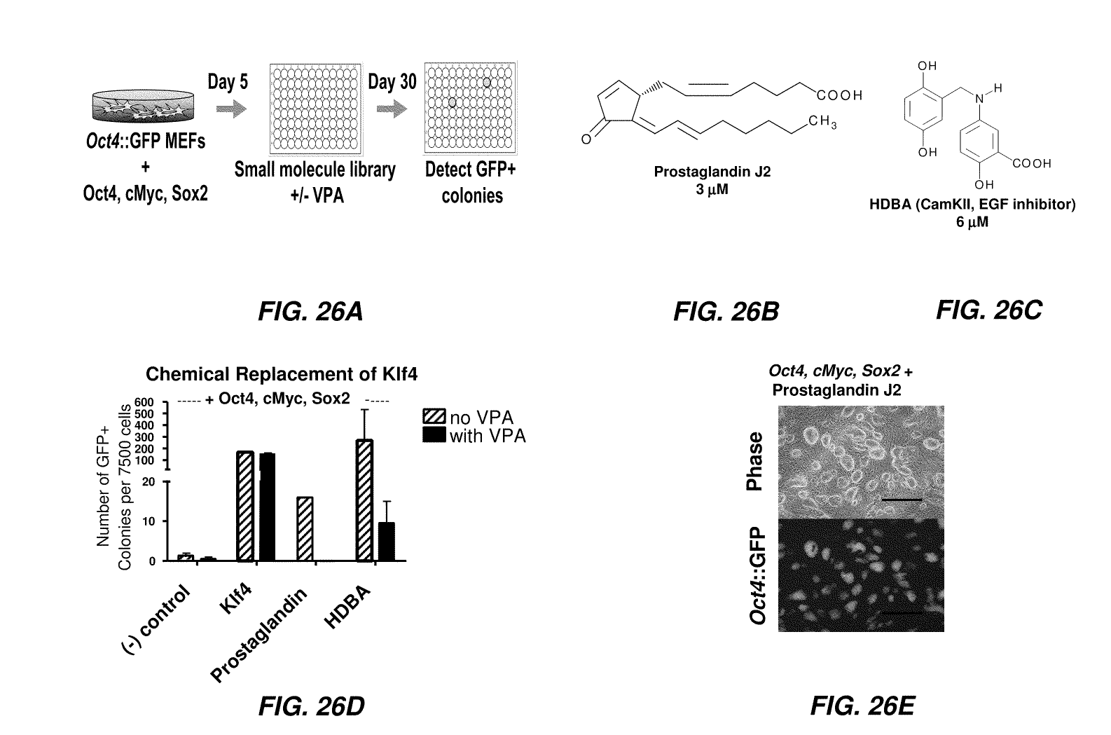

Exemplary compounds of formula (VIII) include Prostaglandin J2 (PGJ₂) compound, also shown herein and in FIG. 26.

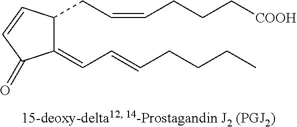

Other Inhibitors of Mek/Erk Cell Signalling

In some embodiments, other non-limiting examples of small molecule agonists of Erk/Mek cell signalling pathway which can be used to replace exogenous members of the Klf family of transcription factors, such as Klf4 are known in the art, and include for example, but are not limited to, exemplary Mek/Erk pathway agonists such as 15-deoxy-Δ^{12,14}-prostaglandin J₂ (15-d-PGJ₂) and thiazolidinediones such as rosiglitazone, pioglitazone, troglitazone, MCC-555, rivoglitazone and ciglitazone, 20-Hydroxyeicosatetraenoic acid (20-HETE), 20-hydroxyeicosa-5(Z),14(Z)-dienoic acid (5,14-20-HEDE) and N-[20-hydroxyeicosa-5(Z),14(Z)-dienoyl]glycine (5, 14-20-HEDGE). In some embodiments, small molecule agonists of Erk/Mek cell signalling pathway include 21 condition compounds, such as a 21 cocktail of Mek1, Mek2, Erk1, Erk2, GSK-3β as disclosed herein in the Examples section (see Example 6) and disclosed in T. S. Mikkelsen et al., Nature 454, 49 (Jul. 3, 2008); J. Silva et al., PLoS Biol 6, e253 (Oct. 21, 2008), which are incorporated herein by reference.

Antibody Inhibitors of Mek/Erk Cell Signalling

In some embodiments, inhibitors of Mek/Erk cell signaling include Mek inhibitors which are neutralizing antibodies, including anti-Mek antibodies which are commercially available. Some examples of commercially available anti-Mek antibodies which can be used as Mek/Erk inhibitors according to the methods as disclosed herein include for example, but not limited to: Cell Signalling antibodies (e.g. Cat Nos: 9124, 2352, 9124); Santa Cruz antibodies (e.g. cat Nos:cs-6250, sc-219; sc-81477, sc-436, sc-81504), and other commercial sources such as Cell Signalling, Invitrogen (e.g., cat nos: 44-452 (clone pS222), 44653G, 18-0376), Sigma, AdD Serotec and the like.

RNAi Inhibitors of Mek/Erk Cell Signalling.

Inhibition of the Mek/Erk signaling pathway can be by RNA interference (RNAi) according to methods commonly known by a skilled artisan. For example, siRNA oligonucleotide duplexes targeted specifically to human Erk (GenBank No: 5594) have been previously used to knockdown Mek/Erk expression.

Mek/Erk mRNA has been successfully targeted using siRNAs; and other siRNA molecules may be readily prepared by those of skill in the art based on the known sequence of the target mRNA. To avoid doubt, the sequence of a human Erk/Mek is provided at, for example, GenBank Accession Nos. 5594. Erk/Mek RNAi agents are also commercially available, such as, for example, from Santa-Cruz Biotechnology) and also from other companies, such as Invitrogen (e.g. cat nos: Hss108535, Hss108536, Hss108537 and VHS403108).

### Inhibitors of EGF Cell Signalling

In some embodiments, a chemically-induced reprogrammed cell be produced by contacting a differentiated cell with an inhibitor of EGF cell signalling pathway. The EGF signaling pathway is involved in many cellular processes in both the adult organism and the developing embryo including cell growth, cell differentiation, apoptosis, cellular homeostasis and other cellular functions.

Thus, one aspect of the present invention relates to reprogramming a differentiated cell, where a member of the Klf family of transcription factors, such as exogenous transcription factor Klf 4 is replaced by an agent which inhibits the EGF signalling pathway, such as a EGF inhibitor or in some embodiments, a EGF receptor (EGFR) inhibitor. In some embodiments, replacement of exogenous transcription factor Klf4 is by any compound with Formula IX. In some embodiments, replacement of exogenous transcription factor Klf4 is by any compound with Formula IX(a). In some embodiments, where a differentiated cell is contacted with an inhibitor of EGF cell signalling, or an inhibitor of EGFR, or a compound with the Formula IX or IX(a), the cell is not contacted with a member of the Klf transcription factor family, such as exogenous Klf4 transcription factor, including a nucleic acid encoding a Klf4 protein or a Kfl4 protein. In some embodiments, replacement of exogenous transcription factor Klf4 is by any compound with Formula IX or IX(a) such as HBDA (also known in the art as Lavendustin C).

Formula IX

In one aspect, the disclosure features a method of producing a reprogrammed cell (i.e. iPS cell or a partially reprogrammed cell) from a differentiated (i.e. somatic) cell, the method comprising:

contacting an isolated differentiated (i.e. somatic) cell with a compound of formula (IX)

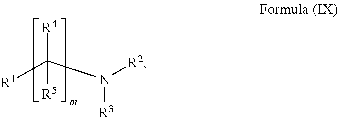

wherein:

R¹ cyclyl, heterocyclcyl, aryl or heteroaryl, each of which can be optionally substituted;

R² cyclyl, heterocyclcyl, aryl or heteroaryl, each of which can be optionally substituted;

R³ is H, C₁-C₆ alkyl, aryl, heteroaryl, cyclyl, heterocyclyl, arylalkyl, heteroarylalkyl, or a nitrogen protecting group, each of which can be optionally substituted;

each R⁴ and R⁵ is independently H, halo, —CN, —NO₂, C₁-C₆ alkyl, haloC₁-C₆alkyl, —CO₂R⁶, —OR⁶ or —N(R⁶)₂, each of which can be optionally substituted;

R⁶ is independently H, C₁-C₆alkyl, aryl, heteroaryl, cyclyl, heterocyclyl or acyl, each of which can be optionally substituted; and

m is 0, 1 or 2.

In one embodiment, the method comprises contacting a plurality of differentiated (i.e. somatic) cells with a compound of formula (IX) to thereby produce a plurality of reprogrammed cells (i.e. iPS cells or partially reprogrammed cells) from the differentiated (i.e. somatic) cells.

In some embodiments, R³ is H.

In some embodiments, m is 1. In some embodiments, R⁴ and R⁵ are both H. In some embodiments, at least one of R⁴ and R⁵ is not H. In some embodiments, at least one of R⁴ and R⁵ is halo.

In some embodiments, the compound of formula (IX) has the structure shown in formula (IXa):

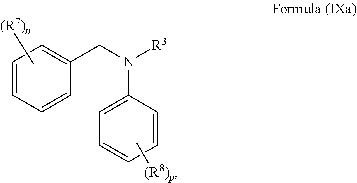

wherein:

each R⁷ and R⁸ is independently halo, —CN, —NO₂, C₁-C₆ alkyl, C₂-C₆ alkenyl, C₁-C₆ alkynyl,haloC₁-C₆alkyl, —CO₂R⁶, —OR⁶, —N(R⁶)₂, each of which can be optionally substituted;

n is 0, 1, 2, 3, 4 or 5; and

p is 0, 1, 2, 3, 4 or 5.

In some embodiments, n is 2. In some embodiments, both R⁷ are —OR⁶. In some embodiments, both R⁷ are —OH.

In some embodiments, n is 2. In some embodiments, one R⁸ is —OR⁶ and the other is CO₂R⁶. In some embodiments, one R⁸ is —OH and the other is —CO₂H.

Exemplary compounds of formula (IX) include:

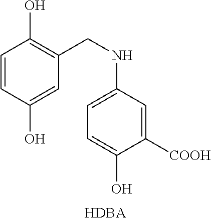

(also known as Lavendustin C);
5-[(2,5-dihydroxybenzyl)-(2-hydroxybenzylamino)]-2-hydroxybenzoic acid;
adamantyl-4-(2,5-dihydroxybenzyalmino)benzoate;
adamantylmethyl-4-(2,5-dihydroxybenzylamino)benzoate;
isoproyl-2-chloro-4-(2,5-dihydroxybenzyl amino)benzoate;
methyl-4-(2,5-dihydroxybenzylamino)-2-hydroxy-benzoate;
isoproyl-4-(4-bromo-2,5-dihydroxybenzyl amino)benzoate;
isoproyl-4-(4-bromo-2,5-dihydroxybenzyl amino)-2-hydroxy-benzoate;
(4-(2-hydroxy-trifluoromethylethyl)phenyl)-2,5-dihydroxybenzylamine;
isoproyl-2-chloro-5-(2-bromo-3,6-dihydroxybenzyl amino)benzoate;
t-butyl-4-(2,5-dihydroxybenzyl amino)benzoate;
N,N-diisoporpyl-4-(2,5-dihydroxybenzyl amino)benzamide;
(4-(1-oxoethyl)phenyl)-2,5-dihydroxybenzylamine;
dimethyl-4-(2,5-dihydroxybenzylamino)phenylphosphonate
methyl-4-4-(1,4-dihydroxynaphthalen-2-yl)methyl)amino)benzoate;
methyl-4-(((naphthoquinonyl)-methyl)amino)benzoate;
methyl-4-(((1,4-benzoquinon-2-yl)methyl)amino)benzoate;
isopropyl-4-(((1,4-benzoquinon-2-yl)methyl)amino)benzoate;
adamantyl-4-(((1,4-benzoquinon-2-yl)methyl)amino)benzoate;
adamantylmethyl-4-(((1,4-benzoquinon-2-yl)methyl)amino)benzoate;
t-butyl-4-(((1,4-benzoquinon-2-yl)methyl)amino)benzoate;
methyl-2-chloro-4-(((1,4-benzoquinon-2-yl)methyl)amino)benzoate; and
isopropyl-2-chloro-4-(((1,4-benzoquinon-2-yl)methyl)amino)benzoate.

Other Inhibitors of EGF Cell Signalling

In some embodiments, other non-limiting examples of small molecule inhibitors of EGF cell signalling pathway which can be used to replace exogenous members of the Klf family of transcription factors, such as Klf4 are known in the art, and include for example, but are not limited to, exemplary inhibitors of EGF cell pathway or inhibitors of EGFR such as inhibitors of EGFR include, but are not limited to, tyrosine kinase inhibitors such as quinazolines, such as PID 153035, 4-(3-chloroanilino) quinazoline, or CP-358,774, pyridopyrimidines, pyrimidopyrimidines, pyrrolopyrimidines, such as CGP 59326, CGP 60261 and CGP 62706, and pyrazolopyrimidines, 4-(phenylamino)-7H-pyrrolo[2,3-d]pyrimidines (Traxler et al., (1996) J. Med Chem 39:2285-2292), curcumin (diferuloyl methane) (Laxmin arayana, et al., (1995), Carcinogen 16:1741-1745), 4,5-bis (4-fluoroanilino) phthalimide (Buchdunger et al. (1995) Clin. Cancer Res. 1:813-821; Dinney et al. (1997) Clin. Cancer Res. 3:161-168); tyrphostins containing nitrothiophene moieties (Brunton et al. (1996) Anti Cancer Drug Design 11:265-295); the protein kinase inhibitor ZD-1 839 (AstraZeneca); CP-358774 (Pfizer, Inc.); PD-01 83805 (Warner-Lambert), EKB-569 (Torrance et al., Nature Medicine, Vol. 6, No. 9, September 2000, p. 1024), HKI-272 and HKI-357 (Wyeth); or as described in International patent application WO05/018677 (Wyeth); WO99/09016 (American Cyanamid); WO98/43960 (American Cyanamid); WO 98/14451; WO 98/02434; WO97/38983 (Warener Labert); WO99/06378 (Warner Lambert); WO99/06396 (Warner Lambert); WO96/30347 (Pfizer, Inc.); WO96/33978 (Zeneca); WO96/33977 (Zeneca); and WO96/33980 (Zeneca), WO 95/19970; U.S. Pat. App. Nos. 2005/0101618 assigned to Pfizer, 2005/0101617, 20050090500 assigned to OSI Pharmaceuticals, Inc.; all herein incorporated by reference. Further useful EGFR inhibitors are described in U.S. Pat. App. No. 2004/0127470, particularly in tables 10, 11, and 12, and are herein incorporated by reference.

In other embodiments, EGFR-inhibiting agents can be used, for example, but are not limited to, Gefitinib (compound ZD1839 developed by AstraZeneca UK Ltd.; available under the tradename IRESSA; hereinafter “IRESSA”) and Erlotinib (compound OSI-774 developed by Genentech, Inc. and OSI Pharmaceuticals, Inc.; available under the tradename TARCEVA; hereinafter “TARCEVA”); the monoclonal antibodies cetuximab (Erbitux; ImmClone Systems Inc/Merck KGaA), matuzumab (Merck KGaA) and anti-EGFR 22Mab (ImClone Systems Incorporated of New York, N.Y., USA), or EGF/R3MAb (Cuban Institute of Oncology; Hybridoma, 2001, Vol. 20, No. 2: 131-136), panitumumab/ABX-EGF (Abgenix/Cell Genesys), nimotuzumab ((TheraCIM-hR3) YM BioSciences Inc. Mississauga, Ontario, Canada), EMD-700, EMD-7200, EMD-5590 (Merck KgaA), E7.6.3 (Abgenix; Cancer Research 59, 1236-1243, 1999), Mab 806 (Ludwig Institute), MDX-103, MDX-447/H-477 (Medarex Inc. of Annandale, N.J., USA and Merck KgaA), and the compounds ZD-1834, ZD-1838 and ZD-1839 (AstraZeneca), PKI-166 (Novartis), PKI-166/CGP-75166 (Novartis), PTK 787 (Novartis), AEE788 (Novartis), CP 701 (Cephalon), leflunomide (Pharmacia/Sugen), CI-1033/PD-169414/PD-183805/Canertinib (Pfizer), CP-358774 (Pfizer), PD-168393, PD-158780, PD-160678 (Parke-Davis), CL-387,785 ((N-[4-[(3-bromophenyl)amino]-6-quinazolinyl]-2-butynamide; C. M. Discafani, et al.; Biochem. Pharmacol. 57:917 (1999)), BBR-1611 (Boehringer Mannheim GmbH/Roche), Naamidine A (Bristol Myers Squibb), RC-3940-II (Pharmacia), BIBX-1382 (Boehringer Ingelheim), OLX-103 (Merck & Co. of Whitehouse Station, N.J., USA), VRCTC-310 (Ventech Research), EGF fusion toxin (Seragen Inc. of Hopkinton, Mass.), DAB-389 (Seragen/Lilgand), ZM-252808 (Imperical Cancer Research Fund), RG-50864 (INSERM), LFM-A12 (Parker Hughes Cancer Center), WHI-P97 (Parker Hughes Cancer Center), GW-282974, GW2016 (Glaxo), KT-8391 (Kyowa Hakko) and EGFR Vaccine (York Medical/Centro de Immunologia Molecular (CIM)), EXEL 7647/EXEL 0999, XL647 (Exelixis), AG1478 (4-(3-Chloroanillino)-6,7-dimethoxyquinazoline), AG879 (3,5-Di-t-butyl-4-hydroxy-benzylidene)thiocyanoacetamide), ICR15, ICR16, and ICR80 (Int J Cancer. 1998 Jan. 19; 75(2):310-6.), ICR62 (Modjtahedi et al. Br J Cancer 1996; 73:228-35.), CGP 59326A (Novartis), BMS-599626 (Bristol-Myers Squibb)). These and other EGFR-inhibiting agents can be used in the present invention.

In an alternative embodiment, the some inhibitors of ErbB2 also inhibit EGFR and can be useful in the methods of the present invention, for use as small molecule inhibitors of EGF cell signalling pathway for the replacement of exogenous members of the Klf family of transcription factors, such as Klf4. Exemplary examples of Erb2 inhibitors include for example include CI-1003, CP-724,714, CP-654577 (Pfizer, Inc.), GW-2016, GW-282974, and lapatinib/GW-572016 (Glaxo Wellcome plc), TAK-165 (Takeda), AEE788 (Novartis), EKB-569, HKI-272 and HKI-357 (Wyeth) (Wyeth-Ayerst), EXEL 7647/EXEL 0999 (EXELIXIS) and the monoclonal antibodies Trastuzumab (tradename HERCEPTIN), 2C4 (Genentech), AR-209 (Aronex Pharmaceuticals Inc. of The Woodlands, Tex., USA), pertuzumab (tradename OMNITARG; Genentech), BMS-599626 (Bristol-Myers Squibb) and 2B-1 (Chiron). For example those indicated in U.S. Pat. Nos. 6,867,201, 6,541,481, 6,284,764, 5,587,458 and 5,877,305; WO 98/02434, WO 99/35146, WO 99/35132, WO 98/02437, WO 97/13760, WO 95/19970, which are all hereby incorporated herein in their entireties by reference. Other inhibitors of the EGFR inhibitors are ERBITUX® (Cetuximab, ImClone), a monoclonal antibody against EGFR and IRESSA® (Gefitinib, AstraZeneca) and TARCEVA® (Erlonitib, Genentech) are small molecule kinase inhibitors of EGFR. The ErbB2 receptor inhibitor compounds and substance described in the aforementioned PCT applications, U.S. patents, and U.S. patent applications, as well as other compounds and substances that inhibit the ErbB2 receptor, can be used in the methods and compositions to reprogram differentiated cells as disclosed herein, for example as small molecule inhibitors of EGF cell signalling pathway which can be used to replace exogenous members of the Klf family of transcription factors, such as Klf4.

In some embodiments, other non-limiting examples of small molecule inhibitors of EGF cell signalling pathway which can be used to replace exogenous members of the Klf family of transcription factors, such as Klf4 are EGFR tyrosine kinase. Exemplary EGFR kinase inhibitors include, for example but are not limited to, quinazoline EGFR kinase inhibitors, pyridopyrimidine EGFR kinase inhibitors, pyrimido-pyrimidine EGFR kinase inhibitors, pyrrolopyrimidine EGFR kinase inhibitors, pyrazolopyrimidine EGFR kinase inhibitors, phenylaminopyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors. EGFR kinase inhibitors are described in U.S. Pat. Nos. 5,217,999; 5,650,415; 5,302,606; 5,196,446; 5,747,498; 5,656,655; 5,418,245; 6,169,099; 6,355,678; 5,789,427; 5,677,329; 6,174,889; 6,207,669; 5,710,158; 6,391,874; 5,616,582 and 5,656,643, and International Patent Publication Nos. WO96/33980; WO96/30347; WO97/30034; WO 97/30044; WO97/38994; WO97/49688; WO98/02434; WO97/38983; WO95/19774; WO95/19970; WO97/13771; WO98/02437; WO98/02438; WO97/32881; WO98/33798; WO97/32880; WO97/03288; WO97/02266; WO97/27199; WO98/07726; WO97/34895; WO96/31510; WO98/14449; WO98/14450; WO98/14451; WO95/09847; WO97/19065; WO98/17662; WO99/35146; WO99/35132; WO99/07701 and WO 92/20642.

Exemplary EGFR kinase inhibitors include, but are not limited to, [6,7-bis(2-methoxyethoxy)-4-quinazolin-4-yl]-(3-ethynylphenyl)amine (erlotinib, OSI-774, Tarceva®), tannic acid, genistein, CI-1033 (PD183805), PD153035, PD-158780, CGP-59326, PKI-166, EKB-569, GW-2016 (GW-572016, lapatinib ditosylate), EKB-569, ZD6474 (Zactima™), BIBW-2992, ARRY-334543, BMS-599626, ZD1839 (gefitinib, Iressa™), IMC-C225 (cetuximab, Erbitx™), ABX-EGF (Abgenix), EMD 72000, RH3, MDX-447 and tyrphostins such as AG-17, AG-18, AG-82, AG-99, AG-123, AG-126, AG-183, AG-490, AG-494, AG-875, AG-1487, RG-13022 and RG-14620.

Antibody Inhibitors EGF Cell Signalling

In some embodiments, the inhibitor of EGF cell signalling used to replace a member of the Klf family of transcription factors, such as Klf4 is an anti-EGF or anti-EGFR antibody. In some embodiments, inhibitors of EGF cell signaling include EGF inhibitors which are neutralizing antibodies, including anti-EGF antibodies which are commercially available. Some examples of commercially available anti-EGF antibodies which can be used as EGF cell signalling inhibitors according to the methods as disclosed herein include for example, but not limited to: Cell Signalling antibodies; Santa Cruz antibodies (e.g. anti-EGFR antibodies which can be used are cat No: sc-71034, sc-03, sc-81449, sc-81450, sc-81451, sc-81452 and sc-81453 etc.), and other commercial sources such as Cell Signalling, Invitrogen (e.g., cat nos: AHG9069, AHG0064), Sigma, AdD Serotec and the like.

RNAi Inhibitors of EGF Cell Signalling

Inhibition of the EGF signaling pathway can be by RNA interference (RNAi) according to methods commonly known by a skilled artisan. For example, siRNA oligonucleotide duplexes targeted specifically to human EGF (GenBank No:1950 or 3229) have been previously used to knockdown EGF expression.

EGF mRNA has been successfully targeted using siRNAs; and other siRNA molecules may be readily prepared by those of skill in the art based on the known sequence of the target mRNA. To avoid doubt, the sequence of a human EGF is provided at, for example, GenBank Accession Nos:1950 or 3229. EGF RNAi agents are also commercially available, such as, for example, from Santa-Cruz Biotechnology, and also from other companies, such as Invitrogen (e.g. cat nos: Hss103099, Hss103100, Hss103101).

Inhibitors of Ca²⁺/Calmodulin Cell Signalling

In some embodiments, the compounds of Formula IX, and IX(a) have more than one function. For example, compounds of Formula IX or IX(a), or any other Formulas as described herein can inhibit or function on more than one pathway. In some embodiments, a compound (e.g. Formula IX) predominantly inhibits only one pathway (e.g. Ca²⁺/Calmodulin pathway), e.g. at least 70% of one pathway is inhibited as compared to other pathways. In some embodiments, a compound of Formula IX or IX(a), can inhibit EGF cell signalling and inhibit Ca²⁺/Calmodulin cell signaling. In some embodiment, a compound of Formula IX or IX(a) predominantly inhibits EGF cell signalling pathway, e.g. at least 70% inhibition as compared to the inhibition of the Ca²⁺/Calmodulin cell signaling pathway. In another embodiment, a compound of Formula IX or IX(a) predominantly inhibits the Ca²⁺/Calmodulin cell signaling pathway, e.g. at least 70% inhibition as compared to the inhibition of the EGF cell signalling pathway. Thus, in some embodiments, a differentiated cell can be reprogrammed by contacting the differentiated cell with any compound with Formula IX or IX(a) and can program the cell by inhibiting multiple cell pathways. In some embodiments, a differentiated cell can be reprogrammed by contacting the differentiated cell with any compound with Formula IX or IX(a) can program the cell by predominantly inhibiting (e.g. at least 70% inhibition) of one pathway, such as the inhibiting the EGF cell signalling pathway. In some embodiments, a differentiated cell can be reprogrammed by contacting the differentiated cell with any compound with Formula IX or IX(a) can program the cell by predominantly inhibiting (e.g. at least 70% inhibition) of the Ca2+/Calmodulin cell signalling pathway.

In some embodiments, a chemically-induced reprogrammed cell be produced by contacting a differentiated cell with an inhibitor of Ca²⁺/Calmodulin cell signalling pathway. The Ca²⁺/Calmodulin signaling pathway is involved in many cellular processes in both the adult organism and the developing embryo including cell growth, cell differentiation, apoptosis, cellular homeostasis and other cellular functions.

In one embodiment, replacement of a member of the Klf family of transcription factors, such as replacement of exogenous transcription factor Klf 4 is by an agent which is an inhibitor of the Ca²⁺/Calmodulin signalling pathway, such as a Ca²⁺/Calmodulin inhibitor. In some embodiments, replacement of exogenous transcription factor Klf4 is by any compound with the Formula IX. In some embodiments, where a differentiated cell is contacted with an inhibitor of the Ca²⁺/Calmodulin signalling pathway, or a compound with the Formula IX, the cell is not contacted with a member of the Klf transcription factor family, such as exogenous Klf4 transcription factor, including a nucleic acid encoding a Klf4 protein or a Klf4 protein. In some embodiments, replacement of exogenous transcription factor Klf4 is by any compound with Formula IX, as disclosed above, such as HBDA.

Ca²⁺/Calmodulin-dependent protein kinase IV (CaMKIV) is a monomeric multifunctional enzyme that is expressed primarily in subanatomical portions of the brain, T lymphocytes and postmeiotic male germ cells. It is present in the nucleus of cells in which it is expressed (Jensen et al, Proc. Natl. Acad. Sci. USA 88:2850 (1991)). CaMKIV phosphorylates and activates the cyclic AMP response element binding proteins CREB and CREM.tau. in a manner analogous to protein kinase A (Matthews et al, Mol. Cell. Biol. 14:6107 (1994); Sun et al, Genes Dev. 8:2527 (1994); Enslen et al, J. Biol. Chem. 269:15320 (1994)).

Other Inhibitors of Ca²⁺/Calmodulin Cell Signalling

In some embodiments, other non-limiting examples of small molecule inhibitors of Ca2+/calmodulin cell signalling pathway which can be used to replace exogenous members of the Klf family of transcription factors, such as Klf4 are known in the art, and include for example, but are not limited to, exemplary inhibitors of Ca2+/calmodulin cell pathway include, but are not limited to Ca2+/calmodulin inhibitors are described in U.S. Pat. Nos. 5,624,902; 4,758,559; 5,182,262; 5,480,903; 5,532,337; 5,840,697; 5,171,152 and 5,386,019, which are incorporated herein by reference. In some embodiments, exemplary Ca2+/calmodulin inhibitors also include, but are not limited to, R24571 (calmidazolium chloride), CaM Kinase II, E⁶ berbamine, Fluphenazine-N-2-chloroethane.2HCl, phenoxybenzamine, trifluoperazine (Stelazine), N-(6-Aminohexyl)-1-naphthalenesulfonamide (W-5), N-(6-Aminohexyl)-5-chloro-1-naphthalenesulfonamide (W-7), W13, sphigosylphosphorylcholine, KN093, CGS 9543B, 1,4-dihydropyridine derivatives such as niphedipine, nicardipine, niludipine, nimopidine, nisoldipine, nitrendipine, milbadipine, dazodipine and ferodipine, N-methyl-Nohomoveratrilamine derivatives such as verapamil, gallopamil and tiapamil, benzothiazepine derivatives such as diltiazem, piperazine derivatives such as cinnarizine, lidoflazine and flunarizine, and diphenylpropiramine derivatives such as prenylamine, terodiline, andphendiline, bepridil and andperhexyline.

Antibody Inhibitors Ca²⁺/Calmodulin Cell Signalling

In some embodiments, an antibody inhibitor of Ca²⁺/Calmodulin cell signalling is disclosed in U.S. Pat. No. 5,789,553 or the Anti-calmodulin antibody from Abcam [Serial No: EP799Y or ab45689), which was produced using the methods disclosed in 5,675,063, which is incorporated herein in its entirety by reference. Other commercially available antibodies which inhibit Ca²⁺/Calmodulin Cell Signalling are known in the art and are encompassed for use in the methods and compositions as disclosed herein for the production or reprogrammed cells as disclosed herein. Some examples of commercially available anti-Calmodulin antibodies which can be used as Ca²⁺/Calmodulin cell signalling inhibitors according to the methods as disclosed herein include for example, but not limited to: Cell Signalling antibodies; Santa Cruz antibodies, Ancan antibodies (e.g. Cat No: AB-45689, Ab38590) other commercial sources such as Cell Signalling, Invitrogen, Epitomics (e.g., cat nos: 1716-1), Novus Biologicals, (e.g. NB110-55649), Millipore, Sigma, AdD Serotec and the like.

RNAi Inhibitors of Ca²⁺/Calmodulin Cell Signalling

Inhibition of the Ca²⁺/Calmodulin signaling pathway can be by RNA interference (RNAi) according to methods commonly known by a skilled artisan. For example, siRNA oligonucleotide duplexes targeted specifically to human Calmodulin (GenBank No: 8536) have been previously used to knockdown Ca²⁺/Calmodulin expression.

Ca²⁺/Calmodulin mRNA has been successfully targeted using siRNAs; and other siRNA molecules may be readily prepared by those of skill in the art based on the known sequence of the target mRNA. To avoid doubt, the sequence of a human Calmodulin is provided at, for example, GenBank Accession Nos: 8536. Calmodulin RNAi agents are also commercially available, such as, for example, from Santa-Cruz Biotechnology, and also from other companies, such as Invitrogen (e.g. cat nos: Hss112474, Hss112474, Hss112474, V49300-05, VHS40124, as disclosed in U.S. Patent Application 2007/0215320, which is incorporated herein in its entirety by reference.

### Chemical Replacement of Oct Family of Transcription Factor

Another aspect of the present invention relates to a method to produce a reprogrammed cell by contacting a differentiated cell with at least one small molecule which replaces a transcription factor from the Oct family of transcription factors. Examples of the Oct family of transcription factors include, for example, Oct3/4, Oct1A, Oct6, and the like. Oct3/4 is a transcription factor belonging to the POU family, and is reported as a marker of undifferentiated cells (Okamoto et al., Cell 60:461-72, 1990). Oct3/4 is also reported to participate in the maintenance of pluripotency (Nichols et al., Cell 95:379-91, 1998). The accession numbers of members of the Oct family of transcription factors are: Oct3/4 NM_{—}013633 (mouse) (SEQ ID NO: 10), NM_{—}002701 (human) (SEQ ID NO: 11); homolog 1, lung carcinoma derived (avian) Oct1A POU domain, class 2, transcription factor 1 NM_{—}198934 (mouse), NM_{—}002697 (human), Oct6 POU domain, class 3, transcription factor 1 NM_{—}011141 (mouse), NM_{—}002699 (human).

In some embodiments, a reprogrammed cell is produced by contacting a cell with two or more small molecule which replaces a transcription factor from the Oct4 family of transcription factors (such as Oct4), and a transcription from the Klf4 family of transcription factors (such as Klf4).

Another aspect of the present invention relates to a method to produce a reprogrammed cell by contacting a differentiated cell with more small molecule which replaces a transcription factor from the Sox family of transcription factors (such as Sox2), and a transcription from the Oct family of transcription factors (such as Oct 3/4).

In one embodiment, any compound selected from any of formula X-XI to reprogram a differentiated cell can be used in any combination of members from one or more transcription factors gene families. For example, a combination of one or more gene products of Klf4, Sox2, and c-Myc.

In one embodiment, replacement of a member of the Oct family of transcription factors, such as replacement of exogenous transcription factor Oct 4 is by an agent which activates the ATP-dependent potassium channels. In some embodiments, replacement of exogenous transcription factor Oct4 is by any compound with the Formula X. In some embodiments, where a differentiated cell is contacted with an agonist of ATP-dependent potassium channels, or a compound with the Formula X, the cell is not contacted with a member of the Oct transcription factor family, such as exogenous Oct4 transcription factor, including a nucleic acid encoding a Oct4 protein or a Oct4 protein. In some embodiments, replacement of exogenous transcription factor Oct4 is by any compound with Formula IX such as Sinomenin.

In one embodiment, replacement of a member of the Oct family of transcription factors, such as replacement of exogenous transcription factor Oct4 is by an agent which is an inhibitor of sodium channels, such as a Na⁺ channel inhibitor. In some embodiments, replacement of exogenous transcription factor Oct4 is by any compound with the Formula X. In some embodiments, where a differentiated cell is contacted with an inhibitor of sodium channels, or a compound with the Formula X, the cell is not contacted with a member of the Oct transcription factor family, such as exogenous Oct4 transcription factor, including a nucleic acid encoding a Oct4 protein or a Oct4 protein. In some embodiments, replacement of exogenous transcription factor Oct4 is by any compound with Formula IX such as Sinomenin.

In one embodiment, replacement of a member of the Oct family of transcription factors, such as replacement of exogenous transcription factor Oct 4 is by an agent which is an agonist of the MAPK signalling pathway, such as a MAPK agonist. In some embodiments, replacement of exogenous transcription factor Oct4 is by any compound with the Formula XI. In some embodiments, where a differentiated cell is contacted with an agonist of the MAPK signalling pathway, or a compound with the Formula XI, the cell is not contacted with a member of the Oct transcription factor family, such as exogenous Oct4 transcription factor, including a nucleic acid encoding a Oct4 protein or a Oct4 protein. In some embodiments, replacement of exogenous transcription factor Oct4 is by any compound with Formula X such as either Ropivacaine or Bupivacaine. In some embodiments, replacement of exogenous transcription factor Oct4 is by Bupivacaine. In some embodiments, where replacement of exogenous transcription factor Oct4 is by Bupivacaine, the differentiated cell is not contacted with an reprogramming enhancing agent as that term is described herein, such as for example a HDAC inhibitor or VPA or the like.

In some embodiments, contact of a differentiated cell with an agent which replaces a member of the Oct family of transcription factors, such as Oct4, (i.e. an agonist of ATP-dependent potassium channels, or a inhibitor of sodium channels, or an agonist of the MAPK signalling pathway or any compound with Formulas X or XI, including but not limited to Sinomenin, Ropivacaine or Bupivacaine), enables reprogramming of differentiated cells by only 3 transcription factors, such as Sox2, Klf-4, and c-Myc without the need for a member of the Oct family of transcription factors, such as Oct4. In some embodiments, contact of a differentiated cell with an agent which replaces Oct4 requires only 2 transcription factors, Sox2 and Klf4 without the need for c-Myc or Oct 4. In some embodiments, contact of a differentiated cell with an agent which replaces Oct4 can also be contacted with at least one agent, preferably two agents which replaces the 2 transcription factors, Sox2 and Klf-4, as disclosed herein, without the need for exogenous Sox-2, Oct4, c-Myc or Klf4 (i.e. where the cell is not contacted with any members of Klf, Sox, Oct or Myc transcription factor families, such as exogenous Klf4, Oct4, Sox2 or c-myc transcription factors, including nucleic acid sequences encoding any one of Klf4, Oct4, Sox2 or c-myc proteins or any combination of Klf4, Oct4, Sox2 or c-myc proteins).

For example, reprogrammed cells (i.e. iPS) were identified in mouse fibroblasts (MEFs) infected by Sox-2, Klf-4, and c-Myc retroviruses together with Sinomenin, or Ropivacaine or Bupivacaine treatment. The number and percentage of reprogrammed cells (i.e. iPS cells or partially reprogrammed cell) colonies was comparable to those in the addition of nucleic acid encoding the Oct4 transgene. Thus, the 3-factor reprogramming efficiency by Sinomenin or Ropivacaine or Bupivacaine treatment is comparable to the induction rate for mouse fibroblasts infected by 4 factors (Oct-4, Klf-4, c-Myc and Sox-2), demonstrating that Sinomenin or Ropivacaine or Bupivacaine treatment effectively replaced the need for exogenous Oct4 transcription factor, including a nucleic acid encoding a oct4 protein or a Oct4 protein. Thus, described herein are methods for producing reprogrammed cells from differentiated cells (i.e. from fibroblasts e.g., MEFs) without using the oncogenes, for example oct4, Klf4, c-Myc or Sox-2.

In some embodiments, a differentiated cell which is contacted with an agent which replaces exogenous Oct4 transcription factor, including a nucleic acid encoding a Oct4 protein or a Oct4 protein (i.e. an agonist of ATP-dependent potassium channels, or a inhibitor of sodium channels, or an agonist of the MAPK signalling pathway or any compound with Formulas X or XI, including but not limited to Sinomenine, Ropivacaine or Bupivacaine), can be reprogrammed with small molecules or other agents which replace exogenous supplied Sox2 and Kfl4, as disclosed herein. Thus, described herein are methods for producing reprogrammed cells from differentiated cells (i.e. from fibroblasts e.g., MEFs) without using the oncogenes, for example c-Myc or oncogenes associated with introduction of nucleic acid sequences encoding the transcription factors Sox-2, Oct-4 or Klf-4 into the differentiated cell to be reprogrammed (i.e. viral oncogenes). For example, the chemical mediated reprogramming of differentiated cells makes it possible to create reprogrammed cells (e.g. iPS cells or partially reprogrammed cells) from small numbers of differentiated cells (e.g., such as those obtained from hair follicle cells from patients, blood samples, adipose biopsy, fibroblasts, skin cells, etc). In one embodiments, the addition of small molecules compounds (e.g., chemicals) allows successful and safe generation of reprogrammed cells (e.g. iPS cells or partially reprogrammed cells) from human differentiated cells, such as skin biopsies (fibroblasts or other nucleated cells) as well as from differentiated cells from all and any other cell type.

Agonist of ATP-Dependent Potassium Channels

In some embodiments, a chemically-induced reprogrammed cell be produced by contacting a differentiated cell with an agonist of ATP-dependent potassium channels.

Thus, one aspect of the present invention relates to reprogramming a differentiated cell, where a member of the Oct family of transcription factors, such as exogenous transcription factor Oct4 is replaced by an agent which activates ATP-dependent potassium channels. In some embodiments, replacement of exogenous transcription factor Oct4 is by any compound with Formula X. In some embodiments, where a differentiated cell is contacted with an agonist of ATP-dependent potassium channels, or a compound with the Formula X, the cell is not contacted with a member of the Oct transcription factor family, such as exogenous Oct4 transcription factor, including a nucleic acid encoding a Oct4 protein or a Oct4 protein. In some embodiments, replacement of exogenous transcription factor Oct4 is by any compound with Formula X such as Sinomenine.

In some embodiments, where a replacement of exogenous transcription factor Oct4 is by any compound with Formula X such as Sinomenine, the differentiated cell is not contacted with a reprogramming efficiency agent, as that term is described herein, such as for example a HDAC inhibitor such as VPA and the like.

ATP-sensitive potassium (K_{ATP}) channels play important roles in a variety of tissues by coupling cellular metabolism to electrical activity. The K_{ATP} channel has been identified as an octameric complex of two unrelated proteins, which assemble in a 4:4 stoichiometry. The first is a pore forming subunit, Kir6.x, which forms an inwardly rectifying K⁺ channel; the second is an ABC (ATP binding cassette) transporter, also known as the sulfonylurea receptor (SURx) (Babenko et al., Annu. Rev. Physiol., 60:667-687 (1998)). The Kir6.x pore forming subunit is common for many types of K_{ATP}, channels, and has two putative transmembrane domains (identified as TM1 and TM2), which are linked by a pore loop (H5). The subunit that comprises the SUR receptor includes multiple membrane-spanning domains and two nucleotide-binding folds.

According to their tissue localization, K_{ATP} channels exist in different isoforms or subspecies resulting from the assembly of the SUR and Kir subunits in multiple combinations. The combination of the SUR1 with the Kir6.2 subunits (SUR1/Kir6.2) typically forms the adipocyte and pancreatic B-cell type K_{ATP} channels, whereas the SUR2A/Kir6.2 and the SUR2B/Kir6.2 or Kir6.1 combinations typically form the cardiac type and the smooth muscle type K_{ATP}, channels, respectively (Babenko et al., Annu. Rev. Physiol., 60:667-687 (1998)). There is also evidence that the channel may include Kir2.x subunits. This class of potassium channels are inhibited by intracellular ATP and activated by intracellular nucleoside diphosphates. Such K_{ATP} channels link the metabolic status of the cells to the plasma membrane potential and in this way play a key role in regulating cellular activity. In most excitatory cells, K_{ATP} channels are closed under normal physiological conditions and open when the tissue is metabolically compromised (e.g. when the (ATP:ADP) ratio falls). This promotes K⁺efflux and cell hyperpolarization, thereby preventing voltage-operated Ca2⁺ channels (VOCs) from opening. (Prog. Res Research, (2001) 31:77-80).

Potassium channel openers (PCOs or KCOs; also referred to as channel activators or channel agonists), are a structurally diverse group of compounds with no apparent common pharmacophore linking their ability to antagonize the inhibition of K_{ATP} channels by intracellular nucleotides. Diazoxide is a PCO that stimulates K_{ATP} channels in pancreatic β-cells (see Trube et al., Pfluegers Arch Eur J Physiol, 407, 493-99 (1986)). Pinacidil and chromakalim are PCOs that activate sarcolemmal potassium channels (see Escande et al., Biochem Biophys Res Commun, 154, 620-625 (1988); Babenko et al., J Biol Chem, 275(2), 717-720 (2000)). Responsiveness to diazoxide has been shown to reside in the 6^{th} through 11^{th} predicted transmembrane domains (TMD6-11) and the first nucleotide-binding fold (NBF1) of the SUR1 subunit.

Any agonist of ATP-dependent potassium channels which can be used to replace members of the Oct transcription factor family, such as Oct4 are listed below, and include any agent with Formula X, as disclosed herein, such as Sinomenine.

Formula X

In one aspect, the disclosure features a method of producing a reprogrammed cell (i.e. an iPS cell or a partially reprogrammed cell) from a differentiated (i.e. somatic cell), the method comprising:

contacting an isolated somatic cell with a compound of formula (X)

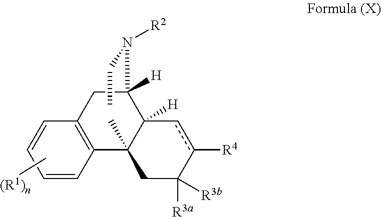

wherein,

each R¹ is independently C₁-C₆ alkyl, C₁-C₆ haloalkyl, —COOR⁵, —OR⁵, —NR⁵₂, —NO₂ or —CN;

R² is hydrogen, C₁-C₆ alkyl, aryl, heteroaryl, cyclyl, heterocyclyl, arylalkyl, or a nitrogen protecting group, each of which is optionally substituted;

each R^{3a} and R^{3b} is independently hydrogen, —COOR⁵ or —OR⁵, or R^{3a} and R^{3b} taken together with the carbon to which they are attached form a carbonyl;

R⁴ is hydrogen, C₁-C₆ alkyl, C₁-C₆ haloalkyl, —COOR⁵, —OR⁵, —NR⁵₂, —NO₂ or —CN;

each R⁵ is independently hydrogen, C₁-C₆ alkyl, aryl, heteroaryl, cyclyl, heterocyclyl or acyl;

n is 0, 1, 2, 3, or 4; and

the dashed line ( - - - ) indicates the presence or absence of a bond;

to thereby produce a reprogrammed cell from the differentiated (i.e. somatic) cell.

In one embodiment, the method comprises contacting a plurality of differentiated cells with a compound of formula (X) to thereby produce a plurality of reprogrammed cells (i.e. an iPS cell or partially reprogrammed cells) from the differentiated cells.

In some embodiments, n is 2. In some embodiments, both R¹ are —OR⁴. In some embodiments, one R¹ is —OH and the other is —OCH₃.

In some embodiments, R² is C₁-C₆ alkyl. In some embodiments, R² is methyl.

In some embodiments, R^{3a} and R^{3b} taken together with the carbon to which they are attached form a carbonyl.

In some embodiments, R⁴ is —OR⁵. In some embodiments, R⁵ is C₁-C₆ alkyl. In some embodiments, R⁴ is —OCH₃.

In some embodiments, the dashed line indicates the presence of a bond.

Exemplary compounds of formula (X) include compound as follows:

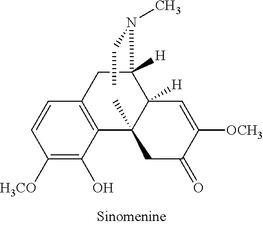

Other Agonists of ATP-Dependent Potassium Channels

In some embodiments, other non-limiting examples of small molecule agonists of ATP-dependent potassium channels, also known in the art as “K_{ATP} channel openers” or “KCO” which can be used to replace exogenous members of the Oct family of transcription factors, such as Oct4 are known in the art, and include for example, but are not limited to, exemplary agonists such as nicorandil, pinacidil, diazooxide, levcromakalim, minoxidil, bimakalim (EMD 52692), cromakalim, lemakalim, iptakalim, L-735334, KR-31378, BPDZ 154, levosimendan and NS1619, ATP-dependent potassium channel agonists ZD6169, celikalim, and WAY-133537. Other examples of ATP-dependent potassium channel agonists are also disclosed in U.S. Pat. Nos. 5,506,252; 6,265,417 and 7,572,789, and U.S. Patent Application 2002/0035106, which are all incorporated herein in their entirety by reference.

Inhibitors of Sodium Channels

In some embodiments, the compounds of Formula X had more than one function. For example, compounds of Formula X can inhibit some channels and activate other channels. For example, in some embodiments, a compound of Formula X predominantly activates (e.g. opens) ATP-dependent K⁺ channels, e.g. at least 70% of ATP-dependent K⁺ channels are activated as compared to inhibition of other channels. In some embodiments, a compound of Formula X, such as for example Sinimenine, can activate ATP-dependent K⁺ channels and inhibit Na⁺ channels. In some embodiment, a compound of Formula X predominantly activates ATP-dependent K⁺ channels, e.g. at least 70% of ATP-dependent K⁺ channels as compared to the inhibition of Na⁺ channels. In another embodiment, a compound of Formula X predominantly inhibits Na⁺ channels, e.g. at least 70% inhibition as compared to the activation of ATP-dependent K⁺ channels. Thus, in some embodiments, a differentiated cell can be reprogrammed by contacting the differentiated cell with any compound with Formula X can reprogram the cell by inhibiting or activating different channels. In some embodiments, a differentiated cell can be reprogrammed by contacting the differentiated cell with any compound with Formula X can program the cell by predominantly inhibiting (e.g. at least 70% inhibition) Na⁺ channels. In some embodiments, a differentiated cell can be reprogrammed by contacting the differentiated cell with any compound with Formula X can program the cell by predominantly activating (e.g. at least 70% activation) of ATP-dependent K⁺ channels

In some embodiments, a chemically-induced reprogrammed cell be produced by contacting a differentiated cell with an inhibitor of sodium channels.

Accordingly, one aspect of the present invention relates to reprogramming a differentiated cell, where a member of the Oct family of transcription factors, such as exogenous transcription factor Oct 4 is replaced by an agent which inhibits sodium channels. In some embodiments, replacement of exogenous transcription factor Oct4 is by any compound with Formula X, such as but not limited to Sinomenine. In some embodiments, where a differentiated cell is contacted with an inhibitor of sodium channels, or a compound with the Formula X, as disclosed above, the cell is not contacted with a member of the Oct transcription factor family, such as exogenous Oct4 transcription factor, including a nucleic acid encoding a Oct4 protein or a Oct4 protein. In some embodiments, replacement of exogenous transcription factor Oct4 is by any compound with Formula X, such as Sinomenine.

There are currently 9 known members of the family of voltage-gated sodium channel (VGSC) alpha subunits. Names for this family include SCNx, SCNAx, and Naᵥx.x. The VGSC family has been phylogenetically divided into two subfamilies Naᵥ1.x (all but SCN6A) and Naᵥ2.x (SCN6A). The Naᵥ1.x subfamily can be functionally subdivided into two groups, those which are sensitive to blocking by tetrodotoxin (TTX-sensitive or TTX-s) and those which are resistant to blocking by tetrodotoxin (TTX-resistant or TTX-r).

Other Inhibitors of Sodium Channels

In some embodiments, other non-limiting examples of small molecule inhibitors of sodium channels which can be used to replace exogenous members of the Oct 4 family of transcription factors, such as Oct4 are known in the art, and include for example, but are not limited to, exemplary inhibitors such as Exemplary sodium channel inhibitors include, but are not limited to, 3′,4′-Dichlorobenzamil, A-803467, ambroxol (a metabolite of bromhexine), astemizole, BIA 2-093, benzamil, benzocaine, benzoylheteratisine, bupivacaine, carbamazepine, dihydroouabain, disopyramide, encamide, flecamide, KR-32568, lappaconitine, lidocaine N-ethyl chloride, lidocaine N-methyl chloride, mepivacaine, metolazone, mexiletine, ouabain octahydrate, procainamide, procaine, R(−)-Me5 hydriodide, ropivacaine, saxitoxin diacetate, tetrodotoxin and tocamide. Other inhibitors of sodium channels are disclosed in WO/2009/012241

Antibody Inhibitors Sodium Channels

In some embodiments, the inhibitor of sodium channels used to replace a member of the Oct family of transcription factors, such as oct4 are pan specific anti-sodium channel antibodies, which are commercially available and known in the art and are encompassed for use in the methods and compositions as disclosed herein for the production or reprogrammed cells as disclosed herein. One example of commercially available pan specific anti-sodium channel antibodies which can be used to inhibit sodium channels according to the methods as disclosed herein include for example, but not limited to: Cell Signalling antibodies; Santa Cruz antibodies, Abeam antibodies, and other commercial sources such as Invitrogen, Epitomics, Novus Biologicals (e.g. Cat No:NB120-3468), Millipore (e.g. Cat No:AB5210), Sigma (e.g. Cat No:S8809), AnaSpec (e.g. Cat No:53852), Thermo Scientific (e.g. Cat No:PA1-38631), Lifespan Biosciences (e.g. Cat No: LS-C33588-50) and the like.

Agonists of MAPK Cell Signalling

In some embodiments, a chemically-induced reprogrammed cell be produced by contacting a differentiated cell with an agonist of MAPK cell signalling pathway. The MAPK signaling pathway is involved in many cellular processes in both the adult organism and the developing embryo including cell growth, cell differentiation, apoptosis, cellular homeostasis and other cellular functions.

In one embodiment, replacement of a member of the Oct family of transcription factors, such as replacement of exogenous transcription factor Oct4 is by an agent which activates the MAPK signalling pathway, such as a p38 protein. In some embodiments, replacement of exogenous transcription factor Oct4 is by any compound with the Formula XI. In some embodiments, where a differentiated cell is contacted with an agonist of MAPK signalling pathway, or a compound with the Formula XI, the cell is not contacted with a member of the Oct4 transcription factor family, such as exogenous Oct4 transcription factor, including a nucleic acid encoding a Oct4 protein or a Oct4 protein. In some embodiments, replacement of exogenous transcription factor Oct4 is by any compound with Formula XI, such as Ropivacaine or Bupivacaine.

In some embodiments, where a replacement of exogenous transcription factor Oct4 is by any compound with Formula XI such as Bupivacaine, the differentiated cell is not contacted with a reprogramming efficiency agent, as that term is described herein, such as for example a HDAC inhibitor such as VPA and the like.

### Formula XI

In one aspect, the disclosure features a method of producing a reprogrammed cell (i.e. an iPS cell or a partially reprogrammed cell) from a differentiated cell (i.e. somatic cell) cell, the method comprising:

contacting an isolated differentiated cell with a compound of formula (XI)

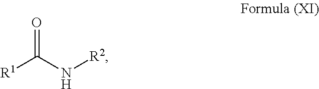

wherein:

R¹ is cyclyl, heterocyclcyl, aryl or heteroaryl, each of which can be optionally substituted;

R² is cyclyl, heterocyclcyl, aryl or heteroaryl, each of which can be optionally substituted,

to thereby produce an reprogrammed cell (e.g iPS cell or partially reprogrammed cell) from the differentiated cell (e.g. somatic cell).

In one embodiment, the method comprises contacting a plurality of somatic cells with a compound of formula (XI) to thereby produce a plurality of reprogrammed cells (i.e. an iPS cells or partially reprogrammed cells) from the somatic cells.

In some embodiments, R¹ is heterocyclyl. In some embodiments, R¹ is a nitrogen containing heterocyclyl (e.g., including 1 or 2 nitrogens). In some embodiments, R¹ is an optionally substituted heterocyclyl. In some embodiments, R¹ is an optionally substituted monocyclic heterocyclyl (e.g., a six membered monocyclic hetercylcyclyl such as piperidinyl or piperazinyl). In some embodiments, R¹ is substituted. Exemplary substituents include halo, C₁-C₆ alkyl, haloC₁-C₆alkyl, C₁-C₆ alkoxy, OH, haloC₁-C₆alkoxy. In some embodiments, R¹ is a nitrogen containing heteroaryl (e.g., including 1, 2, or 3 nitrogens (e.g., 1 or)). In some embodiments, R¹ is a bicyclic heteroaryl. In some embodiments, R¹ is a 6-6 fused heteroaryl. In some embodiments, R¹ is pyridyl, pyrimidyl, pyridazinyl, pyrazinyl, quinolinyl, naphthyridinyl (e.g., 1,5-naphthyridinyl), quinazolinyl, 5,6,7,8-tetrahydroquinazolinyl, 1,3-benzodioxlyl, 1,2,3-benzotriazolyl, benzoxazolyl, benzothiazolyl, 2,1,3-benzooxadiazole, imidazo[1,2-a]pyridinyl, pyrazolo[1,5-a]pyridinyl, [1,2,4]triazolo[1,5-a]pyridinyl, pyrazolo[1,5-a]pyrimidinyl, [1,2,4]triazolo[1,5-a]pyrimidinyl, [1,2,3]triazolo[1,5-a]pyrimidinyl, [1,2,4]triazolo[4,3-a]pyrimidinyl, [1,2,4]triazolo[4,3-a]pyridazinyl. In some embodiments, R¹ is

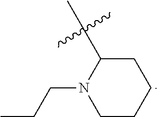

In some embodiments, R¹ is

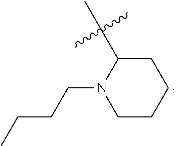

In some embodiment, R² is aryl, e.g., a substituted aryl. In some embodiments, R² is a nitrogen comprising heteroaryl (e.g., including 1, 2 or 3 nitrogens (e.g., 1 or 2)). In some embodiments, R² is an optionally substituted monocyclic heteroaryl (e.g., a six membered heteroaryl such as pyridyl, pyrimidyl, pyridazinyl or pyrazinyl). In some embodiments, R² is substituted. Exemplary substituents include halo, C₁-C₆ alkyl, haloC₁-C₆alkyl, C₁-C₆ alkoxy, OH, haloC₁-C₆alkoxy. In some embodiments, R² is monosubstituted. In some embodiments, R² is substituted with methyl. In some embodiments, R² is an optionally substituted phenyl. In some embodiments, R² is

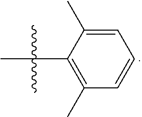

Exemplary compounds of formula (XI) include

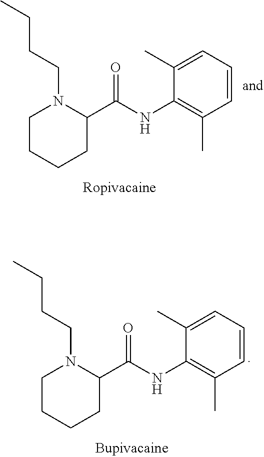

In one embodiment, the method comprises contacting a plurality of differentiated cells with a compound of formula (XI) to thereby produce a plurality of reprogrammed cells (i.e. iPS cells or partially reprogrammed cell) from the differentiated cells. In a particular embodiment, the method comprises contacting a plurality of differentiated cells with a compound with the structure of

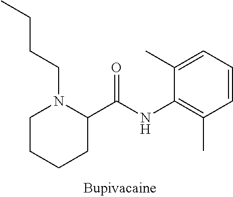

to produce a plurality of reprogrammed cells (i.e. iPS cells or partially reprogrammed cell) from the differentiated cells.

Other Agonists of MAPK Cell Signalling

In some embodiments, other non-limiting examples of small molecule agonists of MAPK cell signalling pathway which can be used to replace exogenous members of the Oct family of transcription factors, such as Oct4 are known in the art, and include for example, but are not limited to, exemplary MAPK agonists such as; anandamide, angiotensin II, anisomycin, aurintricarboxylic acid, 1,1-dimethylbiguanide, interlukin-11, isoproterenol, lactosyl ceramide, leukotriene D₄, lipoxin A₄, platelet activating factor-16, N-acetyl-D-erythro-sphingosine, N-hexanoyl-D-erythro-sphingosine, N-octanoyl-D-erythro-sphingosine, sphingosylphosphorylcholine and TNF-alpha.

### Combinations of Compounds for Reprogramming of Differentiated Cells

Replacement of exogenous Sox and Klf transcription factors: In one embodiment, a method to reprogram a differentiated cell comprises contacting the differentiated cell with a compound which replaces exogenous Sox2 (e.g. any compound with a formula I-VII, such as Repsox, E-616451 or SB431542) and also contacting the differentiated cell with any compound which replaces exogenous Klf4 (e.g. any compound with a formula VIII or IX, such as Prostaglandin 2 or HBDA, respectively).

In one embodiment, a method to reprogram a differentiated cell comprises contacting the differentiated cell with a TGFBR1 inhibitor (e.g. any compound with Formulas I, such as Repsox, E-616451 or SB431542) or a Src inhibitor, such as a compound of Formula II (such as EI-275) and also contacting the differentiated cell with any Mek/Erk agonist, such as a compound of formula VIII, (such as Prostaglandin 2) or a inhibitor of EGF cell signaling or a inhibitor of the Ca²⁺/Calmodulin signaling pathway, such as a compound of Formula IX, such as HBDA. In one embodiment, a method to reprogram a differentiated cell comprises contacting the differentiated cell with Repsox, and also contacting the differentiated cell with Prostaglandin 2. In some such embodiments, the differentiated cell can also be optionally contacted with an exogenous transcription factor of the Oct family of transcription factors, such as Oct3/4. In some such embodiments, the differentiated cell is not contacted with an exogenous transcription factor of the Myc family of transcription factors, such as c-Myc.

Replacement of exogenous Sox and Oct transcription factors: In one embodiment, a method to reprogram a differentiated cell comprises contacting the differentiated cell with a compound which replaces exogenous Sox2 (e.g. any compound with a formula I-VII, such as Repsox, E-616451 or SB431542) and also contacting the differentiated cell with any compound which replaces exogenous Oct4 (e.g. any compound with a formula X or XI, such as Sinimenine or Ripovicane or Bupivacane).

In one embodiment, a method to reprogram a differentiated cell comprises contacting the differentiated cell with a TGFBR1 inhibitor (e.g. any compound with Formulas I, such as Repsox, E-616451 or SB431542) or a Src inhibitor, such as a compound of Formula II (such as EI-275) and also contacting the differentiated cell with any agonist of ATP-dependent K⁺channels, such as a compound of Formula X (e.g. Sinimenine) or any sodium channel inhibitor, such as a compound of Formula X (e.g Sinimenine) or a MAPK agonist such as any compound of Formula XI, XI(a) or XI(b), such as Ripovicane or Bupivacane.

In such an embodiment, the differentiated cell can also be optionally contacted with an exogenous transcription factor of the Klf4 family of transcription factors, such as Klf4. In some such embodiments, the differentiated cell is not contacted with an exogenous transcription factor of the Myc family of transcription factors, such as c-Myc.

Replacement of exogenous Klf and Oct transcription factors: In one embodiment, a method to reprogram a differentiated cell comprises contacting the differentiated cell with a compound which replaces exogenous Klf4 (e.g. any compound with a formula VIII or IX, such as Prostaglandin 2 or HBDA, respectively) and also contacting the differentiated cell with any compound which replaces exogenous Oct4 transcription factor (e.g. any compound with a formula X or XI, such as Sinimenine or Ripovicane or Bupivacane).

In one embodiment, a method to reprogram a differentiated cell comprises contacting the differentiated cell with any Mek/Erk agonist, such as a compound of formula VIII, (such as Prostaglandin 2) or a inhibitor of EGF cell signaling or a inhibitor of the Ca²⁺/Calmodulin signaling pathway, such as a compound of Formula IX, such as HBDA and also contacting the differentiated cell with any agonist of ATP-dependent K⁺channels, such as a compound of Formula X (e.g. Sinimenine) or any sodium channel inhibitor, such as a compound of Formula X (e.g Sinimenine) or a MAPK agonist such as any compound of Formula XI, XI(a) or XI(b), such as Ripovicane or Bupivacane.

In such an embodiment, the cell can also be optionally contacted with an exogenous transcription factor of the Sox family of transcription factors, such as Sox2. In some such embodiments, the differentiated cell is contacted with an exogenous transcription factor of the Myc family of transcription factors, such as c-Myc. In alternative embodiments, the differentiated cell is not contacted with an exogenous transcription factor of the Myc family of transcription factors, such as c-Myc.

Replacement of Exogenous Sox, Klf and Oct Transcription Factors:

In another embodiment, a method to reprogram a differentiated cell comprises contacting the differentiated cell with a compound which replaces exogenous Sox2 (e.g. any compound with a formula I-VII, such as Repsox, E-616451 or SB431542) and contacting the cell with a compound which replaces exogenous Klf4 (e.g. any compound with a formula VIII or IX, such as Prostaglandin 2 or HBDA, respectively), and also contacting the differentiated cell with any compound which replaces exogenous Oct4 transcription factor (e.g. any compound with a formula X or XI, such as Sinimenine or Ripovicane or Bupivacane).

In one embodiment, a method to reprogram a differentiated cell comprises contacting the differentiated cell with a TGFBR1 inhibitor (e.g. any compound with Formulas I, such as Repsox, E-616451 or SB431542) or a Src inhibitor, such as a compound of Formula II (such as EI-275), and also contacting the differentiated cell with any Mek/Erk agonist, such as a compound of formula VIII, (such as Prostaglandin 2) or a inhibitor of EGF cell signaling or a inhibitor of the Ca²⁺/Calmodulin signaling pathway, such as a compound of Formula IX, such as HBDA and also contacting the differentiated cell with any agonist of ATP-dependent K⁺channels, such as a compound of Formula X (e.g. Sinimenine) or any sodium channel inhibitor, such as a compound of Formula X (e.g Sinimenine) or a MAPK agonist such as any compound of Formula XI, XI(a) or XI(b), such as Ripovicane or Bupivacane. In some embodiments, any combination of any compounds selected from any compounds of Formula I-XI, such as, but not limited to Repsox, E-616451, SB431542, EI-275, Prostaglandin 2, HBDA, Sinimenine, Ripovicane and Bupivacane can be added in any order and all possible combinations.

In one embodiment, a method to reprogram a differentiated cell comprises contacting the differentiated cell with at least one compound selected from: Repsox, E-616451, SB431542 or EI-275, and also contacting the differentiated cell with at least one compound selected from Prostaglandin 2 or HBDA, and also contacting the differentiated cell with at least one compound selected from Sinimenine or Ripovicane or Bupivacane.

In another embodiment, a method to reprogram a differentiated cell comprises contacting the differentiated cell with Repsox or SB431542, and also contacting the differentiated cell with Prostaglandin 2, and also contacting the differentiated cell with Ripovicane or Bupivacane. In some embodiments, any combination of any compounds selected from the group of: Repsox, SB431542, Prostaglandin 2, Ripovicane and Bupivacane can be added in any order and all possible combinations. In some such embodiments, the differentiated cell is contacted with an exogenous transcription factor of the Myc family of transcription factors, such as c-Myc. In preferred embodiments, the differentiated cell is not contacted with an exogenous transcription factor of the Myc family of transcription factors, such as c-Myc.

In some embodiments, a differentiated cell is contacted with any and all combinations of compounds with Formulas I-XI, in any order, where the combination includes (but not necessarily in this order): a compound of Formula I (e.g. Repsox, or E-616451); a compound of Formula II (e.g. EI-275), a compound of Formula III (e.g. SB431542), a compound of any of Formulas VI-VII, a compound of Formula VIII (e.g. Prostaglanin 2), a compound of Formula IX (e.g. HDBA), a compound of Formula X (e.g. Sinimenine) or a compound of Formula XI (e.g. Ripovocane or Bupivivane).

In some embodiments, a differentiated cell is contacted with any and all combinations of compounds with Formulas I, III, VIII-XI, in any order, where the combination includes (but not necessarily in this order): a compound of Formula I (e.g. Repsox, or E-616451); a compound of Formula III (e.g. SB431542), a compound of Formula VIII (e.g. Prostaglanin 2), a compound of Formula IX (e.g. HDBA), a compound of Formula X (e.g. Sinimenine) or a compound of Formula XI (e.g. Ripovocane or Bupivivane).

In some embodiments, a differentiated cell is contacted with any and all combinations of compounds with Formulas I, III, VIII and XI, in any order, where the combination includes (but not necessarily in this order): a compound of Formula I (e.g. Repsox, or E-616451); a compound of Formula III (e.g. SB431542), a compound of Formula VIII (e.g. Prostaglanin 2), and a compound of Formula XI (e.g. Ripovocane or Bupivivane).

As discussed herein, in some embodiments, any and all combination of compounds selected from Formula I-XI can be used to reprogram a differentiated cell. In some embodiments, where a differentiated cell is contacted with more than one compound, for example, a Repsox and Bupivocane, each compound can contact the differentiated cell substantially simultaneously (e.g. concurrently or at the same time) or sequentially, in any order.

In some embodiments, a differentiated cell is contacted with a compound of Formula I-XI in a specific order. For example, where a differentiated cell is contacted with at least one compound selected from any compounds with Formula I-VII, and is also contacted with at least one compound selected from the group of compounds with Formulas VIII-IX, and also contacted with a at least one compound with Formula X-XI, the order can be any of the following orders of compounds: (a) I-VII, then VIII-IX, then X-IX, (b) I-VII, then X-IX, then VIII-IX, (c) VIII-IX, then I-VII, then X-IX, (d) V111-IX, then X-IX, then I-VII, (e) X-IX, then I-VII, then VIII-IX, or (f) X-IX, then V111-IX, then I-VII.

By way of an example only, if a differentiated cell is contacted with compound from 3 different formulas, I, VIII and XI, the differentiated cell can be contacted with the compounds in any of the following orders: (a) I (e.g, Repsox), then VIII (e.g. Prostoglandin 2) and then XI (e.g Ripivocane or Bupivicane), (b) I (e.g, Repsox), then XI (e.g Ripivocane or Bupivicane) and then VIII (e.g. Prostoglandin 2), (c) VIII (e.g. Prostoglandin 2), then I (e.g, Repsox) and then XI (e.g Ripivocane or Bupivicane)e, (d) VIII (e.g. Prostoglandin 2), then XI (e.g Ripivocane or Bupivicane) and then I (e.g, Repsox), (e) XI (e.g Ripivocane or Bupivicane), then I (e.g, Repsox) and then VIII (e.g. Prostoglandin 2) (f) XI (e.g Ripivocane or Bupivicane), then VIII (e.g. Prostoglandin 2) and then I (e.g, Repsox).

By way of another example only, if a differentiated cell is contacted with 3 compounds; Repsox, Prostoglandin 2 and Bupivicane, the differentiated cell can be contacted with the compounds in any of the following orders: (a) Repsox, then Prostoglandin 2 and then Bupivicane, (b) Repsox, then Bupivicane and then Prostoglandin 2, (c) Prostoglandin 2, then Repsox and then Bupivicane, (d) Prostoglandin 2, then Bupivicane and then Repsox, (e) Bupivicane, then Repsox and then Prostoglandin 2 (0 Bupivicane, then Prostoglandin 2 and then Repsox.

In some embodiments, a differentiated cell is contacted with any compound which replaces exogenous Oct4 transcription factor (e.g. any compound of Formula X or XI, including Sinomene, Ripivocane or Bupivicane) or any compound which replaces exogenous Klf4 transcription factor (e.g. any compound of Formula VIII-IX, such as prostaglandin J2 or HDBA), prior to being contacted with any agent which replaces exogenous Sox2 transcription factor (e.g. any compound with Formula I-VII, such as Repsox, E-616451, SB431542 or EI-275).

In some embodiments, where the differentiated cell is contacted with more than one compound selected from Formulas I-XI, there may be temporal separation in the time when the differentiated cell is contacted with each of the compounds. In an alternative embodiment, the administration of the compounds to the differentiated cell may be temporally separated. In some embodiments, the temporal separation may range from about less than a minute in time, to about hours or days in time. In some embodiments, the contact can be continious from one compound followed by another compound, and in some embodiments, the contact of the differentiated cell can be intermittent, for example, a differentiated cell is contacted with compound W for a period of time, the not contacted with any compound, then conctacted with compound Y, then not contacted with any compound, then conctacted with compound Z and so on. The determination of the optimal timing and order of administration is readily and routinely determined by one of ordinary skill in the art.

In some embodiments, a differented cell is contacted with a compound of

In some embodiments, the duration of contacting a differentiated cell with any compound of Formulas I-XI is for any period of time which is sufficient to reprogram a differentiated cell into a reprogrammed cell (e.g. a reprogrammed cell in a pluripotent state or a partially reprogrammed cell). Typical durations of contacting a differentiated cell with any compound of Formulas I-XI include, for example, about at least 1 min, at least 5 mins, at least 10 mins, at least about 30 mins, at least about 1 hr, at least about 2 hrs, at least about 3 hrs, at least about 4 hrs, or at least about 5 hrs, or at least about 6 hrs, or at least about 7 hrs, or at least about 8 hrs, or at least about 9 hrs, or at least about 10 hrs, or at least about 11 hrs, or at least about 12 hrs, or at least about 14 hrs, or at least about 16 hrs, or at least about 18 hrs, or at least about 20 hrs, or at least about 24 hrs, or at least about 36 hrs, or at least about 48 hrs or more than 48 hrs. In some embodiments, the duration of contacting a differentiated cell with any compound of Formulas I-XI is longer than 48 hrs, for example, contacting for at least 3 days, or at least about 5 days or at least about 7 days or at least about 10 days.

In some embodiments, where a differentiated cell is contacted Repsox, a differentiated cell is contacted with Repsox for at least about 24 hrs, or at least about 48 hrs, or at least about 96 hrs, or more than 96 hrs, such as for about 7 days or about 9 days, or about 10 days or more than 10 days. In some embodiments, where a partially reprogrammed cell (i.e. a cell which is not fully reprogrammed to a pluripotent state) is contacted Repsox, the partially reprogrammed cell is contacted with Repsox for at least about 24 hrs, or at least about 48 hrs, or at least about 96 hrs, or more than 96 hrs, such as for about 7 days or about 9 days, or about 10 days or more than 10 days.

### Differentiated Cell Types for Reprogramming

The methods described herein can be used, e.g., to chemically reprogram a differentiated cell to a pluripotent state. Such differentiated cells can be obtained, for example from a patient, to prepare patient-specific stem cells (e.g., patient-specific pluripotent stem cells). A variety of differentiated cells can be used, such as, hair follicle cells, a cell from a blood sample, a cell from adipose tissue, a stomach cell, a liver cell, or a cell from skin (e.g., fibroblast or other cell type, e.g., keratinocyte, melanocyte, Langerhans cell, or Merkel cell).

Differentiated cells are any cells forming the body of an organism, as opposed to germline cells. In mammals, germline cells (also known as gametes) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a differentiated cell. For example, internal organs, skin, bones, blood, and connective tissue are all made up of differentiated cells.

Additional differentiated cell types include: a fibroblast (e.g., a primary fibroblast), a muscle cell (e.g., a myocyte), a cumulus cell, a neural cell, a mammary cell, a hepatocyte and a pancreatic islet cell. In some embodiments, the differentiated cell is a primary cell line or is the progeny of a primary or secondary cell line. In one embodiment, the differentiated cell is obtained from a sample, e.g., a hair follicle, a blood sample, a biopsy (e.g., a skin biopsy or an adipose biopsy), a swab sample (e.g., an oral swab sample).

While fibroblasts are preferred, essentially any primary differentiated cells, e.g. a somatic cell type can be used for reprogramming as disclosed herein. Some non-limiting examples of differentiated primary cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a differentiated cell can be a primary cell isolated from any somatic tissue including, but not limited to brain, liver, lung, gut, stomach, intestine, fat, muscle, uterus, skin, spleen, endocrine organ, bone, etc.

Where the cell is maintained under in vitro conditions, conventional tissue culture conditions and methods can be used, and are known to those of skill in the art. Isolation and culture methods for various cells are well within the abilities of one skilled in the art.

Further, the parental cell can be from any mammalian species, with non-limiting examples including a murine, bovine, simian, porcine, equine, ovine, or human cell. In one embodiment, the cell is a human cell. In an alternate embodiment, the cell is from a non-human organism such as e.g., a non-human mammal. The parental cell should not express embryonic stem cell (ES) markers, e.g., Nanog mRNA or other ES markers, thus the presence of Nanog mRNA or other ES markers indicates that a cell has been re-programmed. For clarity and simplicity, the description of the methods herein refers to fibroblasts as the parental cells, but it should be understood that all of the methods described herein can be readily applied to other primary parent cell types.

Where a fibroblast is used, the fibroblast is flattened and irregularly shaped prior to the re-programming, and does not express Nanog mRNA. The starting fibroblast will preferably not express other embryonic stem cell markers. The expression of ES-cell markers can be measured, for example, by RT-PCR. Alternatively, measurement can be by, for example, immunofluorescence or other immunological detection approach that detects the presence of polypeptides that are characteristic of the ES phenotype.

When the reprogrammed cells (e.g. induced pluripotent stem cells) are used for therapeutic treatment of diseases, it is desirable to use differentiated cells (e.g. somatic cells) isolated from patients. For example, differentiated cells (e.g. somatic cells) involved in diseases, somatic cells participating in therapeutic treatment of diseases and the like can be used. In some embodiments, a method for selecting the reprogrammed cells from a heterogeneous population comprising reprogrammed cells and differentiated cells they were derived from can be performed by any well-known means, for example, a drug resistance gene or the like, such as selectable marker genes can be used as a marker gene to isolate the reprogrammed gene using the selectable marker as index.

Various media that can maintain undifferentiated state and pluripotency of ES cells and various media which cannot maintain such properties are known in this field, and reprogrammed cells as disclosed here can be efficiently isolated by using a combination of appropriate media. Differentiation and proliferation abilities of the reprogrammed cells can be easily confirmed by those skilled in the art by using confirmation means widely applied to ES cells.

Thus, one embodiment comprises a reprogrammed cell from a differentiated cell (e.g. a somatic cell) in the absence of eggs, embryos, or embryonic stem (ES) cells.

In some embodiments, the reprogrammed cell (e.g. iPS cell or partially reprogrammed cell) can be a mammalian cell, for example a mouse, human, rat, bovine, ovine, horse, hamster, dog, guinea pig, or ape cell. For example, by chemically reprogramming differentiated cells (e.g. somatic cells) using the methods and compositions as disclosed herein enables the generation of patient- or disease-specific reprogrammed (e.g. iPS) cells, without the need for genetically manipulating the cells (e.g. in the absence of using viral means or other genetic manipulation methods to increase the expression of reprogramming transcription factors). Chemically induced reprogrammed cells (iPS cells) as disclosed herein are indistinguishable from ES cells in morphology, proliferation, gene expression, and teratoma formation. Furthermore, when transplanted into blastocysts, chemically-induced mouse reprogrammed cells (e.g. iPS cells) can give rise to adult chimeras, which are competent for germline transmission (Maherali et al., Cell Stem Cell 1:55-70, 2007; Okita et al., Nature 448:313-17, 2007; Wemig et al., Nature 448:318-324, 2007). Chemically induced human reprogrammed cells are also expandable and indistinguishable from human embryonic stem (ES) cells in morphology and proliferation. Furthermore, these chemically-induced reprogrammed cells can differentiate into cell types of the three germ layers in vitro and in teratomas, as shown in the Examples.

In one embodiment, the somatic cell is a human somatic cell. In one embodiment, the somatic cell is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, or a pancreatic islet cell.

In one embodiment, the somatic cell is a primary cell line or is the progeny of a primary or secondary cell line. In one embodiment, the somatic cell is obtained from a sample, e.g., a hair follicle, a blood sample, a swab sample or an adipose biopsy. In one embodiment, the somatic cell is a healthy cell or a cell containing one or more genetic lesion(s).

In one embodiment, the differentiated cell (e.g. somatic cell) is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, a bone cell, or a pancreatic islet cell. In one embodiment, the differentiated cell is a primary cell line or is the progeny of a primary or secondary cell line. In one embodiment, the differentiated cell is obtained from a sample, e.g., a hair follicle, a blood sample, a swab sample or an adipose biopsy.

In an embodiment, the differentiated cell is obtained from a first individual and the reprogrammed cell derived from the differentiated cell (e.g. the undifferentiated cell or more primitive precursor or a less differentiated cell, e.g., iPS cell or partially reprogrammed cell (or a population thereof)) or a tissue derived from the reprogrammed cell is administered to the same first individual, or to a second individual, e.g., an individual related to said first individual. The second individual can be an individual who carries a different allele for a selected gene than does the first individual. E.g., the first individual can have an allele which does not cause a disease state or unwanted condition and the second individual has the allele which causes the disease state or unwanted condition.

In another embodiment, the differentiated cell is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, a bone cell, or a pancreatic islet cell.

Further, the differentiated cell can be from any mammalian species, with non-limiting examples including a murine, bovine, simian, porcine, equine, ovine, or human cell. In one embodiment, the differentiated cell is a human cell. In an alternate embodiment, the differentiated cell is from a non-human organism such as e.g., a non-human mammal.

As indicated above, the chemicals as disclosed herein can be used to generate reprogrammed cells (e.g. iPS cells or partially reprogrammed cell) from differentiated adult somatic cells. In the preparation of the reprogrammed cells by using the compounds of Formula I-XI as disclosed herein, types of differentiated (e.g. somatic cells) to be reprogrammed are not particularly limited, and any kind of somatic cells may be used. For example, matured somatic cells may be used, as well as somatic cells of an embryonic period. Other examples of cells capable of being generated into iPS cells or partially reprogrammed cells and/or encompassed by the present invention include mammalian cells such as fibroblasts, B cells, T cells, dendritic cells, ketatinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, pancreatic cells, hepatocytes, macrophages, monocytes, mononuclear cells, and gastric cells, including gastric epithelial cells. The cells can be embryonic, or adult somatic cells, differentiated cells, cells with an intact nuclear membrane, non-dividing cells, quiescent cells, terminally differentiated primary cells, and the like.

In some embodiments, the chemicals as disclosed herein can be used to generate reprogrammed cells from partially reprogrammed cells, such as reprogrammed cells which are not fully reprogrammed to a pluripotent state, but rather are stable intermediate non-pluripotent cells as disclosed herein in the Examples.

### Other Molecules to Increase Efficiency of Reprogramming of Differentiated Cells.

The efficiency of reprogramming (e.g., the number of reprogrammed cells) can be enhanced by the addition of various small molecules as shown by Shi, Y., et al (2008) Cell-Stem Cell 2:525-528, Huangfu, D., et al (2008) Nature Biotechnology 26(7):795-797, Marson, A., et al (2008) Cell-Stem Cell 3:132-135, which are incorporated herein by reference in their entirety. It is contemplated that the methods described herein can also be used in combination with additional single small molecule (or a combination of small molecules) that enhances the efficiency of production or a reprogrammed cell. In some embodiments, some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), and trichostatin (TSA), among others. It is also contemplated herein that inhibitors of the TGF-β signaling pathway (e.g. compounds of Formulas I, III, IV, V VI and VII, such as Repsox, E-616451 or SB431542, and anti-TGFβ antibodies or RNAi agents) or inhibitors of SRC signaling, such as compound of Formula II, such as EI-275, or agonist of Mek/Erk cell signalling (e.g. compounds with Formula VIII such as prostaglandin 2); inhibitors of Ca2+/calmodulin signalling or EGF receptor tyrosine kinase inhibitor (e.g. any compound with Formula XI such as HBDA); inhibitors of Na²⁺channels or ATP-dependent potassium channel (e.g. compounds with Formula X, such as Simomenine), or agonists of MAPK signalling pathway (e.g. compounds with Formula XI, such as Ropivocaine or Bupivacaine) can be used either alone or in combination with another small molecule (or combination of small molecules) to enhance or increase the efficiency of producing reprogrammed cells from differentiated cells as disclosed herein. In some embodiments an agent which increase efficiency of production of reprogrammed cells are referred to herein as a “reprogramming enhancing agent”.

In some embodiments a reprogramming enhancing agent as defined herein is not used in reprogramming of a differentiated cell according to the methods as disclosed herein. In some embodiments, a reprogramming enhancing agent is not used where the differentiated cell is contacted with at least one compound selected from the group of; Repsox, prostaglandin or Bupivacaine for reprogramming a differentiated cell.

A reprogramming enhancing agent can increase the efficiency of production of reprogrammed cells or increase the rate of production of reprogrammed cells. By “increasing the efficiency” of reprogrammed cell production is meant that the percentage of reprogrammed cells in a given population is at least 5% higher in populations treated with a such an agent (e.g. reprogramming enhancing agent) than a comparable, control treated population. It is preferred that the percentage of reprogrammed cells in a reprogramming enhancing agent-treated population is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher or more than a control treated population of comparable size and culture conditions. The term “control treated population of comparable size and culture conditions” is used herein to describe a population of cells that has been treated with identical media, viral induction, nucleic acid sequence, temperature, confluency, flask size, pH, etc., with the exception (e.g. absence) of the reprogramming enhancing agent. To be clear, the only difference between a control treated population and a reprogramming enhancing agent-treated cell population is the condition of having been treated with a reprogramming enhancing agent.

By “increasing the rate” of production of reprogrammed cells is meant that the amount of time for the induction of induced pluripotent stem cells is at least 2 days less in a TGβR inhibitor treated cell population than in a control treated population of comparable size and culture conditions; preferably the time needed for pluripotent stem cell induction is at least 3 days less, at least 4 days less, at least 5 days less, at least 6 days less, at least 1 week less, at least 2 weeks less, at least 3 weeks less or more, in the presence of a TGβR inhibitor than in a control treated population.

Histone Deacetylase Inhibitors

In one embodiment, the differentiated cell is further contacted with a HDAC inhibitor, e.g., a HDAC inhibitor described herein or an inhibitor of DNA methyltransferase, e.g., a DNA methyltransferase inhibitor described herein. In one embodiment, the HDAC inhibitor is one or more of valproic acid (VPA), suberoylanilide hydroxamic acid (SAHA) and trichstatin A (TSA). In a preferred embodiment, the method includes contacting a differentiated cell with VPA. In one embodiment the DNA methyltransferase inhibitor is 5-aza-Cytidine (5azaC).

In some embodiments, for example, in a method that includes contacting a cell with an HDAC inhibitor or an inhibitor of DNA methyltransferase, the number of cells produces is greater than the number of cells produced without the HDAC inhibitor (e.g., methods using the compounds of formula I-XI as disclosed herein, without the also contacting of a cell such with an HDAC inhibitor or an inhibitor of DNA methyltransferase. For example, the method can provide 1.25-, 1.5-, 2-, 2.5-, 3-, 4-, 5-, 10-, 15-, 20-, 25-, 30-, 35-, 40-, 50-, 100-, 120-, 130-, 140-, 150-, 200-, 250-, 500-, 750- or 1000-fold greater than the number of reprogrammed cells produced by the methods as disclosed herein.

Histone deacetylases (HDAC) are a class of enzymes that remove acetyl groups from an ε-N-acetyl lysine amino acid on a histone. Exemplary HDACs include those Class I HDAC: HDAC1, HDAC2, HDAC3, HDAC8; and Class II HDACs: HDAC4, HDAC5, HDAC6, HDAC7A, HDAC9, HDAC10. Type I mammalian HDACs include: HDAC1, HDAC2, HDAC3, HDAC8, and HDAC11. Type II mammalian HDACs include: HDAC4, HDAC5, HDAC6, HDAC7, HDAC9, and HDAC1.

A number of structural classes of negative regulators of HDACs (e.g., HDAC inhibitors) have been developed, for example, small molecular weight carboxylates (e.g., less than about 250 amu), hydroxamic acids, benzamides, epoxyketones, cyclic peptides, and hybrid molecules. (See, for example, Drummond D C, Noble C O, Kirpotin D B, Guo Z, Scott G K, et al. (2005) Clinical development of histone deacetylase inhibitors as anticancer agents. Annu Rev Pharmacol Toxicol 45: 495-528, (including specific examples therein) which is hereby incorporated by reference in its entirety). Non-limiting examples of negative regulators of type I/II HDACs include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylureido)caproic hydroxamic acid), AOE (2-amino-8-oxo-9,10-epoxydecanoic acid), CHAP31 and CHAP 50. Other inhibitors include, for example, dominant negative forms of the HDACs (e.g., catalytically inactive forms) siRNA inhibitors of the HDACs, and antibodies that specifically bind to the HDACs. Inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich. In some embodiments, VPA is a preferred histone deacetylase inhibitor.

### Confirmation of the Presence of Reprogrammed Cells:

In some embodiments, the chemically induced reprogrammed cells produces one or more markers indicative of an iPS cell. In some embodiments, the method can include detecting a marker for iPS cells, e.g., for a marker described herein. In some embodiments, the marker can be detected using a reagent, e.g., a reagent for the detection of alkaline phosphatase (AP), NANOG, OCT-4, SOX-2, SSEA4, TRA-1-60 or TRA-1-81, e.g., an antibody against the marker or primers for a RT-PCR or PCR reaction, e.g., a semi-quantitative or quantitative RT-PCR or PCR reaction. Such markers can be used to evaluate whether a reprogrammed cell (e.g. an iPS cell) has been produced. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The progression of a differentiated cell to a reprogrammed cell can be monitored by determining the expression of markers characteristic of reprogrammed cells, such as pluripotent. In some processes, the expression of certain markers is determined by detecting the presence or absence of the marker. Alternatively, the expression of certain markers can be determined by measuring the level at which the marker is present in the cells of the cell culture or cell population. In certain processes, the expression of markers characteristic of pancreatic β-like cell as well as the lack of significant expression of markers characteristic of the cell of endoderm origin from which it was derived is determined.

As described in connection with monitoring the production of a reprogrammed cell from a differentiated cell, qualitative or semi-quantitative techniques, such as blot transfer methods and immunocytochemistry, can be used to measure marker expression, using methods commonly known to persons of ordinary skill in the art. Alternatively, marker expression can be accurately quantitated through the use of technique such as Q-PCR. Additionally, it will be appreciated that at the polypeptide level, many of the markers of pancreatic islet hormone-expressing cells are secreted proteins. As such, techniques for measuring extracellular marker content, such as ELISA, may be utilized.

In other embodiments, the expression of alkaline phosphatase (AP), NANOG, OCT-4, SOX-2, SSEA4, TRA-1-60 or TRA-1-81 in a reprogrammed cells is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher, at least about 500-fold higher, at least about 750-fold higher, at least about 1000-fold higher, at least about 2500-fold higher, at least about 5000-fold higher, at least about 7500-fold higher or at least about 10,000-fold higher than the expression of alkaline phosphatase (AP), NANOG, OCT-4, SOX-2, SSEA4, TRA-1-60 or TRA-1-81 in a differentiated cell from which the reprogrammed cell was derived.

In some embodiments, the method to determine the presence of a reprogrammed cell includes performing an analysis of the karyotype of the iPS cell, for example using a component for karyotyping, e.g., a probe, a dye, a substrate, an enzyme, an antibody or other useful reagents for preparing a karyotype from a cell.

In some embodiments, the presence of a reprogrammed cell in a pluripotent state produced by the methods as disclosed herein is determined using methods which compare the chemically induced reprogrammed cells (iPS cells) from ES cells in morphology, proliferation, gene expression, and teratoma formation according to methods as disclosed herein in the Examples. One can also assess if the reprogrammed cells are in a pluripotent state by assessing if they give rise to adult chimeras which are competent for germline transmission when transplanted into blastocysts, as disclosed in Maherali et al., Cell Stem Cell 1:55-70, 2007; Okita et al., Nature 448:313-17, 2007; Wemig et al., Nature 448:318-324, 2007, which are incorporated herein in their entirety by reference. Additionally, one of ordinary skill in the art can also assess the chemically induced reprogrammed cells to differentiate into cell types of the three germ layers in vitro and in teratomas, as shown herein in the Examples.

In some embodiments, the ability of the reprogrammed cell to form a teratomas, or to differentiated into all three germ layers in vitro is at least about 4-fold higher, at least about 6-fold higher, at least about 8-fold higher, at least about 10-fold higher, at least about 15-fold higher, at least about 20-fold higher, at least about 40-fold higher, at least about 80-fold higher, at least about 100-fold higher, at least about 150-fold higher, at least about 200-fold higher or more than 200-fold higher as compared to the ability of a differentiated cell from which the reprogrammed cell was derived to form a teratomas, or to differentiated into all three germ layers in vitro.

The chemically induced reprogrammed cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; .beta.III-tubulin; .alpha.-smooth muscle actin (.alpha.-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tell); DPPA3/Stella; DPPA4; other general markers for pluripotency, etc. Other markers can include Dnmt3L; Sox15; Stat3; Grb2; SV40 Large T Antigen; HPV16 E6; HPV16 E7, β-catenin, and Bmil. Such cells can also be characterized by the down-regulation of markers characteristic of the differentiated cell from which the iPS cell is induced. For example, iPS cells derived from fibroblasts may be characterized by down-regulation of the fibroblast cell marker Thy1 and/or up-regulation of SSEA-1. It is understood that the present invention is not limited to those markers listed herein, and encompasses markers such as cell surface markers, antigens, and other gene products including ESTs, RNA (including microRNAs and antisense RNA), DNA (including genes and cDNAs), and portions thereof. Markers of partially reprogrammed cells can be used, for example where a partially reprogrammed cell expresses at markers from one or two germ cell layers, but not markers from all three embryonic germ layers (i.e a partially reprogrammed cell does not express markers from all three layers of endoderm, mesoderm or ectoderm layers). Markers of endoderm cells include, Gata4, FoxA2, PDX1, Noda1, Sox7 and Sox17. Markers of mesoderm cells include, Brachycury, GSC, LEF1, Mox1 and Tie1. Markers of ectoderm cells include cripto1, EN1, GFAP, Islet 1, LIM1 and Nestin. Antibodies to markers of the three germ layers are commercially available, such as available from Abeam and other commercial antibody companies.

Enrichment, Isolation and/or Purification of a Population of Reprogrammed Cells Produced by the Methods as Disclosed Herein.

Another aspect of the present invention relates to the isolation of a population of reprogrammed cells from a heterogeneous population of cells, such a mixed population of cells comprising reprogrammed cells and differentiated cells from which the reprogrammed cells were derived. A population of reprogrammed cells produced by any of the above-described processes can be enriched, isolated and/or purified by using any cell surface marker present on the reprogrammed cell which is not present on the differentiated cell from which it was derived. Such cell surface markers are also referred to as an affinity tag which is specific for reprogrammed cells. Examples of affinity tags specific for reprogrammed cells are antibodies, ligands or other binding agents that are specific to a marker molecule, such as a polypeptide, that is present on the cell surface of a reprogrammed cell but which is not substantially present on other cell types (e.g. on differentiated cells). In some processes, an antibody which binds to a cell surface antigen on a reprogrammed cell (e.g. a human reprogrammed cell) is used as an affinity tag for the enrichment, isolation or purification of chemically induced reprogrammed cells produced by the methods described herein. Such antibodies are known and commercially available.

The skilled artisan will readily appreciate that the processes for making and using antibodies for the enrichment, isolation and/or purification of reprogrammed cells are also readily adaptable for the enrichment, isolation and/or purification of reprogrammed cells. For example, in some embodiments, the reagent, such as an antibody, is incubated with a cell population containing reprogrammed cells, wherein the cell population has been treated to reduce intercellular and substrate adhesion. The cell population are then washed, centrifuged and resuspended. In some embodiments, if the antibody is not already labeled with a label, the cell suspension is then incubated with a secondary antibody, such as an FITC-conjugated antibody that is capable of binding to the primary antibody. The reprogrammed cells are then washed, centrifuged and resuspended in buffer. The reprogrammed cell suspension is then analyzed and sorted using a fluorescence activated cell sorter (FACS). Antibody-bound, fluorescent reprogrammed cells are collected separately from non-bound, non-fluorescent cells (e.g. non-reprogrammed cells), thereby resulting in the isolation of reprogrammed cells from differentiated cell types.

In another embodiments of the processes described herein, the isolated cell composition comprising reprogrammed cells can be further purified by using an alternate affinity-based method or by additional rounds of sorting using the same or different markers that are specific for reprogrammed cells. For example, in some embodiments, FACS sorting is used to first isolate a cell which expresses at least one of: alkaline phosphatase (AP), NANOG, OCT-4, SOX-2, SSEA4, TRA-1-60 or TRA-1-81 (or markers of partially reprogrammed cells, such as markers from one or two, but not three of the germ cell layers) from cells that do not express one of those markers (e.g. negative cells) in the cell population. A second FAC sorting, e.g. sorting the positive cells again using FACS to isolate cells that are positive for a different marker than the first sort (e.g. selecting for cells which are positive for at least one of: alkaline phosphatase (AP), NANOG, OCT-4, SOX-2, SSEA4, TRA-1-60 or TRA-1-81, where the selected marker is different to the first sort) enriches the cell population for reprogrammed cells. In other embodiments, FACS sorting is used to separate cells by negatively sorting for a marker that is present on most differentiated cells in the cell population other than the reprogrammed cells.

In some embodiments of the processes described herein, reprogrammed cells are fluorescently labeled without the use of an antibody then isolated from non-labeled cells by using a fluorescence activated cell sorter (FACS). In such embodiments, a nucleic acid encoding GFP, YFP or another nucleic acid encoding an expressible fluorescent marker gene, such as the gene encoding luciferase, is used to label reprogrammed cells using the methods described above. For example, in some embodiments, at least one copy of a nucleic acid encoding GFP or a biologically active fragment thereof is introduced into a differentiated cell which is to be reprogrammed, downstream of a promoter expressed in pluripotent cells, such as the Alkaline phosphatase (AP) promoter such that the expression of the GFP gene product or biologically active fragment thereof is under control of the AP promoter. In some embodiments, the entire coding region of the nucleic acid, which encodes AP is replaced by a nucleic acid encoding GFP or a biologically active fragment thereof. In other embodiments, the nucleic acid encoding GFP or a biologically active fragment thereof is fused in frame with at least a portion of the nucleic acid encoding AP, thereby generating a fusion protein. In such embodiments, the fusion protein retains a fluorescent activity similar to GFP.

In addition to the procedures just described, chemically induced reprogrammed cells may also be isolated by other techniques for cell isolation. Additionally, reprogrammed cells may also be enriched or isolated by methods of serial subculture in growth conditions which promote the selective survival or selective expansion of the reprogrammed cells. Such methods are known by persons of ordinary skill in the art.

Using the methods described herein, enriched, isolated and/or purified populations of reprogrammed cells can be produced in vitro from differentiated cells, which have undergone sufficient reprogramming to produce at least some reprogrammed cells. In a preferred method, the differentiated cells are reprogrammed primarily into reprogrammed cells. Some preferred enrichment, isolation and/or purification methods relate to the in vitro production of reprogrammed cells from human differentiated cells (such as fibroblasts).

Using the methods described herein, isolated cell populations of reprogrammed cells are enriched in reprogrammed content by at least about 2- to about 1000-fold as compared to a population before reprogramming of the differentiated cells. In some embodiments, reprogrammed cells can be enriched by at least about 5- to about 500-fold as compared to a population before reprogramming of the differentiated cells. In other embodiments, reprogrammed cells can be enriched from at least about 10- to about 200-fold as compared to a population before reprogramming of the differentiated cells. In still other embodiments, reprogrammed cells can be enriched from at least about 20- to about 100-fold as compared to a population before reprogramming of the differentiated cells. In yet other embodiments, reprogrammed cells can be enriched from at least about 40- to about 80-fold as compared to a population before reprogramming of the differentiated cells. In certain embodiments, reprogrammed cells can be enriched from at least about 2- to about 20-fold as compared to a population before reprogramming of the differentiated cells.

### Compositions

In another aspect, the disclosure features a population of reprogrammed cells, e.g., undifferentiated cells or a population of undifferentiated cells, produced by a method described herein (e.g. chemically induced reprogrammed cells).

In another aspect, the invention features, a reaction mixture including a differentiated cell and a sufficient amount of TGFBR1 inhibitor(s) such as a compound of Formula I, III-VII as described herein (such as RepSox) or anti-TGF-β-antibody, to convert the differentiated cell to a more primitive precursor or a less differentiated cell, e.g., pluripotent stem cell (or a population thereof). In one embodiment, the differentiated cell is treated with one or more transcription factors, for example, a transcription factor selected from Oct-4, Klf-4, Sox-2 and c-Myc. In some embodiments, the differentiated cell is treated with 2, 3 or 4 transcription factors (e.g., the differentiated cell is treated with Oct-4 and Klf-4, or the differentiated cell is treated with Oct-4, c-Myc, and Klf-4). In some embodiments, the differentiated cell is not treated with c-Myc and/or Sox-2.

In another aspect, the invention features, a reaction admixture or a cell culture comprising a reprogrammed cell (e.g. undifferentiated cell or primitive precursor or a less differentiated cell as compared to a differentiated cell it was derived from), or a population thereof, and one or more TGFBR1 inhibitors as such disclosed herein, such as any compound selected from Formulas I, III-VIII. In one embodiment, a reaction admixture or a cell culture comprises a reprogrammed cell (e.g. undifferentiated cell or primitive precursor or a less differentiated cell as compared to a differentiated cell it was derived from), or a population thereof, and Repsox and/or E-616451 and/or SB43542.

In another aspect, the invention features, a reaction admixture or a cell culture comprising a reprogrammed cell (e.g. undifferentiated cell or primitive precursor or a less differentiated cell as compared to a differentiated cell it was derived from), or a population thereof, and one or more SRC inhibitors as disclosed herein, such as any compound selected from Formula II. In one embodiment, a reaction admixture or a cell culture comprises a reprogrammed cell (e.g. undifferentiated cell or primitive precursor or a less differentiated cell as compared to a differentiated cell it was derived from), or a population thereof, and a compound of Formula II, such as EI-275.

In another aspect of the present invention relates to a composition comprising an isolated population of reprogrammed cells produced by the methods as disclosed herein, for example a chemically induced reprogrammed cell produced by contacting a differentiated cell with (i) at least one compound from any compound which replaces a member of the Sox family of transcription factors (e.g. Sox2), such as any compound with formula I-VII, and (ii) contacting the differentiated cell with at least one compound which replaces a member of the Klf family of transcription factors (e.g. Klf4), such as any compound with VIII-IX, and (iii) contacting the differentiated cell with at least one compound which replaces a member of the Oct family of transcription factors (e.g. Oct 4), such as any compound with X or XI.

In another aspect of the present invention relates to a composition comprising an isolated population of reprogrammed cells produced by the methods as disclosed herein, for example a chemically induced reprogrammed cell produced by contacting a differentiated cell with (i) at least one TGFBR1 inhibitor as disclosed herein, such as a compound such as any compound with formula I, III-VII or an anti-TGFβ antibody or RNAi to TGFBR1 mRNA etc., and/or a inhibitor of Src signaling pathway, such as a compound with Formula II, such as EI-275 or an anti-Src antibody or a RNAi to Src mRNA etc and (ii) where the differentiated cell is also contacted with at least one Mek/Erk agonist, such as a compound with formula VIII (such as prostaglandin J2) and/or at least one inhibitor of EGF cell signalling, such as a compound of formula IX (such as HDBA) or an anti-EGF antibody or RNAi to EGF mRNA, and/or at least one inhibitor of the Ca²⁺/Calmodulin signaling pathway, such as a compound of formula IX (such as HDBA) or an anti-calmodulin antibody or RNAi to calmodulin mRNA, and (iii) where the differentiated cell is also contacted with at least one agonist of ATP-dependent K⁺ channels such as a compound of formula X (such as Sinomenine), and/or at least one sodium channel inhibitor, such as a compound of formula X (such as Sinomenine) or a pan specific anti-Na⁺ channel antibody or RNAi to a sodium channel mRNA, and/or at least one agonist of MAPK signaling pathway, such as any compound with formula XI (such as Ripovocaine or Bupivocaine).

In some embodiments, the composition is a pharmaceutical composition comprising a cell, e.g., a reprogrammed cell (e.g. iPS cell or partially reprogrammed cell, e.g., a reprogrammed cell which has not been fully reprogrammed to a pluripotent state) or a population of reprogrammed cells, produced by a method described herein.

Some embodiments of the present invention relate to cell compositions, such as cell cultures or cell populations, comprising reprogrammed cells, wherein the reprogrammed cells which have been derived from differentiated cells e.g. human differentiated cells. In accordance with certain embodiments, the chemically induced reprogrammed cells are mammalian cells, and in a preferred embodiment, such reprogrammed cells are human reprogrammed cells.

Other embodiments of the present invention relate to compositions, such as cell cultures or cell populations, comprising reprogrammed cells produced by the methods as disclosed herein. In some embodiments of the present invention relate to compositions, such as cell cultures or cell populations, comprising chemically-induced reprogrammed cells produced by the methods as disclosed herein. In such embodiments, the reprogrammed cells comprise less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 12%, less than about 10%, less than about 8%, less than about 6%, less than about 5%, less than about 4%, less than about 3%, less than about 2% or less than about 1% of the total cells in the reprogrammed cell population. In some embodiments, the comprise more than about 90% or the total cells in the cell population, for example about at least 95%, or at least 96%, or at least 97%, or at least 98% or at least about 99%, or about at least 100% of the total cells in the cell population.

Certain other embodiments of the present invention relate to compositions, such as cell cultures or cell populations, comprising an reprogrammed cells and the differentiated cell from which the reprogrammed cells were derived. In some embodiments, the differentiated cells from which the reprogrammed cells are derived comprise less than about 25%, less than about 20%, less than about 15%, less than about 10%, less than about 5%, less than about 4%, less than about 3%, less than about 2% or less than about 1% of the total cells in the culture.

Additional embodiments of the present invention relate to compositions, such as cell cultures or cell populations, produced by the processes described herein and which comprise chemically inducted reprogrammed cells as the majority cell type. In some embodiments, the processes described herein produce cell cultures and/or cell populations comprising at least about 99%, at least about 98%, at least about 97%, at least about 96%, at least about 95%, at least about 94%, at least about 93%, at least about 92%, at least about 91%, at least about 90%, at least about 89%, at least about 88%, at least about 87%, at least about 86%, at least about 85%, at least about 84%, at least about 83%, at least about 82%, at least about 81%, at least about 80%, at least about 79%, at least about 78%, at least about 77%, at least about 76%, at least about 75%, at least about 74%, at least about 73%, at least about 72%, at least about 71%, at least about 70%, at least about 69%, at least about 68%, at least about 67%, at least about 66%, at least about 65%, at least about 64%, at least about 63%, at least about 62%, at least about 61%, at least about 60%, at least about 59%, at least about 58%, at least about 57%, at least about 56%, at least about 55%, at least about 54%, at least about 53%, at least about 52%, at least about 51% or at least about 50% reprogrammed cells. In preferred embodiments, the cells of the cell cultures or cell populations comprise human reprogrammed cells. In other embodiments, the processes described herein produce cell cultures or cell populations comprising at least about 50%, at least about 45%, at least about 40%, at least about 35%, at least about 30%, at least about 25%, at least about 24%, at least about 23%, at least about 22%, at least about 21%, at least about 20%, at least about 19%, at least about 18%, at least about 17%, at least about 16%, at least about 15%, at least about 14%, at least about 13%, at least about 12%, at least about IT %, at least about 10%, at least about 9%, at least about 8%, at least about 7%, at least about 6%, at least about 5%, at least about 4%, at least about 3%, at least about 2% or at least about 1% reprogrammed cells. In preferred embodiments, the cells of the cell cultures or cell populations comprise human reprogrammed cells. In some embodiments, the percentage of reprogrammed cells in the cell cultures or populations is calculated without regard to the feeder cells remaining in the culture.

Still other embodiments of the present invention relate to compositions, such as cell cultures or cell populations, comprising mixtures of reprogrammed cells and differentiated cells. For example, cell cultures or cell populations comprising at least about 5 reprogrammed cells for about every 95 differentiated cell can be produced. In other embodiments, cell cultures or cell populations comprising at least about 95 reprogrammed cells for about every 5 differentiated cell can be produced. Additionally, cell cultures or cell populations comprising other ratios of reprogrammed cells to differentiated cells are contemplated. For example, compositions comprising at least about 1 reprogrammed cell for about every 1,000,000, or at least 100,000 cells, or a least 10,000 cells, or at least 1000 cells or 500, or at least 250 or at least 100 or at least 10 differentiated cell can be produced.

Further embodiments of the present invention relate to compositions, such as cell cultures or cell populations, comprising human cells, including human reprogrammed cells which express at least two or at least 3 or more characteristics of a cell of a pluripotent state as disclosed herein.

In preferred embodiments of the present invention, cell cultures and/or cell populations of reprogrammed cells comprise human reprogrammed, that are non-recombinant cells. In such embodiments, the cell cultures and/or cell populations are devoid of or substantially free of recombinant human reprogrammed cells.

Admixture Compositions.

Another aspect of the present invention relates to an admixture of differentiated cells and at least one compound, such as compounds for reprogramming a differentiated cell selected from the group consisting of compounds with Formula I-XI.

In another aspect of the present invention relates to composition, such as a reaction admixture comprising a differentiated cell, (e.g. a population of differentiated cells for reprogramming) and (i) at least one compound from any compound which replaces a member of the Sox family of transcription factors (e.g. Sox2), such as any compound with formula I-VII, with at least one compound which replaces a member of the Klf family of transcription factors (e.g. Klf4), such as any compound with VIII-IX, and (iii) contacting the differentiated cell with at least one compound which replaces a member of the Oct family of transcription factors (e.g. Oct 4), such as any compound with X or XI.

In another aspect of the present invention relates to a reaction admixture composition comprising a differentiated cell (e.g. a population of differentiated cells for reprogramming) and at least one compound selected from the group of: (i) at least one TGFBR1 inhibitor as disclosed herein, such as a compound such as any compound with formula I, III-VII or an anti-TGFβ antibody or RNAi to TGFBR1 mRNA etc., and/or at least one inhibitor of Src signaling pathway, such as a compound with Formula II, such as EI-275 or an anti-Src antibody or a RNAi to Src mRNA etc, and (ii) at least one Mek/Erk agonist, such as a compound with formula VIII (such as prostaglandin J2) and/or at least one inhibitor of EGF cell signalling, such as a compound of formula IX (such as HDBA) or an anti-EGF antibody or RNAi to EGF mRNA, and/or at least one inhibitor of the Ca²⁺/Calmodulin signaling pathway, such as a compound of formula IX (such as HDBA) or an anti-calmodulin antibody or RNAi to calmodulin mRNA, and (iii) at least one agonist of ATP-dependent K⁺ channels such as a compound of formula X (such as Sinomenine), and/or at least one sodium channel inhibitor, such as a compound of formula X (such as Sinomenine) or a pan specific anti-Na⁺ channel antibody or RNAi to a sodium channel mRNA, and/or at least one agonist of MAPK signaling pathway, such as any compound with formula XI (such as Ripovocaine or Bupivocaine).

In another aspect of the present invention relates to a reaction admixture composition comprising a differentiated cell (e.g. a population of differentiated cells for reprogramming) and at least one of: (i) at least one TGFBR1 inhibitor as disclosed herein, such as a compound such as any compound with formula I, III-VII or an anti-TGFβ antibody or RNAi to TGFBR1 mRNA etc., (ii) at least one inhibitor of Src signaling pathway, such as a compound with Formula II, such as EI-275 or an anti-Src antibody or a RNAi to Src mRNA etc, (iii) at least one Mek/Erk agonist, such as a compound with formula VIII (such as prostaglandin J2), (iv) at least one inhibitor of EGF cell signalling, such as a compound of formula IX (such as HDBA) or an anti-EGF antibody or RNAi to EGF mRNA, (v) at least one inhibitor of the Ca²⁺/Calmodulin signaling pathway, such as a compound of formula IX (such as HDBA) or an anti-calmodulin antibody or RNAi to calmodulin mRNA, and (vi) at least one agonist of ATP-dependent K⁺ channels such as a compound of formula X (such as Sinomenine), (vii) at least one sodium channel inhibitor, such as a compound of formula X (such as Sinomenine) or a pan specific anti-Na⁺ channel antibody or RNAi to a sodium channel mRNA, (viii) at least one agonist of MAPK signaling pathway, such as any compound with formula XI (such as Ripovocaine or Bupivocaine).

In some embodiments, reaction admixture composition comprises a differentiated cell (e.g. a population of differentiated cells for reprogramming) and at least one compound selected from the groups consisting of: (i) at least one TGFBR1 inhibitor as disclosed herein, such as a compound such as any compound with formula I, such as Repsox or E-616451, or formula III (such as SB431542); (ii) at least one Mek/Erk agonist, such as a compound with formula VIII (such as prostaglandin J2) and/or at least one inhibitor of EGF cell signalling or inhibitor of the Ca²⁺/Calmodulin signaling pathway, such as a compound of formula IX (such as HDBA); (iii) at least one agonist of ATP-dependent K+ channels or at least one sodium channel inhibitor, such as a compound of formula X (such as Sinomenine), and/or at least one agonist of MAPK signaling pathway, such as any compound with formula XI (such as Ripovocaine or Bupivocaine).

In some embodiments, reaction admixture composition comprises a differentiated cell (e.g. a population of differentiated cells for reprogramming) and at least one of: (i) at least one TGFBR1 inhibitor as disclosed herein, such as a compound such as any compound with formula I, such as Repsox or E-616451, or formula III (such as SB431542); at least one Src inhibitor selected from a compound with Formula II, such as EI-275; (iii) at least one Mek/Erk agonist, such as a compound with formula VIII (such as prostaglandin J2); (iv) at least one inhibitor of EGF cell signalling or inhibitor of the Ca²⁺/Calmodulin signaling pathway, such as a compound of formula IX (such as HDBA); (v) at least one agonist of ATP-dependent K⁺ channels or at least one sodium channel inhibitor, such as a compound of formula X (such as Sinomenine), (vii) at least one agonist of MAPK signaling pathway, such as any compound with formula XI (such as Ripovocaine or Bupivocaine).

In some embodiments, reaction admixture composition comprises a differentiated cell (e.g. a population of differentiated cells for reprogramming) and at least one of: (i) at least one TGFBR1 inhibitor as disclosed herein, such as a compound such as any compound with formula I, such as Repsox or E-616451, or formula III (such as SB431542); (ii) at least one Mek/Erk agonist, such as a compound with formula VIII (such as prostaglandin J2); (iii) at least one agonist of MAPK signaling pathway, such as any compound with formula XI (such as Ripovocaine or Bupivocaine).

In all aspects of the reaction admixture, the reaction admixture can comprise a reprogramming enhancing agent as that term is described herein, for example, VPA or a HDAC inhibitor and the like. In some embodiments, a reprogramming enhancing agent as that term is described herein, for example, VPA or a HDAC inhibitor is absent in the reaction admixture.

In all aspects of the reaction admixture, the reaction admixture can also comprise a exogenous reprogramming transcription factor, for example as an illustrative example only, where the reaction admixture comprises a differentiated cell and a TGFBR1 inhibitor such as RepSox, the reaction admixture can optionally comprise an exogenous transcription factor (such as a polypeptide or nucleic acid encoding a member of the Oct transcription factor, such as Oct3/4, and/or a member of the Klf family of transcription factors, such as Klf-4). Similarly and by way of example only, where the reaction admixture comprises a differentiated cell, a TGFBR1 inhibitor such as RepSox and a Mek/Erk agonist, such as a compound of Formula VIII (such as prostaglandin 2), the reaction admixture can optionally comprise an exogenous transcription factor (such as a polypeptide or nucleic acid encoding a member of the Oct transcription factor, such as Oct3/4).

In some embodiments, the concentrations of any compound added to the reaction mixture is a sufficient dose for reprogramming a cell, as described herein.

In some embodiments, the composition comprises a concentration of Repsox of about 25 μM, or between 1 μM to 10 μM, or about 1 μM-100 μM. In some embodiments, the composition comprises a concentration of Repsox of at least about 5 μM, at least about 7 μM, at least about 10 μM, at least about 12 μM, at least about 15 μM, at least about 17 μM, at least about 20 μM, at least about 25 μM, at least about 30 μM, at least about 35 μM, at least about 40 μM, at least about 45 μM, at least about 50 μM, at least about 100 μM, or more than 100 μM, or any inter between 10-100 μM or any inter between 1-100 μM, or any integer between 5-25 μM, or any integer between 15 μM-35 μM.

In some embodiments, the composition comprises a concentration of E-616451 of about 3 μM, or between 1 μM to 10 μM, or about 0.1 μM-10 μM. In some embodiments, the composition comprises a concentration of E-616451 at least about 0.1 μM, at least about 0.2 μM, at least about 0.3 μM, at least about 0.4 μM, or at least about 0.5 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, at least about 10 μM, or more than 10 μM, or any inter between 0.1-10 μM or any inter between 1-10 μM, or any integer between 1-3 μM, or any integer between 1 μM-4 μM.

In some embodiments, the composition comprises a concentration of SB431542 of about 25 μM, or between 1 μM to 10 μM, or about 1 μM-100 μM. In some embodiments, the composition comprises a concentration of SB431542 of at least about 5 μM, at least about 7 μM, at least about 10 μM, at least about 12 μM, at least about 15 μM, at least about 17 μM, at least about 20 μM, at least about 25 μM, at least about 30 μM, at least about 35 μM, at least about 40 μM, at least about 45 μM, at least about 50 μM, at least about 100 μM, or more than 100 μM, or any inter between 10-100 μM or any inter between 1-100 μM, or any integer between 5-25 μM, or any integer between 15 μM-3.5 μM.

In some embodiments, the composition comprises a concentration of EI-275 of about 3 μM, or between 1 μM to 10 μM, or about 0.1 μM-10 μM. In some embodiments, the composition comprises a concentration of EI-275 at least about 0.1 μM, at least about 0.2 μM, at least about 0.3 μM, at least about 0.4 μM, or at least about 0.5 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, at least about 10 μM, or more than 10 μM, or any inter between 0.1-10 μM or any inter between 1-10 μM, or any integer between 1-3 μM, or any integer between 1 μM-4 μM.

In some embodiments, the composition comprises a concentration of prostaglandin J2 of about 3 μM, or between 1 μM to 10 μM, or about 0.1 μM-10 μM. In some embodiments, the composition comprises a concentration of prostaglandin J2 at least about 0.1 μM, at least about 0.2 μM, at least about 0.3 μM, at least about 0.4 μM, or at least about 0.5 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, at least about 10 μM, or more than 10 μM, or any inter between 0.1-10 μM or any inter between 1-10 μM, or any integer between 1-3 μM, or any integer between 1 μM-4 μM.

In some embodiments, the composition comprises a concentration of HDBA of about 6 μM, or between 1 μM to 10 μM, or about 0.1 μM-10 μM. In some embodiments, the composition comprises a concentration of HDBA at least about 0.1 μM, at least about 0.2 μM, at least about 0.3 μM, at least about 0.4 μM, or at least about 0.5 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, at least about 10 μM, or more than 10 μM, or any inter between 0.1-10 μM or any inter between 1-10 μM, or any integer between 1-6 μM, or any integer between 5 μM-7 μM.

In some embodiments, the composition comprises a concentration of Sinomenine of about 1 μM, or between 0.1 μM to 1 μM, or about 0.1 μM-10 μM. In some embodiments, the composition comprises a concentration of Sinomenine at least about 0.01 μM, at least about 0.05 μM, at least about 0.075 μM, at least about 0.1 μM, at least about 0.2 μM, at least about 0.3 μM, at least about 0.4 μM, or at least about 0.5 μM, at least about 1 μM, at least about 1.5 μM, at least about 2 μM, at least about 2.5 μM, at least about 4M, at least about 3.5 μM, at least about 4 μM, at least about 4.5 μM, at least about 5 μM, at least about 6 μM, at least about 7 μM, at least about 8 μM, at least about 9 μM, at least about 10 μM, or more than 10 μM, or any inter between 0.01-10 μM or any inter between 0.1-2 μM, or any integer between 0.9-1.5 μM, or any integer between 0.5 μM-1.5 μM.

In some embodiments, the composition comprises a concentration of Ripivocaine of about 1 μM, or between 0.1 μM to 1 μM, or about 0.1 μM-10 μM. In some embodiments, the composition comprises a concentration of Ripivocaine at least about 0.01 μM, at least about 0.05 μM, at least about 0.075 μM, at least about 0.1 μM, at least about 0.2 μM, at least about 0.3 μM, at least about 0.4 μM, or at least about 0.5 μM, at least about 1 μM, at least about 1.5 μM, at least about 2 μM, at least about 2.5 μM, at least about 3 μM, at least about 3.5 μM, at least about 4 μM, at least about 4.5 μM, at least about 5 μM, at least about 6 μM, at least about 7 μM, at least about 8 μM, at least about 9 μM, at least about 10 μM, or more than 10 μM, or any inter between 0.01-10 μM or any inter between 0.1-2 μM, or any integer between 0.9-1.5 μM, or any integer between 0.5 μM-1.5 μM.

In some embodiments, the composition comprises a concentration of Bupivacaine of about 25 μM, or between 1 μM to 10 μM, or about 1 μM-100 μM. In some embodiments, the composition comprises a concentration of Bupivacaine of at least about 5 μM, at least about 7 μM, at least about 10 μM, at least about 12 μM, at least about 15 μM, at least about 17 μM, at least about 20 μM, at least about 25 μM, at least about 30 μM, at least about 35 μM, at least about 40 μM, at least about 45 μM, at least about 50 μM, at least about 100 μM, or more than 100 μM, or any inter between 10-100 μM or any inter between 1-100 μM, or any integer between 5-25 μM, or any integer between 15 μM-35 μM.

### Kits

Another aspect of the present invention relates to kits for practicing methods disclosed herein and for making reprogrammed cells disclosed herein (e.g., iPS cells or partially reprogrammed cell).

In one aspect, a kit includes a differentiated cell and a component described herein such as a TGFBR1 inhibitor(s), e.g., a compound of any of formulas I, III-VIII as described herein (e.g., RepSox or SB-431542), or anti-TGF-β-antibody, or a SRC inhibitor (e.g. a compound of Formula II) and instructions for converting a differentiated cell to a reprogrammed cell using a method described herein. In one embodiment, the differentiated cell is directed to a reprogrammed cell (e.g. an induced pluripotent (iPS) stem cell). In one embodiment, the differentiated cell is directed to a reprogrammed cell which is a partially induced pluripotent cell (e.g. a partially reprogrammed cell, such as a reprogrammed cell which has not been fully reprogrammed to a pluripotent state). In one embodiment, the differentiated cell is directed to a partially reprogrammed cell, (e.g., a reprogrammed cell which has not been fully reprogrammed to a pluripotent state). In one embodiment, a differentiated cell can be used as a control.

In one embodiment, a kit includes at least one of the components listed below. In one preferred embodiment, the kit contains at least two of the components listed below. Any combination of the components described herein can be provided. For example, any combination of the components described herein can be provided.

Another aspect of the present invention relates to kits to produce reprogrammed cells according to the methods as disclosed herein. In some embodiments, the compounds and component described herein (e.g., small molecules of Formulas I-XI (e.g., a TGFBR1 inhibitor(s) of Formulas I, III-VII, including RepSox and/or SB-431542), Src inhibitors (e.g. compounds of Formula II), agonist of MEK or Erk cell signalling (e.g. compounds with Formula VIII, such as Prostaglandin 2); inhibitors of Ca2+/calmodulin signalling or EGF receptor tyrosine kinase inhibitor (e.g. any compound with Formula XI, such as HBDA); inhibitors of Na²⁺channels or ATP-dependent potassium channel (e.g. compounds with Formula X, such as Sinimenine), or agonists of MAPK signalling pathway (e.g. compounds with Formula XI, such as Ropivocaine or Bupivacaine) can be provided singularly or in any combination as a kit. The kit includes (a) the compounds described herein, e.g., a composition(s) that includes a compound(s) described herein, and, optionally (b) informational material.

Exemplary components include the compounds described herein, e.g., a composition(s) that includes a compound(s) described herein, e.g., at least one compound e.g. a TGFBR1 inhibitor (e.g., RepSox or SB-431542) described herein or an anti-TGF-β-antibody.

In some embodiment, the compound in the kit can be provided in a watertight or gas tight container which in some embodiments is substantially free of other components of the kit. The compound can be supplied in more than one container, e.g., it can be supplied in a container having sufficient reagent for a predetermined number of conversions, e.g., 1, 2, 3 or greater. A compound(s) described herein (e.g., compounds of Formula I-XI, such as compounds of Formulas I and III, including RepSox or SB-431542 respectively) or anti-TGF-β-antibody can be provided in any form, e.g., liquid, dried or lyophilized form. It is preferred that a compound(s) described herein be substantially pure and/or sterile. When a compound(s) described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The informational material can be descriptive, instructional, marketing or other material that relates to the methods described herein and/or the use of a compound(s) described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound(s) (e.g., small molecules of Formulas I-XI (e.g., a TGFBR1 inhibitor(s) of Formulas I, III-VII, including RepSox and/or SB-431542), Src inhibitors (e.g. compounds of Formula II), agonist of MEK or Erk cell signalling (e.g. compounds with Formula VIII, such as Prostaglandin 2); inhibitors of Ca2+/calmodulin signalling or EGF receptor tyrosine kinase inhibitor (e.g. any compound with Formula XI, such as HBDA); inhibitors of Na²⁺channels or ATP-dependent potassium channel (e.g. compounds with Formula X, such as Sinimenine), or agonists of MAPK signalling pathway (e.g. compounds with Formula XI, such as Ropivocaine or Bupivacaine) described herein in a suitable manner to perform the methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound(s) described herein, the composition of the kit can include other ingredients, such as a solvent or buffer, a stabilizer, a preservative, a flavoring agent (e.g., a bitter antagonist or a sweetener), a fragrance or other cosmetic ingredient, and/or an additional agent, e.g., for inducing pluripotent stem cells (e.g., in vitro) or for treating a condition or disorder described herein. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

A compound(s) described herein can be provided in any form, e.g., liquid, dried or lyophilized form. It is preferred that a compound(s) described herein be substantially pure and/or sterile. When a compound(s) d described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

In some embodiments, the kit can include a transcription factor, e.g., a transcription factor or combination of transcription factors described herein, e.g., one or more of Oct-4, Klf-4, Sox-2 or c-Myc or a nucleic acid encoding the same transcription factor. In some embodiments, the kit does not include a member of the Sox family of transcription factors, such as exogenous Sox-2 transcription factor. In some embodiments, the kit does not include c-Myc. For example, the kit can provide a vector, e.g., a plasmid or a viral vector, e.g., a retroviral, a lentiviral or an adenoviral vector, which can express one or more of Oct-4, Klf-4, or c-Myc. In some embodiments, the transcription factor is fused to a tag, e.g., a GFP tag, a YFP tag or a RFP tag.

The kit can include a component for the detection of a marker for iPS cells, e.g., for a marker described herein, e.g., a reagent for the detection of alkaline phosphatase (AP), NANOG, OCT-4, SOX-2, SSEA4, TRA-1-60 or TRA-1-81, e.g., an antibody against the marker or primers for a RT-PCR or PCR reaction, e.g., a semi-quantitative or quantitative RT-PCR or PCR reaction. Such markers can be used to evaluate whether an iPS cell has been produced. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

It may be desirable to perform an analysis of the karyotype of the iPS cell. Accordingly, the kit can include a component for karyotyping, e.g., a probe, a dye, a substrate, an enzyme, an antibody or other useful reagents for preparing a karyotype from a cell.

The kit can include an iPS cell, e.g., an iPS cell derived from the same cell type as the somatic cell. In one embodiment, the iPS cell can be for use as a control.

The kit can also include an HDAC inhibitor(s), e.g. VPA. In some embodiments the kit includes a DNA methyltransferase inhibitor (e.g., 5azaC).

The kit can also include informational materials, e.g., instructions, for use of two or more of the components included in the kit.

The informational material can be descriptive, instructional, marketing or other material that relates to the methods described herein and/or the use of a compound(s) described herein for the reprogramming a differentiated cell according to the methods described herein. In one embodiment, the informational material can include information about production of the compound, molecular weight of the compound, concentration, date of expiration, batch or production site information, and so forth. In one embodiment, the informational material relates to methods for culturing the differentiated cell in the presence of a compound. In one embodiment, the informational material can include instructions to culture a differentiated cell in the presence of a compound(s) (e.g., small molecules of Formulas I-XI (e.g., a TGFBR1 inhibitor(s) of Formulas I, 111-VII, including RepSox and/or SB-431542), Src inhibitors (e.g. compounds of Formula II), agonist of MEK or Erk cell signalling (e.g. compounds with Formula VIII, such as Prostaglandin 2); inhibitors of Ca2+/calmodulin signalling or EGF receptor tyrosine kinase inhibitor (e.g. any compound with Formula XI, such as HBDA); inhibitors of Na²⁺channels or ATP-dependent potassium channel (e.g. compounds with Formula X, such as Sinimenine), or agonists of MAPK signalling pathway (e.g. compounds with Formula XI, such as Ropivocaine or Bupivacaine) described herein in a suitable manner to perform the reprogramming methods described herein, e.g., in a suitable dose, dosage form, or mode of administration (e.g., a dose, dosage form, or mode of administration described herein) (e.g., to a cell in vitro or a cell in vivo). In another embodiment, the informational material can include instructions to administer a compound(s) described herein to a suitable subject, e.g., a human, e.g., a human having or at risk for a disorder described herein or to a cell in vitro.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

Some specific embodiments, the kit will provide a differentiated cell, e.g. a somatic cell; at least one compound or any combination of compound selected from any compound with Formulas I-XI (e.g., a TGFBR1 inhibitor(s) of Formulas I, III-VII including RepSox and/or SB-431542), Src inhibitors (e.g. compounds of Formula II), agonist of MEK or Erk cell signalling (e.g. compounds with Formula VIII, such as Prostaglandin 2); inhibitors of Ca2+/calmodulin signalling or EGF receptor tyrosine kinase inhibitor (e.g. any compound with Formula XI, such as HBDA); inhibitors of Na²⁺channels or ATP-dependent potassium channel (e.g. compounds with Formula X, such as Sinimenine), or agonists of MAPK signalling pathway (e.g. compounds with Formula XI, such as Ropivocaine or Bupivacaine); a transcription factor, e.g., a transcription factor or combination of transcription factors described herein, e.g., one or more of Oct-4, Klf-4, Sox-2 or c-Myc or a nucleic acid encoding the same transcription factor; and instructions for use of one or more of the components included in the kit. In some embodiments, the kit does not include a member of the sox transcription factor family, such as Sox-2. In some embodiments, the kit does not include a member of the myc family of transcription factors, such as c-Myc. In some embodiments, the kit does not include a member of the Klf transcription factor family, such as Klf-4. In some embodiments, the kit does not include a member of the Oct transcription factor family, such as Oct 3/4. In some embodiments, the kit does not include any transcription factor selected from the family of transcription factors such as Sox, Myc, Oct and Klf. In some embodiments, one or more of transcription factors Sox2, c-Myc, Oct3/4 and Klf-4 (either polypeptides or nucleic acid sequences encoding the same) are absent in the kit.

In some embodiments, the kit further includes a component for the detection of a marker for iPS cells, e.g., for a marker described herein, e.g., a reagent for the detection of alkaline phosphatase, NANOG, OCT-4, SOX-2, SSEA4, TRA-1-60 or TRA-1-81, e.g., an antibody against the marker.

In another embodiment, the kit further includes a component for preparation of a karyotype from a cell.

In some embodiments, the kit can provide buffers e.g., reaction buffers, solvents, diluents, solutions, stabilizers, preservatives, media, cell lines, vectors, enzymes, secondary antibodies and other materials useful for practicing the methods e.g., a packaging cell line or a packaging vector for virus production, media for culturing iPS cells, or a secondary antibody used for Western analysis or immunofluorescence staining. Alternatively, the other ingredients can be included in the kit, but in different compositions or containers than a compound described herein. In such embodiments, the kit can include instructions for admixing a compound(s) described herein and the other ingredients, or for using a compound(s) described herein together with the other ingredients, e.g., instructions on combining the two agents prior to administration.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

In some embodiments, the kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

In some embodiments, the kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

In one aspect, the invention features a method of instructing an end-user to produce an iPS cell from a somatic cell, the method comprising: (a) providing at least one of the reagents or a kit described herein; and (b) instructing the end-user using an information material e.g., a printed material or a computer readable material, or both.

In another aspect, the invention features a method of instructing an end-user to produce a differentiated cell from an iPS cell, the method comprising: (a) providing at least one of the reagents or a kit described herein; and (b) instructing the end-user using an information material e.g., a printed material or a computer readable material, or both.

### Uses of the Reprogrammed Cells:

In one embodiment, the disclosure features a method of treating a disorder described herein, wherein the method includes: administering a reprogrammed cell or a population of reprogrammed cells, (or differentiated progeny thereof) produced by a method as described herein to a subject, e.g., a subject that suffers from a disorder described herein (e.g., a neurological disorder).

The methods and reprogrammed cells described herein are useful for treating a wide variety of conditions, including hematopoietic conditions (e.g., sickle cell anemia, leukemias, immune deficiencies), cardiac disorders (e.g., myocardial infarcts, and myopathies) and disorders such as liver disease, diabetes, thyroid abnormalities, neurodegenerative/neurological disorders (e.g., Parkinson's, Alzheimer's, stroke injuries, spinal chord injuries), circulatory disorders, respiratory disorders, wound healing and/or repair, bone repair, and enzyme abnormalities.

In one embodiment of the methods described herein, the differentiated cell contains one or more genetic defect, and, e.g., the reprogrammed cell produced by a method described herein includes the genetic defect or defects such as a genetic defect in ALS. In some embodiments, the genetic defect is corrected (e.g., by homologous recombination) in the reprogrammed cell, e.g., to provide a corrected reprogrammed cell. Such cells can be administered by known methods such as the methods described e.g., in U.S. Publication No: 20030228293, the contents of which is incorporated herein by reference. The genetic defect corrected can be, for example, a genetic defect that causes an immune system disorder; a genetic defect that causes a neurological disorder; a genetic defect that causes a cardiac disorder; a genetic defect that causes a circulatory disorder; a genetic defect that causes a metabolic disorder such as diabetes; or a genetic defect that causes a respiratory disorder.

In some embodiments of the methods described herein, the reprogrammed cell or population of reprogrammed cells can be differentiated in vitro into tissue or cell types, for example, useful in treating or studying a condition or disorder. Such differentiation of a reprogrammed cell produces a reprogrammed-differentiated cell, and thus a reprogrammed cell which has been subsequently differentiated is referred to herein as a “reprogrammed-differentiated cell”. For example, a reprogrammed cell (e.g. an iPS cell) as described herein can be differentiated to form a motor neuron. In one embodiment, a reprogrammed cell (e.g. an iPS cell) as described herein can form an embryoid body (e.g., within 2 days). In some embodiments, a reprogrammed cell as described herein can be further differentiated into other cells, for example, a cell that stains positive for Alpha-fetoprotein (AFP, endoderm), skeletal myosin (MF20, mesoderm), or beta-III-tubulin (TuJ1, ectoderm). In one embodiment, the reprogrammed cell or tissues or cell types derived from a reprogrammed cell (e.g. reprogrammed-differentiated cell) can be introduced into a subject, or the same subject from which the differentiated cell was obtained. In one embodiment, the differentiated cell is obtained from a subject having one or more genetic defects and the corrected reprogrammed cell or a tissue of cell type derived from the corrected reprogrammed cell is reintroduced to the subject. Differentiation can be effected by known methods. In one embodiment, the reprogrammed cell, or population of reprogrammed cells are used to produce hematopoietic stem cells (HSC) which are, e.g., useful for transplantation and restoration of immune function in immune deficient recipients.

In one embodiment, the reprogrammed-differentiated cell a chemically induced reprogrammed cell from a subject with a disease, which has then been differentiated into a particular cell type for reimplantation into a subject, for example, as described in the art. Exemplary methods described in the art include, Dimos J T, et al., Induced Pluripotent Stem Cells Generated from Patients with ALS Can Be Differentiated into Motor Neurons. Science. 2008 Jul. 31; Mauritz C, et al., Generation of functional murine cardiac myocytes from induced pluripotent stem cells. Circulation. 2008; 118(5):507-17; Sharma A D, et al., Murine embryonic stem cell-derived hepatic progenitor cells engraft in recipient livers with limited capacity of liver tissue formation. Cell Transplant. 2008; 17(3):313-23; Toh W S, et al., Differentiation of human embryonic stem cells toward the chondrogenic lineage. Methods Mol. Biol. 2007; 407:333-49; Vodyanik M A, et al., Directed differentiation of human embryonic stem cells to dendritic cells. Methods Mol. Biol. 2007; 407:275-93; Roche E, et al., Insulin producing cells from embryonic stem cells: experimental considerations. Methods Mol. Biol. 2007; 407:295-309. Each of these references are incorporated herein by reference.

In another embodiment, the reprogrammed-differentiated cell is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, a bone cell, or a pancreatic islet reprogrammed-differentiated cell.

The methods described herein can further include maintaining the reprogrammed cells under conditions which result in the expansion into additional reprogrammed cells or the differentiation into a desired cell type(s) (e.g., into repaired neurons, cardiac myocytes, blood cell type, bone cell (e.g., osteoblast) or pancreatic cells) to produce reprogrammed-differentiated cell as described herein.

In one aspect, the invention includes a reprogrammed cell (e.g., an iPS) as described herein for the manufacture of a medicament for treating a disorder described herein. The medicament can include other features described herein.

In one embodiment, an isolated population of reprogrammed cells as disclosed herein are administered with a differentiation agent. In one embodiment, the reprogrammed cells are combined with the differentiation agent to administration into the subject. In another embodiment, the reprogrammed cells are administered separately to the subject from the differentiation agent. Optionally, if the reprogrammed cells are administered separately from the differentiation agent, there is a temporal separation in the administration of the reprogrammed cells and the differentiation agent. The temporal separation may range from about less than a minute in time, to about hours or days in time. The determination of the optimal timing and order of administration is readily and routinely determined by one of ordinary skill in the art.

Pharmaceutical compositions of cells:

Pharmaceutical compositions comprising effective amounts of a population of reprogrammed cells are also contemplated by the present invention. These compositions comprise an effective number of reprogrammed cells, optionally, in combination with a pharmaceutically acceptable carrier, additive or excipient. In certain aspects of the present invention, a population of reprogrammed cells are administered to the subject in need of a transplant in sterile saline. In other aspects of the present invention, a population of reprogrammed cells are administered in Hanks Balanced Salt Solution (HBSS) or Isolyte S, pH 7.4. Other approaches may also be used, including the use of serum free cellular media. In one embodiment, a population of reprogrammed cells are administered in plasma or fetal bovine serum, and DMSO. Systemic administration of a population of reprogrammed cells to the subject may be preferred in certain indications, whereas direct administration at the site of or in proximity to the diseased and/or damaged tissue may be preferred in other indications.

In some embodiments, a population of reprogrammed cells can optionally be packaged in a suitable container with written instructions for a desired purpose, such as the reconstitution or thawing (if frozen) of a population of reprogrammed cells prior to administration to a subject.

In one embodiment, an isolated population of reprogrammed cells as disclosed herein are administered with a differentiation agent. In one embodiment, the reprogrammed cells are combined with the differentiation agent to administration into the subject. In another embodiment, the reprogrammed cells are administered separately to the subject from the differentiation agent. Optionally, if the reprogrammed cells are administered separately from the differentiation agent, there is a temporal separation in the administration of the reprogrammed cells and the differentiation agent. The temporal separation may range from about less than a minute in time, to about hours or days in time. The determination of the optimal timing and order of administration is readily and routinely determined by one of ordinary skill in the art.

In some embodiments, a population of reprogrammed cells can be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population. In some embodiments, a population of pancreatic β-like cells may also be modified by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include viral transfection techniques, and more specifically, adeno-associated virus gene transfer techniques, as disclosed in (Walther and Stein, 2000) and (Athanasopoulos et al., 2000). Non-viral based techniques may also be performed as disclosed in (Murarnatsu et al., 1998).

In one aspect of the present invention, a population of reprogrammed cells as disclosed herein are suitable for administering systemically or to a target anatomical site. A population of reprogrammed cells can be grafted into any anatomical site, or may be administered systemically, such as, but not limited to, intra-arterial or intravenous administration. In alternative embodiments, a population of reprogrammed cells of the present invention can be administered in various ways as would be appropriate to implant in the pancreatic or secretory system, including but not limited to parenteral, including intravenous and intraarterial administration, intrathecal administration, intraventricular administration, intraparenchymal, intracranial, intracisternal, intrastriatal, and intranigral administration. Optionally, a population of pancreatic β-like cells are administered in conjunction with an immunosuppressive agent.

In some embodiments, a population of reprogrammed cells can be administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement, including but not limited to improved survival rate or more rapid recovery, or improvement or elimination of symptoms and other indicators as are selected as appropriate measures by those skilled in the art. A population of reprogrammed cells can be administered to a subject the following locations: clinic, clinical office, emergency department, hospital ward, intensive care unit, operating room, catheterization suites, and radiologic suites.

In some embodiments, a population of reprogrammed e cells as disclosed herein may be administered in any physiologically acceptable excipient, where the cells may find an appropriate site for regeneration and differentiation. In some embodiments, a population of reprogrammed cells as disclosed herein can be introduced by injection, catheter, or the like.

In some embodiments, a population of reprogrammed cells as disclosed herein can be frozen at liquid nitrogen temperatures and stored for long periods of time, being capable of use on thawing. If frozen, a population of reprogrammed cells will usually be stored in a 10% DMSO, 50% FCS, 40% RPMI 1640 medium. Once thawed, the cells may be expanded by use of growth factors and/or feeder cells associated with culturing reprogrammed cells as disclosed herein.

In some embodiments, a population of reprogrammed cells as disclosed herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of reprogrammed cells as disclosed herein will be adapted in accordance with the route and device used for administration. In some embodiments, a composition comprising a population of reprogrammed cells can also comprise or be accompanied with one or more other ingredients that facilitate the engraftment or functional mobilization of the reprogrammed cells. Suitable ingredients include matrix proteins that support or promote adhesion of the reprogrammed cells, or complementary cell types, especially endothelial cells. In another embodiment, the composition may comprise resorbable or biodegradable matrix scaffolds.

In other embodiments, a population of reprogrammed cells is stored for later implantation/infusion. A population of reprogrammed cells may be divided into more than one aliquot or unit such that part of a population of reprogrammed cells is retained for later application while part is applied immediately to the subject. Moderate to long-term storage of all or part of the reprogrammed cells in a cell bank is also within the scope of this invention, as disclosed in U.S. Patent Application Serial No. 2003/0054331 and International Patent Application No. WO03024215, and is incorporated by reference in their entireties. At the end of processing, the concentrated cells may be loaded into a delivery device, such as a syringe, for placement into the recipient by any means known to one of ordinary skill in the art.

In some embodiments, a population of reprogrammed cells as disclosed herein may be genetically altered in order to introduce genes useful in the reprogrammed cells, e.g. repair of a genetic defect in an individual, selectable marker, etc., or for the selective suicide of implanted reprogrammed cells. In some embodiments, a population of reprogrammed cells can also be genetically modified to enhance survival, control proliferation, and the like. In some embodiments, a population of reprogrammed cells as disclosed herein can be genetically altering by transfection or transduction with a suitable vector, homologous recombination, or other appropriate technique, so that they express a gene of interest. In one embodiment, a reprogrammed cell is transfected with genes encoding a telomerase catalytic component (TERT), typically under a heterologous promoter that increases telomerase expression beyond what occurs under the endogenous promoter, (see International Patent Application WO 98/14592, which is incorporated herein by reference). In other embodiments, a selectable marker is introduced, to provide for greater purity of the population of reprogrammed cells. In some embodiments, a population of reprogrammed cells may be genetically altered using vector containing supernatants over a 8-16 h period, and then exchanged into growth medium for 1-2 days. Genetically altered reprogrammed cells can be selected using a drug selection agent such as puromycin, G418, or blasticidin, and then recultured.

Gene therapy can be used to either modify a cell to replace a gene product, to facilitate regeneration of tissue, to treat disease, or to improve survival of the cells following implantation into a subject (e.g. prevent rejection).

In an alternative embodiment, a population of reprogrammed cells as disclosed herein can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in the differentiated cell type. Of particular interest are cells that are genetically altered to express one or more growth factors of various types.

### Antibodies in General

Signaling pathways can also be inhibited or activated with peptide bases molecules, e.g., peptide based ligands, antibodies (e.g., monoclonal and polyclonal antibodies) and antibody fragments. Techniques for the production and isolation of antibodies and antibody fragments are well known to one of ordinary skill in the art.

Antibodies that can be used according to the methods described herein include complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone).

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred.

Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites.

Three single chain antibodies can be combined to form triabodies, also known as trivalent trimers. Triabodies are constructed with the amino acid terminus of a VL or VH domain directly fused to the carboxyl terminus of a VL or VII domain, e.g., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific.

Thus, antibodies useful in the methods described herein include, but are not limited to, naturally occurring antibodies, bivalent fragments such as (Fab′)₂, monovalent fragments such as Fab, single chain antibodies, single chain Fv (scFv), single domain antibodies, multivalent single chain antibodies, diabodies, triabodies, and the like that bind specifically with an antigen (e.g., a TGF□R epitope).

Antibodies for use in the methods described herein can be obtained from commercial sources such as AbCam (Cambridge, Mass.), New England Biolabs (Ipswich, Mass.), Santa Cruz Biotechnologies (Santa Cruz, Calif.), Biovision (Mountain View, Calif.), R&D Systems (Minneapolis, Minn.), and Cell Signaling (Danvers, Mass.), among others. Antibodies can also be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., Eds., Antibodies: A Laboratory Manual, Cold Spring Harbor Laboratory, New York (1988), which is hereby incorporated by reference in its entirety.

While both polyclonal and monoclonal antibodies can be used in the methods described herein, it is preferred that a monoclonal antibody is used where conditions require increased specificity for a particular protein.

The term “antibody” describes an immunoglobulin whether natural or partly or wholly synthetically produced. Antibody includes any immunoglobulin, including antibodies and fragments thereof, that binds a specific epitope. The term encompasses polyclonal, monoclonal, recombinant, humanized, and chimeric antibodies. The term also covers any polypeptide or protein having a binding domain which is, or is homologous to, an antibody binding domain. CDR grafted antibodies are also contemplated by this term.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VII domains of a single antibody; (iv) the dAb fragment (Ward, E. S. et al, Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) multivalent antibody fragments (scFv dimers, trimers and/or tetramers (Power and Hudson, J. Immunol. Methods 242: 193-204 9 (2000))(ix) bispecific single chain Fv dimers (PCT/US92/09965) and (x) “diabodies”, multivalent or multispecific fragments constructed by gene fusion (WO94/13804; P. Holliger et al Proc. Natl. Acad. Sci. USA 90 6444-6448, (1993)).

An “antibody combining site” is that structural portion of an antibody molecule comprised of light chain or heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule. Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Antibodies may also be bispecific, wherein one binding domain of the antibody is a specific binding member of the invention, and the other binding domain has a different specificity, e.g. to recruit an effector function or the like. Bispecific antibodies of the present invention include wherein one binding domain of the antibody is a specific binding member of the present invention, including a fragment thereof, and the other binding domain is a distinct antibody or fragment thereof. The other binding domain may be an antibody that recognizes or targets a particular cell type, as in a neural or glial cell-specific antibody. In the bispecific antibodies of the present invention the one binding domain of the antibody of the invention may be combined with other binding domains or molecules which recognize particular cell receptors and/or modulate cells in a particular fashion, as for instance an immune modulator (e.g., interleukin(s)), a growth modulator or cytokine (e.g. tumor necrosis factor (TNF), and particularly, the TNF bispecific modality demonstrated in U.S. Ser. No. 60/355,838 filed Feb. 13, 2002 incorporated herein in its entirety) or a toxin (e.g., ricin) or anti-mitotic or apoptotic agent or factor.

Fab and F(ab′)₂ portions of antibody molecules may be prepared by the proteolytic reaction of papain and pepsin, respectively, on substantially intact antibody molecules by methods that are well-known. See for example, U.S. Pat. No. 4,342,566 to Theofilopolous et al. Fab′ antibody molecule portions are also well-known and are produced from F(ab′)₂ portions followed by reduction of the disulfide bonds linking the two heavy chain portions as with mercaptoethanol, and followed by alkylation of the resulting protein mercaptans with a reagent such as iodoacetamide. An antibody containing intact antibody molecules is preferred herein.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may also contain an antibody molecule having a plurality of antibody combining sites, each immunospecific for a different antigen; e.g., a bispecific (chimeric) monoclonal antibody.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains. Preferably, an antigen binding domain comprises an antibody light chain variable region (VL) and an antibody heavy chain variable region (VH).

RNA Interference

Agonists and inhibitors for use in the present invention can alternatively be peptide or RNA aptamers. Such aptamers can for example interact with the extracellular or intracellular domains of the molecules, e.g., receptors, of interest in cells. An aptamer that interacts with the extracellular domain is preferred as it would not be necessary for such an aptamer to cross the plasma membrane of the target cell. An aptamer could also interact with a ligand such that ligands ability to interact with its receptor is inhibited. Methods for selecting an appropriate aptamer are well known in the art.

Inhibitors for use in the present invention can alternatively be based on oligonucleotides such as antisense oligonucleotides, single and double stranded siRNAs, ribozymes and decoy oligonucleotides. Oligonucleotides, such as antisense and siRNAs, would act to directly block the translation of mRNA by binding thereto and thus preventing protein translation or increasing mRNA degradation, thus decreasing the levels of a receptor or other component of the signaling pathway, and thus activity, in a cell. Methods for using antisense and siRNAs for specifically inhibiting gene expression of genes whose sequence is known are well known in the art.

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B., J. of Virology 76(18):9225 (2002)), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease may be of at least 20%, 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as a nucleic acid-comprising agent which functions to inhibit expression of a target gene, by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, e.g., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be encoded by plasmids, retroviruses, and lentiviruses and expressed from, for example, the pol III U6 promoter, or another promoter (see, e.g., Stewart, et al., RNA April; 9(4):493-501 (2003), incorporated by reference herein in its entirety).

The target gene or sequence of the RNA interfering agent may be a cellular gene or genomic sequence, e.g. the TGF□R sequence. An siRNA may be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical in sequence to its target.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al., Nature Biotechnology 6:635-637 (2003). In addition to expression profiling, one may also screen the potential target sequences for similar sequences in the sequence databases to identify potential sequences which may have off-target effects. For example, according to Jackson et al. (Id.) 15, or perhaps as few as 11 contiguous nucleotides, of sequence identity are sufficient to direct silencing of non-targeted transcripts. Therefore, one may initially screen the proposed siRNAs to avoid potential off-target silencing using sequence identity analysis by any known sequence comparison methods, such as BLAST.

siRNA molecules need not be limited to those molecules containing only RNA, but, for example, further encompasses chemically modified nucleotides and non-nucleotides that effect RNA interference, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group or a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry 42: 7967-7975 (2003). Most of the useful modifications to the siRNA molecules can be introduced using chemistries established for antisense oligonucleotide technology. Preferably, the modifications involve minimal 2′43-methyl modification, preferably excluding such modification. Modifications also preferably exclude modifications of the free 5′-hydroxyl groups of the siRNA.

siRNAs useful for targeting TGF□R or ALK5 expression can be readily designed and tested Chalk et al. (Nucl. Acids Res. 33: D131-D134 (2005)) describe a database of siRNA sequences and a predictor of siRNA sequences. Linked to the sequences in the database is information such as siRNA thermodynamic properties and the potential for sequence-specific off-target effects. The database and associated predictive tools enable the user to evaluate an siRNA's potential for inhibition and non-specific effects. The database is available at on the world wide web at siRNA.cgb.ki.se.

Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al., Nature 411:494-498 (2001); Elbashir, S. M., et al., Genes & Development 15:188-200 (2001); Harborth, J. et al., J. Cell Science 114:4557-4565 (2001); Masters, J. R. et al., Proc. Natl. Acad. Sci., USA 98:8012-8017 (2001); and Tuschl, T. et al., Genes & Development 13:3191-3197 (1999)). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al., Genes Dev. 16:948-958 (2002); McManus, M. T. et al., RNA 8:842-850 (2002); Paul, C. P. et al., Nat. Biotechnol. 20:505-508 (2002); Miyagishi, M. et al., Nat. Biotechnol. 20:497-500 (2002); Sui, G. et al., Proc. Natl. Acad. Sci., USA 99:5515-5520 (2002); Brummelkamp, T. et al., Cancer Cell 2:243 (2002); Lee, N. S., et al., Nat. Biotechnol. 20:500-505 (2002); Yu, J. Y., et al., Proc. Natl. Acad. Sci., USA 99:6047-6052 (2002); Zeng, Y., et al., Mol. Cell. 9:1327-1333 (2002); Rubinson, D. A., et al., Nat. Genet. 33:401-406 (2003); Stewart, S. A., et al., RNA 9:493-501 (2003)).

In the methods described herein, the RNA interference molecule is contacted with a differentiated cell in culture, thus eliminating delivery problems inherent with administering e.g., siRNA in vivo to a patient in need thereof.

In another aspect, a kit contains an reprogrammed cell (e.g. an iPS cell or a partially reprogrammed cell) made by a method described herein, e.g., using one or more component(s) described herein such as a TGFBR1 inhibitor(s) (e.g., RepSox or SB-431542) or an anti-TGF-β-antibody.

In one embodiment, the reprogrammed cell is an iPS cell, or an isolated iPS cell. In one embodiment, the reprogrammed cell is an partially reprogrammed cell, or an isolated partially reprogrammed cell.

In one embodiment, the reprogrammed cell (e.g. an iPS cell or a partially reprogrammed cell) is frozen or in culture.

In another aspect, the invention features a kit comprising an reprogrammed cell (e.g. an iPS cell or a partially reprogrammed cell) made by a method described herein and one or more component(s) for expanding (e.g., multiplying or proliferating) the reprogrammed cell (e.g. an iPS cell or a partially reprogrammed cell). In some embodiments, the kit comprises one or more component(s) for culturing an reprogrammed cell (e.g. an iPS cell or a partially reprogrammed cell) in media thereby expanding the reprogrammed cell (e.g. an iPS cell or a partially reprogrammed cell). In one embodiment, the kit comprises a feeder layer, e.g., an irradiated MEF feeder layer. In one embodiment, the kit comprises hES cell media e.g., hES cell media containing Knockout DMEM supplemented with 10% knockout serum replacement, 10% human plasma fraction, 10 ng/ml bFGF, nonessential amino acids, β-mercaptoethanol, L-glutamine, and/or penicillin/streptomycin. In one embodiment, hES cell media further contain a chemical ROCK (p160-Rho-associated coiled-coil kinase) inhibitor e.g., Y-27632 (see e.g., Watanabe, K. et al. A ROCK inhibitor permits survival of dissociated human embryonic stem cells. Nat. Biotechnol.; 25, 681-686 (2007). In some embodiments, the ROCK inhibitor is at a concentration of from about 1 uM to about 100 um (e.g., at a concentration of e.g., 10 uM). In some embodiments, the ROCK inhibitor is provided in the media for at least about 1 day e.g., for the first two days after passage. In some embodiments, the ROCK inhibitor increases the seeding efficiency of the reprogrammed cell (e.g. an iPS cell or a partially reprogrammed cell).

In another aspect, a kit contains an reprogrammed cell (e.g. an iPS cell or a partially reprogrammed cell), for example, made by a method described herein and instructions for directing an reprogrammed cell (e.g. an iPS cell or a partially reprogrammed cell) to a differentiated cell.

In one embodiment, the reprogrammed cell (e.g. an iPS cell or a partially reprogrammed cell) is made by using one or more component(s) described herein (e.g., an TGFBR1 inhibitor(s) such as a compound described herein (e.g., RepSox) or anti-TGF-β-antibody).

In one embodiment, the differentiated cell is directly differentiated from an reprogrammed cell (e.g. an iPS cell or a partially reprogrammed cell) by a method, for example, described in the art. Exemplary methods described in the art include, Dimos J T, et al., Induced Pluripotent Stem Cells Generated from Patients with ALS Can Be Differentiated into Motor Neurons. Science. 2008; 321(5893):1218-1221; Mauritz C, et al., Generation of functional murine cardiac myocytes from induced pluripotent stem cells. Circulation. 2008; 118(5):507-17; Sharma A D, et al., Murine embryonic stem cell-derived hepatic progenitor cells engraft in recipient livers with limited capacity of liver tissue formation. Cell Transplant. 2008; 17(3):313-23; Toh W S, et al., Differentiation of human embryonic stem cells toward the chondrogenic lineage. Methods Mol. Biol. 2007; 407:333-49; Vodyanik M A, et al., Directed differentiation of human embryonic stem cells to dendritic cells. Methods Mol. Biol. 2007; 407:275-93; Roche E, et al., Insulin producing cells from embryonic stem cells: experimental considerations. Methods Mol. Biol. 2007; 407:295-309. Each of these references are incorporated herein by reference. In another embodiment, the differentiated cell is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, a bone cell, or a pancreatic islet cell. For example, an iPS cell described herein can be differentiated to form a motor neuron. In one embodiment, an iPS cell described herein can form an embryoid body (e.g., within 2 days). In some embodiments, an iPS cell described herein can be further differentiated into other cells, for example, a cell that stains positive for Alpha-fetoprotein (AFP, endoderm), skeletal myosin (MF20, mesoderm), or beta-III-tubulin (TuJ1, ectoderm).

In one aspect, the disclosure features a method of producing a reprogrammed cell (e.g. an iPS cell or a partially reprogrammed cell) from a differentiated (e.g. somatic) cell, the method comprising: contacting an isolated somatic cell with an inhibitor of SRC signaling pathway e.g., a SRC kinase inhibitor (e.g., EI-275 as described in FIG. 1 herein) to thereby produce a reprogrammed cell (e.g. an iPS cell or a partially reprogrammed cell) from the differentiated (e.g. somatic) cell.

In one embodiment, the method comprises contacting a plurality of differentiated cells with an inhibitor of SRC signaling pathway e.g., a SRC kinase inhibitor (e.g., EI-275 as described in FIG. 1D herein) to thereby produce a plurality of reprogrammed cell (e.g. an iPS cell or a partially reprogrammed cell) from the differentiated cells.

In one embodiment, the inhibitor of SRC signaling pathway is a small molecule, an antibody, a double-stranded RNA or any combination thereof. In another embodiment, the inhibitor of SRC signaling pathway is a SRC kinase inhibitor. In one embodiment, the SRC kinase inhibitor is EI-275 as describe in FIG. 1D herein.

In one embodiment, the differentiated (e.g. somatic) cell is a human or mouse differentiated (e.g. somatic) cell.

In one embodiment, the method further comprises treating the differentiated (e.g. somatic) cell with one or more transcription factors (e.g., two, three, or four). In one embodiment, the transcription factor is selected from a group consisting of Oct-4, Klf-4, and c-Myc. In another embodiment, the method comprises treating the somatic cell with two transcription factors (e.g., Oct-4 and Klf-4). In another embodiment, the method comprises contacting the differentiated cell (e.g. somatic cell) with three transcription factors (e.g., Oct-4, Klf-4 and c-Myc). In one embodiment, the method further comprises treating the differentiated cell (e.g. somatic cell) with one or more heterologous nucleic acid sequences encoding at least one transcription factor selected from a group consisting of Oct-4, Klf-4, and c-Myc.

In one embodiment, the method further comprises treating the differentiated cell (e.g. somatic cell) with one or more HDAC inhibitor(s) (e.g., VPA). In one embodiment, the differentiated cell (e.g. somatic cell) is not contacted with one or more HDAC inhibitor(s) (e.g., VPA).

In another embodiment, a plurality of differentiated cell (e.g. somatic cells) are reprogrammed into a plurality of the reprogrammed cells (e.g., iPS cells or partially reprogrammed cells). In one embodiment, the method further comprises isolating a population of the reprogrammed cells (e.g. iPS cells or partially reprogrammed cells) wherein at least 5%, 10%, 15%, 20%, 25%, 30%, 35%, 50%, 75% or greater of cell population are reprogrammed cells.

In one embodiment, the reprogrammed cells (e.g., iPS cells or partially reprogrammed cells) has self-renewal capacity (e.g., survive passaging for at least 1, 2, 5, 10 or greater passages with a growth rate similar to an ES cell and maintain an ES cell-like morphology).

In another embodiment, the expression of a marker (e.g., alkaline phosphatase, NANOG, OCT-4, SOX-2, SSEA4, TRA-1-60 or TRA-1-81) is upregulated to by a statistically significant amount in the reprogrammed cells (e.g., iPS cells) relative to the differentiated cell (e.g. somatic cell) from which it was derived. In some embodiments, where the reprogrammed cells is a partially reprogrammed cell, the partially reprogrammed cell expresses a marker or multiple markers from one or two germ cell layers, but not markers from all three germ cell layers. In another embodiment, the reprogrammed cells (e.g., iPS cells or partially reprogrammed cells) maintains a normal karyotype.

In another embodiment, the reprogrammed cells (e.g., iPS cells or partially reprogrammed cells) has the ability to differentiate in vitro (e.g., to a motor neuron). In one embodiment, the reprogrammed cells (e.g., iPS cells or partially reprogrammed cells) has the ability to differentiate in vivo. In one embodiment, the expression of a marker (e.g., AFP, MF20 or TUJI) is upregulated to by a statistically significant amount in the differentiated cell relative to the reprogrammed cell (e.g., iPS cells or partially reprogrammed cell).

In one embodiment, the differentiated (e.g. somatic cell) is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, a bone cell, or a pancreatic islet cell. In another embodiment, the somatic cell is a primary cell line or is the progeny of a primary or secondary cell line. In another embodiment, the differentiated cell (e.g. somatic cell) is obtained from a sample, e.g., a hair follicle, a blood sample, a swab sample or an adipose biopsy. In one embodiment, the method further comprises implanting the reprogrammed cells (e.g., iPS cells or partially reprogrammed cells) into a subject (e.g., a subject suffering from a disorder). In another embodiment, the reprogrammed cells (e.g., iPS cells or partially reprogrammed cells) is from a donor different than the subject (e.g., a relative of the subject).

In another aspect, the disclosure features a reaction mixture comprising a more primitive precursor or a less differentiated cell compared to a somatic cell it was derived, and an exogenously produced SRC kinase inhibitor such as EI-275 as described in FIG. 1D herein, or a combination thereof. In one preferred embodiment, the less differentiated cell is an iPS cell.

In some embodiments, the present invention provides a kit comprising: a differentiated cell (e.g. a somatic cell); a component described herein; and instructions for converting a differentiated cell to a reprogrammed cell (e.g., iPS cell or partially reprogrammed cell) using the method described herein.

In one embodiment, the kit of comprises at least one of the following: (1) at least one compound e.g., a SRC kinase inhibitor (e.g., Compound of Formula II, e.g. compound EI-275 as shown in FIG. 1D) as described herein; (2) a transcription factor, e.g., a transcription factor or combination of transcription factors described herein, e.g., one or more of Oct-4, Klf-4, Sox-2 or c-Myc or a nucleic acid encoding the same transcription factor; (3) a component for the detection of a marker for the reprogrammed cells (e.g., iPS cells or partially reprogrammed cells), e.g., for a marker described herein, e.g., a reagent for the detection of alkaline phosphatase, NANOG, OCT-4, SOX-2, SSEA4, TRA-1-60 or TRA-1-81, e.g., an antibody against the marker; a component for preparation of a karyotype from a cell; (4) an iPS cell, e.g., an iPS cell derived from the same cell type as the somatic cell, e.g., for use as a control; (5) an HDAC inhibitor e.g., VPA; (5) and instructions for use of one or more of the components included in the kit.

In one aspect, the disclosure features a kit comprising a reprogrammed cell (e.g., iPS cell or partially reprogrammed cell) made by the methods as described herein. In one embodiment, the reprogrammed cell (e.g., iPS cell or partially reprogrammed cell) is made by using one or more components described herein e.g., any compound or combination of compounds selected from Formula I-XI as described herein, e.g. any compound selected from the group of RepSox, E-616451, SB431542, EI-275, Prostaglandin J2, HDBA, Sinimenine, Ripovicane, Bupivacaine, as disclosed herein. In another embodiment, the reprogrammed cell (e.g., iPS cell or partially reprogrammed cell) is an isolated population of reprogrammed cells (e.g., iPS cells or partially reprogrammed cells). In another embodiment, the reprogrammed cell (e.g., iPS cell or partially reprogrammed cell) is frozen or in culture.

In one aspect, the disclosure features a kit comprising a reprogrammed cell (e.g., iPS cells or partially reprogrammed cell); and instructions for directing the reprogrammed cell to a differentiated cell. In one embodiment, the reprogrammed cell (e.g., iPS cell or partially reprogrammed cell) is made by the method described herein. In one embodiment, the reprogrammed cell (e.g., iPS cell or partially reprogrammed cell) is made by using one or more components described herein e.g., e.g., any compound or combination of compounds selected from Formula I-XI as described herein, e.g. any compound selected from the group of RepSox, E-616451, SB431542, EI-275, Prostaglandin J2, HDBA, Sinimenine, Ripovicane, Bupivacaine as disclosed herein. In another embodiment, the reprogrammed cell (e.g., iPS cell or partially reprogrammed cell) is an isolated reprogrammed cell (e.g., iPS cell or partially reprogrammed cell). In one embodiment, the reprogrammed cell (e.g., iPS cell or partially reprogrammed cell) is frozen or in culture. In another embodiment, the differentiated cell is directed from the reprogrammed cell (e.g., iPS cell or partially reprogrammed cell) by a method, e.g. described in the art. In one embodiment, the differentiated cell is selected from a fibroblast (e.g., primary fibroblast), a muscle cell (e.g., a myocyte), a cumulus cell, a neural cell, a liver cell (e.g., a hepatocyte), a GI tract cell, a mammary cell, a kidney cell, a blood cell, a vascular cell, a skin cell, an immune system cell (e.g., a lymphocyte), a lung cell, a bone cell or a pancreatic islet cell.

The methods described herein can improve the efficiency of creating iPS cells from fibroblasts (e.g., MEFs) and are useful for making induced stem cells from other cell types without using the oncogenes, for example c-Myc or Sox-2. For example, these chemicals may make it possible to create reprogrammed cell (e.g., iPS cell or partially reprogrammed cell) from small numbers of cells (e.g., such as those obtained from hair follicle cells from patients, blood samples, adipose biopsy, etc. Thus, the addition of small molecules compounds (e.g., chemicals) can increase the probability of success when trying to make reprogrammed cell (e.g., iPS cell or partially reprogrammed cell) from human skin biopsies (fibroblasts or other nucleated cells) and may be helpful in creating iPS cells from any other cell types.

### Methods to Identify Small Molecule Reprogramming Agents

In another aspect, the disclosure features a method of identifying a compound that will producing a reprogrammed cell (e.g. an iPS cell or partially reprogrammed cell) from a differentiated cell (e.g. somatic cell), the method comprising; providing a differentiated cell; culturing the differentiated cell in the presence of the compound, and evaluating the cultured cell to determine if an iPS cell was produced, thereby determining whether the compound produced the iPS cell.

In one embodiment, the method comprises providing a plurality of differentiated cells; culturing the differentiated cells in the presence of the compound, and evaluating the cultured cells to determine if iPS cells were produced, thereby determining whether the compound produced the iPS cells.

In one embodiment, the differentiated cell was contacted with at least one of Sox-2, Oct-4, Klf-4, c-Myc, Nanog, or Lin-28. In one embodiment, the differentiated cell was contacted with only three of the following four transcription factors: Sox-2, Oct-4, Klf-4, and c-Myc. In one embodiment, the differentiated cell was contacted with only two of the following four transcription factors: Sox-2, Oct-4, Klf-4, and c-Myc. In one embodiment, the differentiated cell was contacted with only one of the following four transcription factors: Sox-2, Oct-4, Klf-4, and c-Myc. In one embodiment, the differentiated cell was cultured in the absence of the following four transcription factors: Sox-2, Oct-4, Klf-4, and c-Myc. In one embodiment, the differentiated cell was contacted with only three of the following four transcription factors: Sox-2, Oct-4, Nanog, or Lin-28. In one embodiment, the differentiated cell was contacted with only two of the following four transcription factors: Sox-2, Oct-4, Nanog, or Lin-28. In one embodiment, the differentiated cell was contacted with only one of the following four transcription factors: Sox-2, Oct-4, Nanog, or Lin-28. In one embodiment, the differentiated cell was cultured in the absence of the following four transcription factors: Sox-2, Oct-4, Nanog, or Lin-28.

In another aspect, the disclosure features a method of treating a subject for a disorder, e.g. a degenerative disorder or cancer, the method comprising administering to the subject a compound identified herein.

As used herein, the term Transforming Growth Factor Receptor (TGFBR) refers to a Transforming Growth Factor Receptor Type I kinase. Exemplary TGFBRs are disclosed herein.

As used herein, the term TGFBR inhibitor refers to a compound that inhibits a Transforming Growth Factor Receptor Type I kinase. In some embodiments, the compound selectively inhibits a Type I TGFBR.

By “selective” is meant at least 20%, 50%, 75%, 2-fold, 3-fold, 4-fold, 5-fold, 6-fold, or 10-fold greater inhibition of a Transforming Growth Factor Receptor kinase over another enzyme, for example a TGFBR2 kinase. Thus, in some embodiments, the agent is selective for TGFBR over a Type II TGFBR. In some embodiment the inhibitor is specific for a Type I TGFBR and thus does not significantly inhibit TGFBRs of other types.

As used herein, a heterologous nucleic acid, is a nucleic acid other than a native endogenous sequence for that gene. E.g., an additional copy of a gene inserted into a chromosome, or a copy on a vector, e.g., a replicative on non replicative vector which has not integrated into the chromosome.

Other features and advantages of the instant invention will become more apparent from the following detailed description and claims. Embodiments of the invention can include any combination of features described herein. In no case does the term “embodiment” necessarily exclude one or more other features disclosed herein, e.g., in another embodiment. The contents of all references, patent applications and patents, cited throughout this application are hereby expressly incorporated by reference.

Stem Cells

Stem cells are cells that retain the ability to renew themselves through mitotic cell division and can differentiate into a diverse range of specialized cell types. The two broad types of mammalian stem cells are: embryonic stem cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Stem cells can be used, e.g., in bone marrow transplants to treat leukemia. Stem cells can be used to treat diseases including cancer, Parkinson's disease, muscle damage, burns, heart disease, diabetes, osteoarthritis, rheumatoid arthritis, hematopoietic conditions (e.g., sickle cell anemia, leukemia, lymphoma, inherited blood disorders), immune deficiencies), cardiac disorders (e.g., myocardial infarcts, and myopathies) and disorders such as liver disease, diabetes, thyroid abnormalities, neurodegenerative/neurological disorders (e.g., Parkinson's Disease, Alzheimer's Disease, stroke injuries, spinal chord injuries), Crohn's Disease, circulatory disorders, respiratory disorders, wound healing and/or repair, bone repair, and enzyme abnormalities.

The present invention may be as defined in any one of the following numbered paragraphs.
1. A method of reprogramming a differentiated cell comprising contacting an isolated differentiated cell with an inhibitor of a TGF-β signaling pathway to thereby produce a reprogrammed cell.
2. The method of paragraph 1, wherein the inhibitor of the TGF-β signaling pathway is an inhibitor of a TGF-β Receptor.
3. The method of paragraph 2, wherein the TGF-β Receptor inhibitor inhibits TGF-β Receptor Type I or TGF-β Receptor Type II.
4. The method of paragraph 3, wherein the inhibitor inhibits TGF-β Receptor Type I and comprises the structure:

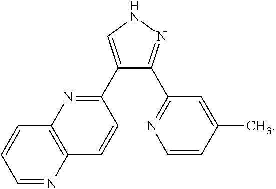

5. The method of paragraph 3, wherein the inhibitor inhibits TGF-β Receptor Type I and comprises the structure:

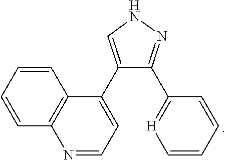

6. The method of paragraph 3, wherein the inhibitor inhibits TGF-β Receptor Type I and comprises the structure:

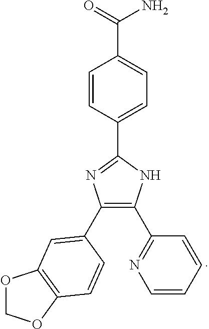

7. A method of reprogramming a differentiated cell comprising contacting an isolated differentiated cell with an inhibitor of Src signalling pathway to thereby produce a reprogrammed cell.
8. A method of paragraph 7, wherein the inhibitor of Src signalling pathway comprises the structure:

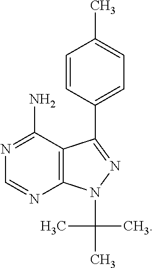

9. A method of reprogramming a differentiated cell comprising contacting an isolated differentiated cell with the compound of Formula I to thereby produce a reprogrammed cell, wherein the compound of Formula I is:

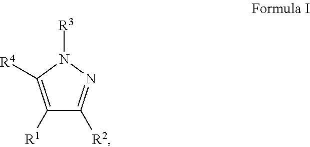

wherein:
R¹ cyclyl, heterocyclcyl, aryl or heteroaryl, each of which can be optionally substituted;
R² cyclyl, heterocyclcyl, aryl or heteroaryl, each of which can be optionally substituted;
R³ is H, C₁-C₆ alkyl, arylC₁-C₆, or a nitrogen protecting group, each of which can be optionally substituted; and
R⁴ is H, optionally substituted C₁-C₆ alkyl, optionally substituted C₂-C₆ alkenyl, optionally substituted C₂-C₆ alkynyl, or R³ and R⁴ together with the atoms they are attached to form a cyclyl, heterocyclyl, aryl or heteroaryl, each of which can be optionally substituted.
10. A method of paragraph 9, wherein an isolated differentiated cell is contacted with the compound of Formula I(a) or Formula I(b) to thereby produce a reprogrammed cell, wherein the compound of Formula I(a) is:

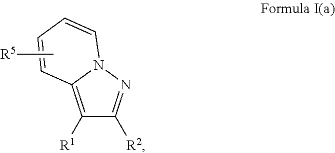

wherein:
R¹ cyclyl, heterocyclcyl, aryl or heteroaryl, each of which can be optionally substituted;
R² cyclyl, heterocyclcyl, aryl or heteroaryl, each of which can be optionally substituted; and
R⁵ is R⁵ is H, benzyl, aryl, heteroaryl, C₁-C₆alkyl, alkenyl, alkynyl, halogen, amino, —C(O)-amino, —SO₂-alkyl, —O-alkyl or acyl, each of which can be optionally substituted, and the compound of Formula I(b) is:

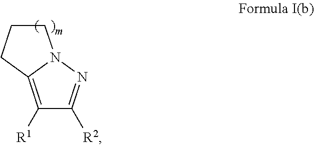

wherein:
R¹ cyclyl, heterocyclcyl, aryl or heteroaryl, each of which can be optionally substituted;
R² cyclyl, heterocyclcyl, aryl or heteroaryl, each of which can be optionally substituted; and
m is 1, 2 or 3.
11. A method of reprogramming a differentiated cell comprising contacting an isolated differentiated cell with the compound of Formula II to thereby produce a reprogrammed cell, wherein the compound of Formula II is:

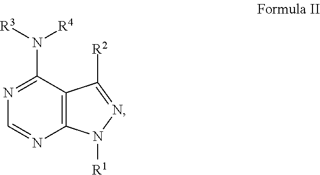

wherein:
R¹ is H, C₁-C₆ alkyl, or C₁-C₆ haloalky;
R² is optionally substituted aryl or optionally substituted heteroaryl; and
each R³ and R⁴ is independently H, C₁-C₆ alkyl, arylC₁-C₆alklyl, or a nitrogen protecting group.
12. A method of reprogramming a differentiated cell comprising contacting an isolated differentiated cell with the compound of Formula III to thereby produce a reprogrammed cell, wherein the compound of Formula III is:

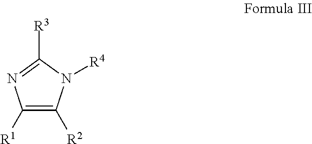

R¹ is cyclyl, heterocyclcyl, aryl or heteroaryl, each of which can be optionally substituted;
R² is cyclyl, heterocyclcyl, aryl or heteroaryl, each of which can be optionally substituted;
R³ is cyclyl, heterocyclcyl, aryl, heteroaryl or —S(O)alkyl, each of which can be optionally substituted; and
R⁴ is H, optionally substituted C₁-C₆ alkyl, optionally substituted C₂-C₆ alkenyl, optionally substituted C₂-C₆ alkynyl, or R³ and R⁴ together with the atoms they are attached to form a cyclyl, heterocyclyl, aryl or heteroaryl, each of which can be optionally substituted.
13. A method of paragraph 12, wherein an isolated differentiated cell is contacted with the compound of Formula III(a) or Formula III(b) to thereby produce a reprogrammed cell, wherein the compound of Formula III(a) is:

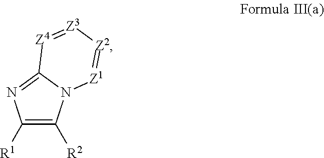

wherein:
R¹ is cyclyl, heterocyclcyl, aryl or heteroaryl, each of which can be optionally substituted;
R² is cyclyl, heterocyclcyl, aryl or heteroaryl, each of which can be optionally substituted;
z¹-z⁴ independently CR⁵ or N, provided that no two N are next to each other; and
R⁵ is H, benzyl, aryl, heteroaryl, C₁-C₆alkyl, alkenyl, alkynyl, halogen, amino, —C(O)-amino, —SO₂-alkyl, —O-alkyl or acyl, each of which can be optionally substituted, and the compound of Formula III(b) is:

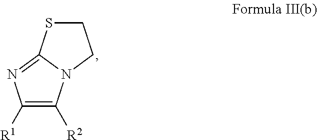

wherein:
R¹ is cyclyl, heterocyclcyl, aryl or heteroaryl, each of which can be optionally substituted; and R² is cyclyl, heterocyclcyl, aryl or heteroaryl, each of which can be optionally substituted.
14. A method of reprogramming a differentiated cell comprising contacting an isolated differentiated cell with a compound of any of Formula IV-VII to thereby produce a reprogrammed cell, wherein the compound of Formula IV is Formula IV(a) or Formula IV(b), wherein the compound of Formula IV(a) is:

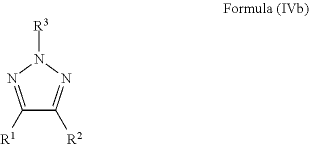

and the compound of Formula IV(b) is:

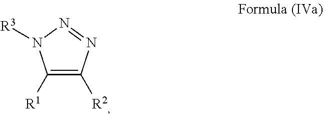

wherein, R¹ is cyclyl, heterocyclcyl, aryl or heteroaryl, each of which can be optionally substituted;
R² is cyclyl, heterocyclcyl, aryl or heteroaryl, each of which can be optionally substituted; and
R³ is R³ is H, C₁-C₆ alkyl, C₂-C₆ alkenyl, C₂-C₆ alkynyl, aryl, heteroaryl, cyclyl, heterocyclyl, acyl or a nitrogen protecting group, each of which can be optionally substituted, and
wherein the compound of Formula V is:

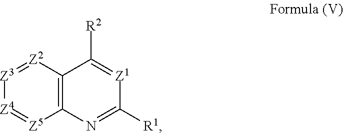

wherein:
R¹ is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;
R² is H, C₁-C₆ alkyl, C₁-C₆ alkenyl, C₁-C₆ alkynyl, aryl, heteroaryl, cyclyl, optionally substituted heterocyclyl, acyl or amino (e.g., N(R³)₂), each of which can be optionally substituted;
R³ is independently for each occurrence H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;
Z¹, Z², Z³, Z⁴ and Z⁵ are each independently N or CR³, provided that at least two of Z², Z³, Z⁴ and Z⁵ are CR³, and further provided that two adjacent Z positions are not N; and
wherein the compound of Formula VI is:

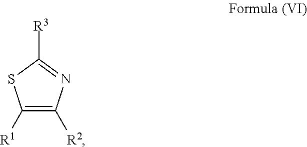

wherein:
R¹ is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;
R² is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;
R³ is H, C₁-C₆ alkyl, C₁-C₆ alkenyl, C₁-C₆ alkynyl, aryl, heteroaryl, cyclyl, optionally substituted heterocyclyl, acyl or amino, each of which can be optionally substituted, and
wherein the compound of Formula VII is:

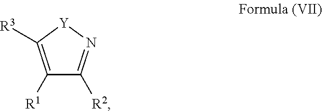

wherein:
X is O, S or CH₂;
R¹ is H, C₁-C₆ alkyl, C₁-C₆ alkenyl, C₁-C₆ alkynyl, aryl, heteroaryl, cyclyl, heterocyclyl, acyl, amino, or amide (e.g., —CO₂NH₂), each of which can be optionally substituted;
R² is C₁-C₆ alkyl, —O-alkyl, amino, acyl, aryl, heteroaryl, cyclyl or heterocyclyl, each of which can be optionally substituted;
R³ is H, C₁-C₆ alkyl, —O-alkyl, amino, amide, —NHC(O)NH-alkyl, acyl, aryl, heteroaryl, cyclyl, heterocyclyl, each of which can be optionally substituted.
15. The method of any of paragraphs 1-14, wherein the isolated cell is not contacted with an exogenous Sox2 transcription factor or an exogenous Nanog transcription factor.
16. The method of any of paragraphs 1-15, wherein the isolated cell is not contacted with an exogenous c-myc transcription factor.
17. The method of any of paragraphs 1-15, further comprising contacting the isolated differentiated cell with one or more exogenous transcription factors.
18. The method of paragraph 17, wherein the transcription factor is selected from the group consisting of Oct-4, Klf-4, c-Myc, lin-28 and Nanog.
19. The method of paragraphs 17 or 18, wherein the isolated differentiated cell is contacted with an exogenous Oct-4 transcription factor or an exogenous Klf-4 transcription factor.
20. The method of any of paragraphs 17 to 19, wherein an exogenous transcription factor is a nucleic acid encoding at least one transcription factor selected from the group consisting of Oct-4, Klf-4, c-Myc, lin-28 and Nanog or a biologically active polypeptide of at least one transcription factor selected from the group consisting of Oct-4, Klf-4, c-Myc, lin-28 and Nanog.
21. The method of any of claims 1 to 17, wherein the isolated differentiated cell is not contacted with an exogenous Klf-4 transcription factor.
22. The method of any of claims 1 to 17, wherein the isolated differentiated cell is not contacted with an exogenous Oct-4 transcription factor.
23. The method of any of paragraphs 1 to 15, 21 or 22, wherein the isolated cell is not contacted with an exogenous c-myc transcription factor or an exogenous lin-28 transcription factor.
24. A method of reprogramming a differentiated cell comprising contacting an isolated differentiated cell with an agonist of the Mek/Erk signalling pathway to produce a reprogrammed cell.
25. A method of reprogramming a differentiated cell comprising contacting an isolated differentiated cell with an inhibitor of Ca²⁺/calmodulin to produce a reprogrammed cell.
26. A method of reprogramming a differentiated cell comprising contacting an isolated differentiated cell with an inhibitor of EGF signaling to produce a reprogrammed cell.
27. A method of reprogramming a differentiated cell comprising contacting an isolated differentiated cell with a compound of Formula VIII to produce a reprogrammed cell, wherein the Formula VIII is:

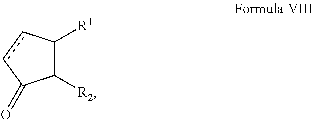

wherein:
R¹ is optionally substituted C₄-C₁₀ alkyl, C₄-C₁₀ alkenyl or C₄-C₁₀ alkynyl;
R² is optionally substituted C₄-C₁₀ alkyl, C₄-C₁₀ alkenyl or C₄-C₁₀ alkynyl; and
The dashed line ( - - - ) indicates the presence or absence of a bond.
28. A method of paragraph 27, wherein an isolated differentiated cell is contacted with a compound comprising the structure:

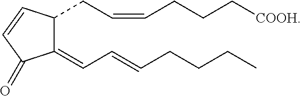

29. A method of reprogramming a differentiated cell comprising contacting an isolated differentiated cell with a compound of Formula IX to produce a reprogrammed cell, wherein the Formula IX is:

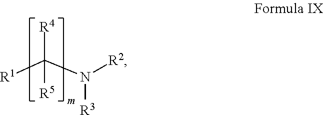

wherein:
R¹ cyclyl, heterocyclcyl, aryl or heteroaryl, each of which can be optionally substituted;
R² cyclyl, heterocyclcyl, aryl or heteroaryl, each of which can be optionally substituted;
R³ is H, C₁-C₆ alkyl, aryl, heteroaryl, cyclyl, heterocyclyl, arylalkyl, heteroarylalkyl, or a nitrogen protecting group, each of which can be optionally substituted;
each R⁴ and R⁵ is independently H, halo, —CN, —NO₂, C₁-C₆ alkyl, haloC₁-C₆alkyl, —CO₂R⁶, —OR⁶ or —N(R⁶)₂, each of which can be optionally substituted;
R⁶ is independently H, C₁-C₆alkyl, aryl, heteroaryl, cyclyl, heterocyclyl or acyl, each of which can be optionally substituted; and
m is 0, 1 or 2.
30. A method of reprogramming a differentiated cell comprising contacting an isolated differentiated cell with a compound of Formula IX(a) to produce a reprogrammed cell, wherein the Formula IX(a) is:

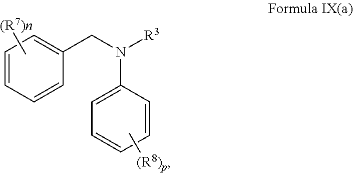

wherein:
R³ is H, C₁-C₆ alkyl, aryl, heteroaryl, cyclyl, heterocyclyl, arylalkyl, heteroarylalkyl, or a nitrogen protecting group, each of which can be optionally substituted;
each R⁷ and R⁸ is independently halo, —CN, —NO₂, C₁-C₆ alkyl, C₂-C₆ alkenyl, C₁-C₆ alkynyl, haloC₁-C₆alkyl, —CO₂R⁶, —OR⁶, —N(R⁶)₂, each of which can be optionally substituted;
n is 0, 1, 2, 3, 4 or 5; and
p is 0, 1, 2, 3, 4 or 5.
31. A method of paragraphs 29 or 30, wherein an isolated differentiated cell is contacted with a compound comprising the structure:

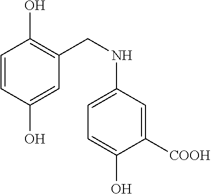

32. The method of any of paragraphs 24 to 31, wherein the isolated differentiated cell is not contacted with an exogenous Klf-4 transcription factor.
33. The method of any of paragraphs 24 to 31, further comprising contacting the isolated differentiated cell with one or more exogenous transcription factors.
34. The method of paragraph 33, wherein the transcription factor is selected from the group consisting of Sox2, Oct-4, c-Myc, lin-28 and Nanog.
35. The method of paragraphs 33 or 34, wherein the isolated differentiated cell is contacted with an exogenous Sox2 transcription factor or an exogenous Oct-4 transcription factor.
36. The method of any of paragraphs 33 to 34, wherein an exogenous transcription factor is a nucleic acid encoding at least one transcription factor selected from the group consisting of Sox2, Oct-4, c-Myc, lin-28 and Nanog or a biologically active polypeptide of at least one transcription factor selected from the group consisting of Sox2, Oct-4, c-Myc, lin-28 and Nanog.
37. The method of any of paragraphs 24 to 33, wherein the isolated differentiated cell is not contacted with an exogenous Sox2 transcription factor.
38. The method of any of paragraphs 24 to 33, wherein the isolated differentiated cell is not contacted with an exogenous Oct-4 transcription factor.
39. A method of reprogramming a differentiated cell comprising contacting an isolated differentiated cell with an agonist of ATP-dependent potassium channels to produce a reprogrammed cell.
40. A method of reprogramming a differentiated cell comprising contacting an isolated differentiated cell with an inhibitor of Sodium channels to produce a reprogrammed cell.
41. A method of reprogramming a differentiated cell comprising contacting an isolated differentiated cell with a MAPK agonist to produce a reprogrammed cell.
42. A method of reprogramming a differentiated cell comprising contacting an isolated differentiated cell with a compound of Formula X to produce a reprogrammed cell, wherein the Formula X is:

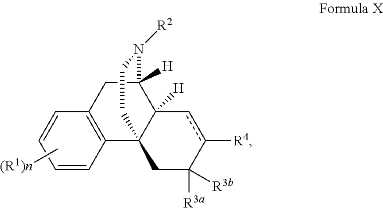

wherein:
each R¹ is independently C₁-C₆ alkyl, C₁-C₆ haloalkyl, —COOR⁵, —OR⁵, —NR⁵₂, —NO₂ or —CN;
R² is hydrogen, C₁-C₆ alkyl, aryl, heteroaryl, cyclyl, heterocyclyl, arylalkyl, or a nitrogen protecting group, each of which is optionally substituted;
Each R^{3a} and R^{3b} is independently hydrogen, —COOR⁵ or —OR⁵, or R^{3a} and R^{3b} taken together with the carbon to which they are attached form a carbonyl;
R⁴ is hydrogen, C₁-C₆ alkyl, C₁-C₆ haloalkyl, —COOR⁵, —OR⁵, —NR⁵₂, —NO₂ or —CN;
Each R⁵ is independently hydrogen, C₁-C₆ alkyl, aryl, heteroaryl, cyclyl, heterocyclyl or acyl;
n is 0, 1, 2, 3 or 4; and
the dashed line ( - - - ) indicates the presence or absence of a bond.
43. A method of paragraph 42, wherein an isolated differentiated cell is contacted with a compound comprising the structure:

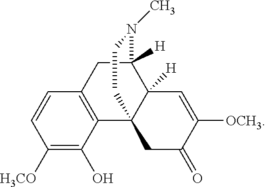

44. A method of reprogramming a differentiated cell comprising contacting an isolated differentiated cell with a compound of Formula XI to produce a reprogrammed cell, wherein the Formula XI is:

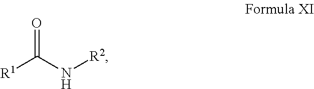

wherein:
R¹ is cyclyl, heterocyclcyl, aryl or heteroaryl, each of which can be optionally substituted; and R² is cyclyl, heterocyclcyl, aryl or heteroaryl, each of which can be optionally substituted.
45. The method of paragraph 44, wherein the isolated differentiated cell is contacted with Ripivacaine or Bupivacaine, or both Ripivacaine and Bupivacaine, wherein Ripivacaine is a compound comprising the structure:

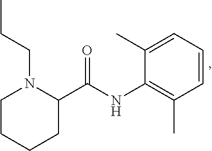

and Bupivacaine is a compound comprising the structure of:

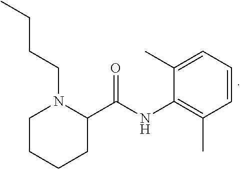

46. The method of any of paragraphs 39 to 45, wherein the isolated differentiated cell is not contacted with an exogenous Oct-4 transcription factor.
47. The method of any of paragraphs 39 to 46, further comprising contacting the isolated differentiated cell with one or more exogenous transcription factors.
48. The method of paragraph 47, wherein the transcription factor is selected from the group consisting of Sox2, Klf-4, c-Myc, lin-28 and Nanog.
49. The method of paragraphs 47 or 48, wherein the isolated differentiated cell is contacted with an exogenous Sox2 transcription factor or an exogenous Klf-4 transcription factor, or an exogenous Sox2 transcription factor and an exogenous Klf-4 transcription factor.
50. The method of any of paragraphs 47 to 49, wherein an exogenous transcription factor is a nucleic acid encoding at least one transcription factor selected from the group consisting of Sox2, Klf-4, c-Myc, lin-28 and Nanog or a biologically active polypeptide of at least one transcription factor selected from the group consisting of Sox2, Klf-4, c-Myc, lin-28 and Nanog.
51. The method of any of paragraphs 39 to 47, wherein the isolated differentiated cell is not contacted with an exogenous Sox2 transcription factor.
52. The method of any of paragraphs 39 to 47, wherein the isolated differentiated cell is not contacted with an exogenous Klf-4 transcription factor.
53. The method of paragraphs 21 or 52, wherein the isolated differentiated cell is contacted with a agonist of the Mek/Erk signaling pathway.
54. The method of paragraphs 21 or 52, wherein the isolated differentiated cell is contacted with an inhibitor of Ca²⁺/calmodulin signaling pathway.
55. The method of paragraphs 21 or 52, wherein the isolated differentiated cell is contacted with an inhibitor of EGF signaling pathway signaling pathway.
56. The method of paragraphs 24 or 53, wherein the agonist of the Mek/Erk signaling pathway is selected from any compound of Formula VIII, wherein Formula VIII is:

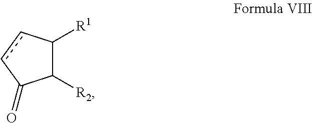

wherein:
R¹ is optionally substituted C₄-C₁₀ alkyl, C₄-C₁₀ alkenyl or C₄-C₁₀ alkynyl;
R² is optionally substituted C₄-C₁₀ alkyl, C₄-C₁₀ alkenyl or C₄-C₁₀ alkynyl; and
the dashed line ( - - - ) indicates the presence or absence of a bond.
57. The method of any of paragraphs 21, 24, 53 or 56, wherein the agonist of the Mek/Erk signaling pathway is 15-deoxy-delta^{12,14}-Prostaglandin J₂ (PGJ₂), wherein deoxy-delta^{12,14}-Prostaglandin (PGJ₂) has the following structure

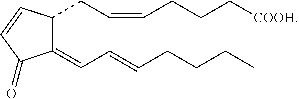

58. The method of paragraphs 25 or 54, wherein the agonist of the inhibitor of Ca²⁺/Calmodulin signalling pathway is selected from any compound of Formula IX, wherein Formula IX is:

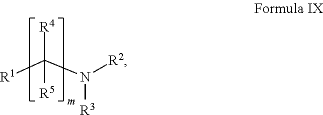

wherein:
R¹ cyclyl, heterocyclcyl, aryl or heteroaryl, each of which can be optionally substituted;
R² cyclyl, heterocyclcyl, aryl or heteroaryl, each of which can be optionally substituted;
R³ is H, C₁-C₆ alkyl, aryl, heteroaryl, cyclyl, heterocyclyl, arylalkyl, heteroarylalkyl, or a nitrogen protecting group, each of which can be optionally substituted;
each R⁴ and R⁵ is independently H, halo, —CN, —NO₂, C₁-C₆ alkyl, haloC₁-C₆alkyl, —CO₂R⁶, —OR⁶ or —N(R⁶)₂, each of which can be optionally substituted;
R⁶ is independently H, C₁-C₆alkyl, aryl, heteroaryl, cyclyl, heterocyclyl or acyl, each of which can be optionally substituted; and
m is 0, 1 or 2.
59. The method of any of paragraphs 26 or 55, wherein the inhibitor of EGF signalling pathway is selected from any compound of Formula IX, wherein Formula IX is:

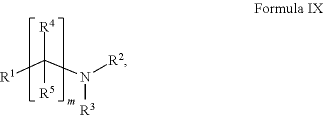

wherein:
R¹ cyclyl, heterocyclcyl, aryl or heteroaryl, each of which can be optionally substituted;
R² cyclyl, heterocyclcyl, aryl or heteroaryl, each of which can be optionally substituted;
R³ is H, C₁-C₆ alkyl, aryl, heteroaryl, cyclyl, heterocyclyl, arylalkyl, heteroarylalkyl, or a nitrogen protecting group, each of which can be optionally substituted;
each R⁴ and R⁵ is independently H, halo, —CN, —NO₂, C₁-C₆ alkyl, haloC₁-C₆alkyl, —CO₂R⁶, —OR⁶ or —N(R⁶)₂, each of which can be optionally substituted;
R⁶ is independently H, C₁-C₆alkyl, aryl, heteroaryl, cyclyl, heterocyclyl or acyl, each of which can be optionally substituted; and
m is 0, 1 or 2.
60. The method of paragraph 58 or 59, wherein compound of Formula IX has the structure shown in Formula IX (A):

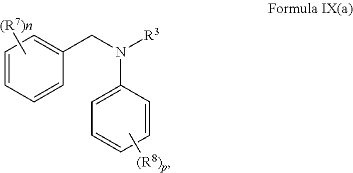

wherein:
R³ is H, C₁-C₆ alkyl, aryl, heteroaryl, cyclyl, heterocyclyl, arylalkyl, heteroarylalkyl, or a nitrogen protecting group, each of which can be optionally substituted;
each R² and R⁸ is independently halo, —CN, —NO₂, C₁-C₆ alkyl, C₂-C₆ alkenyl, C₁-C₆ alkynyl,haloC₁-C₆alkyl, —CO₂R⁶, —OR⁶, —N(R⁶)₂, each of which can be optionally substituted;
n is 0, 1, 2, 3, 4 or 5; and
p is 0, 1, 2, 3, 4 or 5.
61. The method of any of paragraphs 21, 25, 26, 54, 55, and 58-60, wherein the isolated differentiated cell is contacted with HBDA, wherein HBDA has the following structure:

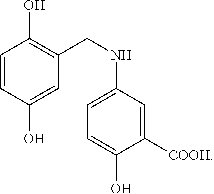

62. The method of paragraphs 22 or 38, wherein the isolated differentiated cell is contacted with an agonist of ATP-dependent potassium channels.
63. The method of paragraphs 22 or 38, wherein the isolated differentiated cell is contacted with a sodium channel inhibitor.
64. The method of paragraphs 22 or 38, wherein the isolated differentiated cell is contacted with a MAPK agonist.
65. The method of paragraphs 22, 38, 62 or 63, wherein the isolated differentiated cell is contacted with a compound of Formula X, wherein Formula X is

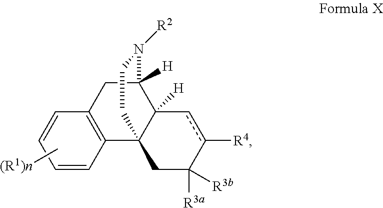

wherein:
each R¹ is independently C₁-C₆ alkyl, C₁-C₆ haloalkyl, —COOR⁵, —OR⁵, —NR⁵₂, —NO₂ or —CN;
R² is hydrogen, C₁-C₆ alkyl, aryl, heteroaryl, cyclyl, heterocyclyl, arylalkyl, or a nitrogen protecting group, each of which is optionally substituted;
Each R^{3a} and R^{3b} is independently hydrogen, —COOR⁵ or —OR⁵, or R^{3a} and R^{3b} taken together with the carbon to which they are attached form a carbonyl;
R⁴ is hydrogen, C₁-C₆ alkyl, C₁-C₆ haloalkyl, —COOR⁵, —OR⁵, —NR⁵₂, —NO₂ or —CN;
Each R⁵ is independently hydrogen, C₁-C₆ alkyl, aryl, heteroaryl, cyclyl, heterocyclyl or acyl;
n is 0, 1, 2, 3 or 4; and
the dashed line ( - - - ) indicates the presence or absence of a bond.
66. The method of any of paragraphs 22, 38, 62, 63 or 65, wherein the isolated differentiated cell is contacted with a sinomenine compound, wherein sinomenine has the following structure:

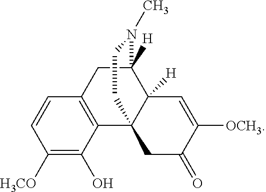

67. The method of any of paragraphs 22 or 38, wherein the isolated differentiated cell is contacted with a MAPK agonist.
68. The method of any of paragraphs 22, 38, 64 or 67, wherein the MAPK agonist is selected from any compound of Formula XI, wherein Formula XI is

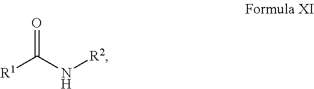

wherein:
R¹ is cyclyl, heterocyclcyl, aryl or heteroaryl, each of which can be optionally substituted; and R² is cyclyl, heterocyclcyl, aryl or heteroaryl, each of which can be optionally substituted.
69. The method of any of paragraphs 22, 38, 64, 67 or 68, wherein the isolated differentiated cell is contacted with Ropivocaine or Bupivocaine, or Ropivocaine and Bupivocaine, wherein Ropivocaine has the structure:

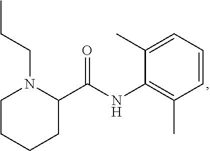

and Bupivocane has the structure:

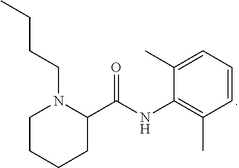

70. The method of any of paragraphs 1-69, further comprising contacting the differentiated cell with an histone deacetylase (HDAC) inhibitor or with a DNA methyltransferase inhibitor.
71. The method of any of paragraphs 1-69, wherein the differentiated cell is not contacted with a histone deacetylase (HDAC) inhibitor or with a DNA methyltransferase inhibitor.
72. The method of any of paragraphs 1-69 and 71, wherein the differentiated cell is not contacted with VPA.
73. The method of any of paragraphs 1-72, wherein the differentiated cell is reprogrammed to a pluripotent state.
74. The method of any of paragraphs 1-73, wherein the differentiated cell is reprogrammed to a multipotent state.
75. The method of any of paragraphs 1-74, wherein the reprogrammed cell has self-renewal capacity.
76. The method of any of paragraphs 1-75, wherein the expression of a marker selected from the group consisting of alkaline phosphatase, NANONG, OCT-4, SOX-2, SSEA4, TRA-1-60 and TRA-1-81 is increased by a statistically significant amount in the reprogrammed cell relative to the differentiated cell.
77. The method of any of paragraphs 1-76, wherein the differentiated cell is a mammalian somatic cell.
78. The method of any of paragraphs 1-77, wherein the differentiated cell is selected from the group consisting of a fibroblast, a muscle cell (e.g., a myocyte), a cumulus cell, a neural cell, a liver cell, a GI tract cell, a mammary cell, a kidney cell, a blood cell, a vascular cell, a skin cell, an immune system cell (e.g., a lymphocyte), a lung cell, a bone cell, or a pancreatic islet cell.
79. The method of any of paragraphs 1-78, further comprising differentiating the reprogrammed cell whereby the expression of a marker selected from the group consisting of AFP, MF20 and TUJI, is increased by a statistically significant amount in the differentiated cell relative to the reprogrammed cell.
80. The method of any of paragraphs 1-79, further comprising administering the reprogrammed cell a subject.
81. The method of any of paragraphs 1-80, wherein a plurality of differentiated cells are reprogrammed into to a plurality of reprogrammed cells.
82. The method of paragraph 81, further comprising isolating a population of reprogrammed cells.
83. The method of any of claim 1 to 17, 21 or 22, wherein the isolated differentiated cell is not contacted with an exogenous Klf-4 transcription factor or an exogenous Oct-4 transcription factor, or not contacted with both with an exogenous Klf-4 transcription factor or an exogenous Oct-4 transcription factor.
84. The method of paragraph 83, wherein the isolated differentiated cell is not contacted with any exogenous transcription factor selected from the group consisting of: Oct-4, Klf-4, c-Myc, lin-28 and Nanog.
85. The method of paragraphs 83 or 84, wherein the isolated differentiated cell is contacted with at least one compound selected from the group comprising; an agonist of Mek/Erk signaling pathway, an inhibitor of Ca²⁺/Calmodulin signaling pathway, an inhibitor of EGF signaling pathway; and at least one compound selected from the group comprising; an agonist of ATP-dependent-potassium channels, a sodium channel inhibitor or a MAPK agonist.
86. The method of paragraph 85, wherein the agonist of Mek/Erk signalling pathway is any compound according to any of paragraphs 56-57.
87. The method of paragraph 85, wherein the an inhibitor of Ca²⁺/Calmodulin signaling pathway is any compound according to any of paragraphs 58, 60 or 62.
88. The method of paragraph 85, wherein the an inhibitor of EGF signaling pathway is any compound according to any of paragraphs 59, 60 or 61.
89. The method of paragraph 85, wherein the agonist of ATP-dependent-potassium channels is any compound according to any of paragraphs 62, 65 or 66.
90. The method of paragraph 85, wherein the a sodium channel inhibitor is any compound according to any of paragraphs 63, 65 to 66.
91. The method of paragraph 85, wherein the MAPK agonist is any compound according to any of paragraphs 64, 67 to 69.
92. The method of any of paragraphs 1-91, wherein the contacting of an isolated differentiated cell with more than one compound can be contacting in any combination and in any order.
93. The method of any of paragraphs 1-92, wherein the contacting of an isolated differentiated cell with more than one compound can be contacting with any combination of compounds substantially simultaneously or sequentially, and in any order.
94. A reprogrammed cell produced by the method of any of claims 1-93.
95. A population of reprogrammed cells produced by the method of any of claims 1-93.
96. A reaction admixture comprising a more primitive precursor or less differentiated cell compared to the differentiated cell from which it was derived, and at least one compound selected from the group consisting of;
   a) a TGF-β Receptor Type I inhibitor, wherein the TGF-β Receptor Type I inhibitor substitutes for exogenously Sox2 transcription factor, and wherein exogenous Sox2 transcription factor is not present; (e.g. RepSox, or SB43142, or E-616451)
   b) an inhibitor of Src signaling pathway, wherein the inhibitor of Src signaling pathway substitutes for exogenously Sox2 transcription factor, and wherein exogenous Sox2 transcription factor is not present; (e.g. EI-275)
   c) an agonist of the Mek/Erk signaling pathway, wherein agonist of the Mek/Erk signaling pathway substitutes for exogenously Klf-4 transcription factor, and wherein exogenous Klf-4 transcription factor is not present; (e.g. PGJ₂)
   d) an inhibitor of Ca²⁺/calmodulin, wherein the inhibitor of Ca²⁺/calmodulin signaling pathway substitutes for exogenously Klf-4 transcription factor, and wherein exogenous Klf-4 transcription factor is not present; (e.g. HBDA)
   e) an inhibitor of EGF signaling, wherein the inhibitor of EGF signaling pathway substitutes for exogenously Klf-4 transcription factor, and wherein exogenous Klf-4 transcription factor is not present; (e.g. HBDA)
   j) an agonist of ATP-dependent potassium channels, wherein the agonist of ATP-dependent potassium channels substitutes for exogenously Oct-4 transcription factor, and wherein exogenous Oct-4 transcription factor is not present; (e.g. Simomenine)
   g) a sodium channel inhibitor, wherein the inhibitor of sodium channels substitutes for exogenously Oct-4 transcription factor, and wherein exogenous Oct-4 transcription factor is not present; (e.g. Simomenine)
   h) an MAPK agonist, wherein the MAPK agonist substitutes for exogenously Oct-4 transcription factor, and wherein exogenous Oct-4 transcription factor is not present; (e.g. Ropivocaine or Bupivicanine).
97. The reaction admixture of paragraph 96, wherein the TGF-β Receptor Type I inhibitor is selected from any compound of Formula I or III.
98. The reaction admixture of paragraphs 96 or 97, wherein a TGF-β Receptor Type I inhibitor is selected from the group consisting of; RepSox or E-616452 or SB431542.
99. The reaction admixture of any of paragraphs 96 to 98, wherein a TGF-β Receptor Type I inhibitor is

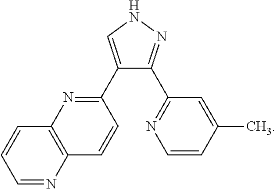

100. The reaction admixture of paragraph 96, wherein the inhibitor of Src signaling pathway is selected from any compound of Formula II.
101. The reaction admixture of paragraphs 96 or 100, wherein the inhibitor of Src signaling pathway is EI-275.
102. The reaction admixture of paragraph 96, wherein the agonist of the Mek/Erk signaling pathway is selected from any compound of Formula VIII.
103. The reaction admixture of paragraphs 96 or 102, wherein the agonist of the Mek/Erk signaling pathway is deoxy-delta¹²′¹⁴-Prostaglandin J₂ (PGJ₂).
104. The reaction admixture of paragraph 96, wherein the inhibitor of Ca2+/calmodulin signaling pathway is selected from any compound of Formula IX.
105. The reaction admixture of paragraph 96, wherein the inhibitor of EGF signalling pathway is selected from any compound of Formula IX.
106. The reaction admixture of any of paragraphs 96, 104 or 105, wherein the inhibitor of Ca2+/calmodulin signaling pathway or inhibitor of EGF signalling pathway is HBDA.
107. The reaction admixture of paragraph 96, wherein the agonist of ATP-dependent potassium channels is selected from any compound of Formula X.
108. The reaction admixture of paragraph 96, wherein the inhibitor of sodium channels is selected from any compound of Formula X.
109. The reaction admixture of any of paragraphs 96, 107 or 108, wherein the agonist of ATP-dependent potassium channels or inhibitor of sodium channels is Sinomenine.
110. The reaction admixture of paragraph 96, wherein the MAPK agonist is selected from any compound of Formula XI.
111. The reaction admixture of paragraphs 96 or 110, wherein the MAPK agonist is selected from Ropivocaine or Bupivocaine.
112. The reaction admixture of any of paragraphs 96 to 111 comprising at least two compounds selected from the group consisting of: RepSox, SB43142, E-616451, EI-275; PGJ₂, HBDA, Simomenine, Ropivocaine and Bupivicanine.
113. The reaction admixture of any of paragraphs 96 to 112 comprising at least two compounds selected from the group consisting of: RepSox, PGJ₂ and Bupivicanine.
114. The reaction admixture of any of paragraphs 96 to 113 comprising RepSox, PGJ₂ and Bupivicanine.
115. The reaction admixture of paragraphs 96 to 114, wherein the reaction admixture does not comprise one or both of a histone deacetylase (HDAC) inhibitor or a DNA methyltransferase inhibitor.
116. The reaction admixture of paragraphs 96 to 114, further comprising one or both of a histone deacetylase (HDAC) inhibitor or a DNA methyltransferase inhibitor.
117. The reaction admixture of paragraphs 96 to 116, wherein the reaction admixture does not comprise any exogenous transcription factor selected from the group consisting of: an exogenous Sox2 transcription factor, exogenous Klf-4 transcription factor or an exogenous Oct-4 transcription factor, an exogenous c-myc transcription factor, a exogenous lin-28 transcription factor, an exogenous Nanog transcription factor.
118. The reaction admixture of paragraphs 96 to 117, wherein the reaction admixture does not comprise any exogenous transcription factor selected from the group consisting of: an exogenous Sox2 transcription factor, exogenous Klf-4 transcription factor or an exogenous Oct-4 transcription factor.
119. A kit for reprogramming a differentiated cell comprising at least one at least one compound selected from the group consisting of;
   a) a TGF-β Receptor Type I inhibitor, wherein the TGF-β Receptor Type I inhibitor substitutes for exogenously Sox2 transcription factor, and wherein the kit does not comprise an exogenous Sox2 transcription factor; (e.g. RepSox, or SB43142, or E-616451)
   b) an inhibitor of Src signaling pathway, wherein the inhibitor of Src signaling pathway substitutes for exogenously Sox2 transcription factor, and wherein the kit does not comprise an exogenous Sox2 transcription factor; (e.g EI-275)
   c) an agonist of the Mek/Erk signaling pathway, wherein agonist of the Mek/Erk signaling pathway substitutes for exogenously Klf-4 transcription factor, and wherein the kit does not comprise an exogenous Klf-4 transcription factor; (e.g. PGJ₂)
   d) an inhibitor of Ca²⁺/calmodulin, wherein the inhibitor of Ca²⁺/calmodulin signaling pathway substitutes for exogenously Klf-4 transcription factor, and wherein the kit does not comprise an exogenous Klf-4 transcription factor; (e.g. HBDA)
   e) an inhibitor of EGF signaling, wherein the inhibitor of EGF signaling pathway substitutes for exogenously Klf-4 transcription factor factor, and wherein the kit does not comprise an exogenous Klf-4 transcription factor; (e.g. HBDA)
   f) an agonist of ATP-dependent potassium channels, wherein the agonist of ATP-dependent potassium channels substitutes for exogenously Oct-4 transcription factor, and wherein the kit does not comprise an exogenous Oct-4 transcription factor; (e.g. Simomenine)
   g) a sodium channel inhibitor, wherein the inhibitor of sodium channels substitutes for exogenously Oct-4 transcription factor, and wherein the kit does not comprise an exogenous Oct-4 transcription factor; (e.g. Simomenine)
   h) an MAPK agonist, wherein the MAPK agonist substitutes for exogenously Oct-4 transcription factor, and wherein the kit does not comprise an exogenous Oct-4 transcription factor; (e.g. Ropivocaine or Bupivicanine).
120. The kit of paragraph 119, wherein the kit comprises a TGF-β Receptor Type I inhibitor and an agonist of the Mek/Erk signaling pathway or an inhibitor of Ca²⁺/calmodulin or an inhibitor of EGF signaling and an agonist of ATP-dependent potassium channel or a sodium channel inhibitor or a MAPK agoinist.
121. The kit of paragraphs 119 or 120, wherein the kit comprises at least two of the following compounds selected from the group of: RepSox; SB43142, E-616451, EI-275; PGJ₂, HBDA, Simomenine, Ropivocaine and Bupivicanine.
122. The kit of any of paragraphs 119 to 121, wherein the kit comprises at least three of the following compounds selected from the group of: RepSox; SB43142, E-616451, EI-275; PGJ₂, HBDA, Simomenine, Ropivocaine and Bupivicanine.
123. The kit of any of paragraphs 119 to 122, wherein the kit comprises at least three of: RepSox; PGJ₂, HBDA, Simomenine, Ropivocaine and Bupivicanine.
124. The kit of any of paragraphs 119 to 123, wherein the kit comprises: RepSox; HBDA, and Bupivicanine.
125. The kit of any of paragraphs 119 to 124, wherein the kit further comprises one or both of a histone deacetylase (HDAC) inhibitor or a DNA methyltransferase inhibitor.
126. The kit of any of paragraphs 119 to 125, wherein the kit further comprises an antibody or fragment thereof to identify the reprogrammed cell.
127. The kit of paragraph 126, wherein the antibody identifies a marker expressed by the reprogrammed cell.
128. The kit of paragraph 127, wherein the marker is selected from the group selected from the group consisting of alkaline phosphatase, NANONG, OCT-4, SOX-2, SSEA4, TRA-1-60 and TRA-1-81.
129. The kit of any of paragraphs 119 to 128, wherein the differentiated cell is a mammalian somatic cell.
130. The kit of any of paragraphs 119 to 129, wherein the mammalian somatic cell is a human somatic cell.
131. Use of the reaction admixture of any of claims 96-118 for reprogramming a differentiated cell.
132. The use of the reaction admixture of paragraph 131, wherein the differentiated cell is a mammalian somatic cell.
133. The use of reaction admixture of paragraph 133, wherein the mammalian somatic cell is a human somatic cell.
134. Use of the kit of any of claims 119-130 for reprogramming a differentiated cell.
135. A clonal cell line produced by the method of any of claims 1-95.

### Methods

### EXAMPLES

One embodiments relates to the discovery that compounds that can specifically replace members of the Sox transcription factor family (e.g. Sox-2), members of the Klf transcription factor family (e.g. Klf4) and members of the Oct transcription factor family (e.g. Oct4) to produce reprogrammed cells from differentiated cells or partially reprogrammed cells. In particular, in one embodiment, the invention relates to the discovery that small molecules which functions by inhibiting TGF-beta signaling or inhibition of Src cell signalling to replace members of the Sox transcription factor family (e.g. Sox-2). These inhibitors, such as for example, RepSox, E-616451, SB431542 and EI-275 do not increase the efficiency of reprogramming, and therefore specifically replaces exogenous Sox-2 (e.g. nucleic acid encoding Sox2 or a polypeptide of Sox2). In another embodiment, the invention relates to the discovery of small molecules which functions by activating Mek/Erk cell signalling (e.g. Prostaglandin J2) or inhibitors of EGF cell signalling (e.g. HDBA) or inhibitors of Ca2+/Calmodulin cell signalling (e.g. HDBA) to replace members of the Klf transcription factor family (e.g. Klf4). These inhibitors, such as for example, Prostaglandin J2 and HDBA do not increase the efficiency of reprogramming, and therefore specifically replaces exogenous Klf4 (e.g. nucleic acid encoding Klf4 or a polypeptide of Klf4). In another embodiment, the invention relates to the discovery of small molecules which functions by activating ATP-dependent potassium channels (e.g. Sinimonine) or inhibitors of sodium channels (e.g. Sinimonine) or activators of MAPK cell signalling (e.g. Ripivocaine or Bupivocaine) to replace members of the Oct transcription factor family (e.g. Oct4). These inhibitors, such as for example, Sinimonine, Ripivocaine and Bupivocaine do not increase the efficiency of reprogramming, and therefore specifically replaces exogenous Oct4 (e.g. nucleic acid encoding Oct4 or a polypeptide of Oct4). Thus, the inventors demonstrate herein that small molecule modulators of cell signaling pathways can specifically replace reprogramming transgenes Oct4, Sox2, Klf4 and in some embodiments, c-Myc. Thus, in some embodiments, the small molecules disclosed herein can be used in methods for purely chemical-based reprogramming of differentiated cells.

### Materials and Methods

Derivation of MEFs and Cell Culture.

MEFs were derived from E12.5 embryos hemizygous for the Oct4::GFP transgenic allele. Gonads and internal organs were removed before processing the embryos for MEF isolation. MEFs were grown in DMEM supplemented with 10% FBS and penicillin/streptomycin. Low passage (up to passage 3) MEFs were used for generation of iPS cells.

Retroviral Infection.

Moloney-based retroviral vectors (pMXs) expressing the murine complementary DNAs of Oct4, Sox2, c-Myc, and Klf4 were obtained from Addgene. These plasmids were transfected separately into individual populations of Plat-E packaging cells using Fugene 6, with 27 μl of Fugene 6 and 9 μg of DNA per 10 cm dish of Plat-E cells. Viral supernatants were obtained 48-72 hours post-transfection, filtered through a 0.22 μm filter, diluted 1:1 in MEF growth media, and supplemented with polybrene at a final concentration of 5 μg/ml. The supernatants for the four factors were mixed in an equimolar ratio, and media was used in place of a factor when it was omitted from the infection. MEFs were infected with two to three pools of viral supernatant during a 72-hour period. The first day that viral supernatant was termed “day 1 post-infection.”

Small Molecule Screens.

On day 4 post-infection, infected MEFs were trypsinized and re-seeded on irradiated feeders in 96-well plates at 2000 cells/well and cultured in mouse ES cell media (Knockout DMEM supplemented with 15% Hyclone FBS, L-glutamine, penicillin/streptomycin, nonessential amino acids, β-mercaptoethanol, and 1000 U/ml LIF). The next day, compound stock solutions diluted in DMSO and, where applicable, VPA (Sigma), were added at a final concentration of 1 μM and 2 mM, respectively. VPA was removed after 1 week, and compound was re-applied every other day with each media change. Plates were scored for GFP+colonies after 11 of compound treatment.

Lead Compound Titrations to Determine Optimal Dosage.

Infections and VPA/compound addition was done as in the original chemical screen, and wells were scored for GFP+ colonies on day 25 after compound addition.

Derivation of Tail Tip Fibroblasts, Cell Culture, and Retroviral Infection.

Fibroblasts were isolated from tails of 8-week old Oct4::GFP and cultured in DMEM supplemented with 40% fetal bovine serum and penicillin/streptomycin. For reprogramming experiments, P2 fibroblasts were infected by the same method as described for MEFs.

Quantification of Oct4::GFP+ iPS Cells Generated Small Molecules Hits, SB-431542, and TGF-βantibodies.

Retroviral infection and compound or antibody treatment was performed as in the original chemical screen. To quantify the numbers of GFP+colonies produced in

different conditions, the number of colonies in each well was counted and at least 2 different wells were counted and averaged. Concentrations of compounds and antibodies were the following: VPA (Sigma)-2 mM, RepSox (Calbiochem)-25 μM, E-616451 (Calbiochem)-3 μM, EI-275 (Biomol)-3 SB-431542 (Sigma)-25 μM, TgfβIIspecific antibody (R&D Systems, AB-12-NA)-10 μg/ml, pan-Tgβ antibody (R&D Systems, AB-100-NA)-10 μg/ml. Unless otherwise noted, all chemical treatments were continuous from initial administration at day 4-5 post-infection until GFP+colonies were scored. Fresh chemical was added at each media change.

Generation of Reprogrammed or iPS Cells.

GFP+ P0 colonies were picked manually and incubated in 0.25% trypsin (Gibco) for 20 minutes at room temperature before plating on a feeder layer in mES cell media. This process was repeated until passage 3, at which time colonies were trypsinized and passaged in bulk and maintained on feeders in mES cell media.

Antibody Staining for Sox2 and Nanog and Alkaline Phosphatase Staining.

iPS cells were cultured on irradiated MEF feeders in chamber slides, fixed with 4% PFA and stained with primary antibodies against mSox2 (Santa Cruz, sc-17320), mNanog (CosmoBio, REC-RCAB0002 PF), followed by staining with the appropriate secondary antibodies conjugated to Alexa Fluor 546 (Invitrogen). Nuclei were counterstained with Hoechst33342 (Sigma). iPS cells were assayed for alkaline phosphatase activity using the Vector Red alkaline phosphatase assay kit from Vector Laboratories.

Whole-Genome Expression Analysis.

Cells were grown to near confluence on an irradiated layer and RNA was isolated with Trizol (Invitrogen). RNA was amplified and labeled with biotin using the Illumina Total Prep RNA Amplification Kit from Ambion, hybridized to Illumina Whole-Genome Expression BeadChips (MouseRef-8), and analyzed by an Illumina Beadstation 500. All lines were analyzed in biological duplicate or triplicate. Data were processed using Resolver software.

Spontaneous Differentiation of iPS Cells In Vitro.

iPS cells were grown to 70-80% confluence in 10-cm plates (Falcon) in mES cell medium. To form embryoid bodies, cells were washed once with PBS to eliminate mES cell medium and then incubated with 1 ml of 0.25% trypsin (GIBCO) for 5-10 min at room temperature (21-25° C.). Cells were then resuspended in 10 ml of DM1 medium (DMEM-F12, GIBCO), 10% knockout serum (GIBCO), penicillin, streptomycin, glutamine (GIBCO) and 2-mercaptoethanol (GIBCO), counted, and plated at a concentration of 200,000 cells per ml in Petri dishes (Falcon). Two days later, embryoid bodies were split from one dish into four Petri dishes containing DM1 medium and the medium was changed after 3-4 d. On day 10 the embryoid bodies were collected in a 15-ml Falcon tube, washed once with PBS and then fixed in PFA 4% at 4 C for 1 hour. The EBs were then washed 4 times in PBS to remove the residual PFA and incubated overnight in a solution of 30% of sucrose. The next day, the cells were embedded in OCT and frozen at −80C. The block containing EBs were then sectioned with a cryostat into 10 μm sections. The sections were stained with primary antibodies against Alphafetoprotein (AFP)(Dakocytomation, A0008), Skeletal Myosin (MF20) (Developmental Studies Hybridoma Bank, MF20), or Beta-III-tubulin (TUJ1)(Sigma, T2200), and visualized by staining with a secondary antibody conjugated to Alexa Fluor 546 (Invitrogen).

Directed Differentiation of iPS Cells into Motor Neurons.

iPS and mES (V6.5) cells were differentiated into motor neurons according to methods previously described for mouse ES cells differentiation [27]. The iPS and mES cells were grown to 70-80% confluence in 10-cm plates (Falcon) in mES cell medium. To form embryoid bodies, cells were washed once with PBS to eliminate mES cell medium and then incubated with 1 ml of 0.25% trypsin (GIBCO) for 5-10 min at room temperature (21-25° C.). Cells were then resuspended in 10 ml of DM1 medium (DMEM-F12, GIBCO), 10% knockout serum (GIBCO), penicillin, streptomycin, glutamine (GIBCO) and 2-mercaptoethanol (GIBCO), counted and plated at a concentration of 200,000 cells per ml in Petri dishes (Falcon). Two days later, embryoid bodies were split from one dish into four Petri dishes containing DM1 medium supplemented with RAc (100 nM; stock: 1 mM in DMSO, Sigma) and Shh (300 nM, R&D Systems). Medium was changed after 3-4 d. On day 7, the embryoid bodies were dissociated into single-cell suspensions. The suspensions were pelleted in a 15-ml Falcon tube, washed once with PBS, and incubated in Earle's balanced salt solution with 20 units of papain and 1,000 units of DNase I (Worthington Biochemical) for 30-60 min at 37° C. The mixture was then triturated with a 10-ml pipette and centrifuged for 5 min at 300×g. The resulting cell pellet was washed with PBS and resuspended in F12 medium (F12 medium, GIBCO) with 5% horse serum (GIBCO), B-27 supplement (GIBCO), N2 supplement (GIBCO) with neurotrophic factors (GDNF and BDNF, 10 ng ml-1, R&D Systems). The cells were counted and plated on poly-D-lysine/laminin culture slides (BD Biosciences) or on a layer of primary glial cells. 3-5 days later, the cultures were fixed with PFA and stained with primary antibodies against TUJ1 (Sigma, T2200) and HB9 (Developmental Studies Hybridoma Bank, 81.5C10), and visualized by staining with secondary antibodies conjugated to Alexa Fluor 488 and Alexa Fluor 546 (Invitrogen). For counting HB9+cells, motor neurons were differentiated as above except in embryoid body culture without dissociation and plating. Embryoid bodies were sectioned as above and stained with the TUJ1 and HB9 antibodies along with the Alexa Fluor 488 and Alexa Fluor 546 secondary antibodies. Cultures were counterstained with Hoechst 33342 and HB9+ and total nuclei were counted. Numbers were derived from at least 3 different embryoid bodies per cell line.

Teratoma Production and Analysis.

A confluent 10 cm dish of iPS cells was trypsinized, pelleted, resuspended in 0.2 mls of mES media, and injected subcutaneously into a CD1-Nude mouse. 3-4 weeks later, teratomas were harvested, fixed overnight with 4% paraformaldehyde, embedded in paraffin, sectioned, HE stained, and analyzed.

Production of Chimeric Mice.

Female ICR mice were superovulated with PMS and hCG and mated to ICR stud males. 24-hours after hCG injection, zygotes were isolated from vaginally plugged females. After culture in KSOM media for 3 days, the resulting blastocysts were injected with ˜5-10 iPS cells from a C57BL6 background pre-labeled with a lentivirus constitutively expressing the red fluorescent protein tdTomato and transferred into pseudopregnant females. Embryos were either harvested at day E13.5 or allowed to develop to term. Chimeric embryos were visualized on a Leica MZ16FA dissecting microscope using RFP and bright field channels. For 8-cell stage injections, zygotes were developed in vitro to the 8-cell stage, injected with iPS cells, further developed in vitro to the blastocyst stage, and visualized.

Chemical Reprogramming of Stable Intermediate Cell Lines.

Oct4::GFP-negative colonies in Oct4, Klf4, and cMyc or Oct4, Klf4, cMyc, and Sox2-infected MEF cultures were picked, plated on irradiated feeders, and single colonies were picked after 1 week. The resulting cell lines were passaged with trypsin and grown in mES media on feeders until passage 4, at which time they were treated with RepSox (25 μM), AZA (500 μM), or both for 48 hours. Oct4::GFP+colonies were scored 12 days after the beginning of chemical treatment.

Cell Proliferation Assays.

Cells were plated at a density of 5000 cells per well of a 6-well dish on gelatin in mES media. At the designated time points, cells were trypsinized and counted.

shRNA-Mediated Knockdown of Nanog and Sox2

OKM 10 cells or MEFs transduced 4 days earlier with Oct4, Klf4, cMyc, and Sox2 were transduced with shRNA constructs in the lentiviral vector pLK0.1 that were specific to murine Nanog (5′-CCGGCCTGAGCTATAAGCAGGTTAACTCGAGTTAACCTGCTTATAGCTCAGGTTTTTG) (SEQ ID NO: 12) or Sox2 (5′-CCGGCGAGATAAACATGGCAATCAACTCGAGTTGATTGCCATGTTTATCTCGTTTTTG) (SEQ ID NO: 13) (Open Biosystems). Lentiviruses were packed by co-transfection of pLKO.1-shRNA plasmids with VSVG envelope and delta 8.9 plasmids into 293T cells using Fugene 6. Starting two days after infection, the population was enriched for transduced cells by selection with 4 μg/mlpuromycin for three days. For OKM 10 cells, RepSox treatment was initiated after puromycin selection.

Reprogramming of Stable Intermediate Cell Lines by Viral Transduction.

Oct4::GFP-negative cell lines were transduced using the same methodology and reagents as MEFs were in the original screen. Cells were infected with three rounds of viral supernatant diluted 1:8 in MEF media in a 48-hour period on gelatin. Two days after the last viral supernatant was added, the cells were trypsinized and replated onto feeders. The media was changed to mES media containing knockout serum replacement (KSR) instead of FBS on the following day.

Reprogramming of MEFs Using Nanog

MEFs were infected as described for the original screen, except that murine Nanog cDNA was cloned into the pMXs retroviral vector and used instead of pMXs-Sox2. Two days after the last viral supernatant was added, the cells were trypsinized and replated onto feeders. The media was changed to mES media containing knockout serum replacement (KSR) instead of FBS on the following day.

### Example 1

A Screen for Chemical Mediators of Reprogramming. In order to identify small molecules that function in reprogramming, the inventors transduced fibroblasts with viral vectors encoding Oct4, Klf4, and cMyc and then screened for compounds that allowed reprogramming to proceed in the absence of Sox2. This approach prevented bias with respect to the mechanism by which a given chemical functioned. In this sense, the approach used by the inventors is similar to a chemical genetic screen that would not only deliver chemical compounds with translational utility, but is useful to provide novel insights into the pathways and mechanisms controlling reprogramming.

Activation of an Oct4::GFP reporter gene and formation of colonies with an ES cell morphology has previously been demonstrated to be a stringent assay for reprogramming (23). Furthermore, it has been shown that supplementing the culture medium with VPA can improve reprogramming efficiency (24). In mES culture medium supplemented with VPA, retroviral transduction of 7500 Oct4::GFP transgenic mouse embryonic fibroblasts (MEFs) with Oct4, Klf4, cMyc, and Sox2 (25) routinely generated 100-200 GFP+colonies (FIG. 1A). The inventors expected that omission of one of the critical reprogramming factors, such as Sox2, would significantly reduce or even eliminate the appearance of these colonies. Indeed, no GFP+colonies were observed when Sox2 was omitted from the same transduction (Oct4::GFP+colonies form readily in Oct4, Klf4, cMyc, and Sox2-infected MEF cultures, but do not form in Oct4, Klf4, and cMyc-infected MEF cultures. Data not shown). The inventors used this robust difference in the number of colonies expressing GFP to identify small molecules that can replace Sox2 in reprogramming.

To facilitate the identification of cellular targets and signaling pathways that were affected by the compounds which were discovered the inventors, the inventors screened a library of small molecules that had known pharmacological targets, including kinases, ion channels, and extracellular receptors. Transduced Oct4::GFP MEFs were screened with Oct4, Klf4, and cMyc, and then plated 2000 cells per well in 96-well format. To each well one of 200 distinct compounds was added for 7 or 11 days, treating with 2 mM VPA for the first 7 days (FIG. 1A). The inventors expected this approach would identify both compounds that required widespread chromatin remodeling to induce reprogramming (24) and compounds that did not. After 16 days, the inventors scored each well for the presence of GFP+colonies with a mES-like morphology (FIG. 1B) and identified 3 independent compounds that induced GFP+colonies (FIG. 1C). Two of these compounds were distinct Transforming Growth Factor-13 Receptor 1 (Tgfβr1) kinase inhibitors (E-616452 and E-616451 (FIG. 1D) (26)), while the third was a Src-family kinase inhibitor (EI-275 (FIG. 1D) (27)).

### Example 2

### Efficient Small Molecule Replacement of Sox2

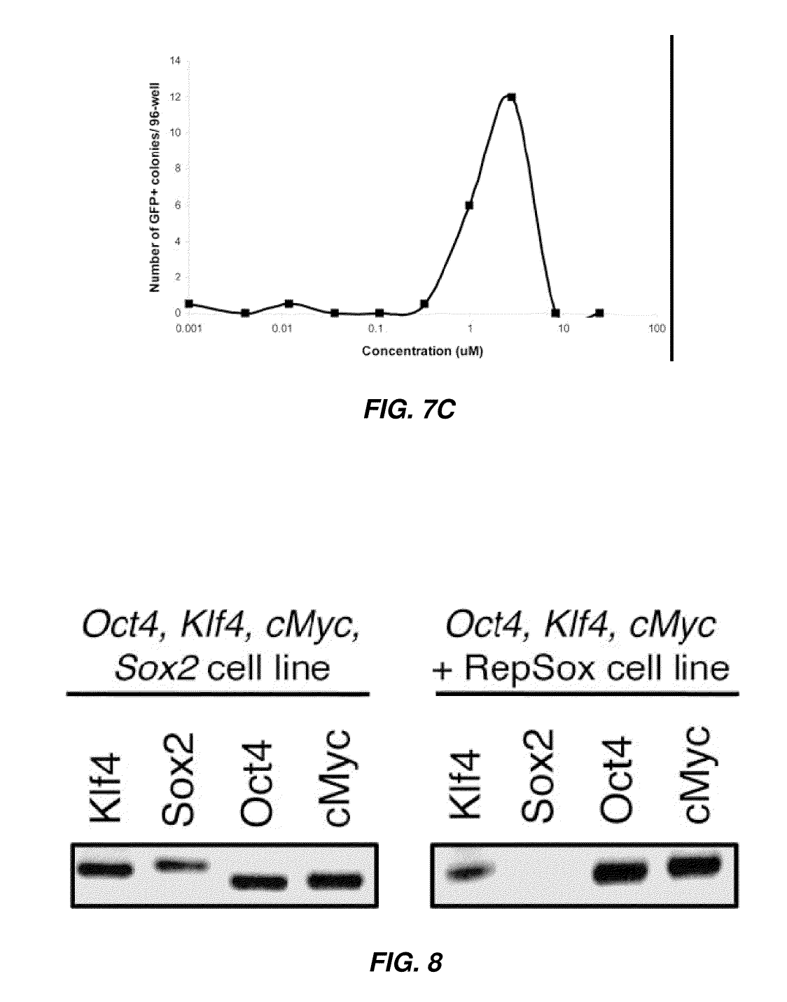

Next, the inventors optimized the effective concentration for each molecule (FIG. 7A-7C) and quantified the efficiency at which each of the hit compounds synergized with VPA to replace Sox2. When 1500 MEFs were transduced with only Oct4, Klf4, and cMyc and then treated with VPA, no GFP+colonies were observed (FIG. 1E). However, the addition of E-616452 (Repsox) (25 μM), E-616451 (3 μM), or EI-275 (3 μM), led to the formation of GFP+colonies with an ES cell morphology at a rate that was comparable to normal retroviral transduction with Sox2 (FIG. 1E).

Since the three compounds were identified in the presence of VPA, the inventors next determined whether these molecules were dependent on this HDAC inhibitor for their reprogramming activities. The inventors determined that E-616451 and EI-275 could not induce the appearance of GFP+colonies in the absence of VPA (FIG. 1E), while E-616452 could do so and at a rate that was similar to a positive control transduced with the Sox2 retrovirus (FIG. 1E).

Although cMyc does increase the efficiency of reprogramming, it is not required for the generation of iPS cells (6). Since the elimination of cMyc from the reprogramming cocktail is an important step towards reducing the risk of tumor formation, the inventors tested whether E-616452 (Repsox) could function in the absence of this oncogene. When added to MEFs transduced with only Oct4 and Klf4, E-616452 (Repsox) induced the formation of GFP+colonies at a high efficiency that was similar to the positive control transduced with Sox2 (FIG. 1F). Thus, the Repsox chemical can replace the critical reprogramming factor Sox2 without compromising reprogramming efficiency.

Previous reports on small molecules that affect reprogramming have focused on MEFs or neural stem cells (NSCs). These cells may be reprogrammed more easily due to either their proliferative capacity or ongoing expression of iPS factors from their endogenous loci (19, 24, 28, 29). However, it may be that chemical modulation of gene expression is cell-type specific. This, the inventors determined if the reprogramming compound Repsox functioned in a more patient-relevant cell type. When adult tail tip fibroblasts were infected with Oct4, Klf4, and cMyc alone, no Oct4::GFP+colonies were detected. However, when Oct4, Klf4, and cMyc-transduced fibroblasts were treated with E-616452 (Repsox), the inventors demonstrate significant production of Oct4::GFP+colonies (an Oct4::GFP+iPS line that was derived from a culture of RepSox treated Oct4, Klf4, and cMyc-infected MEFs (OKM+RepSox line 1) displays the characteristic mES-like morphology and self-renewal properties. Data not shown). The Oct4::GFP+colonies could be picked, and the resulting cell lines maintained homogenous Oct4::GFP expression and self-renewed similarly to mES and 4-factor iPS control lines (data not shown). Thus the inventors have demonstrated that E-616452 (Repsox) can replace Sox2 in the reprogramming of both mouse embryonic and adult fibroblasts. Because E-616452 (Repsox) could efficiently replace transgenic Sox2, even in the absence of VPA and cMyc, as well as in both embryonic and adult fibroblasts, the inventors chose to further characterize E-616452 and named it “RepSox”, for Replacement of Sox2.

### Example 3

### RepSox-reprogrammed cells are iPS cells

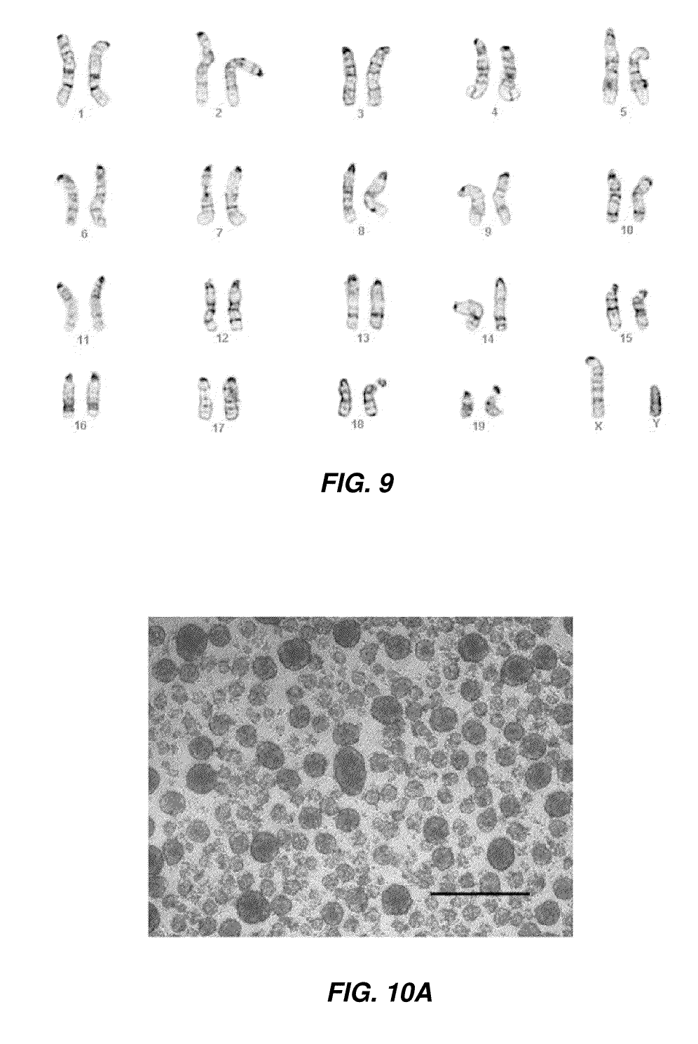

Investigation of self-renewal capacity, gene expression program, and pluripotency demonstrated that Oct4::GFP+ cells induced by the RepSox replacement of Sox2 were bona fide iPS cells. The inventors demonstrated that a RepSox-reprogrammed cell line self-renewed for more than 10 passages with a growth rate similar to that of mES cells, while maintaining a mES cell-like morphology and expression of the Oct4::GFP transgene (An Oct4::GFP+iPS line that was derived from a culture of RepSox treated Oct4, Klf4, and cMyc-infected MEFs (OKM+RepSox line 1) displays the characteristic mES-like morphology and self-renewal properties—data not shown). PCR with primers specific to the Oct4, Klf4, cMyc, and Sox2 transgenes confirmed that this cell line did not harbor transgenic Sox2 (FIG. 8). Chromosomal analysis indicated it was karyotypically normal (FIG. 9). Antibody staining revealed that the Oct4::GFP positive cells co-expressed the endogenous alleles of the Nanog and Sox2 genes, demonstrating a pluripotent transcriptional program had been established (Antibody staining of OKM+RepSox line 1 cells shows that these cells express markers of pluripotent stem cells Sox2 and Nanog—data not shown). In addition, all cell lines expressed the enzymatic activity alkaline phosphatase, as do mouse ES cells and iPS cells (FIG. 30A).

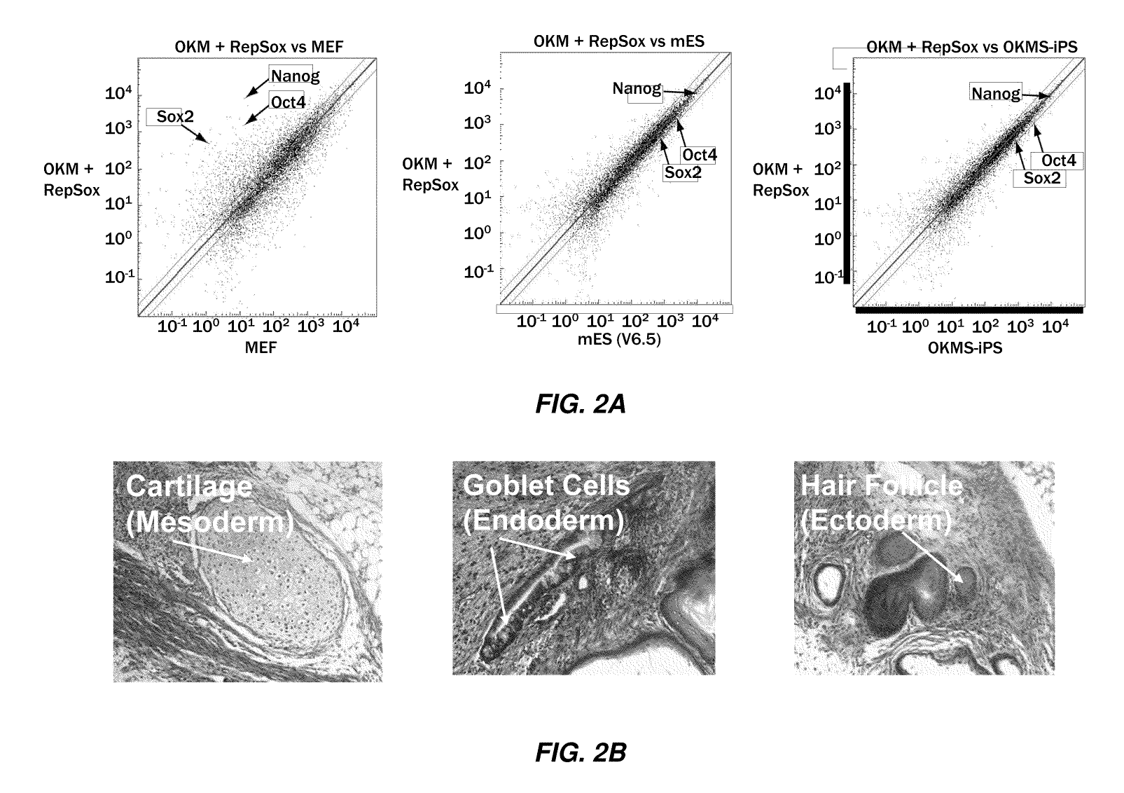

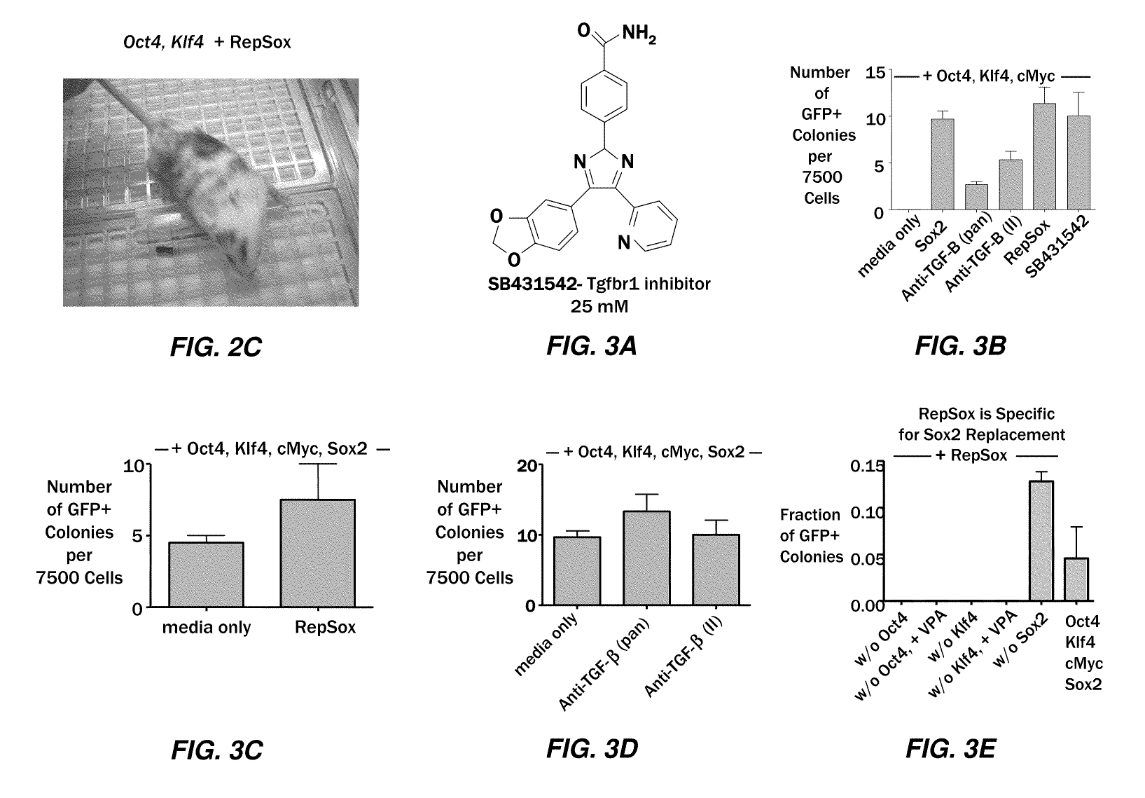

The global transcriptional profile of a cell line reprogrammed with Oct4, Klf4, cMyc, and RepSox was similar to those observed in a mouse ES cell line and an iPS cell line produced with all four transgenes, while it differed significantly from that of the somatic MEFs (FIG. 2A). Pearson correlation coefficient analysis confirmed that iPS cells produced with RepSox were as similar to mouse ES cells (Pearson correlation coefficient=0.95-0.97) as two mouse ES cell lines were to each other (Pearson correlation coefficient=0.96) (Table 1).

TABLE 1
Pearson correlation coefficients between mES cell lines, Oct4, Klf4,
cMyc, and Sox2 iPS line 1 (OKMS − iPS), Oct4, Klf4, and cMyc +
RepSox iPS line 1 (OKM + RepSox), and Oct4::GFP MEFs (MEF).
Pearson (R)

| | mES1 | mES2 | OKMS − | OKM + | |
|---|---|---|---|---|---|
| | (R1) | (V6.5) | iPS | RepSox | MEF |
| mES1 (R1) | 1.00 | 0.96 | 0.98 | 0.96 | 0.80 |
| mES2 (V6.5) | | 1.00 | 0.99 | 0.97 | 0.81 |
| OKMS − iPS | | | 1.00 | 0.97 | 0.82 |
| OKM + RepSox | | | | 1.00 | 0.79 |
| MEF | | | | | 1.00 |

In order to demonstrate that the chemically reprogrammed cell lines share the pluripotent characteristics of mES and iPS cells, the inventors tested their ability to spontaneously differentiate in vitro into cells types derived from the three embryonic germ layers. Cells produced with Oct4, Klf4, and cMyc and RepSox readily formed embryoid bodies (FIG. 10A-10B) and differentiated into cells that expressed markers found in the endodermal (Alpha-fetoprotein, AFP), the mesodermal (Skeletal Myosin, MF20), and the ectodermal lineages (Beta-III-tubulin, TUJ1). The inventors determined that spontaneously differentiated OKM+RepSox line 1 cells and OK

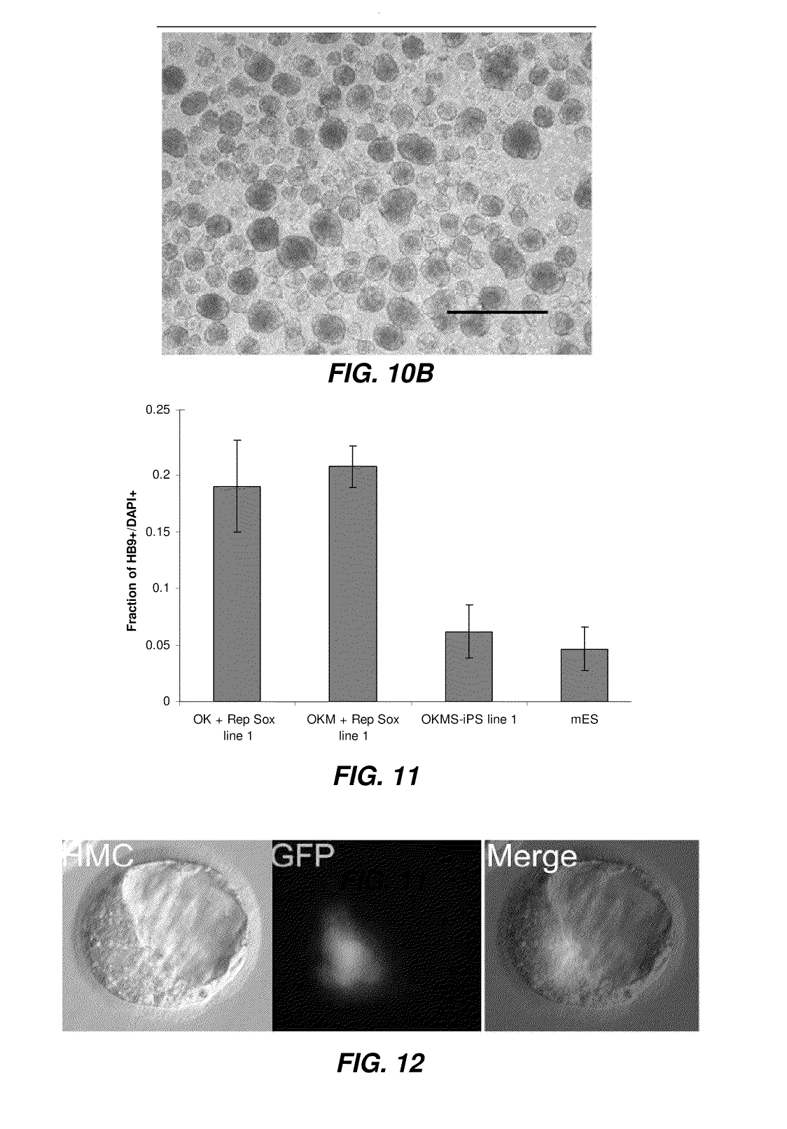

+RepSox line 1 cells express neuronal marker TUJ1 (Beta III tubulin, ectoderm), MF20 (Myosin heavy chain, mesoderm), and AFP (Alpha fetoprotein, endoderm) (data not shown). Oct4::GFP+ areas of the EBs were discovered to be undifferentiated and do not overlap with the TUJ1+, MF20+, or AFP+ regions—data not shown). In addition, the inventors discovered that cells reprogrammed with RepSox could respond to directed differentiation signals in vitro and could be robustly differentiated into HB9+/TUJ1+motor neurons. In particular, the OK+RepSox line 1 was discovered to express HB9 and TUJI (data not shown). Quantification of this differentiation revealed that cells reprogrammed with RepSox formed motor neurons at a 20% efficiency, which was similar to differentiating mES cells and control iPS cell lines (FIG. 11) (30, 31).

A cell line reprogrammed with RepSox was also injected into nude mice to asses its capacity to form teratomas. The inventors demonstrated the that teratomas were readily formed that contained cell types from each of the three embryonic germ layers (FIG. 2B).

In order to definitively confirm the pluripotency of cells reprogrammed with RepSox, the inventors also tested their ability to contribute to chimeric embryos in vivo. The inventors labeled iPS cell lines produced with RepSox using a lentiviral transgene encoding the red fluorescent Tomato-protein and injected them into blastocysts. These injections resulted in chimeric embryos and adult mice with significant contribution from the iPS cells (FIG. 2C). A E12.5 chimeric mouse embryo showed a high amount of contribution from OKM+RepSox line 1 cells, and constitutively expressed the dTomato red fluorescent protein (data not shown). The inventors also demonstrated that iPS cells produced using RepSox could contribute Oct4::GFP+ cells to the genital ridges of embryonic chimeras, demonstrating contribution of these pluripotent cells to the germ-line (OKM+RepSox cell line-derived Oct4::GFP+germline cells are present in the genital ridge of a male embryo at 13.5 d.p.c (data not shown)). In addition, cells reprogrammed with RepSox that were injected into 8-cell stage embryos appropriately migrated to the inner cell mass at the blastocyst stage (FIG. 12). Together, these results demonstrate that the RepSox-reprogrammed cells are indeed pluripotent cells (e.g. iPS cells).

### Example 4

### RepSox Specifically Replaces Sox2 by Inhibiting TGF-β Signaling

Previous studies with a compound similar to RepSox suggest that it can act as an inhibitor of the Transforming Growth Factor Receptor type I (Tgfbr1) kinase (26). Therefore, the inventors investigated whether the mechanism by which RepSox functions to replace Sox2 is through the inhibition of Tgf-β signaling. If Tgfbr1 is the functional target of RepSox, then a structurally unrelated inhibitor of Tgf-β signaling might also replace Sox2 in reprogramming experiments. The small molecule SB-431542 (FIG. 3A) is also known to inhibit Tgfbr1 kinase and is structurally distinct from RepSox (32). When the inventors treated fibroblasts transduced with Oct4, Klf4, and cMyc with 25 μM SB-431542, −10 GFP+colonies were observed per 7500 cells plated (FIG. 3B), while in contrast, no GFP+colonies were observed in transductions without SB-431542. Thus, two distinct small molecule inhibitors of Tgf-β signaling, such as Repsox (E-616452) and SB-431542 can substitute for Sox2.

If RepSox functions by inhibiting Tgf-β signaling, then removal of functional Tgf-β ligands from the culture medium might also permit the omission of transgenic Sox2 from the reprogramming cocktail. To test this idea, the inventors transduced MEFs with Oct4, Klf4, and cMyc, and then cultured them with either an antibody that binds to a variety of Tgf-β ligands (R&D Systems, AB-100-NA) (e.g. a pan specific TGFβ neutralizing antibody) or an antibody specific to Tgf-β II (R&D Systems, AB-12-NA). The inventors demonstrated that both inhibiting antibodies induced the generation of Oct4::GFP+colonies in the absence of exogenous Sox2, at a rate that was slightly lower but still comparable to RepSox and compound SB-431542 (FIG. 3B). Thus, the inventors have demonstrated that one mechanism by which RepSox replaces Sox2 in reprogramming is through the inhibition of Tgf-β signaling

The inventors specifically sought to identify molecules that specifically replace Sox2 instead of generally increasing reprogramming efficiency, so that such compounds will effectively synergize with molecules that replace the remaining transgenic factors, such as Oct4 and Klf4. If RepSox acts specifically to replaces Sox2, it would not be expect it to stimulate reprogramming in the presence transgenic Sox2. When RepSox or Tgf-β antibody-treated MEFs were transduced with Oct4, Klf4, cMyc and Sox2, the inventors demonstrated less than a 2-fold increase in the number of GFP+colonies over the untreated control (FIGS. 3C, D). The magnitude by which RepSox stimulated reprogramming in this context was significantly less than the greater than 10-fold increase that was detected following treatment with VPA, a compound known to increase reprogramming efficiency (FIG. 1E). The inventors observed a small induction with RepSox treatment, which is likely consistent with the promotion of reprogramming in a subset of cells with insufficient levels of transgenic Sox2 expression. Thus the inventors demonstrate that RepSox does not dramatically increase the efficiency of reprogramming with all four transgenic factors and demonstrate that the mechanism of Sox2 replacement is more specific in nature.

In order to further investigate the specificity of Sox2 replacement by RepSox, the inventors tested the ability of this molecule to individually replace Oct4, Klf4, and cMyc in reprogramming. The inventors determined that RepSox could not induce GFP+colonies in the absence of either Oct4 or Klf4, even in the presence of VPA (FIG. 3E). In contrast, the inventors demonstrated that RepSox did increase the number of Oct4::GFP+colonies by 20-fold in the absence of cMyc, thereby fully replacing c-Myc in reprogramming (FIG. 3F). In addition, the structurally distinct Tgf-β inhibitor SB431542 and a Tgf-β-specific neutralizing antibody also both increased reprogramming efficiency in the absence of cMyc (FIG. 3G). Interestingly, while a concentration of 25 μM RepSox was necessary for optimal Sox2-replacement (FIG. 7A), it was discovered that 1 μM RepSox was as effective at replacing cMyc (FIG. 3G), demonstrating that a more complete inhibition of Tgf-β signaling may be required for Sox2 replacement. Indeed, small molecule inhibitors of Tgf-β signaling with IC₅₀ measurements similar to that of RepSox, such as SB431542, require concentrations greater than 1 μM for complete Tgf-β inhibition in cellular growth assays (32). From these experiments, the inventors have demonstrated that RepSox specifically enables the replacement of the reprogramming activities provided by transgenic Sox2 and that it can also compensate for the omission of cMyc. In both cases, replacement of exogenous Sox2 or c-Myc transcription factors in the reprogramming activities of Repsox (or SB431542) is mediated through the inhibition of Tgf-β signaling.

### Example 5

### RepSox Acts on Intermediate Cell Types Formed During the Reprogramming Process to Replace Sox2

The development of cocktails of small molecules that can effectively reprogram differentiated (e.g. somatic) cells may be able to replace all exogenous transcription factors and produce reprogrammed cells from differentiated cells.

Therefore, for Repsox to be used in cocktails of small molecules for chemical based reprogramming of differentiated cells, the inventors determined the optimal and minimal durations of time for which inhibition of Tgf-β signaling using RepSox could induce reprogramming in the absence of Sox2.

In order to precisely determine the time point in reprogramming at which RepSox treatment was most effective, the inventors varied the time of RepSox administration. Initially, the inventors pretreated MEFs with RepSox, applying the chemical for three days and then removing it at the time of transduction with Oct4, Klf4, and cMyc. In these experiments, no Oct4::GFP+colonies were formed (FIG. 4A), demonstrating that RepSox does not act on the initial differentiated (e.g. somatic) cells to replace Sox2. Consistent with this result, the inventors did not detect a significant increase in expression of endogenous Sox2 or closely related Sox family members in MEFs upon RepSox treatment (data not shown). In addition, RepSox treatment of MEFs did not decrease the expression of the mesenchymal gene Snail (data not shown), which is downregulated by 5-40-fold by transduction of the 4 reprogramming factors (21). Thus RepSox does not destabilize the pre-existing MEF transcriptional program.

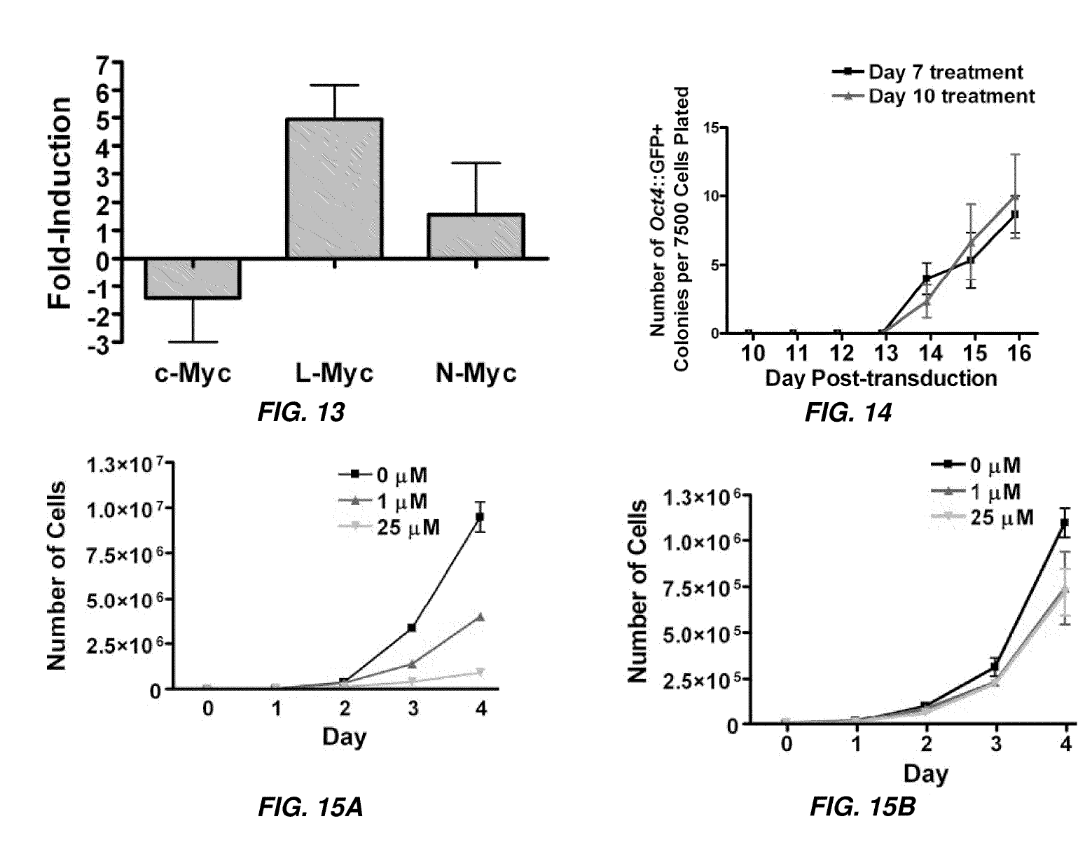

The inventors demonstrated that RepSox did, however, strongly increase the expression of L-Myc in MEFs. Within 7 days of RepSox treatment, L-Myc expression increased 5-fold (FIG. 13). L-Myc is a close homolog of cMyc that can functionally replace it in reprogramming (6), demonstrating that RepSox may complement the omission of cMyc from the reprogramming cocktail by increasing the expression of L-Myc in the starting MEF population. Together, the inventors have demonstrated that although RepSox may function at the level of the starting MEF population to replace cMyc, it does not act on the starting MEF population to replace Sox2.

Because RepSox did not seem to act on the initial population of fibroblasts to replace Sox2, the inventors then investigated whether or not it functioned on intermediates that arose during reprogramming. The inventors transduced 7500 MEFs with Oct4, Klf4, and cMyc, waited for 4 days, and then began to treat cultures with RepSox for various lengths of time. While the maximum number of reprogrammed cells was obtained when the treatment lasted from day 4 until day 16 (FIG. 4A), the inventors demonstrated that treatment from day 4 until day 7 was also sufficient to induce about the half-maximal number of GFP+colonies (FIG. 4A). Even one day of treatment from day 4 until day 5 was sufficient to induce a limited amount of reprogramming (FIG. 4B) and indicated that a short pulse with RepSox was sufficient to replace Sox2. This discovery differs strikingly from the 5-10 day period during which transgene expression is normally required for successful reprogramming (2, 3, 33) and demonstrates that RepSox could trigger a switch that activates reprogramming.

To determine when RepSox could most efficiently function to replace Sox2 during reprogramming, the inventors then transduced differentiated somatic cells with Oct4, Klf4, and cMyc then treated the resulting cultures with the small molecule at various time-points. The inventors demonstrated that delaying the start of RepSox treatment increased its reprogramming potency, with optimal treatment beginning at 10 days post transduction (FIG. 4A). When treatment with RepSox was delayed until the 13^{th} day after transduction, however, a reduced number of Oct4::GFP+colonies formed (FIG. 4A). Consistent with these results, treatments with RepSox in short 24-hour pulses generated the greatest number of Oct4::GFP+colonies when applied at 10-11 days post transduction (FIG. 4B) and Oct4::GFP+colonies appeared at the same time, at day 14, in RepSox treatments beginning either at day 7 or day 10 (FIG. 14). Thus, as well as reprogramming differentiated cells, RepSox was also determined to act on intermediate cell types that accumulate during the reprogramming process.

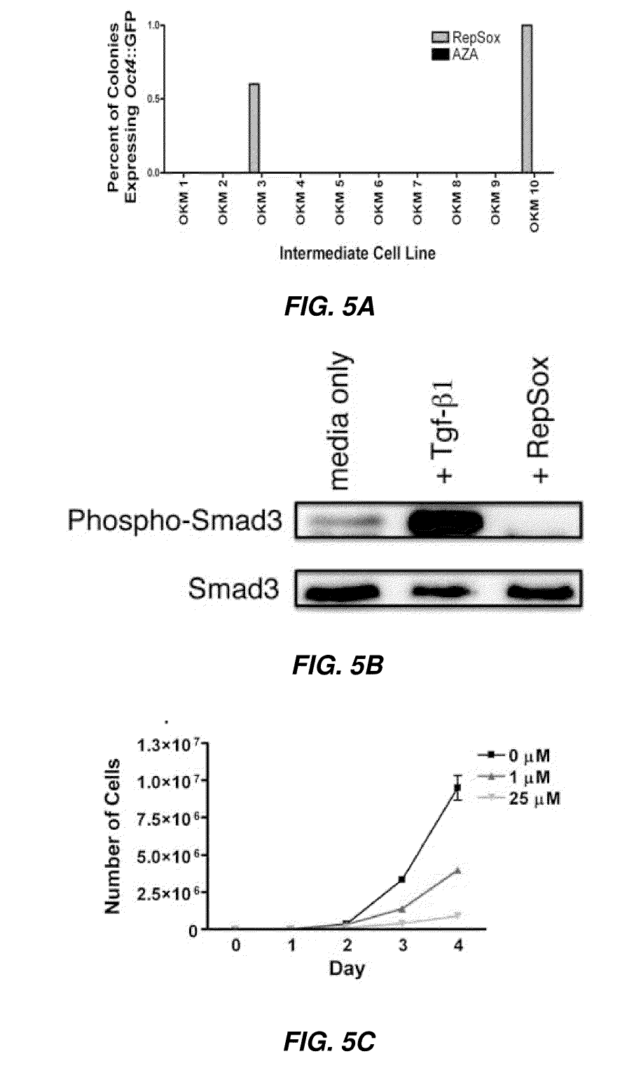

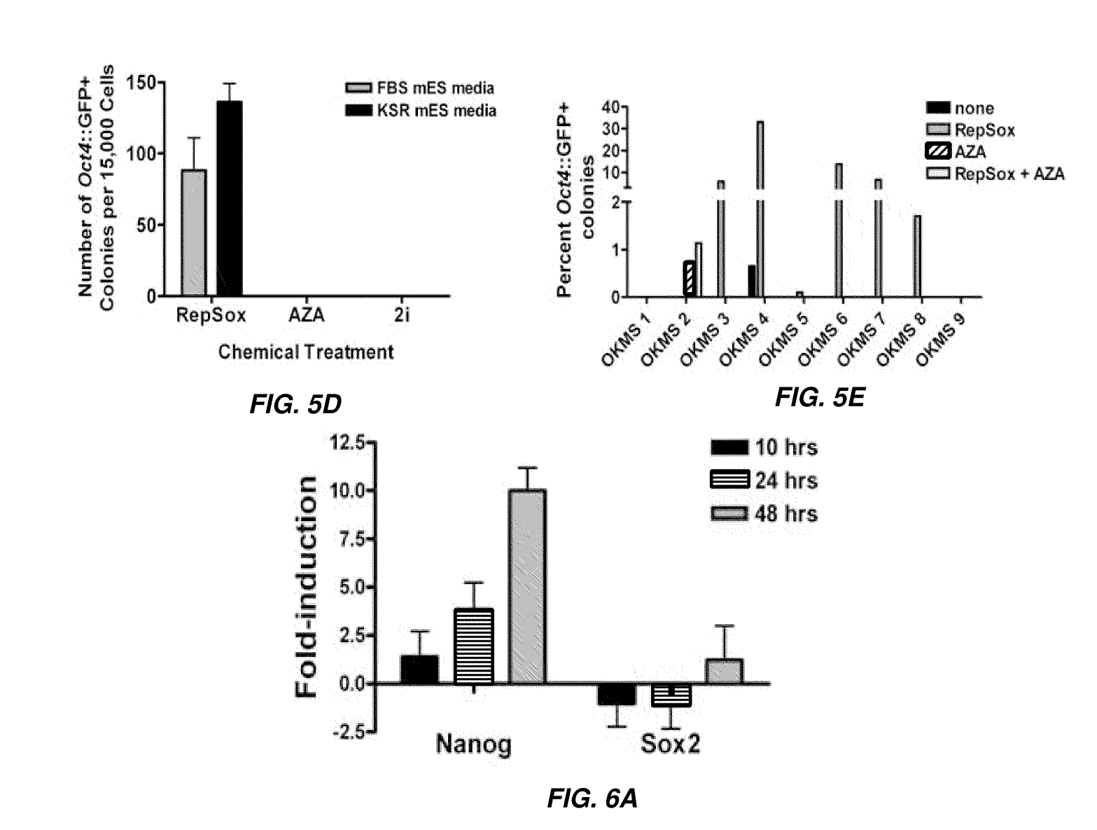

If RepSox acts to reprogram intermediate cell-types that accumulate in the absence of retroviral Sox2 expression, the inventors next assessed if Repsox might also reprogram clonally expanded lines derived from such intermediates. To test this, the inventors transduced Oct4::GFP MEFs with Oct4, Klf4, and cMyc, waited 10-14 days and then clonally expanded 10 iPS-like, GFP-negative colonies (Stable Oct4::GFP-negative cell lines derived from Oct4::GFP negative colonies in Oct4, Klf4, and cMyc-infected MEF cultures can be reprogrammed by RepSox. Oct4::GFP-negative colonies were picked at day 14 post-infection, propagated, treated with 25 μMRepSox for 48 hours at passage 4, and scored for Oct4::GFP+colonies 12 days after RepSox treatment—data not shown). These cell lines continued to proliferate for at least 4 passages and either maintained an iPS-like morphology (data not shown) or a more granular non-iPS-like morphology (data not shown). In all cases, these cultures failed to further activate expression of Oct4::GFP. However, when treated these cell lines with a 48 hour pulse of RepSox, 5-10% of the colonies in 2 of the 10 lines became Oct4::GFP+ (FIG. 5A, and data not shown). These results demonstrated that partially reprogrammed cells can accumulate in the absence of Sox2 and that some but not all of these cells can be completely reprogrammed by RepSox.

As the inventors had demonstrated that the RepSox reprogramming molecule seems to replace Sox2 through the inhibition of Tgf-β signaling, the inventors next assessed whether RepSox treatment affected downstream signal transduction pathways in a responsive, partially reprogrammed cell lines. To this end, the inventors determined the levels of phosphorylated Smad3 by western blot in cell line OKM 10 both with and without RepSox treatment. Without RepSox treatment, the inventors demonstrated relatively high levels of phosphorylated Smad3, demonstrating that Tgf-β signaling was active (FIG. 5B). In contrast, treatment with 25 μM RepSox almost completely eliminated phosphorylation of Smad3 (FIG. 5B), indicating that RepSox strongly inhibited Tgf-β signaling in these cells.

Because an increase in cell proliferation can also increase reprogramming efficiency (34-38) and possibly contribute to the replacement of transgenic Sox2, the inventors next determined the proliferation rate of OKM 10 cells both with and without RepSox. Addition of 25 μM RepSox to the media resulted in 10-fold reduction in cell number when compared to the untreated control over a 4 day time period (FIG. 15A), demonstrating that RepSox does not increase the proliferation rate of the majority of the intermediate cells. Furthermore, RepSox treatment of mES cells caused a reduction in cell number (FIG. 15B), demonstrating that Repsox does not enhance the proliferation rate of pluripotent cells during reprogramming of the OKM 10 cell line. To further confirm that treatment with RepSox did not increase the proliferation rate of partially reprogrammed cells, the inventors treated OKM 10 cells with RepSox and then performed a cell cycle analysis using propidium iodide. Treatment with RepSox decreased the proportion of cells in G2/M phase of the cell cycle (FIG. 5C).

### Example 6

### Partially Reprogrammed Cells that Respond to Repsox Treatment are Distinct from Previously Described Intermediates

It has been shown that certain non-pluripotent, partially reprogrammed cell lines derived from MEFs transduced with Oct4, Klf4, cMyc, and Sox2 can be fully reprogrammed with 5-aza-cytidine (AZA) or the combination of chemical inhibitors of Glycogen synthase kinase 3 (GSK3) and the Mek signaling pathway (21 conditions) (21, 39). If the RepSox-responsive cell lines generated by overexpression of Oct4, Klf4, and cMyc were similar to these 4-factor cell lines, then they should also be reprogrammed by AZA or 21. When the inventors treated the 10 stable intermediate lines with AZA for 48 hours, the inventors discovered that none of the 10 stable intermediate cell lines tested became reprogrammed after 14 additional days in culture (FIG. 5D). Next, the inventors treated a RepSox-responsive cell line containing transgenic Oct4, Klf4, and cMyc (OKM 10) with AZA or 21 for 48 hours, then cultured the cells for 14 days either with or without further passage. Neither chemical induced formation of Oct4::GFP+colonies (FIG. 5D), demonstrating that the RepSox-responsive stable intermediates are distinct from both the AZA-responsive and 21-responsive partially reprogrammed cell lines described previously (21). Consistent with these results, in vitro assays of kinase activity revealed that RepSox does not inhibit the targets of the 21 cocktail, namely Mek 1 and 2, Erk 1 and 2, and GSK-313 (Table 2). This demonstrates that RepSox responsive cells are not trapped in a nearly-pluripotent state, and that they could be urged into pluripotency by global demethylation or inhibitors of cell-signaling that maintain pluripotency in ES cells.

TABLE 2
In vitro assays of kinase inhibition activity for RepSox show that RepSox
does not inhibit the kinase targets of the 2i cocktail. Assays were
performed in duplicate using the Z′-LYTE system (Invitrogen).

| | Average | Standard |
|---|---|---|
| | % inhibition | error |
| Mek1 | 3 | 1 |
| Mek2 | −4 | 1 |
| Erk1 | 7 | 0 |
| Erk2 | −2 | 2 |
| GSK-3β | 1 | 0 |

The inventor also assessed of some of the non-pluripotent cells derived from MEFs transduced with Oct4, Klf4, cMyc, and Sox2 are potentially held in a non-pluripotent state due to inappropriate levels of Oct4 and Klf4 transgene expression, and therefore might also be reprogrammed with RepSox treatment. To test this hypothesis, the inventors transduced Oct4::GFP MEFs with Oct4, Klf4, cMyc, and Sox2, then picked and clonally expanded 9 GFP-negative colonies at day 14 after transduction (Stable Oct4::GFP-negative cell lines derived from Oct4::GFP negative colonies in Oct4, Klf4, cMyc and Sox2-infected MEF cultures can be reprogrammed by RepSox. Oct4::GFP-negative colonies were picked at day 14 post-infection, propagated, treated with 25 μMRepSox for 48 hours at passage 4, and scored for Oct4::GFP+colonies 12 days after RepSox treatment—data not shown). After treatment with RepSox, 5 of the 9 cell lines yielded reprogrammed colonies, with 2-33% of the colonies in each line becoming Oct4::GFP+ (FIG. 5E and data not shown). These results demonstrate that like the stable intermediate cells generated with only Oct4, Klf4, and cMyc, incompletely reprogrammed cells generated by Oct4, Klf4, cMyc, and Sox2 transductions can also be reprogrammed by RepSox.

Next, in order to determine if these RepSox-responsive intermediate cell lines derived after Oct4, Klf4, cMyc, and Sox2 transduction were similar to or distinct from previously described partially reprogrammed cell lines (21), the inventors applied AZA to all 6 lines. After 48 hours of AZA treatment and 12 subsequent days in culture, none of the RepSox-responsive cell lines expressed Oct4::GFP (FIG. 5E). However, one of the lines that had been refractory to RepSox treatment did express Oct4::GFP after AZA treatment, demonstrating that it had undergone complete reprogramming (FIG. 5E). Thus, the inventors have demonstrated that there are a variety of intermediates that can form following retroviral transduction and that they vary in their responsiveness to reprogramming molecules.

### Example 7

### RepSox Replaces Sox2 by Inducing Nanog Expression

The causal molecular events that drive reprogramming can be difficult to detect because following retroviral transduction only 0.1-1% of somatic cells are successfully reprogrammed (40). In contrast, when the inventors administered RepSox to cell lines that had been partially reprogrammed by retroviral transduction, Oct4::GFP expression was induced in up to 33% of the resulting colonies (FIG. 5E). The inventors used this more efficient reprogramming system to identify the changes in gene expression induced by RepSox that enable Repsox treated cells to bypass the requirement for transgenic Sox2 expression.

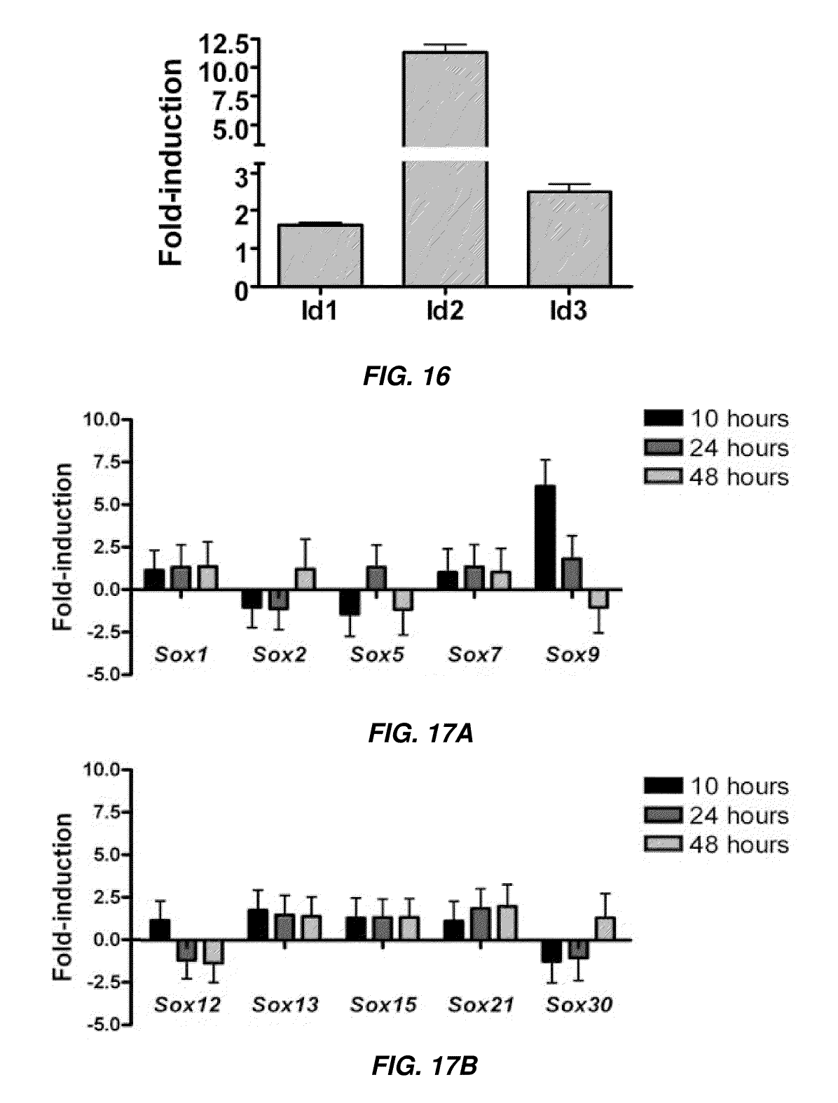

The inventors treated an Oct4::GFP-negative, partially reprogrammed cell line (OKMS 6) with RepSox for 48 hours and performed global gene expression analysis at 10, 24, and 48 hours following the initiation of treatment. First, to confirm that RepSox was inhibiting Tgf-β signaling in this intermediate cell line, the inventors investigated expression changes in known Tgf-β-responsive genes after RepSox treatment. The Inhibition of Differentiation genes Id1, Id2, and Id3 are repressed by Tgf-β signaling in mouse ES cells (41). After treating the RepSox-responsive intermediate line OKM 10 with RepSox for 24 hours, the inventors observed increased expression of Id1, Id2, and Id3 (FIG. 16).

To assess of RepSox functions to replace transgenic Sox2 by inducing the expression of endogenous Sox2 or expression of a Sox-family member, such as Sox1 or Sox3, which can substitute for Sox2 in reprogramming (6), the inventors assessed the expression of all members of the Sox-family of transcription factors on Repsox treatment. The inventors did not observe a significant increase in the expression of Sox1, Sox2, Sox3, or any of the remaining Sox-family transcription factors within the first 48 hours of RepSox treatment (FIG. 16). Consistent with these data, small hairpin RNA-mediated knockdown of Sox2 and Sox/, the most potent members of the Sox-family (6), did not affect the rate of reprogramming of intermediate line OKM 10 or Oct4, Klf4, cMyc-transduced MEFs in the presence of RepSox (FIG. 17). Thus, the inventors demonstrate that RepSox does not replace Sox2 by directly activating endogenous Sox2 or other closely related genes.

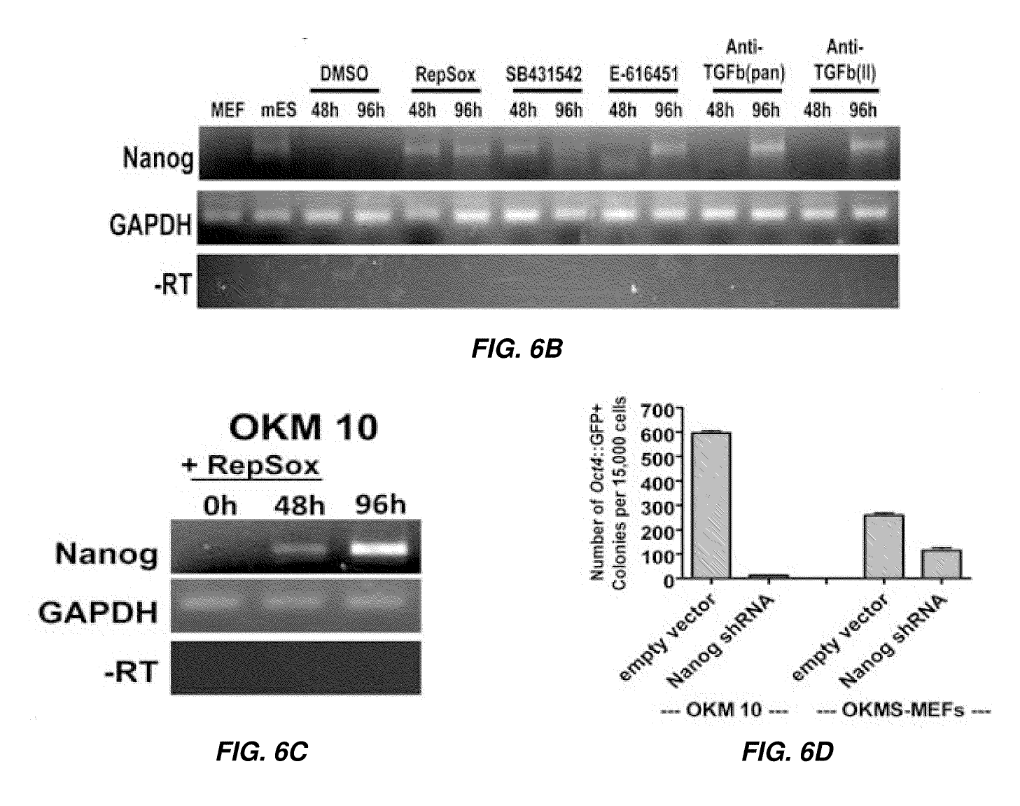

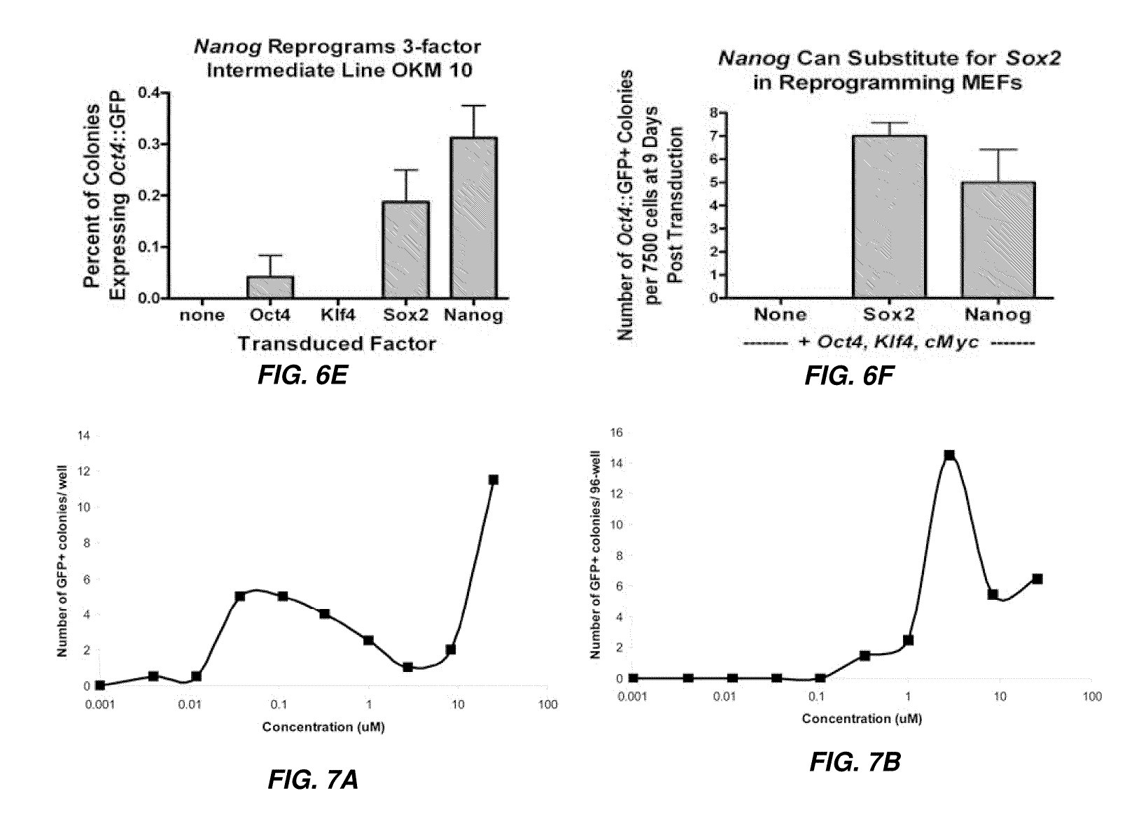

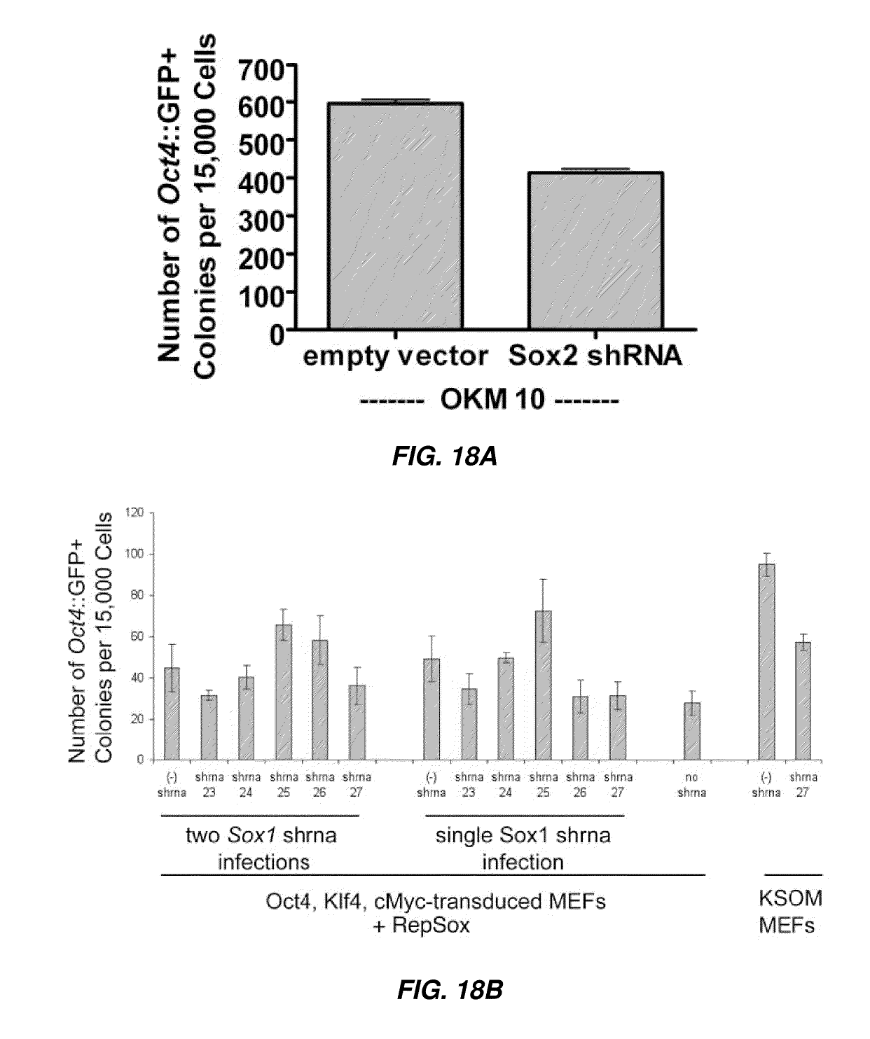

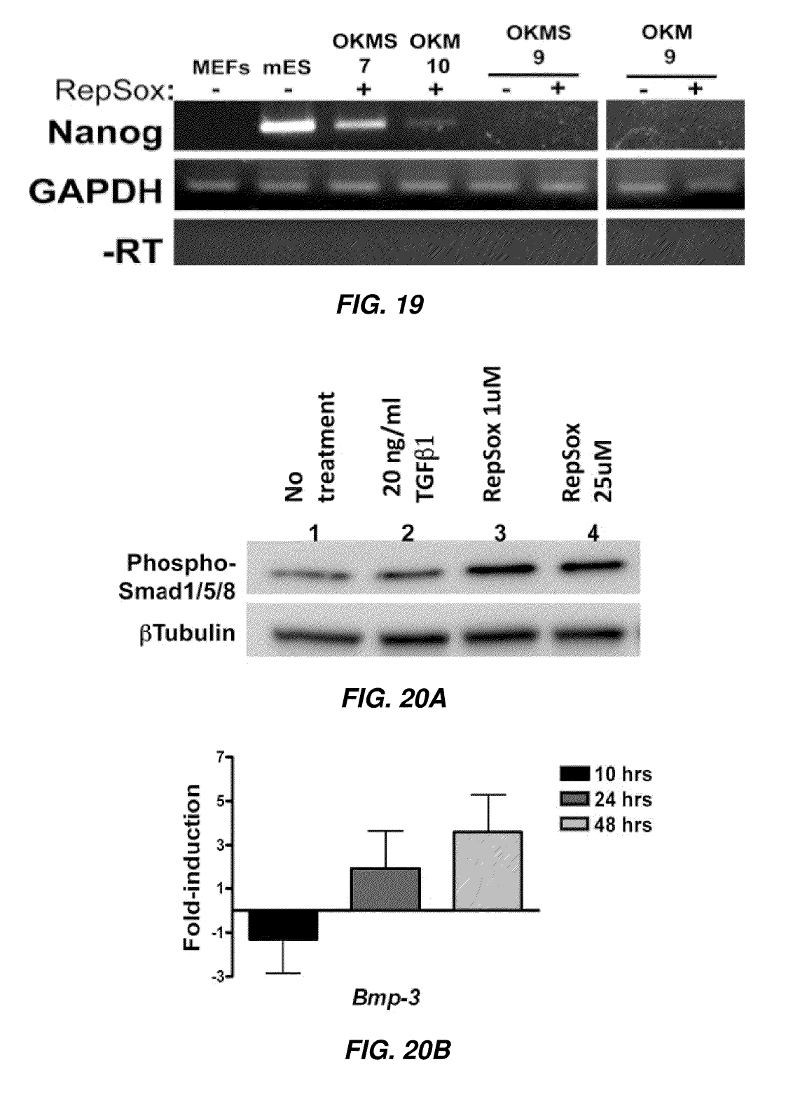

Next, the inventors investigated changes in transcription factor gene expression following chemical treatment with Repsox. The inventors did not observe an increase in endogenous Oct4 or Klf4 expression at early timepoints following RepSox treatment. However, the inventors discovered that the expression of the homeodomain factor Nanog was among the most increased transcription factors following RepSox treatment. Relative to untreated controls, Nanog transcription increased 4-fold within 24 hours and 10-fold after 48 hours of treatment with RepSox (FIG. 6A). In contrast, the inventors did not observe a rapid increase in Nanog expression in Oct4::GFP-negative intermediate cell lines generated with either Oct4, Klf4, and cMyc, or Oct4, Klf4, cMyc, and Sox2 that could not be fully reprogrammed using RepSox (FIG. 18A-18B). Therefore, the inventors next determined if RepSox replaces Sox2 by inducing Nanog expression.

Since the inventors determined that inhibition of Tgf-β signaling by several different small molecules (such as Repsox, SB431542 and E-616451), and anti-TGFβ antibodies can replace Sox2, the inventors next determined if other inhibitors of Tgf-β signaling also upregulate Nanog, and thus determined if the increase in Nanog expression was critical for Sox2 replacement. Thus, the inventors treated the RepSox-responsive intermediate cell lines OKM 10 and OKMS 7 with RepSox, SB431542, or neutralizing antibodies specific for Tgf-β and analyzed Nanog expression after 48 hours. In all cases, Nanog expression was strongly increased as compared to an untreated control at 48 hours (FIG. 6B). Thus, the inventors have determined that the increase in Nanog expression on Repsox treatment is due to inhibition of Tgf-β signaling.

Because the inventors have also discovered that a short pulse of RepSox can reprogram cells in the absence of Sox2 (see FIG. 4), if RepSox functions by increasing Nanog expression then a short pulse RepSox treatment should induce a persistent increase in Nanog expression. Thus, the inventors assessed if a short pulse RepSox treatment induced persistent increase in Nanog expression. The inventors treated the RepSox-responsive intermediate cell line OKM 10 with RepSox for 48 hours, withdrew RepSox and then analyzed Nanog expression 48 hours later. A control timepoint taken just before RepSox withdrawal showed a significant increase in Nanog transcription (FIG. 6C). 48 hours after RepSox removal (96 hours after the initiation of treatment), Nanog expression continued to increase (see FIG. 6C). Thus, the inventors demonstrate that briefly exposing responsive cell lines to RepSox results in a persistent increase in Nanog expression.

The inventors next assessed if RepSox replaces Sox2 by increasing Nanog expression by assessing the forced knockdown of Nanog expression, and determined if knockdown of Nanog reduced or prevented reprogramming with RepSox. To test this, the inventors transduced the RepSox-responsive cell line with a lentivirus encoding a short-hairpin RNA specific for Nanog before treating with RepSox. The cells transduced with the Nanog shRNA construct reprogrammed at a rate that was 50-fold lower than cells transduced with an empty control vector (FIG. 6D). This effect was not due to a general decrease in reprogramming efficiency or differentiation of reprogrammed cells due to Nanog depletion; MEFs transduced with Oct4, Klf4, cMyc, Sox2, and the Nanog shRNA construct only suffered a 50% loss in reprogramming efficiency as compared to MEFs transduced with the empty vector control lentivirus (FIG. 6D). Thus, the inventors demonstrate that increased Nanog expression is only necessary for the replacement of Sox2 by RepSox.

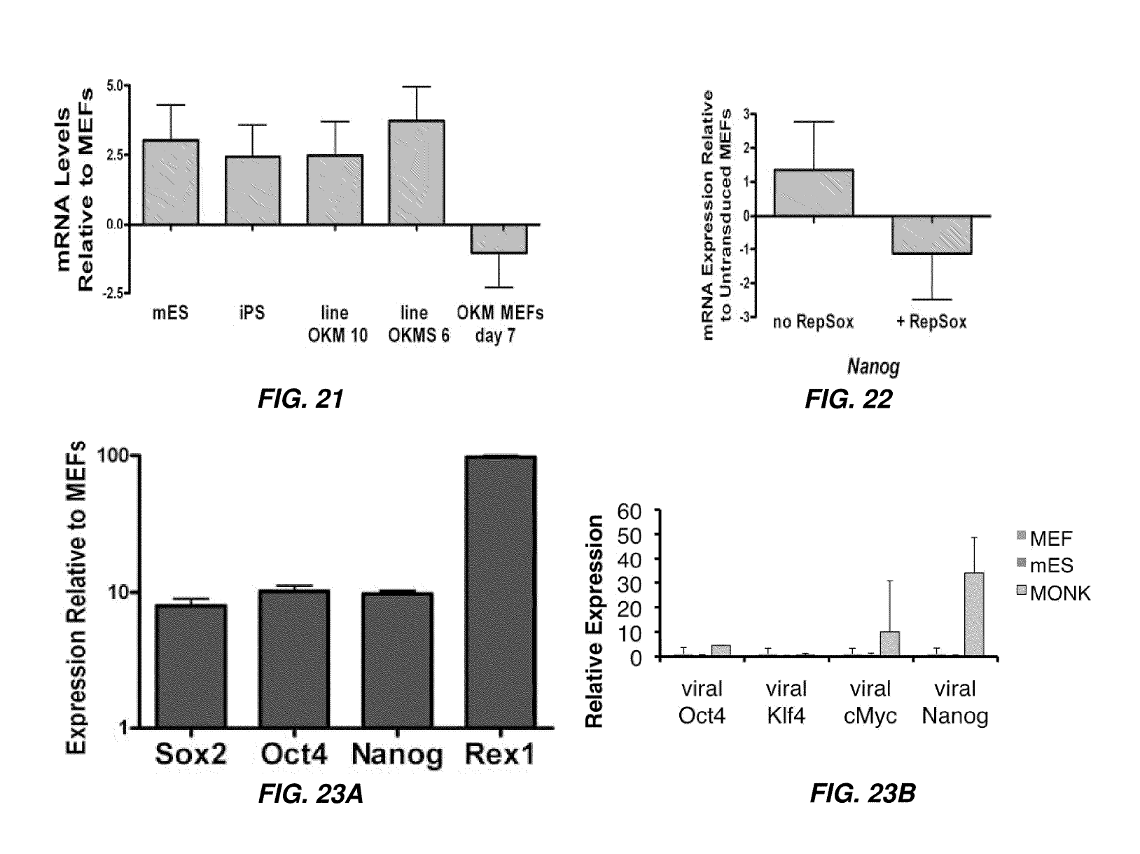

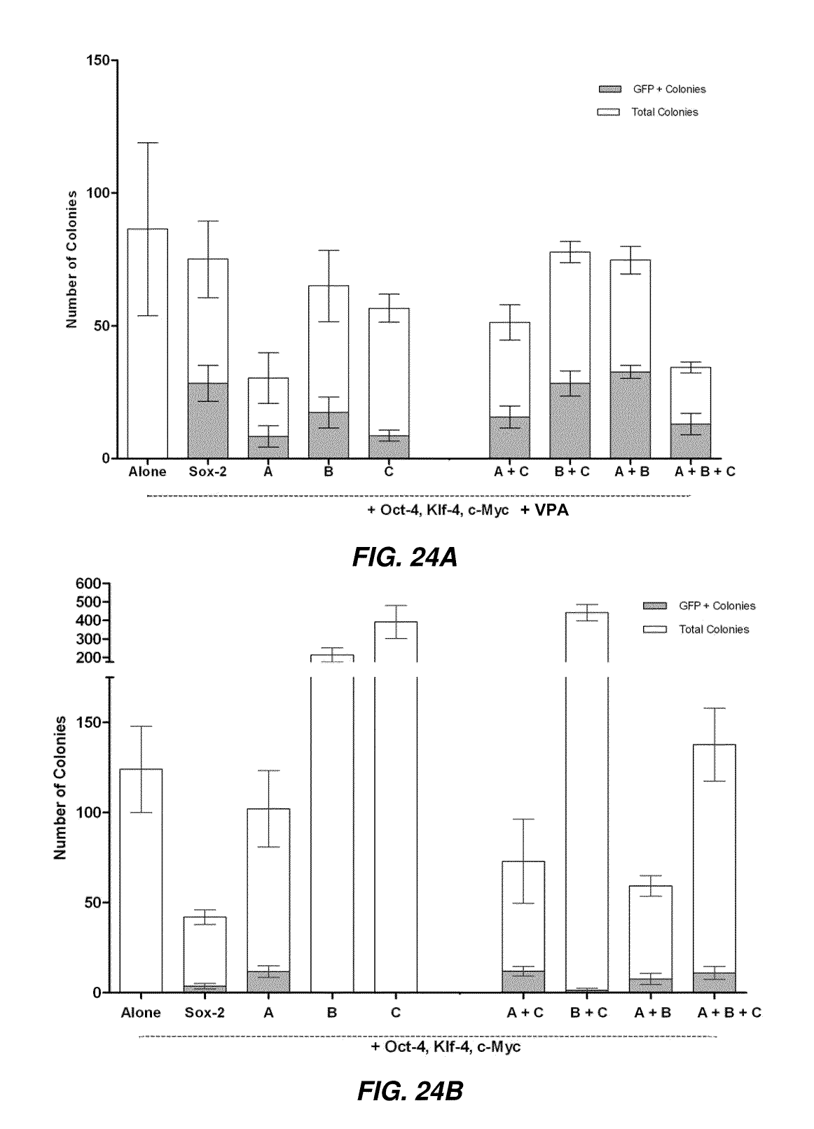

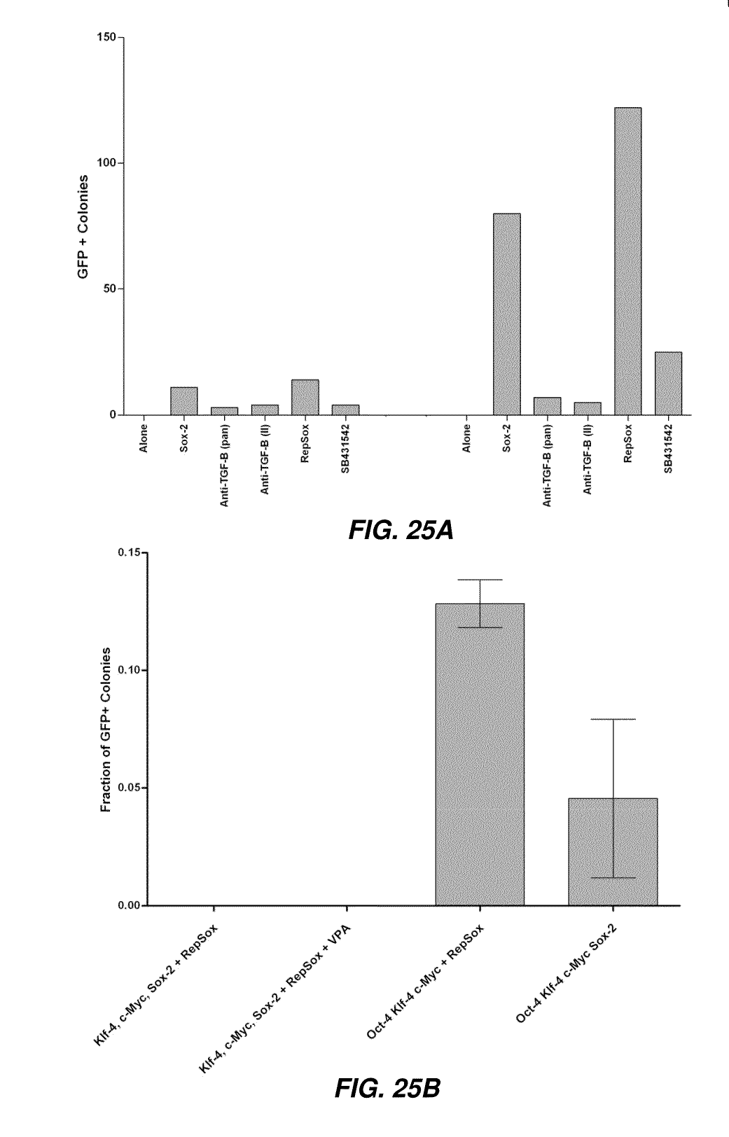

Previous reports have shown that chemical inhibition of Tgf-β signaling by SB431542 increases Bmp signaling in embryonic stem cells (42). It has separately been shown that Bmp signaling in the presence of Stat3 induces Nanog expression in mouse ES cells (43). The cross-talk between the Tgf-β and bone morphogenetic protein (Bmp) signaling pathways may be the result of a common requirement for Smad 4, which mediates transcriptional events in the nucleus (44). Consistent with this model, the inventors determined an increase in the levels of phosphorylated Smad1 protein and Bmp-3 mRNA in incompletely reprogrammed intermediates following RepSox treatment (FIG. 20A, 20B). Furthermore, the stable, partially reprogrammed cells that responded to RepSox expressed the LIF receptor at levels equivalent to those found in mES cells (FIG. 21). Expression of the LIF receptor indicates that its downstream signal transduction pathway is active in these cells, and could result in the presence of activated Stat3, which is known to induce Nanog expression in conjunction with Bmp signaling

Since the inventors discovered that RepSox does not act on the starting fibroblast cells, but rather stable intermediate, or partially reprogrammed cells, it is unlikely that Nanog is upregulated in RepSox-treated MEFs. Indeed, in MEFs that had been transduced with Oct4, Klf4, and cMyc for 7 days or less, the inventors did not observe an increase in Nanog expression within 48 hours of RepSox treatment (FIG. 22). This may be explained in part by the observation that the LIF receptor, and thus activated Stat3, were not highly expressed in untransduced or freshly transduced (<7 days) Oct4, Klf4, and cMyc-infected MEFs (FIG. 21). Because Nanog plays a key role in maintaining embryonic stem cells in an undifferentiated state (45-47) and has been shown to enhance the efficiency of reprogramming (48, 49), the inventors next assessed whether Nanog could directly replace Sox2 in reprogramming.

The inventors assessed if RepSox replaces Sox2 by inducing Nanog expression by determining if retroviral transduction of RepSox-responsive intermediate cells (line OKM10) with Nanog results in reprogramming the OKM 10 cells (i.e. partially reprogrammed, stable intermediate) to pluripotency (FIGS. 5A, 5B and data not shown). When the inventors transduced line OKM 10 with Sox2 as a control, 0.2% of the colonies expressed Oct4::GFP after 10 days, demonstrating that reprogramming could be induced in this cell line by the addition of Sox2 (FIG. 6E, and data not shown 6F). Next, the inventors assessed if Nanog could also induce reprogramming in these cells. When the inventors transduced the same stable intermediate cell line with Nanog, the cells could also be reprogrammed, with 0.3% of the colonies expressing Oct4::GFP+ after 10 days (FIG. 6E, and data not shown). In contrast, transductions with Oct4 or Klf4 resulted in only 0.04% and 0% of cells expressing Oct4::GFP, respectively (FIG. 6E). These results demonstrate that Nanog can functionally replace Sox2 and induce reprogramming in these stable intermediates formed from Oct4, Klf4, and cMyc-transduced MEFs.

The inventors next assessed if Nanog can indeed genetically complement for the omission of Sox2 in defined factor reprogramming by determining if MEFs transduced with Oct4, Klf4, cMyc, and Nanog can be efficiently reprogrammed to a similar level as MEFs transduced with Oct4, Klf4, cMyc, and Sox2. When the inventors transduced MEFs with Oct4, Klf4, cMyc, and Sox2 then scored cultures 9 days later, an average of 7 Oct4::GFP+colonies appeared for every 7500 cells plated (FIG. 6F). A control transduction with only Oct4, Klf4, and cMyc yielded no Oct4::GFP+colonies (FIG. 6F). Strikingly similar to the positive control transduction, MEFs transduced with Oct4, Klf4, cMyc, and Nanog gave rise to an average of 5 Oct4::GFP+colonies for every 7500 cells plated (FIGS. 6F, 6G). These colonies could be picked and expanded and remained Oct4::GFP+ over at least 5 passages (data not shown). Immunocytochemistry demonstrated that these cells strongly activated Sox2 expression from the endogenous allele (Figure data not shown) and could readily form embryoid bodies in vitro (Figure data not shown). Importantly, QPCR analysis demonstrated that these cells had activated endogenous Oct4, Klf4, Nanog, and Rex1 (FIG. 23A) and silencing of retroviral transgenes (FIG. 23B), demonstrated a pluripotent gene expression program had been established. Leaky expression from the transgenic Nanog, which is a potent inhibitor of embryonic stem cell differentiation (45, 46), reduced the amount of differentiation in vitro (FIG. 23B). The inventors anticipate that efficient differentiation of cells created with Oct4, Klf4, cMyc, and Nanog will require the use of an excisable transgenic Nanog cassette to completely remove ectopic Nanog expression. Based on these data, the inventors determined that Nanog expression is sufficient to replace Sox2 in defined factor reprogramming. Thus, the inventors have demonstrate that RepSox inhibition of Tgf-β signaling strongly induces Nanog expression, which then bypasses the need for Sox2 in defined-factor reprogramming.

As shown herein, the inventors have used a phenotypic chemical screen to identify compounds with known pharmacological activities that can replace the key reprogramming transcription factor Sox2. Furthermore, the inventors have demonstrated the mechanism by which the most potent Sox2 replacement compound acts: RepSox replaces Sox2 by inhibiting the broadly expressed Tgf-β signaling pathway (44) in cultures containing stable intermediate cells that are trapped in a partially reprogrammed state. This inhibition in turn leads to sustained transcription of Nanog, which then compensates for the absence of Sox2. Thus the inventors have demonstrated the feasibility of specifically replacing the central reprogramming transgene, such as Sox2 with small molecules that modulate specific cellular pathways or processes rather than by globally altering gene expression or chromatin structure. Furthermore, the inventors have demonstrated the mechanisms by which these molecules act in reprogramming can be distinct from those of the factor(s) that they replace.

Importantly, and unlike many other studies (19, 21, 28), one advantage of the present invention is that the inventors have demonstrated replacing Sox2 without relying on the procurement of a highly specialized or rare cell type. While the chemical screens were performed in mouse embryonic fibroblasts, the inventors have also demonstrated that RepSox is capable of replacing Sox2 in the reprogramming of adult tail tip fibroblasts. Furthermore, treatment with RepSox allowed the generation of iPS cells with a frequency comparable to that of retroviral transduction with Sox2. Thus, reprogramming efficiency does not need to be compromised by the small molecule replacement of transgenic factors.

The inventors have clearly demonstrated that RepSox functions to replace Sox2 via, at least partially, inhibition of Tgf-β signaling and have also demonstrated more broadly that small molecule replacement of transgenic reprogramming factors can be used to identify intracellular pathways that modulate reprogramming. Interestingly, the rate at which GFP+colonies were induced using the Tgf-β neutralizing antibodies was lower as compared the treatment of RepSox. This may be due to neutralizing antibodies may be cell-toxic at the concentrations used in the inventor's studies, or the neutralizing anti-TGFβ antibodies may be less potent inhibitors of Tgf-β cell signalling than RepSox.

In addition, the inventors demonstrate that instead of reprogramming differentiated cells (e.g. the initial fibroblast population) to replace Sox2, RepSox acts on cellular intermediates formed by overexpression of Oct4, Klf4, and cMyc. Without RepSox treatment, these intermediates are trapped in an unproductive, non-pluripotent state and do not escape to pluripotency even after several passages. Unlike the previously described partially reprogrammed cells derived from MEFs transduced with Oct4, Klf4, cMyc, and Sox2 or Sox2-expressing neural progenitor cells transduced with Oct4, Klf4, and cMyc (21, 39, 50), the RepSox-responsive intermediates derived from MEFs transduced with Oct4, Klf4, and cMyc do not respond to AZA or 21 treatment, demonstrating that they are distinct. In addition, the inventors also discovered that RepSox does not target any of the kinases inhibited by the 21 cocktail, demonstrating that Repsox works through a different mechanism. Furthermore, 4-factor intermediates that reprogram with RepSox treatment are not responsive to AZA, also demonstrating that they also are distinct from previously described 4-factor intermediates.

The inventors demonstrated that a 24-hr treatment of RepSox can relieve the requirement for transgenic Sox2 (e.g. exogenous Sox2 transcription factor, such as nucleic acid encoding Sox2 or the Sox2 polypeptide). Thus, using Repsox is unlike reprogramming using transgenic Oct4, Klf4, and Sox2, where each transgene must be expressed for several days (33, 51). Thus, small molecule replacement of Sox2 using Repsox can act as switches to induce stable changes in gene expression that promote the completion of reprogramming.

The inventors surprisingly discovered that Nanog was not included in the initial set of defined reprogramming factors (52), which is surprising given its critical role in maintaining pluripotency in ES cells (45, 53) and its ability to stimulate reprogramming by cell-fusion (48). However, previous reports by Takahashi and Yamanaka report that a combination of 9 factors that included Oct4, Klf4, cMyc, and Nanog, but not Sox2, generated iPS colonies at a detectable rate (52). However, this combination of factors included at least 5 other genes. This report did not identify, or suggest or demonstrate that the combination of Oct4, Klf4, cMyc, and Nanog could be used to reprogram cells.

It is well known that approximately 90% of genes with promoters that are bound by Oct4 and Sox2 in hES cells are also bound by Nanog (53). The inventors also demonstrate that either Nanog or Sox2 are sufficient to collaborate with Oct4 to modulate these genes and productively drive reprogramming. Although Nanog is not required for pluripotency, it safeguards ES cells against neurectodermal and, to a more limited extent, mesodermal differentiation (46, 47). Therefore, it is possible that Nanog may function in reprogramming by repressing differentiation signals, assisting in the transition to an undifferentiated state.

Interestingly, the inventors demonstrate that RepSox is also able to functionally replace cMyc in reprogramming. Therefore, the inventors have discovered one small molecule, such as Repsox can compensate for the removal of two different transgenic reprogramming factors by two distinct mechanisms. A 25-fold lower concentration of RepSox is sufficient for cMyc replacement as compared to Sox2 replacement, demonstrating that less complete inhibition of TGF-β signaling is required to elicit the gene expression changes needed to compensate for cMyc removal. Because the inventors also demonstrated that RepSox does not improve the reprogramming efficiency with all four transgenic factors, Repsox is likely to replaces cMyc in a more specific manner. This is demonstrated by the inventors discovery that L-Myc expression increases by 5-fold in MEFs after RepSox treatment. Together, the inventors have discovered the fact that small molecules, such as Repsox can functionally replace reprogramming transcription factors at either early or late stages of the reprogramming process, and the small molecules such as Repsox can act by two different mechanisms—by inducing the expression of the gene itself or a closely related family member as in the case of cMyc or by inducing the expression of an unrelated gene that can functionally rescue the omission of the reprogramming transcription factor, as is the case of Sox2 replacement by Nanog induction.

The inventors have shown small molecule-mediated perturbation of a broadly known cell signaling pathway can functionally replace the forced overexpression of an Sox-2 in the direct reprogramming process. This process does not require procurement of a highly specialized or scarce cell population or use of generally acting chemicals that may produce undesirable effects on the recipient cells. Furthermore, treatment with Rep Sox is as effective as transduction with the Sox-2 retrovirus, indicating that efficiency is not compromised by small molecule replacement of the transgene.

Repsox can also replace Sox2 and reprogram human fibroblasts, as (i) human neonatal fibroblasts express the TGF-beta type 1 receptor [6]. (ii) RepSox inhibits the human version of TGFBR1 [27]. (iii) TGF-beta signaling is highly conserved among vertebrates [29].

The inventors have used a functional chemical screen to identify compounds that specifically replace Sox-2 in direct reprogramming. Furthermore, the inventors have determined the mechanism by which the most potent compound acts. RepSox replaces Sox-2 by inhibiting TGF-beta signaling, a broadly expressed cell signaling pathway [29]. These results demonstrate that it is possible to specifically replace one of the critical reprogramming transgenes with a small molecule that specifically modulates a cell signaling pathway and does not globally alter gene expression or chromatin structure. The inventors' work suggests that it will be possible to replace the remaining iPS transgenes with small molecules that perturb cell signaling pathways. Also, these results suggest that the functional screening method we used to replace one transgene at a time is robust enough to identify these molecules. This approach will be vital to achieving virus-free, chemical reprogramming because one can direct it towards replacement of any gene.

### Example 8

### Small Molecule Replacement of Klf-4

The inventors expected that omission of one of the critical reprogramming factors, such as Klf4, would significantly reduce or even eliminate the appearance of GFP+colonies in this assay. Indeed, the inventors observed an average of only one GFP+colony when Klf4 was omitted from the same transduction (Fig. xA). The inventors used this robust difference in the number of colonies expressing GFP to identify small molecules that can replace Klf4 in reprogramming.

To ease the identification of cellular targets and signaling pathways that were affected by the molecules the inventors discovered, the inventors selected a library of small molecules that had known pharmacological targets, including kinases, ion channels, and extracellular receptors. This library was supplemented with compounds known to directly affect the self-renewal and differentiation of pluripotent stem cells (Desbordes et al., Cell Stem Cell 2, 602, 2008) as the inventors felt that they, like the pluripotency genes, might play a role in reprogramming.

The inventors transduced Oct4::GFP MEFs with Oct4, cMyc, and Sox2 and then plated 2000 cells per well in 96-well format. To each well we added one of 800 distinct compounds for 25 days, treating one replicate of the screen with 200 nM VPA for the first 7 days (FIG. 26B). This approach would allow the inventors to identify both compounds that required widespread chromatin remodeling to induce reprogramming (Huangfu et al., Nat. Biotechnol. 26, 795, 2008) and compounds that did not. After 30 days, the inventors scored each well for the presence of GFP+colonies with a mES-like morphology (data not shown) and identified two hit compounds: 15-deoxy-Δ^{12,14}-prostaglandin J₂ (Prostaglandin J2) and HDBA (FIGS. 26B, 26C). Following the screen, a literature search revealed that Prostaglandin J2 had previously been shown to up-regulate Klf4 expression in both mouse and human cells through activation of the MEK/ERK signaling pathway (Chen et al., Mol. Pharmacol. 68, 1203, 2005). In contrast, HDBA inhibits Ca²⁺/calmodulin kinase II (CaMKII), the EGF receptor tyrosine kinase, and Src kinase activities (O'Dell et al., Nature 353, 558, 1991).

The inventors next validated the ability of these two compounds to replace Klf4 at a larger scale either in the presence or absence of VPA. Again, Prostaglandin J2 induced GFP+colonies, although only in the absence of VPA, while HDBA was capable of inducing GFP+colonies both with and without VPA (FIG. 26D). Furthermore, the efficiency of reprogramming with HDBA in the absence of transgenic Klf4 was similar to a positive control transduced with retroviruses encoding all four reprogramming factors (FIG. 26D). These results strongly demonstrate that the inventors have identified two distinct molecules that function by divergent mechanisms to replace Klf4 in defined factor reprogramming.

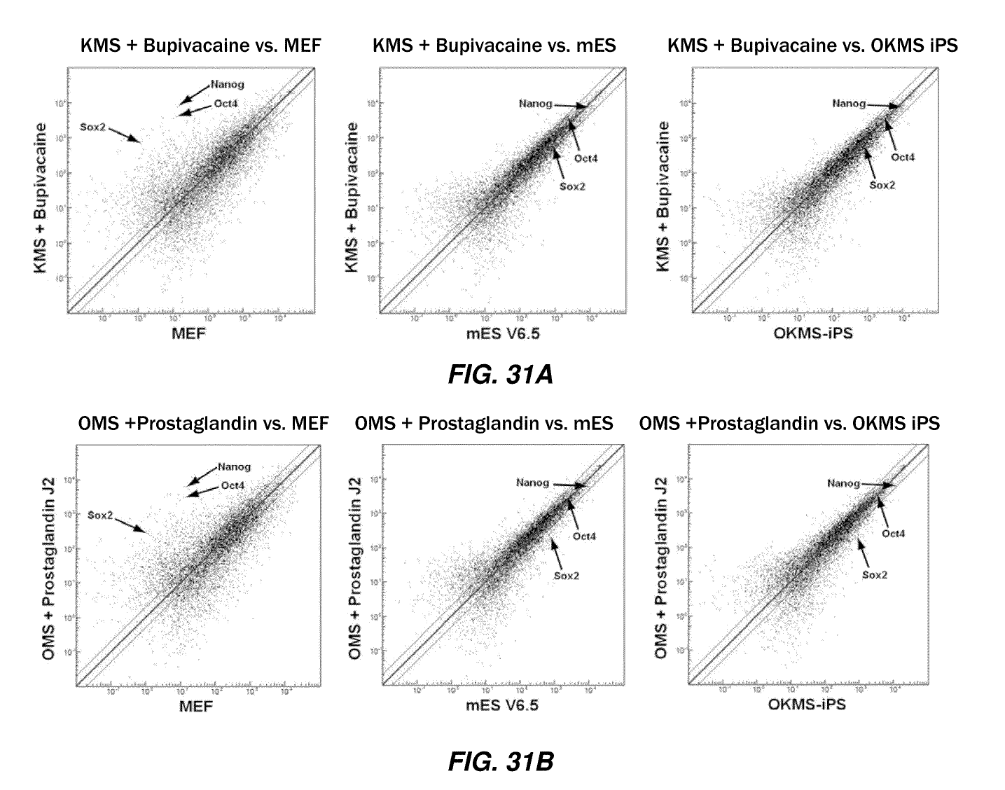

Investigation of self-renewal capacity, gene expression program, and pluripotency demonstrated that GFP+ cells induced by the Prostaglandin J2 replacement of Klf4 were bona fide iPS cells. The inventors demonstrated that the reprogrammed cell lines self-renewed for more than 10 passages with a growth rate similar to mES cells (data not shown), while maintaining a mES cell-like morphology (FIG. 26E) and expression of the Oct4::GFP transgene (data not shown). Antibody staining indicated that the Oct4::GFP positive cells co-expressed the endogenous alleles of the Nanog and Sox2 or Oct4 genes (FIG. 31B), demonstrating a pluripotent transcriptional program had been established. In addition, all cell lines expressed the embryonic enzymatic activity alkaline phosphatase (FIG. 30C), as do mouse ES cells and iPS cells.

The global transcriptional profiles of cell lines reprogrammed with Prostaglandin J2 or HBDA were similar to a mouse ES cell line and to iPS cell lines produced with all four transgenes, while they differed significantly from that of the somatic MEFs (FIG. 31B). Pearson correlation coefficient analysis confirmed that iPS cells produced with Prostaglandin J2 were as similar to mouse ES cells (Pearson correlation coefficient=0.95-0.97) as two mouse ES cell lines were to each other (Pearson correlation coefficient=0.96) (Table 3).

TABLE 3
Pearson correlation coefficients between mES cell lines, Oct4, Klf4, cMyc,
Sox2 (OKMS)-iPS line 1, Oct4, Klf4, cMyc (OKM) + RepSox iPS line 1,
Klf4, cMyc, Sox2 (KMS) + Bupivacaine iPS line 1, Oct4, cMyc, Sox2
(OMS) + Prostaglandin J2 iPS line 1, and Oct4::GFP MEFs.
Pearson (R)

| | | | OKMS- | OMS + | KMS + | OKM + | |
|---|---|---|---|---|---|---|---|
| | mES1 | mES2 | iPS | Prostaglandin J2 | Bupivacaine | RepSox | MEF |
| mES1 | 1.00 | 0.96 | 0.98 | 0.94 | 0.94 | 0.96 | 0.80 |
| mES2 | | 1.00 | 0.99 | 0.95 | 0.95 | 0.97 | 0.81 |
| OKMS-iPS | | | 1.00 | 0.95 | 0.95 | 0.97 | 0.82 |
| OMS + Prostaglandin J2 | | | | 1.00 | 0.96 | 0.94 | 0.85 |
| KMS + Bupivacaine | | | | | 1.00 | 0.94 | 0.79 |
| OKM + RepSox | | | | | | 1.00 | 0.79 |
| MEF | | | | | | | 1.00 |

In order to demonstrate that the chemically reprogrammed cell lines reprogrammed with prostaglandin J2 and Oct4, cMyc, Sox2 (OMS) share the pluripotent characteristics of mES and iPS cells, the inventors demonstrated their ability to spontaneously differentiate in vitro into cells types derived from the three embryonic germ layers (data not shown). The inventors also demonstrated that cells reprogrammed with Prostaglandin J2 could respond to directed differentiation signals in vitro (data not shown). These data demonstrate that the iPS cells generated with Prostaglandin J2 are pluripotent and equivalent to other iPS cells with respect to their differentiation in vitro.

### Example 9

### Small Molecule Replacement of Oct4

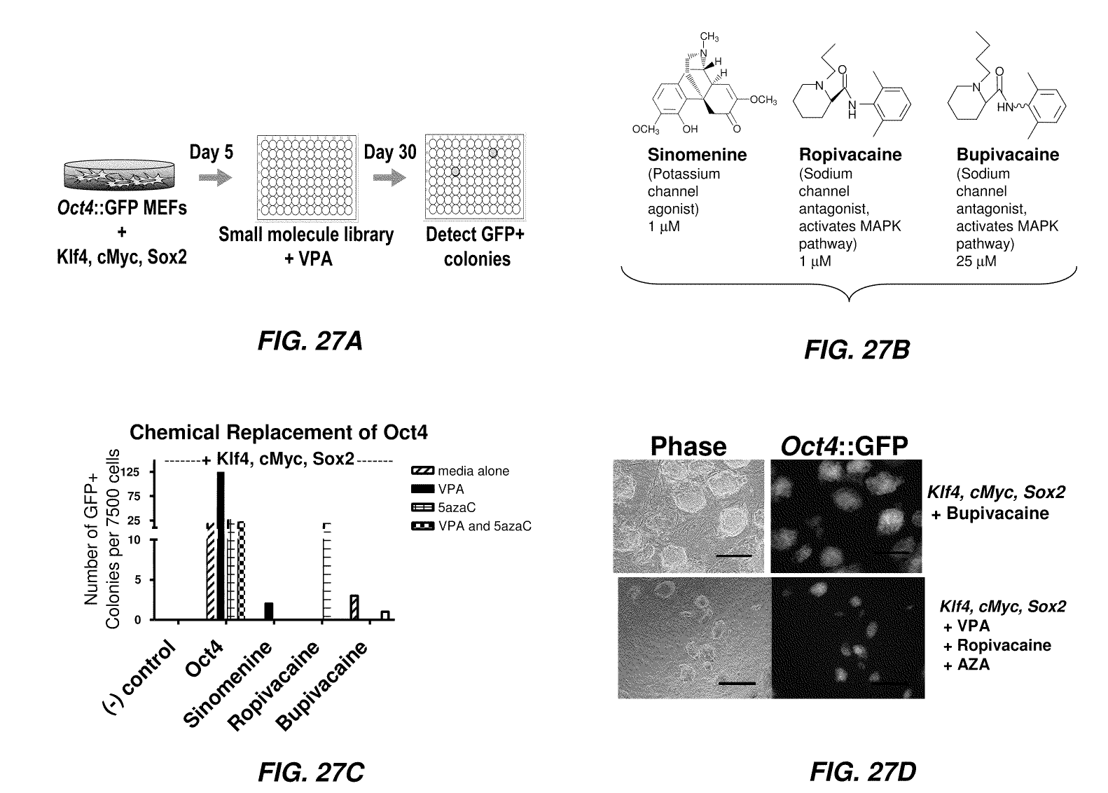

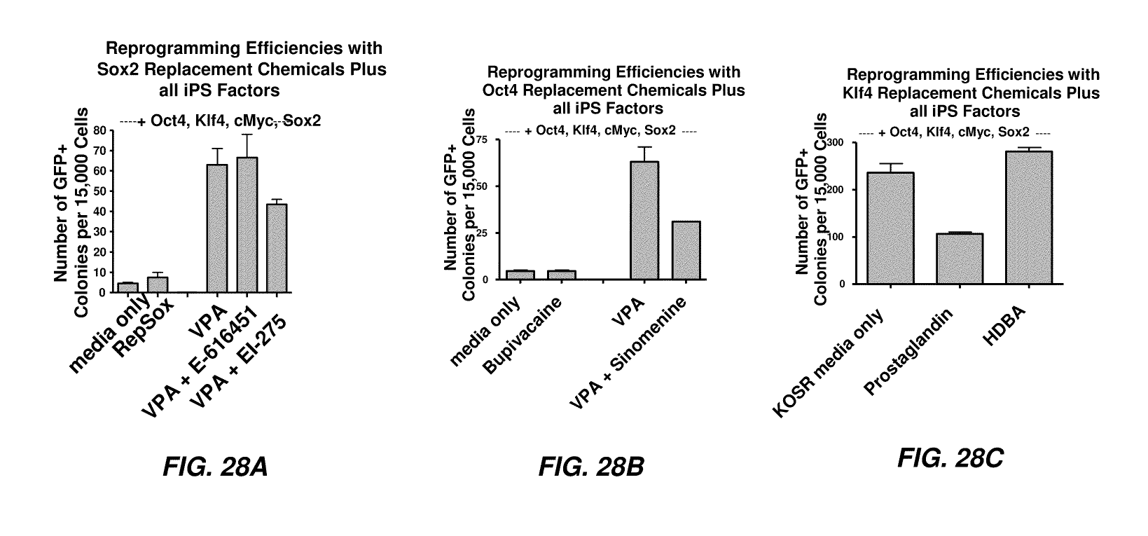

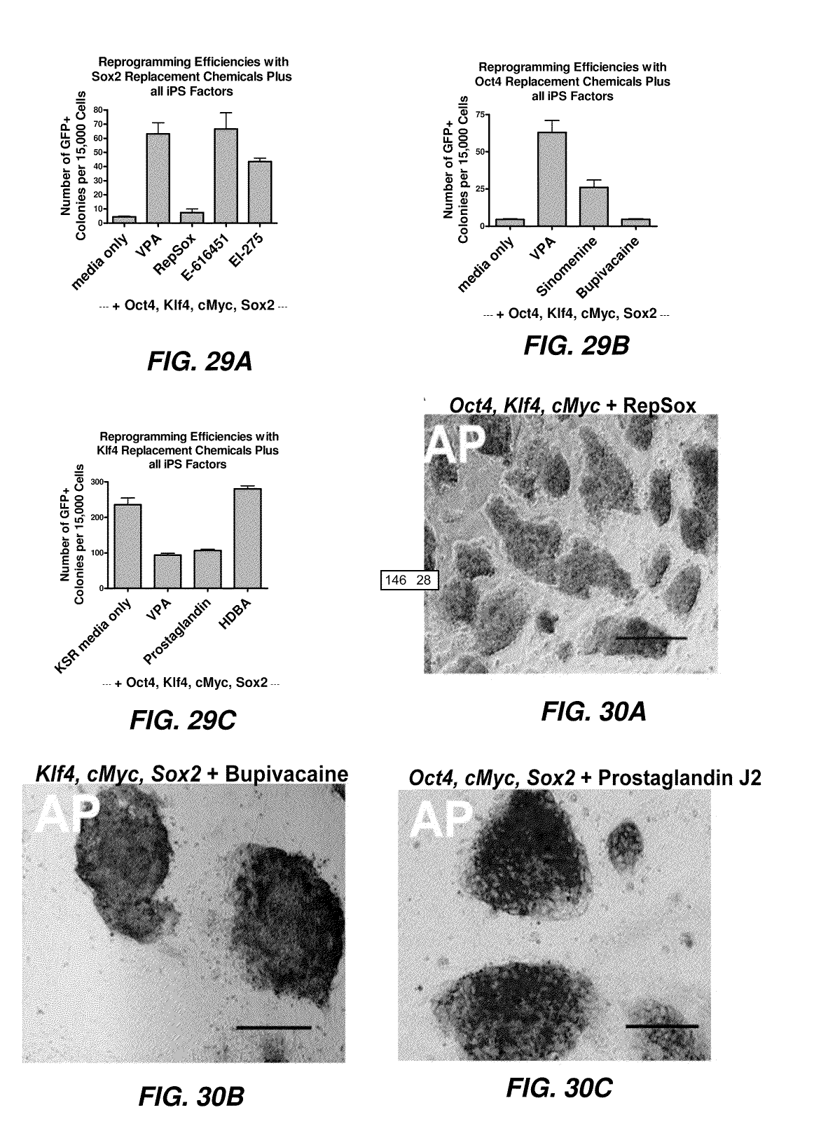

The inventors re-iterated their screen to find small molecules that could replace Oct4 (FIG. 27A). The inventors modified their approach slightly and screened in normal mES culture media containing a higher concentration of VPA (2 mM). After transducing 7500 MEFs with the four reprogramming transgenes and treating with VPA, the inventors observed only ˜125 GFP+ colonies (FIG. 27C). This reduced number of colonies was still significantly higher then the number of colonies in control transductions lacking Oct4, where the inventors never observed the formation of a GFP+colony (FIG. 27C). Therefore we opted to continue the screen under these conditions.

Following transduction with only Klf4, cMyc, and Sox2, 2000 MEFS were cultured for 25 days in the presence of VPA and each compound from the chemical library (FIG. 27A). When the inventors analyzed this screen, the inventors found that Sinomenine (FIG. 27B), an ATP-dependent potassium channel agonist (Lee et al., Clin. Exp. Pharmacol. Physiol. 34, 979, 2007) that is known to promote self-renewal in human embryonic stem cells (Desbordes et al., Cell Stem Cell 2, 602, 2008) induced GFP+colonies. Additionally, Ropivacaine, a compound that both antagonizes Na⁺-channels and that also activates p38 mitogen-activated protein kinase (MAPK)(25)(FIG. 27B), induced more than 10 colonies that had mES-like morphologies, but were not GFP+(FIG. 27C). The inventors hypothesized that these colonies arising from Ropivicaine treatment were only partially reprogrammed and that they might be fully reprogrammed by treatment with 5-aza-cytidine (5-aza C) as previously reported (Mikkelsen et al., Nature 454, 49, 2008) Indeed, after treatment with 5-aza-cytidine for 48 hours, the majority of these colonies became Oct4::GFP+ (FIG. 27C). These colonies could be picked and expanded into mESC-like cell lines that were uniformly GFP+ (FIG. 27D), demonstrating the multi-chemical treatment had fully reprogrammed the cells in the absence of transgenic Oct4.

In order to validate and better characterize the putative Oct4 replacement compounds, the inventors optimized their concentrations for reprogramming and then again tested their ability to replace Oct4 at a larger scale. Because these Oct4 replacement molecules were both identified in the presence of VPA and because VPA itself can help reduce the number of transgenic factors required to generate iPS cells (Huangfu et al., Nat. Biotechnol. 26, 795, 2008), it was possible that the mechanism by which these compounds replaced Oct4 was dependent on the inhibitory activity of VPA on histone deacetylases. The inventors therefore tested the ability of these newly identified compounds to replace Oct4 in the absence of VPA. Additionally, the inventors tested the ability of Bupivacaine, a structural and functional analog of Ropivacaine, to replace Oct4 (FIG. 27B). Incubation with Sinomenine again induced Oct4::GFP+colonies in the presence of VPA, but it failed to induce Oct4::GFP+colonies in the absence of VPA (FIG. 27C). Ropivacaine also induced the formation of many GFP+colonies, but only when used in conjunction with VPA and 5-aza-cytidine (FIG. 27C). In contrast, Bupivacaine independently induced GFP+colonies in the absence of both VPA and 5-aza-cytidine (FIG. 27C, 27D), demonstrating that even alone Bupivacaine is capable of replacing exogenous Oct4 expression in reprogramming. Together, the inventors have demonstrated that at least two distinct classes of small molecule compounds, Sinimenine or Ropivacaine/Bupicacaine can allow reprogramming to go forward in the absence of Oct4.

Investigation of self-renewal capacity, gene expression program, and pluripotency demonstrated that GFP+ cells induced by the Bupivacaine replacement of Oct4 were bona fide iPS cells. The inventors found that the reprogrammed cell lines self-renewed for more than 10 passages with a growth rate similar to mES cells, while maintaining a mES cell-like morphology and expression of the Oct4::GFP transgene (FIG. 27D). Antibody staining indicated that the Oct4::GFP positive cells co-expressed the endogenous alleles of the Nanog and Sox2 or Oct4 genes (FIG. 31A), demonstrating a pluripotent transcriptional program had been established. In addition, all cell lines expressed the embryonic enzymatic activity alkaline phosphatase, as do mouse ES cells and iPS cells (FIG. 30B).

The global transcriptional profiles of cell lines reprogrammed with Bupivacaine were similar to a mouse ES cell line and to iPS cell lines produced with all four transgenes, while they differed significantly from that of the somatic MEFs (FIG. 31A). Pearson correlation coefficient analysis confirmed that iPS cells produced with Bupivacaine were as similar to mouse ES cells (Pearson correlation coefficient=0.95-0.97) as two mouse ES cell lines were to each other (Pearson correlation coefficient=0.96) (table 3).

In order to demonstrate that the chemically reprogrammed cell lines share the pluripotent characteristics of mES and iPS cells, the inventors demonstrated their ability to spontaneously differentiate in vitro into cells types derived from the three embryonic germ layers (data not shown). The inventors found that cells reprogrammed with Bupivacaine could respond to directed differentiation signals in vitro (data not shown). Bupivacaine is pluripotent and equivalent to other iPS cells with respect to their differentiation in vitro.

The inventors have definitively demonstrated that small molecules can replace a critical reprogramming factor through a mechanism that is distinct from genome-wide chromatin remodeling. As disclosed herein, there need not always be a discrete, one to one, mapping between the functions of the reprogramming factors and their chemical replacements.

The practice of the present invention will employ, unless otherwise indicated, conventional techniques of mouse genetics, developmental biology, cell biology, cell culture, molecular biology, transgenic biology, microbiology, recombinant DNA, and immunology, which are within the skill of the art. Such techniques are described in the literature. See, for example, Current Protocols in Cell Biology, ed. by Bonifacino, Dasso, Lippincott-Schwartz, Harford, and Yamada, John Wiley and Sons, Inc., New York, 1999; Manipulating the Mouse Embryos, A Laboratory Manual, 3^{rd} Ed., by Hogan et al., Cold Spring Contain Laboratory Press, Cold Spring Contain, New York, 2003; Gene Targeting: A Practical Approach, IRL Press at Oxford University Press, Oxford, 1993; and Gene Targeting Protocols, Human Press, Totowa, N.J., 2000. All patents, patent applications and references cited herein are incorporated in their entirety by reference.

The present invention is well adapted to carry out the objects and obtain the ends and advantages mentioned, as well as those inherent therein. The methods, systems and kits are representative of certain embodiments, are exemplary, and are not intended as limitations on the scope of the invention. Modifications therein and other uses are also contemplated herein. These modifications are encompassed within the spirit of the invention and are defined by the scope of the claims. Varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention can be explicitly excluded from the claims, regardless of whether the specific exclusion is recited in the specification. For example, any differentiated cell, any agent, any reprogrammed cell, any reprogramming agent, etc., may be excluded.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” is intended to encompass numbers that fall within a range of ±10% of a number, in some embodiments within ±5% of a number, in some embodiments within ±1%, in some embodiments within ±0.5% of a number, in some embodiments within ±0.1% of a number unless otherwise stated or otherwise evident from the context (except where such number would impermissibly exceed 100% of a possible value).

Certain claims are presented in dependent form for the sake of convenience, but Applicant reserves the right to rewrite any dependent claim in independent form to include the limitations of the independent claim and any other claim(s) on which such claim depends, and such rewritten claim is to be considered equivalent in all respects to the dependent claim in whatever form it is in (either amended or unamended) prior to being rewritten in independent format. It should also be understood that, unless clearly indicated to the contrary, in any methods claimed herein that include more than one act, the order of the acts of the method is not necessarily limited to the order in which the acts of the method are recited, but the invention includes embodiments in which the order is so limited.

### REFERENCES

All references cited herein are incorporated herein by reference in their entirety as if each individual publication or patent or patent application was specifically and individually indicated to be incorporated by reference in its entirety.

Many modifications and variations of this invention can be made without departing from its spirit and scope, as will be apparent to those skilled in the art. The specific embodiments described herein are offered by way of example only, and the invention is to be limited only in terms of the appended claims, along with the full scope of equivalents to which such claims are entitled.
1. K. Okita, T. Ichisaka, S. Yamanaka, Nature 448, 313 (Jul. 19, 2007).
2. N. Maherali et al., Cell Stem Cell 1, 55 (Jun. 7, 2007).
3. M. Wernig et al., Nature 448, 318 (Jul. 19, 2007).
4. K. Takahashi et al., Cell 131, 861 (Nov. 30, 2007).
5.1. H. Park et al., Nature 451, 141 (Jan. 10, 2008).
6. M. Nakagawa et al., Nat Biotechnol 26, 101 (January, 2008).
7. S. Hacein-Bey-Abina et al., Science 302, 415 (Oct. 17, 2003).
8. A. J. a. G. Thrasher, H. B., http://www.asgtorg/UserFiles/XSCIDstatement.pdf, (2007).
9. M. Stadtfeld, M. Nagaya, J. Utikal, G. Weir, K. Hochedlinger, Science, (Sep. 25, 2008).
10. F. Soldner et al., Cell 136, 964 (Mar. 6, 2009).
11. K. Kaji et al., Nature, (Mar. 1, 2009).
12. K. Woltjen et al., Nature, (Mar. 1, 2009).
13. K. Okita, M. Nakagawa, H. Hyenjong, T. Ichisaka, S. Yamanaka, Science, (Oct. 9, 2008).
14. A. Harui, S. Suzuki, S. Kochanek, K. Mitani, J Virol 73, 6141 (July, 1999).
15. H. Zhou et al., Cell Stem Cell 4, 381 (May 8, 2009).
16. S. Baqir, L. C. Smith, Cloning Stem Cells 8, 200 (Fall, 2006).
17. G. Heller et al., Cancer Res 68, 44 (Jan. 1, 2008).
18. D. Huangfu et al., Nat Biotechnol, (Jun. 22, 2008).
19. Y. Shi et al., Cell Stem Cell 2, 525 (Jun. 5, 2008).
20. D. Huangfu et al., Nat Biotechnol, (Oct. 12, 2008).
21. T. S. Mikkelsen et al., Nature 454, 49 (Jul. 3, 2008).
22. L. L. Rubin, Cell 132, 549 (Feb. 22, 2008).
23. A. Meissner, M. Wernig, R. Jaenisch, Nat Biotechnol 25, 1177 (Oct, 2007).
24. D. Huangfu et al., Nat Biotechnol 26, 795 (July, 2008).
25. M. Boiani, J. Kehler, H. R. Scholer, Methods Mol Biol 254, 1 (2004).
26. F. Gellibert et al., J Med Chem 47, 4494 (Aug. 26, 2004).
27. J. H. Hanke et al., J Biol Chem 271, 695 (Jan. 12, 1996).
28. Y. Shi et al., Cell Stem Cell 3, 568 (Nov. 6, 2008).
29. J. B. Kim et al., Cell 136, 411 (Feb. 6, 2009).
30. H. Wichterle, I. Lieberam, J. A. Porter, T. M. Jessell, Cell 110, 385 (Aug. 9, 2002).
31. F. P. Di Giorgio, M. A. Carrasco, M. C. Siao, T. Maniatis, K. Eggan, Nat Neurosci 10, 608 (May, 2007).
32. G. J. Inman et al., Mol Pharmacol 62, 65 (July, 2002).
33. R. Sridharan et al., Cell 136, 364 (Jan. 23, 2009).
34. H. Hong et al., Nature 460, 1132 (Aug. 27, 2009).
35. J. Utikal et al., Nature 460, 1145 (Aug. 27, 2009).
36. R. M. Marion et al., Nature 460, 1149 (Aug. 27, 2009).
37. H. Li et al., Nature 460, 1136 (Aug. 27, 2009).
38. T. Kawamura et al., Nature 460, 1140 (Aug. 27, 2009).
39. J. Silva et al., PLoS Biol 6, e253 (Oct. 21, 2008).
40. G. Amabile, A. Meissner, Trends Mol Med 15, 59 (February, 2009).
41. Q. L. Ying, J. Nichols, I. Chambers, A. Smith, Cell 115, 281 (Oct. 31, 2003).
42. R. H. Xu et al., Cell Stem Cell 3, 196 (Aug. 7, 2008).
43. A. Suzuki et al., Proc Natl Acad Sci USA 103, 10294 (Jul. 5, 2006).
44. L. Attisano, J. L. Wrana, Science 296, 1646 (May 31, 2002).
45.1. Chambers et al., Cell 113, 643 (May 30, 2003).
46.1. Chambers et al., Nature 450, 1230 (Dec. 20, 2007).
47. L. Vanier et al., Development 136, 1339 (April, 2009).
48. J. Silva, I. Chambers, S. Pollard, A. Smith, Nature 441, 997 (Jun. 22, 2006).
49. J. Yu et al., Science 318, 1917 (Dec. 21, 2007).
50. J. Silva et al., Cell 138, 722 (Aug. 21, 2009).
51. M. Stadtfeld, N. Maherali, D. T. Breault, K. Hochedlinger, Cell Stem Cell 2, 230 (Mar. 6, 2008).
52. K. Takahashi, S. Yamanaka, Cell 126, 663 (Aug. 25, 2006).
53. L. A. Boyer et al., Cell 122, 947 (Sep. 23, 2005).
54. S. Masui et al., Nature Cell Biology 9, 11 (June 2007, 2007).
55. A. Marson et al., Cell Stem Cell 3, 132 (Aug. 7, 2008).

1-135. (canceled)

136. A method of reprogramming a differentiated cell in the absence of exogenous Sox2 transcription factor, comprising contacting an isolated differentiated cell with the compound of any of Formula I, II, or III to thereby produce a reprogrammed cell, wherein the compound of Formula I is: 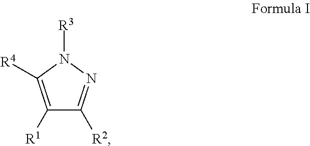
wherein:
R¹ cyclyl, heterocyclcyl, aryl or heteroaryl, each of which can be optionally substituted;
R² cyclyl, heterocyclcyl, aryl or heteroaryl, each of which can be optionally substituted;
R³ is H, C₁-C₆ alkyl, arylC₁-C₆, or a nitrogen protecting group, each of which can be optionally substituted; and
R⁴ is H, optionally substituted C₁-C₆ alkyl, optionally substituted C₂-C₆ alkenyl, optionally substituted C₂-C₆ alkynyl, or R³ and R⁴ together with the atoms they are attached to form a cyclyl, heterocyclyl, aryl or heteroaryl, each of which can be optionally substituted;
wherein the compound of Formula II is: 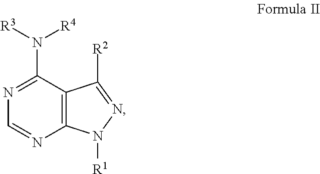
wherein:
R¹ is H, C₁-C₆ alkyl, or C₁-C₆ haloalky;
R² is optionally substituted aryl or optionally substituted heteroaryl;
each R³ and R⁴ is independently H, C₁-C₆ alkyl, arylC₁-C₆alklyl, or a nitrogen protecting group; and
wherein the compound of Formula III is: 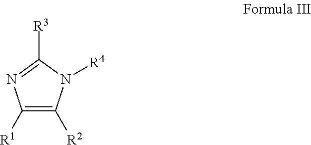
R¹ is cyclyl, heterocyclcyl, aryl or heteroaryl, each of which can be optionally substituted;
R² is cyclyl, heterocyclcyl, aryl or heteroaryl, each of which can be optionally substituted;
R³ is cyclyl, heterocyclcyl, aryl, heteroaryl or —S(O)alkyl, each of which can be optionally substituted; and
R⁴ is H, optionally substituted C₁-C₆ alkyl, optionally substituted C₂-C₆ alkenyl, optionally substituted C₂-C₆ alkynyl, or R³ and R⁴ together with the atoms they are attached to form a cyclyl, heterocyclyl, aryl or heteroaryl, each of which can be optionally substituted.

137. The method of claim 136, wherein the compound of formula (I) comprises the structure:

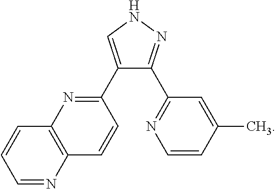

138. The method of claim 136, wherein the compound of formula (I) comprises the structure:

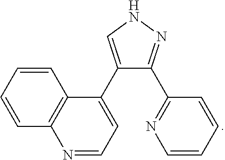

139. The method of claim 136, wherein an isolated differentiated cell is contacted with the compound of Formula I(a) or Formula I(b) to thereby produce a reprogrammed cell, wherein the compound of Formula I(a) is: 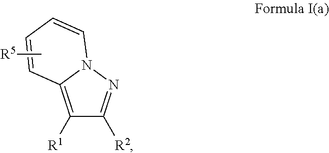
wherein:
R¹ cyclyl, heterocyclcyl, aryl or heteroaryl, each of which can be optionally substituted;
R² cyclyl, heterocyclcyl, aryl or heteroaryl, each of which can be optionally substituted; and
R⁵ is R⁵ is H, benzyl, aryl, heteroaryl, C₁-C₆alkyl, alkenyl, alkynyl, halogen, amino, —C(O)-amino, —SO₂-alkyl, —O-alkyl or acyl, each of which can be optionally substituted, and
the compound of Formula I(b) is: 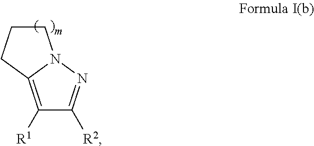
wherein:
R¹ cyclyl, heterocyclcyl, aryl or heteroaryl, each of which can be optionally substituted;
R² cyclyl, heterocyclcyl, aryl or heteroaryl, each of which can be optionally substituted; and
m is 1, 2 or 3.

140. The method of claim 136, wherein the compound of formula (II) comprises the structure:

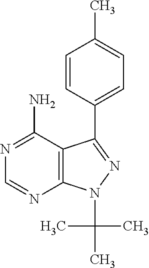

141. The method of claim 136, wherein the compound of formula (III) comprises the structure

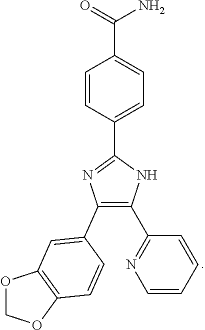

142. The method of claim 136, wherein an isolated differentiated cell is contacted with the compound of Formula III(a) or Formula III(b) to thereby produce a reprogrammed cell, wherein the compound of Formula III(a) is: 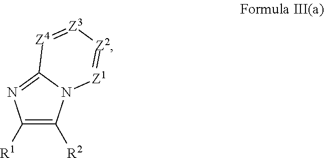
wherein:
R¹ is cyclyl, heterocyclcyl, aryl or heteroaryl, each of which can be optionally substituted;
R² is cyclyl, heterocyclcyl, aryl or heteroaryl, each of which can be optionally substituted;
z¹-z⁴ are independently CR⁵ or N, provided that no two N are next to each other; and
R⁵ is H, benzyl, aryl, heteroaryl, C₁-C₆alkyl, alkenyl, alkynyl, halogen, amino, —C(O)-amino, —SO₂-alkyl, —O-alkyl or acyl, each of which can be optionally substituted, and the compound of Formula III(b) is: 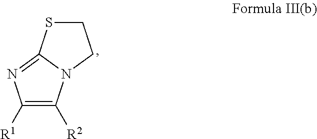
wherein:
R¹ is cyclyl, heterocyclcyl, aryl or heteroaryl, each of which can be optionally substituted; and R² is cyclyl, heterocyclcyl, aryl or heteroaryl, each of which can be optionally substituted.

143. A method of reprogramming a differentiated cell in the absence of exogenous Sox2 transcription factor comprising contacting an isolated differentiated cell with a compound of any of Formula IV-VII to thereby produce a reprogrammed cell, wherein the compound of Formula IV is Formula IV(a) or Formula IV(b), wherein the compound of Formula IV(a) is: 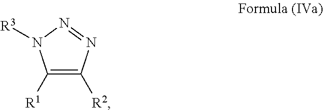
and the compound of Formula IV(b) is: 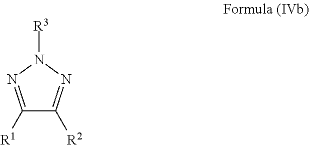
wherein, R¹ is cyclyl, heterocyclcyl, aryl or heteroaryl, each of which can be optionally substituted;
R² is cyclyl, heterocyclcyl, aryl or heteroaryl, each of which can be optionally substituted; and
R³ is R³ is H, C₁-C₆ alkyl, C₂-C₆ alkenyl, C₂-C₆ alkynyl, aryl, heteroaryl, cyclyl, heterocyclyl, acyl or a nitrogen protecting group, each of which can be optionally substituted, and
wherein the compound of Formula V is: 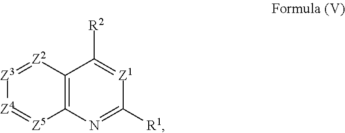
wherein:
R¹ is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;
R² is H, C₁-C₆ alkyl, C₁-C₆ alkenyl, C₁-C₆ alkynyl, aryl, heteroaryl, cyclyl, optionally substituted heterocyclyl, acyl or amino (e.g., N(R³)₂), each of which can be optionally substituted;
R³ is independently for each occurrence H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;
Z¹, Z², Z³, Z⁴ and Z⁵ are each independently N or CR³, provided that at least two of
Z², Z³, Z⁴ and Z⁵ are CR³, and further provided that two adjacent Z positions are not N; and
wherein the compound of Formula VI is: 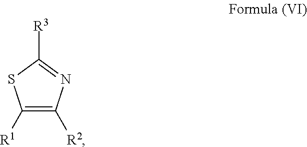
wherein:
R¹ is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;
R² is H, C₁-C₆ alkyl, C₁-C₆ alkenyl, C₁-C₆ alkynyl, aryl, heteroaryl, cyclyl, optionally substituted heterocyclyl or acyl, each of which can be optionally substituted;
R³ is H, C₁-C₆ alkyl, C₁-C₆ alkenyl, C₁-C₆ alkynyl, aryl, heteroaryl, cyclyl, optionally substituted heterocyclyl, acyl or amino, each of which can be optionally substituted, and
wherein the compound of Formula VII is: 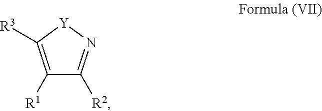
wherein:
X is O, S or CH₂;
R¹ is H, C₁-C₆ alkyl, C₁-C₆ alkenyl, C₁-C₆ alkynyl, aryl, heteroaryl, cyclyl, heterocyclyl, acyl, amino, or amide (e.g., —CO₂NH₂), each of which can be optionally substituted;
R² is C₁-C₆ alkyl, —O-alkyl, amino, acyl, aryl, heteroaryl, cyclyl or heterocyclyl, each of which can be optionally substituted;
R³ is H, C₁-C₆ alkyl, —O-alkyl, amino, amide, —NHC(O)NH-alkyl, acyl, aryl, heteroaryl, cyclyl, heterocyclyl, each of which can be optionally substituted.

144. The method of claim 136 wherein the isolated differentiated cell is contacted with one or more exogenous transcription factors selected from the group consisting of a nucleic acid encoding at least one transcription factor selected from the group consisting of Oct-4, Klf-4, c-Myc, lin-28 and Nanog or a biologically active polypeptide of at least one transcription factor selected from the group consisting of Oct-4, Klf-4, c-Myc, lin-28 and Nanog.

145. A method of reprogramming a differentiated cell in the absence of exogenous Klf4, comprising contacting an isolated differentiated cell with any compound of Formula VIII or IX to produce a reprogrammed cell, wherein a compound of Formula VIII is: 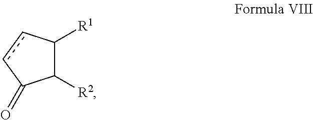
wherein:
R¹ is optionally substituted C₄-C₁₀ alkyl, C₄-C₁₀ alkenyl or C₄-C₁₀ alkynyl;
R² is optionally substituted C₄-C₁₀ alkyl, C₄-C₁₀ alkenyl or C₄-C₁₀ alkynyl; and
The dashed line ( - - - ) indicates the presence or absence of a bond;
wherein a compound of Formula IX is: 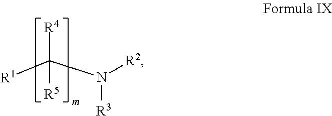
wherein:
R¹ cyclyl, heterocyclcyl, aryl or heteroaryl, each of which can be optionally substituted;
R² cyclyl, heterocyclcyl, aryl or heteroaryl, each of which can be optionally substituted;
R³ is H, C₁-C₆ alkyl, aryl, heteroaryl, cyclyl, heterocyclyl, arylalkyl, heteroarylalkyl, or a nitrogen protecting group, each of which can be optionally substituted;
each R⁴ and R⁵ is independently H, halo, —CN, —NO₂, C₁-C₆ alkyl, haloC₁-C₆alkyl, —CO₂R⁶, —OR⁶ or —N(R⁶)₂, each of which can be optionally substituted;
R⁶ is independently H, C₁-C₆alkyl, aryl, heteroaryl, cyclyl, heterocyclyl or acyl, each of which can be optionally substituted; and
m is 0, 1 or 2.

146. The method of claim 145, wherein an isolated differentiated cell is contacted with a compound of Formula VIII comprising the structure:

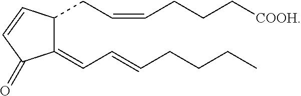

147. The method of claim 145, wherein an isolated differentiated cell is contacted with a compound of Formula IX(a) to produce a reprogrammed cell, wherein the Formula IX(a) is: 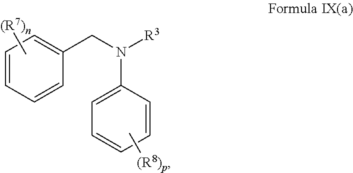
wherein:
R³ is H, C₁-C₆ alkyl, aryl, heteroaryl, cyclyl, heterocyclyl, arylalkyl, heteroarylalkyl, or a nitrogen protecting group, each of which can be optionally substituted;
each R⁷ and R⁸ is independently halo, —CN, —NO₂, C₁-C₆ alkyl, C₂-C₆ alkenyl, C₁-C₆ alkynyl, haloC₁-C₆alkyl, —CO₂R⁶, —OR⁶, —N(R⁶)₂, each of which can be optionally substituted;
n is 0, 1, 2, 3,4 or 5; and
p is 0, 1, 2, 3,4 or 5.

148. The method of claim 146, wherein an isolated differentiated cell is contacted with a compound comprising the structure: [chemical structure]
